SEQUENCE LISTING (640 sequences)

SEQ ID NO: 1 - protein, 127 aa, synthetic construct
  REGION 1..127: note = VH of FM 7B
EVQLVQSGAEVKKPGASVKVSCKASGYTFTSYAMHWVRQAPGQRLEWMGWINTGNGNTKYSQKFQGRVTITRDTSASTAYMELSSLRSEDTAVYYCARDPTYCSSTSCYPFSWFDPWGQGTLVTVSS

SEQ ID NO: 2 - protein, 109 aa, synthetic construct
  REGION 1..109: note = VL of FM 7B
NFMLTQPPSVSVAPGQTARITCGGNNIGSKSVHWYQQKPGQAPVLVVYDDSDRPSGIPERFSGSNSGNTATLTISRVEAGDEADYYCQVWDSTGDHSWVFGGGTKLTVL

SEQ ID NO: 3 - protein, 131 aa, synthetic construct
  REGION 1..131: note = VH of FD 8B
EVQLLESGAEVKKPGASVKVSCKVSGYTLTELSMHWVRQAPGKGLEWMGGFDPEDGETIYAQKFQGRVTMTEDTSTDTAYMELSSLRSEDTAVYYCATAAAINCSSTSCYYYYYYYGMDVWGQGTTVTVSS

SEQ ID NO: 4 - protein, 112 aa, synthetic construct
  REGION 1..112: note = VL of FD 8B
DIVMTQTPLSSPVTLGQPASISCRSSQSLVHSDGNTYLSWLQQRPGQPPRLLIYKISSRFSGVPDRFSGSGAGTDFTLKISRVEAEDVGVYYCTQATQFPYTFGQGTKVDIK

SEQ ID NO: 5 - protein, 133 aa, synthetic construct
  REGION 1..133: note = VH of EW 9B
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREDGVVPAANLMISLEDYYYYGMDVWGQGTTVTVSS

SEQ ID NO: 6 - protein, 107 aa, synthetic construct
  REGION 1..107: note = VL of EW 9B
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVDIK

SEQ ID NO: 7 - protein, 130 aa, synthetic construct
  REGION 1..130: note = VH of FN12A
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARSGCSSTSCPSNLYYYYYGMDVWGQGTTVTVSS

SEQ ID NO: 8 - protein, 110 aa, synthetic construct
  REGION 1..110: note = VL of FN 12A
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSALVFGGGTKLTVL

SEQ ID NO: 9 - protein, 123 aa, synthetic construct
  REGION 1..123: note = VH of FG 12C
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDMRVHDYGDYYFDYWGQGTLVTVSS

SEQ ID NO: 10 - protein, 108 aa, synthetic construct
  REGION 1..108: note = VL of FG 12C
SYELTQPPSVSVAPGKTARITCGGNNIGSKSVHWYQQKPGQAPVLVIYYDSDRPSGIPERFSGSNSGNTATLTISRVEAGDEADYYCQVWDSSSDHPVFGGGTKLTVL

SEQ ID NO: 11 - protein, 120 aa, synthetic construct
  REGION 1..120: note = VH of FI 1C
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARRSNRFLIAFDIWGQGTMVTVSS

SEQ ID NO: 12 - protein, 111 aa, synthetic construct
  REGION 1..111: note = VL of FI 1C
QSALTQPASVSGSPGQTITISCTGTSSDVGGYNYVSWYQQHPGKAPKLMIYEVSNRPSGVSNRFSGSKSGNTASLTISGLQAEDEADYYCSSYTSSSTLVVFGGGTKLTVP

SEQ ID NO: 13 - protein, 118 aa, synthetic construct
  REGION 1..118: note = VH of FI 4A
EVQLVESGGGLVKPGGSLRLSCAASGFTFSRFSMNWVRQAPGKGLEWVSSISSSGSYIYFADSVKGRFTISRDNAKNSLYLQMNSLRAEDTAIYYCATYLFGDSHTYWGQGTLVTVSS

SEQ ID NO: 14 - protein, 112 aa, synthetic construct
  REGION 1..112: note = VL of FI 4A
QSVLTQPHSVSESPGKTVTISCTGSSGSIASNYVQWYQQRPGSAPTTVIYEDNQRPSGVPDRFSGSIDSSSNSASLTISGLKTEDEADYYCQSYDSSNLHWVFGGGTKLTVL

SEQ ID NO: 15 - protein, 125 aa, synthetic construct
  REGION 1..125: note = VH of FD 1E
EVQLLESGGGLVKPGGSLRLSCAASGFTFSSYSMNWVRQAPGKGLEWVSSISSSSSYIYYADSVKGRFTISRDNAKNSLYLQMNSLRAEDTAVYYCASLAAAGPETYYYYGMDVWGQGTMVTVSS

SEQ ID NO: 16 - protein, 105 aa, synthetic construct
  REGION 1..105: note = VL of FD 1E
SYELTQPPSVSVSPGQTASITCSGDKLGDKYACWYQQKPGQSPVLVIYQDSKRPSGIPERFSGSNSGNTATLTISGTQAMDEADYYCQAWDSSVVFGGGTKLTVL

SEQ ID NO: 17 - protein, 125 aa, synthetic construct
  REGION 1..125: note = VH of FD 11E
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCASLAAAGPETYYYYGMDVWGQGTMVTVSS

SEQ ID NO: 18 - protein, 105 aa, synthetic construct; identical to SEQ ID NO: 16
  REGION 1..105: note = VL of FD 11E

SEQ ID NO: 19 - protein, 127 aa, synthetic construct
  REGION 1..127: note = VH of FN 2C
EVQLLESGGGVVQPGRSLRLSCAASGFNFNNYGMHWVRQAPGKGLEWVATISYEGSKKFYVDSVKGRFTISKDNSKNTLYLQMNSLRVDDTAFYYCAKRREIFWLGEPPLSDAFDFWGQGTMVTVSS

SEQ ID NO: 20 - protein, 111 aa, synthetic construct
  REGION 1..111: note = VL of FN 2C
QSVLTQPPSVSGAPGQRVTISCTGSGSNIGAGYDVHWYQFLPGTAPQLLIYGNNNRPSGVPDRFSGSKSGTSASLAITGLQAEDEADYYCQSYDSSLSGSVFGGGTKLTVL

SEQ ID NO: 21 - protein, 121 aa, synthetic construct
  REGION 1..121: note = VH of EY 6A
EVQLVESGGGVVQPGRSLRLSCAASAFTFSSYDMHWVRQAPGKGLEWVAVISYDGSNKYYADSVKGRFTISRDNSKNTLYLQMNSLRAEDTAVYYCAKDGGKLWVYYFDYWGQGTTVTVSS

SEQ ID NO: 22 - protein, 108 aa, synthetic construct
  REGION 1..108: note = VL of EY 6A
DIQMTQSPSSLSASVGDRVTITCRASQSISSYLNWYQQKPGKAPKLLIYAASSLQSGVPSRFSGSGSGTDFTLTISSLQPEDFATYYCQQSYSTLALTFGGGTKVDIK

SEQ ID NO: 23 - protein, 121 aa, synthetic construct
  REGION 1..121: note = VH of EY 6A-1
EVQLVESGGGVVQPGRSLRLSCAASAFTFSSYDMHWVRQAPGKGLEWVAVISYDGSNKYYADSVKGRFTISRDNSKNTLYLQMNSLRAEDTAVYYCAKDGGKLWVYYFDYWGQGTLVTVSS

SEQ ID NO: 24 - protein, 108 aa, synthetic construct
  REGION 1..108: note = VL of EY 6A-1
DIQMTQSPSSLSASVGDRVTITCRASQSISSYLNWYQQKPGKAPKLLIYAASSLQSGVPSRFSGSGSGTDFTLTISSLQPEDFATYYCQQSYSTLALTFGGGTKVEIK

SEQ ID NO: 25 - protein, 128 aa, synthetic construct
  REGION 1..128: note = VH of FD 11D
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKEGAGSGWYRHHKPGYYFDYWGQGTLVTVSS

SEQ ID NO: 26 - protein, 108 aa, synthetic construct
  REGION 1..108: note = VL of FD 11D
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 27 - protein, 125 aa, synthetic construct
  REGION 1..125: note = VH of EW 9C
EVQLVESGGGVVQPGASLRLSCVASGFTFNNFGFHWVRQAPGKGLEWLAVISYEGSKTYYAESLKGRFTISRDTSKNTVYLQMNSLRAEDTAVYYCARATSIFWFGEGRNWFDPWGQGTMVTVSS

SEQ ID NO: 28 - protein, 107 aa, synthetic construct
  REGION 1..107: note = VL of EW 9C
EIVLTQSPGTVSLSAGERATLSCRASQNVGTDLAWYVQRPGQAPRLLIYDTSNKATGIPARFSGRGSGTDFTLTIDSLEPEDFAVYYCQQRSNWPLTFGGGTKVEIR

SEQ ID NO: 29 - protein, 125 aa, synthetic construct
  REGION 1..125: note = VH of EW 9C-1
EVQLVESGGGVVQPGASLRLSCVASGFTFNNFGFHWVRQAPGKGLEWLAVISYEGSKTYYAESLKGRFTISRDTSKNTVYLQMNSLRAEDTAVYYCARATSIFWFGEGRNWFDPWGQGALVTVSS

SEQ ID NO: 30 - protein, 107 aa, synthetic construct
  REGION 1..107: note = VL of EW 9C-1
EIVMTQSPGTVSLSAGERATLSCRASQNVGTDLAWYVQRPGQAPRLLIYDTSNRATGIPARFSGRGSGTDFTLTIDSLEPEDFAVYYCQQRSNWPLTFGGGTKVEIK

SEQ ID NO: 31 - protein, 121 aa, synthetic construct
  REGION 1..121: note = VH of FD 1D
EVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARAGSGSYLNWFDPWGQGTLVTVSS

SEQ ID NO: 32 - protein, 107 aa, synthetic construct
  REGION 1..107: note = VL of FD 1D
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 33 - protein, 121 aa, synthetic construct
  REGION 1..121: note = VH of FG 7A
EVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARSHSGSYRASLDYWGQGTTVTVSS

SEQ ID NO: 34 - protein, 109 aa, synthetic construct
  REGION 1..109: note = VL of FG 7A
DIQLTQSPGTLSLSPGERATLSCRASQSVSSSYLAWYQQKPGQAPRLLIYGASSRATGIPDRFSGSGSGTDFTLTISRLEPEDFAVYYCQQYGSSPLYTFGQGTKVEIK

SEQ ID NO: 35 - protein, 121 aa, synthetic construct
  REGION 1..121: note = VH of FM 1A
EVQLLESGGGVVQPGRSLRLSCAASGFTFSSYGMHWVRQAPGKGLEWVAVIWYDGSNKYYADSVKGRFTISRDNSKNTLYLQMNSLRAEDTAVYYCAREGAVGATRGFDYWGQGTLVTVSS

SEQ ID NO: 36 - protein, 108 aa, synthetic construct
  REGION 1..108: note = VL of FM 1A
SYELTQPPSVSVAPGQTARITCGGNNIGSKSVHWYQQKPGQAPVLVVYDDSDRPSGIPERFSGSNSGNTATLTISRVEAGDEADYYCQVWDSSSDQGVFGGGTKLTVL

SEQ ID NO: 37 - protein, 127 aa, synthetic construct
  REGION 1..127: note = VH of FD 11A
EVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAKGPDILTGYYNYYYYGMDVWGQGTMVTVSS

SEQ ID NO: 38 - protein, 113 aa, synthetic construct
  REGION 1..113: note = VL of FD 11A
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGFYVVFGGGTKLTVL

SEQ ID NO: 39 - protein, 128 aa, synthetic construct
  REGION 1..128: note = VH of FN 8C
QVQLVESGGGVVQPGRSLRLSCAASGFTFSSYGMHWVRQAPGKGLEWVAVISYDGSNKYYADTVKGRFTISRDNSKNTLYLQMNSLRAEDTAVYYCARERTYYDILTGYRHYYGMDVWGQGTTVTVSS

SEQ ID NO: 40 - protein, 108 aa, synthetic construct
  REGION 1..108: note = VL of FN 8C
SYELTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVL

SEQ ID NO: 41 - protein, 122 aa, synthetic construct
  REGION 1..122: note = VH of FD 5E
EVQLVESGGVVVQPGGSLRLSCAASGFTFDDYAMHWVRQAPGKGLEWVSLISWDGGSTYYADSVKGRFTISRDNSKNSLYLQMNSLRAEDTALYYCAKDSVRFRYYYGMDVWGQGTTVTVSS

SEQ ID NO: 42 - protein, 107 aa, synthetic construct
  REGION 1..107: note = VL of FD 5E
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGPGTKVDIK

SEQ ID NO: 43 - protein, 129 aa, synthetic construct
  REGION 1..129: note = VH of FD 5D
EVQLVESGGGLVQPGGSLRLSCAASGFTFSSYSMNWVRQAPGKGLEWVSYISSSSSTIYYADPVKGRFTISRDNAKNSLYLQMNSLRAEDTAVYYCASPGGITAAGTSVLFGYYGMDVWGQGTMVTVSS

SEQ ID NO: 44 - protein, 114 aa, synthetic construct
  REGION 1..114: note = VL of FD 5D
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPITWTFGQGTKVDIK

SEQ ID NO: 45 - protein, 117 aa, synthetic construct
  REGION 1..117: note = VH of FI 3A
EVQLLESGGGLIQPGGSLRLSCAASGFTVSSNYMSWVRQAPGKGLEWVSVIYSGGSTYYADSVKGRFTISRDNSKNTLYLQMNSLRAEDTAVYYCARDHVRPGMNIWGQGTMVTVSS

SEQ ID NO: 46 - protein, 107 aa, synthetic construct
  REGION 1..107: note = VL of FI 3A
AIRMTQSPSSLSASVGDRVTITCQASQDISNYLNWYQQKPGKAPKLLIYDASNLETGVPSRFSGSGSGTDFTFTISSLQPEDIATYYCQQYDNLPVTFGGGTKVDIK

SEQ ID NO: 47 - protein, 114 aa, synthetic construct
  REGION 1..114: note = VH of FD 10A
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCANMAFDIWGQGTMVTVSS

SEQ ID NO: 48 - protein, 113 aa, synthetic construct
  REGION 1..113: note = VL of FD 10A
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGQGTRLEIK

SEQ ID NO: 49 - protein, 127 aa, synthetic construct
  REGION 1..127: note = VH of FJ 4E
QVQLVESGPGLVKPSETLSLTCSVSGGSISSGTYYWSWIRQQPGKGLEWIGYIYNTGRPYYNPFLKSRITISVDSSKNQFSLKLTSVTAADTAVYYCARDEYDSSDSGIQGHWFDPWGQGTLVTVSS

SEQ ID NO: 50 - protein, 107 aa, synthetic construct
  REGION 1..107: note = VL of FJ 4E
AIRMTQSPSSLSASVGDRVTITCRTSQNINSFLNWYQQKPGKGPNLLIYGAFTLQSGVPSRFSGSGSGTDFTLTISSLQPEDFATYFCQQSYSTPWTFGQGTKVEIK

SEQ ID NO: 51 - protein, 126 aa, synthetic construct
  REGION 1..126: note = VH of FJ 1C
QVQLQESGPGLVKPSETLSLTCTVSGYSISSGYYWGWIRQPPGKGLEWIGSIYHSGSTYYNPSLKSRVTISVDTSKNQFSLKLSSVTAADTAVYYCARDKALLWFGELFTNLFDYWGQGTLVTVSS

SEQ ID NO: 52 - protein, 110 aa, synthetic construct
  REGION 1..110: note = VL of FJ 1C
QSALTQPASVSGSPGQSITISCTGTSSDVGGYNYVSWYQQHPGKAPKLMIYEVSNRPSGVSNRFSGSKSGNTASLTISGLQAEDEANYYCSSYTSSSTLVFGGGTKLTVL

SEQ ID NO: 53 - protein, 119 aa, synthetic construct
  REGION 1..119: note = VH of EW 8B
VQLVQESGPGLVKPSETLSLTCTVSGGSISSSGYYWGWIRQPPGKGLEWIANFYFTGSVYSNPSLKSRVTISVDTSKNQLSLKLSYLTAADTAVYYCARQEVWGGFDIWGQGTMVTVSS

SEQ ID NO: 54 - protein, 107 aa, synthetic construct
  REGION 1..107: note = VL of EW 8B
EIVLTQSPGTLSLSPGERVTLSCRASQSLGSSYLAWYQQKPGQAPRLLIYGASSRATGIPDRFSGSGSGTDFTLTISRLEPEDFAVYFCQQYGSSPTFGQGTKVEIK

SEQ ID NO: 55 - protein, 129 aa, synthetic construct
  REGION 1..129: note = VH of FD 11C
EVQLLESGPGLVKPSETLSLTCTVSGGSISSSSYYWGWIRQPPGKGLEWIGSIYYSGSTYYNPSLKSRVTISVDTSKNQFSLKLSSVTAADTAVYYCAREYYYGSETKKYYYYYGMDVWGQGTTVTVSS

SEQ ID NO: 56 - protein, 105 aa, synthetic construct
  REGION 1..105: note = VL of FD 11C
SYELTQPPSVSVSPGQTASITCSGDKLGDKYACWYQQKPGQSPVLVIYQDSKRPSGIPERFSGSNSGNTATLTISGTQAMDEADYYCQAWDSSTVFGGGTKLTVL

SEQ ID NO: 57 - protein, 124 aa, synthetic construct
  REGION 1..124: note = VH of FD 7C
QLQLQESGPGLVKPSETLSLTCTVSGGSISSYYWSWIRQPPGKGLEWIGYIYYSGSTNYNPSLKSRVTISVDTSKNQFSLKLSSVTAADTAVYYCARDYRFGELFGRFAWFDPWGQGTLVTVSS

SEQ ID NO: 58 - protein, 107 aa, synthetic construct
  REGION 1..107: note = VL of FD 7C
DIQLTQSPATLSVSPGERATLSCRASQSVSSNLAWYQQKPGQAPRLLIYGASTRATGIPARFSGSGSGTEFTLTISSLQSEDFAVYYCQQYNNWPRAFGPGTKVEIR

SEQ ID NO: 59 - protein, 126 aa, synthetic construct
  REGION 1..126: note = VH of FD 7D
EVQLVESGAEVKKPGESLRISCKGSGYSFTSYWISWVRQMPGKGLEWMGRIDPSDSYTNYSPSFQGHVTISADKSISTAYLQWSSLKASDTAMYYCARHSDCSSTSCYFVDAFDIWGQGTMVTVSS

SEQ ID NO: 60 - protein, 108 aa, synthetic construct
  REGION 1..108: note = VL of FD 7D
SYELTQPPSVSVSPGQTARITCSGDALPKQYAYWYQQKPGQAPVLVIYKDSERPSGIPERFSGSSSGTTVTLTISGVQAEDEADYYCQSADSSGTYVVFGGGTKLTVL

SEQ ID NO: 61 - protein, 123 aa, synthetic construct
  REGION 1..123: note = VH of FJ 10B
QVQLVQSGAEVKKPGESLRISCKGSGYSFTSYWISWVRQMPGKGLEWMGRIDPSDSYTNYSPSFQGHVTISADKSISTAYLQWSSLKASDTAMYYCARLDPRYGPDYYGMDVWGQGTTVTVSS

SEQ ID NO: 62 - protein, 107 aa, synthetic construct
  REGION 1..107: note = VL of FJ 10B
AIRMTQSPSSLSASVGDRVTITCRASQSISSYLNWYQQKPGKAPKLLIHAASSLQSGVPSRFSGSGSGTDFTLTMSSLQPEDFATYYCQQSYSTPLTFGGGTKVEIR

SEQ ID NO: 63 - protein, 129 aa, synthetic construct
  REGION 1..129: note = VH of FB 9D
QVQLVESGAEVKKPGESLKISCQGSGYRFNSYWIAWVRQMPGKGLEWMGSIFPTDSDVRYNPSFQGQVTISADKSISFAYLQWSSLKASDTAIYYCARHWASMVRGVIRASHYYGMDVWGQGTTVTVSS

SEQ ID NO: 64 - protein, 112 aa, synthetic construct
  REGION 1..112: note = VL of FB 9D
QPVLTQPPSASGSPGQSVTISCSGASSDIGKYNYVSWYQQLPGKAPKLLIYEVTKRPSGVPDRFSGSKSGNTASLTVSGLQADDEADYYCSSYAFGGSDTRVFGGGTKLTVL

SEQ ID NO: 65 - protein, 129 aa, synthetic construct
  REGION 1..129: note = VH of FB 1E
EVQLVESGAEVKKPGESLKISCQGSGYRFNSYWIAWVRQMPGKGLEWMGSIFPTDSDIRYNPSFQGQVTISADKSISIAYLQWSSLKASDTAIYYCARHWASMVRGVIRASHYYGMDVWGQGTTVTVSS

SEQ ID NO: 66 - protein, 112 aa, synthetic construct
  REGION 1..112: note = VL of FB 1E
QSVLTQPPSASGSPGQSVTISCSGASSDIGDYNYVSWYQQLPGKAPKLLIYEVTKRPSGVPDRFSGSKSGNTASLTVSGLQADDEADYYCSSYAFGGSDIRVFGGGTKLAVL

SEQ ID NO: 67 - protein, 123 aa, synthetic construct
  REGION 1..123: note = VH of EZ 7A
EVQLVESGAEVKKPGESLKISCKGSGYSFTSYWIGWVRQMPGKGLEWMGIIYPGDSDTRYSPSFQGQVTISADKSISTAYLQWSSLKASDTAMYYCARGWVYRGFPYYGMDVWGQGTMVTVSS

SEQ ID NO: 68 - protein, 110 aa, synthetic construct
  REGION 1..110: note = VL of EZ 7A
QSALTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTLVFGGGTKLTVL

SEQ ID NO: 69 - protein, 8 aa, synthetic construct
  REGION 1..8: note = HCDR1 of FM 7B
GYTFTSYA

SEQ ID NO: 70 - protein, 8 aa, synthetic construct
  REGION 1..8: note = HCDR2 of FM 7B
INTGNGNT

SEQ ID NO: 71 - protein, 20 aa, synthetic construct
  REGION 1..20: note = HCDR3 of FM 7B
ARDPTYCSSTSCYPFSWFDP

SEQ ID NO: 72 - protein, 6 aa, synthetic construct
  REGION 1..6: note = LCDR1 of FM 7B
NIGSKS

SEQ ID NO: 73 - skipped

SEQ ID NO: 74 - protein, 12 aa, synthetic construct
  REGION 1..12: note = LCDR3 of FM 7B
QVWDSTGDHSWV

SEQ ID NO: 75 - protein, 8 aa, synthetic construct
  REGION 1..8: note = HCDR1 of FD 8B
GYTLTELS

SEQ ID NO: 76 - protein, 8 aa, synthetic construct
  REGION 1..8: note = HCDR2 of FD 8B
FDPEDGET

SEQ ID NO: 77 - protein, 24 aa, synthetic construct
  REGION 1..24: note = HCDR3 of FD 8B
ATAAAINCSSTSCYYYYYYYGMDV

SEQ ID NO: 78 - protein, 11 aa, synthetic construct
  REGION 1..11: note = LCDR1 of FD 8B
QSLVHSDGNTY

SEQ ID NO: 79 - skipped

SEQ ID NO: 80 - protein, 9 aa, synthetic construct
  REGION 1..9: note = LCDR3 of FD 8B
TQATQFPYT

SEQ ID NO: 81 - protein, 8 aa, synthetic construct
  REGION 1..8: note = HCDR1 of EW 9B
GYTFTSYY

SEQ ID NO: 82 - protein, 8 aa, synthetic construct
  REGION 1..8: note = HCDR2 of EW 9B
INPSGGST

SEQ ID NO: 83 - protein, 14 aa, synthetic construct
  REGION 1..14: note = HCDR3 of EW 9B
MISLEDYYYYGMDV

SEQ ID NO: 84 - protein, 6 aa, synthetic construct
  REGION 1..6: note = LCDR1 of EW 9B
QSVSSY

SEQ ID NO: 85 - skipped

SEQ ID NO: 86 - protein, 9 aa, synthetic construct
  REGION 1..9: note = LCDR3 of EW 9B
QQRSNWPLT

SEQ ID NO: 87 - protein, 8 aa, synthetic construct
  REGION 1..8: note = HCDR1 of FN 12A
GGTFSSYA

SEQ ID NO: 88 - protein, 8 aa, synthetic construct
  REGION 1..8: note = HCDR2 of FN 12A
IIPILGIA

SEQ ID NO: 89 - protein, 23 aa, synthetic construct
  REGION 1..23: note = HCDR3 of FN 12A
ARSGCSSTSCPSNLYYYYYGMDV

SEQ ID NO: 90 - protein, 8 aa, synthetic construct
  REGION 1..8: note = LCDR1 of FN 12A
SSNIGNNY

SEQ ID NO: 91 - skipped

SEQ ID NO: 92 - protein, 11 aa, synthetic construct
  REGION 1..11: note = LCDR3 of FN 12A
GTWDSSLSALV

SEQ ID NO: 93 - protein, 8 aa, synthetic construct
  REGION 1..8: note = HCDR1 of FG 12C
GFTFDDYA

SEQ ID NO: 94 - protein, 8 aa, synthetic construct
  REGION 1..8: note = HCDR2 of FG 12C
ISWNSGSI

SEQ ID NO: 95 - protein, 16 aa, synthetic construct
  REGION 1..16: note = HCDR3 of FG 12C
AKDMRVHDYGDYYFDY

SEQ ID NO: 96 - protein, 6 aa, synthetic construct
  REGION 1..6: note = LCDR1 of FG 12C
NIGSKS

SEQ ID NO: 97 - skipped

SEQ ID NO: 98 - protein, 11 aa, synthetic construct
  REGION 1..11: note = LCDR3 of FG 12C
QVWDSSSDHPV

SEQ ID NO: 99 - protein, 8 aa, synthetic construct
  REGION 1..8: note = HCDR1 of FI 1C
GFTFSDYY

SEQ ID NO: 100 - protein, 8 aa, synthetic construct
  REGION 1..8: note = HCDR2 of FI 1C
ISSSGSTI

SEQ ID NO: 101 - protein, 13 aa, synthetic construct
  REGION 1..13: note = HCDR3 of FI 1C
ARRSNRFLIAFDI

SEQ ID NO: 102 - protein, 9 aa, synthetic construct
  REGION 1..9: note = LCDR1 of FI 1C
SSDVGGYNY

SEQ ID NO: 103 - skipped

SEQ ID NO: 104 - protein, 11 aa, synthetic construct
  REGION 1..11: note = LCDR3 of FI 1C
SSYTSSSTLVV

SEQ ID NO: 105 - protein, 8 aa, synthetic construct
  REGION 1..8: note = HCDR1 of FI 4A
GFTFSRFS

SEQ ID NO: 106 - protein, 8 aa, synthetic construct
  REGION 1..8: note = HCDR2 of FI 4A
ISSSGSYI

SEQ ID NO: 107 - protein, 11 aa, synthetic construct
  REGION 1..11: note = HCDR3 of FI 4A
ATYLFGDSHTY

SEQ ID NO: 108 - protein, 8 aa, synthetic construct
  REGION 1..8: note = LCDR1 of FI 4A
SGSIASNY

SEQ ID NO: 109 - skipped

SEQ ID NO: 110 - protein, 11 aa, synthetic construct
  REGION 1..11: note = LCDR3 of FI 4A
QSYDSSNLHWV

SEQ ID NO: 111 - protein, 8 aa, synthetic construct
  REGION 1..8: note = HCDR1 of FD 1E
GFTFSSYS

SEQ ID NO: 112 - protein, 8 aa, synthetic construct
  REGION 1..8: note = HCDR2 of FD 1E
ISSSSSYI

SEQ ID NO: 113 - protein, 18 aa, synthetic construct
  REGION 1..18: note = HCDR3 of FD 1E
ASLAAAGPETYYYYGMDV

SEQ ID NO: 114 - protein, 6 aa, synthetic construct
  REGION 1..6: note = LCDR1 of FD 1E
KLGDKY

SEQ ID NO: 115 - skipped

SEQ ID NO: 116 - protein, 8 aa, synthetic construct
  REGION 1..8: note = LCDR3 of FD 1E
QAWDSSVV

SEQ ID NO: 117 - protein, 8 aa, synthetic construct
  REGION 1..8: note = HCDR1 of FD 11E
GFTFSSYS

SEQ ID NO: 118 - protein, 8 aa, synthetic construct
  REGION 1..8: note = HCDR2 of FD 11E
ISSSSSYI

SEQ ID NO: 119 - protein, 18 aa, synthetic construct
  REGION 1..18: note = HCDR3 of FD 11E
ASLAAAGPETYYYYGMDV

SEQ ID NO: 120 - protein, 6 aa, synthetic construct
  REGION 1..6: note = LCDR1 of FD 11E
KLGDKY

SEQ ID NO: 121 - skipped

SEQ ID NO: 122 - protein, 8 aa, synthetic construct
  REGION 1..8: note = LCDR3 of FD 11E
QAWDSSVV

SEQ ID NO: 123 - protein, 8 aa, synthetic construct
  REGION 1..8: note = HCDR1 of FN 2C
GFNFNNYG

SEQ ID NO: 124 - protein, 8 aa, synthetic construct
  REGION 1..8: note = HCDR2 of FN 2C
ISYEGSKK

SEQ ID NO: 125 - protein, 20 aa, synthetic construct
  REGION 1..20: note = HCDR3 of FN 2C
AKRREIFWLGEPPLSDAFDF

SEQ ID NO: 126 - protein, 9 aa, synthetic construct
  REGION 1..9: note = LCDR1 of FN 2C
GSNIGAGYD

SEQ ID NO: 127 - skipped

SEQ ID NO: 128 - protein, 11 aa, synthetic construct
  REGION 1..11: note = LCDR3 of FN 2C
QSYDSSLSGSV

SEQ ID NO: 129 - protein, 8 aa, synthetic construct
  REGION 1..8: note = HCDR1 of EY 6A
AFTFSSYD

SEQ ID NO: 130 - protein, 8 aa, synthetic construct
  REGION 1..8: note = HCDR2 of EY 6A
ISYDGSNK

SEQ ID NO: 131 - protein, 14 aa, synthetic construct
  REGION 1..14: note = HCDR3 of EY 6A
AKDGGKLWVYYFDY

SEQ ID NO: 132 - protein, 6 aa, synthetic construct
  REGION 1..6: note = LCDR1 of EY 6A
QSISSY

SEQ ID NO: 133 - skipped

SEQ ID NO: 134 - protein, 10 aa, synthetic construct
  REGION 1..10: note = LCDR3 of EY 6A
QQSYSTLALT

SEQ ID NO: 135 - protein, 8 aa, synthetic construct
  REGION 1..8: note = HCDR1 of EY 6A-1
AFTFSSYD

SEQ ID NO: 136 - protein, 8 aa, synthetic construct
  REGION 1..8: note = HCDR2 of EY 6A-1
ISYDGSNK

SEQ ID NO: 137 - protein, 14 aa, synthetic construct
  REGION 1..14: note = HCDR3 of EY 6A-1
AKDGGKLWVYYFDY

SEQ ID NO: 138 - protein, 6 aa, synthetic construct
  REGION 1..6: note = LCDR1 of EY 6A-1
QSISSY

SEQ ID NO: 139 - skipped

SEQ ID NO: 140 - protein, 10 aa, synthetic construct
  REGION 1..10: note = LCDR3 of EY 6A-1
QQSYSTLALT

SEQ ID NO: 141 - protein, 8 aa, synthetic construct
  REGION 1..8: note = HCDR1 of FD 11D
GFTFSSYG

SEQ ID NO: 142 - protein, 8 aa, synthetic construct
  REGION 1..8: note = HCDR2 of FD 11D
ISYDGSNK

SEQ ID NO: 143 - protein, 21 aa, synthetic construct
  REGION 1..21: note = HCDR3 of FD 11D
AKEGAGSGWYRHHKPGYYFDY

SEQ ID NO: 144 - protein, 7 aa, synthetic construct
  REGION 1..7: note = LCDR1 of FD 11D
QSVSSSY

SEQ ID NO: 145 - skipped

SEQ ID NO: 146 - protein, 9 aa, synthetic construct
  REGION 1..9: note = LCDR3 of FD 11D
QQYGSSPLT

SEQ ID NO: 147 - protein, 8 aa, synthetic construct
  REGION 1..8: note = HCDR1 of EW 9C
GFTFNNFG

SEQ ID NO: 148 - protein, 8 aa, synthetic construct
  REGION 1..8: note = HCDR2 of EW 9C
ISYEGSKT

SEQ ID NO: 149 - protein, 18 aa, synthetic construct
  REGION 1..18: note = HCDR3 of EW 9C
ARATSIFWFGEGRNWFDP

SEQ ID NO: 150 - protein, 6 aa, synthetic construct
  REGION 1..6: note = LCDR1 of EW 9C
QNVGTD

SEQ ID NO: 151 - skipped

SEQ ID NO: 152 - protein, 9 aa, synthetic construct
  REGION 1..9: note = LCDR3 of EW 9C
QQRSNWPLT

SEQ ID NO: 153 - protein, 7 aa, synthetic construct
  REGION 1..7: note = HCDR1 of EW 9C-1
GFTFNNF

SEQ ID NO: 154 - protein, 8 aa, synthetic construct
  REGION 1..8: note = HCDR2 of EW 9C-1
ISYEGSKT

SEQ ID NO: 155 - protein, 18 aa, synthetic construct
  REGION 1..18: note = HCDR3 of EW 9C-1
ARATSIFWFGEGRNWFDP

SEQ ID NO: 156 - protein, 6 aa, synthetic construct
  REGION 1..6: note = LCDR1 of EW 9C-1
QNVGTD

SEQ ID NO: 157 - skipped

SEQ ID NO: 158 - protein, 9 aa, synthetic construct
  REGION 1..9: note = LCDR3 of EW 9C-1
QQRSNWPLT

SEQ ID NO: 159 - protein, 8 aa, synthetic construct
  REGION 1..8: note = HCDR1 of FD 1D
GFTFSSYA

SEQ ID NO: 160 - protein, 8 aa, synthetic construct
  REGION 1..8: note = HCDR2 of FD 1D
ISYDGSNK

SEQ ID NO: 161 - protein, 14 aa, synthetic construct
  REGION 1..14: note = HCDR3 of FD 1D
ARAGSGSYLNWFDP

SEQ ID NO: 162 - protein, 6 aa, synthetic construct
  REGION 1..6: note = LCDR1 of FD 1D
QSVSSY

SEQ ID NO: 163 - skipped

SEQ ID NO: 164 - protein, 9 aa, synthetic construct
  REGION 1..9: note = LCDR3 of FD 1D
QQRSNWPIT

SEQ ID NO: 165 - protein, 8 aa, synthetic construct
  REGION 1..8: note = HCDR1 of FG 7A
GFTFSSYA

SEQ ID NO: 166 - protein, 8 aa, synthetic construct
  REGION 1..8: note = HCDR2 of FG 7A
ISYDGSNK

SEQ ID NO: 167 - protein, 14 aa, synthetic construct
  REGION 1..14: note = HCDR3 of FG 7A
ARSHSGSYRASLDY

SEQ ID NO: 168 - protein, 7 aa, synthetic construct
  REGION 1..7: note = LCDR1 of FG 7A
QSVSSSY

SEQ ID NO: 169 - skipped

SEQ ID NO: 170 - protein, 10 aa, synthetic construct
  REGION 1..10: note = LCDR3 of FG 7A
QQYGSSPLYT

SEQ ID NO: 171 - protein, 8 aa, synthetic construct
  REGION 1..8: note = HCDR1 of FM 1A
GFTFSSYG

SEQ ID NO: 172 - protein, 8 aa, synthetic construct
  REGION 1..8: note = HCDR2 od FM 1A
IWYDGSNK

SEQ ID NO: 173 - protein, 14 aa, synthetic construct
  REGION 1..14: note = HCDR3 of FM 1A
AREGAVGATRGFDY

SEQ ID NO: 174 - protein, 6 aa, synthetic construct
  REGION 1..6: note = LCDR1 of FM 1A
NIGSKS

SEQ ID NO: 175 - skipped

SEQ ID NO: 176 - protein, 11 aa, synthetic construct
  REGION 1..11: note = LCDR3 of FM 1A
QVWDSSSDQGV

SEQ ID NO: 177 - protein, 8 aa, synthetic construct
  REGION 1..8: note = HCDR1 of FD 11A
GFTFSSYG

SEQ ID NO: 178 - protein, 8 aa, synthetic construct
  REGION 1..8: note = HCDR2 of FD 11A
IWYDGSNK

SEQ ID NO: 179 - protein, 20 aa, synthetic construct
  REGION 1..20: note = HCDR3 of FD 11A
AKGPDILTGYYNYYYYGMDV

SEQ ID NO: 180 - protein, 9 aa, synthetic construct
  REGION 1..9: note = LCDR1 of FD 11A
SSNIGAGYD

SEQ ID NO: 181 - skipped

SEQ ID NO: 182 - protein, 13 aa, synthetic construct
  REGION 1..13: note = LCDR3 of FD 11A
QSYDSSLSGFYVV

SEQ ID NO: 183 - protein, 8 aa, synthetic construct
  REGION 1..8: note = HCDR1 of FN 8C
GFTFSSYG

SEQ ID NO: 184 - protein, 8 aa, synthetic construct
  REGION 1..8: note = HCDR2 of FN 8C
ISYDGSNK

SEQ ID NO: 185 - protein, 21 aa, synthetic construct
  REGION 1..21: note = HCDR3 of FN 8C
ARERTYYDILTGYRHYYGMDV

SEQ ID NO: 186 - protein, 6 aa, synthetic construct
  REGION 1..6: note = LCDR1 of FN 8C
NIGSKS

SEQ ID NO: 187 - skipped

SEQ ID NO: 188 - protein, 11 aa, synthetic construct
  REGION 1..11: note = LCDR3 of FN 8C
QVWDSSSDHWV

SEQ ID NO: 189 - protein, 8 aa, synthetic construct
  REGION 1..8: note = HCDR1 of FD 5E
GFTFDDYA

SEQ ID NO: 190 - protein, 8 aa, synthetic construct
  REGION 1..8: note = HCDR2 of FD 5E
ISWDGGST

SEQ ID NO: 191 - protein, 15 aa, synthetic construct
  REGION 1..15: note = HCDR3 of FD 5E
AKDSVRFRYYYGMDV

SEQ ID NO: 192 - protein, 6 aa, synthetic construct
  REGION 1..6: note = LCDR1 of FD 5E
QSVSSY

SEQ ID NO: 193 - skipped

SEQ ID NO: 194 - protein, 9 aa, synthetic construct
  REGION 1..9: note = LCDR3 of FD 5E
QQRSNWPLT

SEQ ID NO: 195 - protein, 8 aa, synthetic construct
  REGION 1..8: note = HCDR1 of FD 5D
GFTFSSYS

SEQ ID NO: 196 - protein, 8 aa, synthetic construct
  REGION 1..8: note = HCDR2 of FD 5D
ISSSSSTI

SEQ ID NO: 197 - protein, 22 aa, synthetic construct
  REGION 1..22: note = HCDR3 of FD 5D
ASPGGITAAGTSVLFGYYGMDV

SEQ ID NO: 198 - protein, 11 aa, synthetic construct
  REGION 1..11: note = LCDR1 of FD 5D
QSLLHSNGYNY

SEQ ID NO: 199 - skipped

SEQ ID NO: 200 - protein, 11 aa, synthetic construct
  REGION 1..11: note = LCDR3 of FD 5D
MQALQTPITWT

SEQ ID NO: 201 - protein, 8 aa, synthetic construct
  REGION 1..8: note = HCDR1 of FI 3A
GFTVSSNY

SEQ ID NO: 202 - protein, 7 aa, synthetic construct
  REGION 1..7: note = HCDR2 of FI 3A
IYSGGST

SEQ ID NO: 203 - protein, 11 aa, synthetic construct
  REGION 1..11: note = HCDR3 of FI 3A
ARDHVRPGMNI

SEQ ID NO: 204 - protein, 6 aa, synthetic construct
  REGION 1..6: note = LCDR1 of FI 3A
QDISNY

SEQ ID NO: 205 - skipped

SEQ ID NO: 206 - protein, 9 aa, synthetic construct
  REGION 1..9: note = LCDR3 of FI 3A
QQYDNLPVT

SEQ ID NO: 207 - protein, 8 aa, synthetic construct
  REGION 1..8: note = HCDR1 of FD 10A
GFTFSSYW

SEQ ID NO: 208 - protein, 8 aa, synthetic construct
  REGION 1..8: note = HCDR2 of FD 10A
INSDGSST

SEQ ID NO: 209 - protein, 7 aa, synthetic construct
  REGION 1..7: note = HCDR3 of FD 10A
ANMAFDI

SEQ ID NO: 210 - protein, 12 aa, synthetic construct
  REGION 1..12: note = LCDR1 of FD 10A
QSVLYSSNNKNY

SEQ ID NO: 211 - skipped

SEQ ID NO: 212 - protein, 9 aa, synthetic construct
  REGION 1..9: note = LCDR3 of FD 10A
QQYYSTPIT

SEQ ID NO: 213 - protein, 10 aa, synthetic construct
  REGION 1..10: note = HCDR1 of FJ 4E
GGSISSGTYY

SEQ ID NO: 214 - protein, 7 aa, synthetic construct
  REGION 1..7: note = HCDR2 of FJ 4E
IYNTGRP

SEQ ID NO: 215 - protein, 19 aa, synthetic construct
  REGION 1..19: note = HCDR3 of FJ 4E
ARDEYDSSDSGIQGHWFDP

SEQ ID NO: 216 - protein, 6 aa, synthetic construct
  REGION 1..6: note = LCDR1 of FJ 4E
QNINSF

SEQ ID NO: 217 - skipped

SEQ ID NO: 218 - protein, 9 aa, synthetic construct
  REGION 1..9: note = LCDR3 of FJ 4E
QQSYSTPWT

SEQ ID NO: 219 - protein, 9 aa, synthetic construct
  REGION 1..9: note = HCDR1 of FJ 1C
GYSISSGYY

SEQ ID NO: 220 - protein, 7 aa, synthetic construct
  REGION 1..7: note = HCDR2 of FJ 1C
IYHSGST

SEQ ID NO: 221 - protein, 19 aa, synthetic construct
  REGION 1..19: note = HCDR3 of FJ 1C
ARDKALLWFGELFTNLFDY

SEQ ID NO: 222 - protein, 9 aa, synthetic construct
  REGION 1..9: note = LCDR1 of FJ 1C
SSDVGGYNY

SEQ ID NO: 223 - skipped

SEQ ID NO: 224 - protein, 10 aa, synthetic construct
  REGION 1..10: note = LCDR3 of FJ 1C
SSYTSSSTLV

SEQ ID NO: 225 - protein, 10 aa, synthetic construct
  REGION 1..10: note = HCDR1 of EW 8B
GGSISSSGYY

SEQ ID NO: 226 - protein, 7 aa, synthetic construct
  REGION 1..7: note = HCDR2 of EW 8B
FYFTGSV

SEQ ID NO: 227 - protein, 11 aa, synthetic construct
  REGION 1..11: note = HCDR3 of EW 8B
ARQEVWGGFDI

SEQ ID NO: 228 - protein, 7 aa, synthetic construct
  REGION 1..7: note = LCDR1 of EW 8B
QSLGSSY

SEQ ID NO: 229 - skipped

SEQ ID NO: 230 - protein, 8 aa, synthetic construct
  REGION 1..8: note = LCDR3 of EW 8B
QQYGSSPT

SEQ ID NO: 231 - protein, 10 aa, synthetic construct
  REGION 1..10: note = HCDR1 of FD 11C
GGSISSSSYY

SEQ ID NO: 232 - protein, 7 aa, synthetic construct
  REGION 1..7: note = HCDR2 of FD 11C
IYYSGST

SEQ ID NO: 233 - protein, 21 aa, synthetic construct
  REGION 1..21: note = HCDR3 of FD 11C
AREYYYGSETKKYYYYYGMDV

SEQ ID NO: 234 - protein, 6 aa, synthetic construct
  REGION 1..6: note = LCDR1 of FD 11C
KLGDKY

SEQ ID NO: 235 - skipped

SEQ ID NO: 236 - protein, 8 aa, synthetic construct
  REGION 1..8: note = LCDR3 of FD 11C
QAWDSSTV

SEQ ID NO: 237 - protein, 8 aa, synthetic construct
  REGION 1..8: note = HCDR1 of FD 7C
GGSISSYY

SEQ ID NO: 238 - protein, 7 aa, synthetic construct
  REGION 1..7: note = HCDR2 of FD 7C
IYYSGST

SEQ ID NO: 239 - protein, 18 aa, synthetic construct
  REGION 1..18: note = HCDR3 of FD 7C
ARDYRFGELFGRFAWFDP

SEQ ID NO: 240 - protein, 6 aa, synthetic construct
  REGION 1..6: note = LCDR1 of FD 7C
QSVSSN

SEQ ID NO: 241 - skipped

SEQ ID NO: 242 - protein, 9 aa, synthetic construct
  REGION 1..9: note = LCDR3 of FD 7C
QQYNNWPRA

SEQ ID NO: 243 - protein, 8 aa, synthetic construct
  REGION 1..8: note = HCDR1 of FD 7D
GYSFTSYW

SEQ ID NO: 244 - protein, 8 aa, synthetic construct
  REGION 1..8: note = HCDR2 of FD 7D
IDPSDSYT

SEQ ID NO: 245 - protein, 19 aa, synthetic construct
  REGION 1..19: note = HCDR3 of FD 7D
ARHSDCSSTSCYFVDAFDI

SEQ ID NO: 246 - protein, 6 aa, synthetic construct
  REGION 1..6: note = LCDR1 of FD 7D
ALPKQY

SEQ ID NO: 247 - skipped

SEQ ID NO: 248 - protein, 11 aa, synthetic construct
  REGION 1..11: note = LCDR3 of FD 7D
QSADSSGTYVV

SEQ ID NO: 249 - protein, 8 aa, synthetic construct
  REGION 1..8: note = HCDR1 of FJ 10B
GYSFTSYW

SEQ ID NO: 250 - protein, 8 aa, synthetic construct
  REGION 1..8: note = HCDR2 of FJ 10B
IDPSDSYT

SEQ ID NO: 251 - protein, 16 aa, synthetic construct
  REGION 1..16: note = HCDR3 of FJ 10B
ARLDPRYGPDYYGMDV

SEQ ID NO: 252 - protein, 6 aa, synthetic construct
  REGION 1..6: note = LCDR1 of FJ 10B
QSISSY

SEQ ID NO: 253 - skipped

SEQ ID NO: 254 - protein, 9 aa, synthetic construct
  REGION 1..9: note = LCDR3 of FJ 10B
QQSYSTPLT

SEQ ID NO: 255 - protein, 8 aa, synthetic construct
  REGION 1..8: note = HCDR1 of FB 9D
GYRFNSYW

SEQ ID NO: 256 - protein, 8 aa, synthetic construct
  REGION 1..8: note = HCDR2 of FB 9D
IFPTDSDV

SEQ ID NO: 257 - protein, 22 aa, synthetic construct
  REGION 1..22: note = HCDR3 of FB 9D
ARHWASMVRGVIRASHYYGMDV

SEQ ID NO: 258 - protein, 9 aa, synthetic construct
  REGION 1..9: note = LCDR1 of FB 9D
SSDIGKYNY

SEQ ID NO: 259 - skipped

SEQ ID NO: 260 - protein, 12 aa, synthetic construct
  REGION 1..12: note = LCDR3 of FB 9D
SSYAFGGSDTRV

SEQ ID NO: 261 - protein, 8 aa, synthetic construct
  REGION 1..8: note = HCDR1 of FB 1E
GYRFNSYW

SEQ ID NO: 262 - protein, 8 aa, synthetic construct
  REGION 1..8: note = HCDR2 of FB 1E
IFPTDSDI

SEQ ID NO: 263 - protein, 22 aa, synthetic construct
  REGION 1..22: note = HCDR3 of FB 1E
ARHWASMVRGVIRASHYYGMDV

SEQ ID NO: 264 - protein, 9 aa, synthetic construct
  REGION 1..9: note = LCDR1 of FB 1E
SSDIGDYNY

SEQ ID NO: 265 - skipped

SEQ ID NO: 266 - protein, 12 aa, synthetic construct
  REGION 1..12: note = LCDR3 of FB 1E
SSYAFGGSDIRV

SEQ ID NO: 267 - protein, 8 aa, synthetic construct
  REGION 1..8: note = HCDR1 of EZ 7A
GYSFTSYW

SEQ ID NO: 268 - protein, 8 aa, synthetic construct
  REGION 1..8: note = HCDR2 of EZ 7A
IYPGDSDT

SEQ ID NO: 269 - protein, 16 aa, synthetic construct
  REGION 1..16: note = HCDR3 of EZ 7A
ARGWVYRGFPYYGMDV

SEQ ID NO: 270 - protein, 9 aa, synthetic construct
  REGION 1..9: note = LCDR1 of EZ 7A
SSDVGGYNY

SEQ ID NO: 271 - skipped

SEQ ID NO: 272 - protein, 10 aa, synthetic construct
  REGION 1..10: note = LCDR3 of EZ 7A
CSYAGSYTLV

SEQ ID NO: 273 - protein, 120 aa, synthetic construct
  REGION 1..120: note = VH of EZ 9B
EVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCAREGPTVTWWFDPWGQGTLVTVSS

SEQ ID NO: 274 - protein, 110 aa, synthetic construct
  REGION 1..110: note = VL of EZ 9B
SYELTQPPSVSVAPGQTARITCGGNNIGSKSVHWYQQKPGQAPVLVVYDDSDRPSGIPERFSGSNSGNTATLTISRVEAGDEADYYCQVWDSSSDHPSWVFGGGTKLTVL

SEQ ID NO: 275 - protein, 122 aa, synthetic construct
  REGION 1..122: note = VH of EZ 11C
EVQLVESGAEVKKPGASVKVSCKVSGYTLTELSMHWVRQAPGKGLEWMGGFDPEDGETIYAQKFQGRVTMTEDTSTDTAYMELSSLRSEDTAVYYCATTTVTTPTANWFDPWGQGTLVTVSS

SEQ ID NO: 276 - protein, 110 aa, synthetic construct
  REGION 1..110: note = VL of EZ 11C
QSALTQPPSVSAAPGQKVTISCSGSSSNIGNNYVSWYQQLPGTAPKLLIYENNKRPSGIPDRFSGSKSGTSATLGITGLQTGDEADYYCGTWDSSLRQVVFGGGTKLTVL

SEQ ID NO: 277 - protein, 117 aa, synthetic construct
  REGION 1..117: note = VH of FD 9B
EVQLVESGGGLVQPGGSLRLSCAGTGFSFSRYWMNWVRQAPGKGLEWVANMDPDGGAKYYLDSVKGRFTISGDNAKNSLYLQMNRLRAEDTAVYYCVKFGRSEGLFWGRGTLVTVSS

SEQ ID NO: 278 - protein, 110 aa, synthetic construct
  REGION 1..110: note = VL of FD 9B
QAVVTQEPSLTVSPGGTVTLTCGSSTGAVTSGHYPYWFQQKPGQAPRTLIYDTSNKHSWTPARFSGSLLGGKAALTLSGAQPEDEADYYCFLTYVGARRLFGGGTKLTVL

SEQ ID NO: 279 - protein, 122 aa, synthetic construct
  REGION 1..122: note = VH of EZ 11A
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARDDYSGSYYWEFDYWGQGTLVTVSS

SEQ ID NO: 280 - protein, 111 aa, synthetic construct
  REGION 1..111: note = VL of EZ 11A
QPVLTQSPSASASLGASVKLTCTLSSGHSSYAIAWHQQQPEKGPRYLMKLNSDGSHSKGDGIPDRFSGSSSGAERYLTISSLQSEDEADYYCQTWGTGIWVFGGGTKLTVL

SEQ ID NO: 281 - protein, 115 aa, synthetic construct
  REGION 1..115: note = VH of EW 10C
EVQLVESGGGLVQPGGSLRLSCAASGFTFNKYKMTWVRQAPGKGLEWVANIKEDGSEKNYVDSVKGRFTISRDNARNSVYMHLNNLRVEDTAVYYCARGRTLGDWGQGTTVTVSS

SEQ ID NO: 282 - protein, 113 aa, synthetic construct
  REGION 1..113: note = VL of EW 10C
AIRMTQSPLSLPVTLGQPASISCRSSQSLVHSDGNTYLNWFQQRPGQSPRRLIYKVSNRDSGAPDRFSGSGSGTDFTLKISRVEAEDVGVYYCMQGTHWPPITFGPGTKVDIK

SEQ ID NO: 283 - protein, 121 aa, synthetic construct
  REGION 1..121: note = VH of EY 12B
EVQLVESGGDLVQPGRSLRLSCAASGFTFDYFAMHWVRQVPGKGLEWVSGIRWNSETIGYADSVKGRFTISRDNAKKSLYLEMNSLRSEDTAFYYCAKGRSGYGHTAFDVWGQGTMVTVSS

SEQ ID NO: 284 - protein, 111 aa, synthetic construct
  REGION 1..111: note = VL of EY 12B
QSALTQPPSVSGAPGQRVTISCTGSSSNIGANYDVHWYQRLPGTAPKLLIYDNNNRPSGVPDRFSGSKSGTSASLAIAGLQAEDEADYYCQSYDSSLSASVFGGGTKLTVL

SEQ ID NO: 285 - protein, 122 aa, synthetic construct
  REGION 1..122: note = VH of EZ 8C
EVQLVESGGGLVKPGGSLRLSCAASGFTFSSYTMNWVRQAPGKGLEWVSSFIANSDYKFYADSVKGRFTISRDNAKSSLYLQMNSLRAEDTAVYYCARELTSYGSHDAFDIWGQGTMVTVSS

SEQ ID NO: 286 - protein, 110 aa, synthetic construct
  REGION 1..110: note = VL of EZ 8C
QSALTQPASVSGSPGQSITISCTGTSSDVGGYNYVSWYQQHPGKAPKLLIYEVTNRPSGVSDRFSGSKSANVASLTISGLQAEDEADYYCSSYTTTDSVVFGGGTKLTVL

SEQ ID NO: 287 - protein, 116 aa, synthetic construct
  REGION 1..116: note = VH of EY 9C
EVQLVESGGGLVKPGGSLRLSCAASEFTFSSYTLNWVRQAPGKGLEWVSSISTSSAYIYYADSVKGRFTISRDNAKKSLSLQMNSLTAEDTAVYYCATWGGAPFDYWGQGTMVTVS

SEQ ID NO: 288 - protein, 112 aa, synthetic construct
  REGION 1..112: note = VL of EY 9C
QSVLTQPASVSGSPGRSITISCTETSSDVGTYNLVSWYQQHPGKAPKLMIFDDNKRPSGVSNRFSGSKSGNTASLTISGLQAEDEADYYCCSYAGGRTFNVLFGGGTKLTVL

SEQ ID NO: 289 - protein, 119 aa, synthetic construct
  REGION 1..119: note = VH of EZ 8B
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDLGYYGSGSSWGQGTLVTVSS

SEQ ID NO: 290 - protein, 106 aa, synthetic construct
  REGION 1..106: note = VL of EZ 8B
SYELTQPPSVSVSPGQTASITCSGDKLGDKYACWYQQKPGQSPVLVIYQDSKRPSGIPERFSGSNSGNTATLTISGTQAMDEADYYCQAWDSSTAVFGGGTKLTVL

SEQ ID NO: 291 - protein, 133 aa, synthetic construct
  REGION 1..133: note = VH of FD 3E
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDPHYYGSGSYYNQLRGYYYYGMDVWGQGTTVTVSS

SEQ ID NO: 292 - protein, 111 aa, synthetic construct
  REGION 1..111: note = VL of FD 3E
DIVMTQTPLSLSVTPGQPASISCKSSQSLLHSDGKTYLYWYLQKPGQSPQLLIYEVSSRFSGVPDRFSGSGSGTDFTLKISRVEAEDVGVYYCMQGIHLPHFRRRDQVEIK

SEQ ID NO: 293 - protein, 121 aa, synthetic construct
  REGION 1..121: note = VH of EW 1A
EVQLVESGGGVVQPGSSLRLSCETSGFTFSGHAMHWVRQAPGKGLEWLAQIWFDGSEKYYADSVKGRFTISRDNSKKILYMQMNSLRVQDTAVYYCARDGQHLAPFAMDVWGQGTMVTVSS

SEQ ID NO: 294 - protein, 110 aa, synthetic construct
  REGION 1..110: note = VL of EW 1A
QSVLTQPASVSGSPGQSITISCTGTSSDVGASNRVSWYQHSPGEAPKLIIYQVTVRPSGVSDRFSGSKSGNTASLTISGLRTEDEAEYYCNSFVSGDSWVFGGGTKVTVL

SEQ ID NO: 295 - protein, 122 aa, synthetic construct
  REGION 1..122: note = VH of FD 5B
EVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDERRESYNFVLDYWGQGTLVTVSS

SEQ ID NO: 296 - protein, 111 aa, synthetic construct
  REGION 1..111: note = VL of FD 5B
QSVLTQPPSASGSPGQSVTISCTGTSSDAGGYNYVSWYQQHPGKAPKLMIYEVSKRPSGVPDRFSGSKSGNTASLTVSGLQAEDEADYYCSSYAGSNNPFVFGTGTKVTVL

SEQ ID NO: 297 - protein, 132 aa, synthetic construct
  REGION 1..132: note = VH of EW 9A
EVQLLESGGGVVQPGRSLRLSCAASGFTFSSYGMHWVRQAPGKGLEWVAVIWYDGSNKYYADSVKGRFTISRDNSKNTLYLQMNSLRAEDTAVYYCAKDMWALYDILTGYYTPYYYYGMDVWGQGTTVTVSS

SEQ ID NO: 298 - protein, 108 aa, synthetic construct
  REGION 1..108: note = VL of EW 9A
SYELTQPPSVSVSPGQTARITCSADALPKQYAYWYQQKPGQAPVLVIYKDSERPSGIPERFSGSSSGTTVTLTISGVQAEDEADYYCQSADSSGTYWVFGGGTKLTVL

SEQ ID NO: 299 - protein, 122 aa, synthetic construct
  REGION 1..122: note = VH of FD 8C
EVQLVESGGGLVKPGRSLRLSCTASGFTFGDYAMSWFRQAPGKGLEWVGFIRSKAYGGTTEYAASVKGRFTISRDDSKSIAYLQMNSLKTEDTAVYYCTRNDFWSGYYPDYWGQGTLVTVSS

SEQ ID NO: 300 - protein, 113 aa, synthetic construct
  REGION 1..113: note = VL of FD 8C
DIVMTQSPLSLPVTLGQPASISCRSSQSLVHSDGNTYLNWFQQRPGQSPRRLIYKVSNRDSGVPDRFSGSGSGTDFTLKISRVEAEDVGVYYCMQGTHWPRSLSAEGPKWISN

SEQ ID NO: 301 - protein, 120 aa, synthetic construct
  REGION 1..120: note = VH of EW 5A
EVQLVESGGGLVQPGGSLRLSCSVSGFPFGTYAMHWVRQAPGKGLDYVSAINNDGSITYYADSVRGRFTVSRDNSENTLYLRLSGLRPDDTAIYYCVKDRGSVIRDFDVWGRGTLVTVSS

SEQ ID NO: 302 - protein, 109 aa, synthetic construct
  REGION 1..109: note = VL of EW 5A
QTVVTQEPSLTVSPGGTVTLTCASSTGTVTSDYYPNWFQQKPGQAPRPLIFGTAYRHSWTPARFSGSLLGGKAALTVSDVQPEDEADYYCLLYCGGGQLFGGGTKLTVL

SEQ ID NO: 303 - protein, 122 aa, synthetic construct
  REGION 1..122: note = VH of EZ 9C
EVQLVESGGGLVQPGGSLRLSCSASGFTLNGYAMHWVRQAPGKGLEYVSSLSNRGDNTRYAESVKGRFLISRDIAKDTLYLQMSSLRPEDTAVYYCGKGLLSASGGLPIDDWGQGTLVTVSS

SEQ ID NO: 304 - protein, 110 aa, synthetic construct
  REGION 1..110: note = VL of EZ 9C
QSVLTQPPSVSAAPGQKVTISCSGSSSNIGNNYVSWYQQFPGTAPKLLIYDNNKRPSGIPDRFSGSKSGTSATLGITGLQTGDEAAYYCATWDSSLSAGVFGGGAKLTVL

SEQ ID NO: 305 - protein, 117 aa, synthetic construct
  REGION 1..117: note = VH of EZ 9A
EVQLVESGGGLVQPGESLRVSCAASGFTFSNYWMHWVRQVPGKGPVWVSIINTDGSITRYVDSVRGRFTISRDNAKNTVHLQMNSLTAEDTAIYYCARDVNRYPDYWGQGTLVTVSS

SEQ ID NO: 306 - protein, 110 aa, synthetic construct
  REGION 1..110: note = VL of EZ 9A
QSALTQPASVSGSPGQSITISCTGTYSDVGYYNYVSWYQQQPGKAPKVIIHGDINRPFGVSNRFSGSKSGNTASLTISGLQAEDEADYFCCSYVNNGAWVFGGGTKLTVL

SEQ ID NO: 307 - protein, 130 aa, synthetic construct
  REGION 1..130: note = VH of EY 12A
EVQLVESGPGLVKPSQTLSLTCAVSGGSISSGGYYWSWIRQPPGKGLELIGYTDYTGKTLYNPSLKSRLTISVDTSKNQFSLKLRSVTAADTAVYYCARGMTQDDILTGFNRPHWYFDLWGRGSLVTVSS

SEQ ID NO: 308 - protein, 112 aa, synthetic construct
  REGION 1..112: note = VL of EY 12A
QSVLTQPPSVSGAPGQRVTISCTGSSSNIGAGYDVHWYQQLPGTAPKVVIYRNMNRPSGVPDRFSGSKSGTSASLAITGLQAEDEADYYCQSFDSSLSDFVVFGGGTKLTVL

SEQ ID NO: 309 - protein, 121 aa, synthetic construct
  REGION 1..121: note = VH of FD 4C
EVQLVESGPGLVKPSQTLSLTCTVSGGSISSGGYYWSWIRQHPGKGLEWIGYIYYSGSTYYNPSLKSRVTISVDTSKNQFSLKLSSVTAADTAVYYCARVRSSSSWYFDYWGQGTLVTVSS

SEQ ID NO: 310 - protein, 108 aa, synthetic construct
  REGION 1..108: note = VL of FD 4C
QSVLTQPASVSGSPGQSITISCTGTSSDVGGYNYVSWYQQHPGKAPKLMIYDVSKRPSGVSNRFSGSKSGNTASLTISGLQAEDEADYYCSSYTSKWVFGGGTKLTVL

SEQ ID NO: 311 - protein, 116 aa, synthetic construct
  REGION 1..116: note = VH of EW 4C
EVQLVESGPGLVKPSETLSLTCSVSADSFKKGYYWGWIRQPPGKGLESIVNSFDSGTTRYNPSLWGRATVSDMSKWHFSLKLTSVTAADTAVYYCVRGTYGSGLHWGQGILVTVSS

SEQ ID NO: 312 - protein, 109 aa, synthetic construct
  REGION 1..109: note = VL of EW 4C
QTVVTQEPSLTVSPGGTVTLTCGSSVGTVASGHYPYWVQQKPGQAPRTLIYDTDNKQSWTPARFSGSLLGGKAALTLSGAQPEDEADYYCFLSHNDAWVFGGGTKLTVL

SEQ ID NO: 313 - protein, 126 aa, synthetic construct
  REGION 1..126: note = VH of FD 6D
EVQLVESGPGLVKVSETLFLTCTVSGYSIGSGNYWGWIRQPPGKVLEWIGSTYHSGTTYYNPSLKSRVTISVDSSKNQFSLKLTSVTAADTAVYYCARDRLLAVHYDSRGYLVDYWGQGTMVTVSS

SEQ ID NO: 314 - protein, 113 aa, synthetic construct
  REGION 1..113: note = VL of FD 6D
AIRMTQSPDSLAVSLGERATINCKSSQSIFYSSNNKNYLAWYQHKPGQPPKLLIYWASTRESGVPDRFSGSGSGTDFTLTISSLQAEDVAIYYCQQYYDIPRTFGQGTKVEIR

SEQ ID NO: 315 - protein, 120 aa, synthetic construct
  REGION 1..120: note = VH of EY 5A
EVQLVESGPGLVKPSGTLFLTCTVSGGSISNYYWSWIRQPPGKALEWIGFIYSNGNTDYNPSLQSRVTISGDTSKNQFSLNLRSVTAADTAVYYCARGPGPATGGSLDYWGQGTMVTVSS

SEQ ID NO: 316 - protein, 110 aa, synthetic construct
  REGION 1..110: note = VL of EY 5A
NFMLTQPPSASGTPGQRVTISCSGSSSNIGINTVNWYQHLPGTAPKLLIYGNNQRPSGVPDRFSGSKSGTSTSLAISGLQSEDEADYYCSAWDDSLNGPVFGGGTQLTVL

SEQ ID NO: 317 - protein, 131 aa, synthetic construct
  REGION 1..131: note = VH of EZ 7B
EVQLVESSPGLVKPSETLSLTCTVSGGSISSYYWSWIRQPPGKGLEWIGYIYYSGSTNYNPSLKSRVTISVDTSKNQFSLKLSSVTAADTAVYYCARRVFGPVLPSKLGGSYWGGGAFDIWGQGTMVTVSS

SEQ ID NO: 318 - protein, 113 aa, synthetic construct
  REGION 1..113: note = VL of EZ 7B
DIQLTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGPGTKVEIR

SEQ ID NO: 319 - protein, 130 aa, synthetic construct
  REGION 1..130: note = VH of EZ 4C kappa
EVQLVESGPGLVKPSETLSLTCTVSGGSVSSGNNYWSWIRQPPGKALEWIGYIYYSGSTKYNPSLKSRVTMSVGTSKNQFSLKVNSVTAADTAMYYCARAPSAPFGGLFDWILPKGINNWGQGTLVTVSS

SEQ ID NO: 320 - protein, 107 aa, synthetic construct
  REGION 1..107: note = VL of EZ 4C kappa
EIVLTQSPSTLSASVGDRVTITCRTSQSIRSWLAWYQQKPGKAPKLLIFEASTLESGVPERFSGSGSGAEFTLTISSLQPDDFATYYCQQYNGYSYSFGQGTKVEIR

SEQ ID NO: 321 - protein, 130 aa, synthetic construct
  REGION 1..130: note = VH of EZ 4C lambda
VQLVQESGPGLVKPSETLSLTCTVSGGSVSSGNNYWSWIRQPPGKALEWIGYIYYSGSTKYNPSLKSRVTMSVDTSKNQFSLKVNSVTAADTAMYYCARAPSAPFGGLFDWILPKGIDNWGQGTLVTVSS

SEQ ID NO: 322 - protein, 105 aa, synthetic construct
  REGION 1..105: note = VL of EZ 4C lambda
QSALTQPPSVSAAPGQKVTISYSGNSSDMGTYAVSWQRLPGTAPKLFICEENKRPPGIPDRFSGSKSGTSATLGITGLWPEDEADYCIAHSSPRLGCSAEGPSPS

SEQ ID NO: 323 - protein, 130 aa, synthetic construct
  REGION 1..130: note = VH of FD 4B
QLQLQESGPGLVKPSETLSLTCTVSGGSVSSGNNYWSWIRQPPGKALEWIGYLYYSGSTKYNPSLKSRVTMSVDTSKNQFSLKVNSVTAADTAMYYCARAPSAPFGGLFDWILPKGIDSWGQGTLVTVSS

SEQ ID NO: 324 - protein, 105 aa, synthetic construct; identical to SEQ ID NO: 322
  REGION 1..105: note = VL of FD 4B

SEQ ID NO: 325 - protein, 128 aa, synthetic construct
  REGION 1..128: note = VH of EY 8A
EVQLVESGPGLVKPSQTLSLTCTVSGGSISSGSYYWSWIRQPAGKGLEWIGRIYTSGSTNYNPSLKSRVTISVDTSKNQFSLKLSSVTAADTAVYYCAKGHVISGYDDYYYYYGMDVWGQGTTVTVSS

SEQ ID NO: 326 - protein, 108 aa, synthetic construct
  REGION 1..108: note = VL of EY 8A
SYELTQPPSVSVSPGQTARITCSGDALPKQYAYWYQQKPGQAPVLVIYKDSERPSGIPERFSGSSSGTTVTLTISGVQAEDEADYYCQSADSSGTYWVFGGGTKLTVL

SEQ ID NO: 327 - protein, 130 aa, synthetic construct
  REGION 1..130: note = VH of EY 2A
QVQLVESGAEVTKPGDSLIISCKGSGYAFTQYWIGWVRQKPGKGLEWMAMVYPDSSAVFAGGASGVRYRPPFQGQVTISADTSVNTAYLQWDSLKASDTAMYYCVRQERGSNTWYAGNSWGQGTLVTVSS

SEQ ID NO: 328 - protein, 112 aa, synthetic construct
  REGION 1..112: note = VL of EY 2A
DIVMTQSPLSLPVSPGEPASISCRSSQSLLHTDAYNYLDWYLQKPGQSPQLLIYLGSTRASGVPDRFSGSGSGTDFTLKISSVKAEDVGVYYCMQALQTPGTFGQGTKLEIK

SEQ ID NO: 329 - protein, 130 aa, synthetic construct
  REGION 1..130: note = VH of EY 3B
EVQLVESGAEVTKPGESLIISCKGSGYAFTQYWIGWVRQKPGKGLEWMAMVYPDSSAVFAGGASGVRYRPPFQGQVTISADTSVNTAYLQWDSLKASDTAMYYCVRQERGSNTWYAGNSWGQGTLVTVFS

SEQ ID NO: 330 - protein, 112 aa, synthetic construct
  REGION 1..112: note = VL of EY 3B
EIVLTQSPLSLPVSPGEPASISCRSSQSLLHTDAYNYLDWYLQKPGQSPQLLIYLGSTRASGVPDRFSGSGSGTDFTLKISSVKAEDVGVYYCMQALQTPGTFGQGTKVEIR

SEQ ID NO: 331 - protein, 119 aa, synthetic construct
  REGION 1..119: note = VH of EZ 4A
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARSPIAADLFDYWGQGTLVTVSS

SEQ ID NO: 332 - protein, 107 aa, synthetic construct
  REGION 1..107: note = VL of EZ 4A
DIQLTQSPSSLSASVGDRVTITCQASQDISNYLNWYQQKPGKAPKLLIYDASNLETGVPSRFSGSGSGTDFTFTISSLQPEDIATYYCQQYDNLLFTFGPGTKVEIK

SEQ ID NO: 333 - protein, 8 aa, synthetic construct
  REGION 1..8: note = HCDR1 of EZ 9B
GYTFTGYY

SEQ ID NO: 334 - protein, 8 aa, synthetic construct
  REGION 1..8: note = HCDR2 of EZ 9B
INPNSGGT

SEQ ID NO: 335 - protein, 13 aa, synthetic construct
  REGION 1..13: note = HCDR3 of EZ 9B
AREGPTVTWWFDP

SEQ ID NO: 336 - protein, 6 aa, synthetic construct
  REGION 1..6: note = LCDR1 of EZ 9B
NIGSKS

SEQ ID NO: 337 - skipped

SEQ ID NO: 338 - protein, 13 aa, synthetic construct
  REGION 1..13: note = LCDR3 of EZ 9B
QVWDSSSDHPSWV

SEQ ID NO: 339 - protein, 8 aa, synthetic construct
  REGION 1..8: note = HCDR1 of EZ 11C
GYTLTELS

SEQ ID NO: 340 - protein, 8 aa, synthetic construct
  REGION 1..8: note = HCDR2 of EZ 11C
FDPEDGET

SEQ ID NO: 341 - protein, 15 aa, synthetic construct
  REGION 1..15: note = HCDR3 of EZ 11C
ATTTVTTPTANWFDP

SEQ ID NO: 342 - protein, 8 aa, synthetic construct
  REGION 1..8: note = LCDR1 of EZ 11C
SSNIGNNY

SEQ ID NO: 343 - skipped

SEQ ID NO: 344 - protein, 11 aa, synthetic construct
  REGION 1..11: note = LCDR3 of EZ C
GTWDSSLRQVV

SEQ ID NO: 345 - protein, 8 aa, synthetic construct
  REGION 1..8: note = HCDR1 of FD 9B
GFSFSRYW

SEQ ID NO: 346 - protein, 8 aa, synthetic construct
  REGION 1..8: note = HCDR2 of FD 9B
MDPDGGAK

SEQ ID NO: 347 - protein, 10 aa, synthetic construct
  REGION 1..10: note = HCDR3 of FD 9B
VKFGRSEGLF

SEQ ID NO: 348 - protein, 9 aa, synthetic construct
  REGION 1..9: note = LCDR1 of FD 9B
TGAVTSGHY

SEQ ID NO: 349 - skipped

SEQ ID NO: 350 - protein, 10 aa, synthetic construct
  REGION 1..10: note = LCDR3 of FD 9B
FLTYVGARRL

SEQ ID NO: 351 - protein, 8 aa, synthetic construct
  REGION 1..8: note = HCDR1 of EZ 11A
GFTFSSYW

SEQ ID NO: 352 - protein, 8 aa, synthetic construct
  REGION 1..8: note = HCDR2 of EZ 11A
IKQDGSEK

SEQ ID NO: 353 - protein, 15 aa, synthetic construct
  REGION 1..15: note = HCDR3 of EZ 11A
ARDDYSGSYYWEFDY

SEQ ID NO: 354 - protein, 7 aa, synthetic construct
  REGION 1..7: note = LCDR1 of EZ 11A
SGHSSYA

SEQ ID NO: 355 - protein, 7 aa, synthetic construct
  REGION 1..7: note = LCDR2 of EZ 11A
LNSDGSH

SEQ ID NO: 356 - protein, 9 aa, synthetic construct
  REGION 1..9: note = LCDR3 of EZ 11A
QTWGTGIWV

SEQ ID NO: 357 - protein, 8 aa, synthetic construct
  REGION 1..8: note = HCDR1 of EW 10C
GFTFNKYK

SEQ ID NO: 358 - protein, 8 aa, synthetic construct
  REGION 1..8: note = HCDR2 of EW 10C
IKEDGSEK

SEQ ID NO: 359 - protein, 8 aa, synthetic construct
  REGION 1..8: note = HCDR3 of EW 10C
ARGRTLGD

SEQ ID NO: 360 - protein, 11 aa, synthetic construct
  REGION 1..11: note = LCDR1 of EW 10C
QSLVHSDGNTY

SEQ ID NO: 361 - skipped

SEQ ID NO: 362 - protein, 10 aa, synthetic construct
  REGION 1..10: note = LCDR3 of EW 10C
MQGTHWPPIT

SEQ ID NO: 363 - protein, 8 aa, synthetic construct
  REGION 1..8: note = HCDR1 of EY 12B
GFTFDYFA

SEQ ID NO: 364 - protein, 8 aa, synthetic construct
  REGION 1..8: note = HCDR2 of EY 12B
IRWNSETI

SEQ ID NO: 365 - protein, 14 aa, synthetic construct
  REGION 1..14: note = HCDR3 of EY 12B
AKGRSGYGHTAFDV

SEQ ID NO: 366 - protein, 9 aa, synthetic construct
  REGION 1..9: note = LCDR1 of EY 12B
SSNIGANYD

SEQ ID NO: 367 - skipped

SEQ ID NO: 368 - protein, 11 aa, synthetic construct
  REGION 1..11: note = LCDR3 of EY 12B
QSYDSSLSASV

SEQ ID NO: 369 - protein, 8 aa, synthetic construct
  REGION 1..8: note = HCDR1 of EZ 8C
GFTFSSYT

SEQ ID NO: 370 - protein, 8 aa, synthetic construct
  REGION 1..8: note = HCDR2 of EZ 8C
FIANSDYK

SEQ ID NO: 371 - protein, 15 aa, synthetic construct
  REGION 1..15: note = HCDR3 of EZ8C
ARELTSYGSHDAFDI

SEQ ID NO: 372 - protein, 9 aa, synthetic construct
  REGION 1..9: note = LCDR1 of EZ 8C
SSDVGGYNY

SEQ ID NO: 373 - skipped

SEQ ID NO: 374 - protein, 10 aa, synthetic construct
  REGION 1..10: note = LCDR3 of EZ 8C
SSYTTTDSVV

SEQ ID NO: 375 - protein, 8 aa, synthetic construct
  REGION 1..8: note = HCDR1 of EY 9C
EFTFSSYT

SEQ ID NO: 376 - protein, 8 aa, synthetic construct
  REGION 1..8: note = HCDR2 of EY 9C
ISTSSAYI

SEQ ID NO: 377 - protein, 10 aa, synthetic construct
  REGION 1..10: note = HCDR3 of EY 9C
ATWGGAPFDY

SEQ ID NO: 378 - protein, 9 aa, synthetic construct
  REGION 1..9: note = LCDR1 of EY 9C
SSDVGTYNL

SEQ ID NO: 379 - skipped

SEQ ID NO: 380 - protein, 12 aa, synthetic construct
  REGION 1..12: note = LCDR3 of EY 9C
CSYAGGRTFNVL

SEQ ID NO: 381 - protein, 8 aa, synthetic construct
  REGION 1..8: note = HCDR1 of EZ 8B
GFTFSSYA

SEQ ID NO: 382 - protein, 8 aa, synthetic construct
  REGION 1..8: note = HCDR2 of EZ 8B
ISGSGGST

SEQ ID NO: 383 - protein, 12 aa, synthetic construct
  REGION 1..12: note = HCDR3 of EZ 8B
AKDLGYYGSGSS

SEQ ID NO: 384 - protein, 6 aa, synthetic construct
  REGION 1..6: note = LCDR1 of EZ 8B
KLGDKY

SEQ ID NO: 385 - skipped

SEQ ID NO: 386 - protein, 9 aa, synthetic construct
  REGION 1..9: note = LCDR3 of EZ 8B
QAWDSSTAV

SEQ ID NO: 387 - protein, 8 aa, synthetic construct
  REGION 1..8: note = HCDR1 of FD 3E
GFTFSSYG

SEQ ID NO: 388 - protein, 8 aa, synthetic construct
  REGION 1..8: note = HCDR2 of FD 3E
ISYDGSNK

SEQ ID NO: 389 - protein, 26 aa, synthetic construct
  REGION 1..26: note = HCDR3 of FD 3E
AKDPHYYGSGSYYNQLRGYYYYGMDV

SEQ ID NO: 390 - protein, 11 aa, synthetic construct
  REGION 1..11: note = LCDR1 of FD 3E
QSLLHSDGKTY

SEQ ID NO: 391 - skipped

SEQ ID NO: 392 - protein, 8 aa, synthetic construct
  REGION 1..8: note = LCDR3 of FD 3E
MQGIHLPH

SEQ ID NO: 393 - protein, 8 aa, synthetic construct
  REGION 1..8: note = HCDR1 of EW 1A
GFTFSGHA

SEQ ID NO: 394 - protein, 8 aa, synthetic construct
  REGION 1..8: note = HCDR2 of EW 1A
IWFDGSEK

SEQ ID NO: 395 - protein, 14 aa, synthetic construct
  REGION 1..14: note = HCDR3 of EW 1A
ARDGQHLAPFAMDV

SEQ ID NO: 396 - protein, 9 aa, synthetic construct
  REGION 1..9: note = LCDR1 of EW 1A
SSDVGASNR

SEQ ID NO: 397 - skipped

SEQ ID NO: 398 - protein, 10 aa, synthetic construct
  REGION 1..10: note = LCDR3 of EW 1A
NSFVSGDSWV

SEQ ID NO: 399 - protein, 8 aa, synthetic construct
  REGION 1..8: note = HCDR1 of FD 5B
GFTFSSYG

SEQ ID NO: 400 - protein, 8 aa, synthetic construct
  REGION 1..8: note = HCDR2 of FD 5B
IWYDGSNK

SEQ ID NO: 401 - protein, 15 aa, synthetic construct
  REGION 1..15: note = HCDR3 of FD 5B
ARDERRESYNFVLDY

SEQ ID NO: 402 - protein, 9 aa, synthetic construct
  REGION 1..9: note = LCDR1 of FD 5B
SSDAGGYNY

SEQ ID NO: 403 - skipped

SEQ ID NO: 404 - protein, 11 aa, synthetic construct
  REGION 1..11: note = LCDR3 of FD 5B
SSYAGSNNPFV

SEQ ID NO: 405 - protein, 8 aa, synthetic construct
  REGION 1..8: note = HCDR1 of EW 9A
GFTFSSYG

SEQ ID NO: 406 - protein, 8 aa, synthetic construct
  REGION 1..8: note = HCDR2 of EW 9A
IWYDGSNK

SEQ ID NO: 407 - protein, 25 aa, synthetic construct
  REGION 1..25: note = HCDR3 of EW 9A
AKDMWALYDILTGYYTPYYYYGMDV

SEQ ID NO: 408 - protein, 6 aa, synthetic construct
  REGION 1..6: note = LCDR1 of EW 9A
ALPKQY

SEQ ID NO: 409 - skipped

SEQ ID NO: 410 - protein, 11 aa, synthetic construct
  REGION 1..11: note = LCDR3 of EW 9A
QSADSSGTYWV

SEQ ID NO: 411 - protein, 8 aa, synthetic construct
  REGION 1..8: note = HCDR1 of FD 8C
GFTFGDYA

SEQ ID NO: 412 - protein, 10 aa, synthetic construct
  REGION 1..10: note = HCDR2 of FD 8C
IRSKAYGGTT

SEQ ID NO: 413 - protein, 13 aa, synthetic construct
  REGION 1..13: note = HCDR3 of FD 8C
TRNDFWSGYYPDY

SEQ ID NO: 414 - protein, 11 aa, synthetic construct
  REGION 1..11: note = LCDR1 of FD 8C
QSLVHSDGNTY

SEQ ID NO: 415 - skipped

SEQ ID NO: 416 - protein, 10 aa, synthetic construct
  REGION 1..10: note = LCDR3 of FD 8C
MQGTHWPRSL

SEQ ID NO: 417 - protein, 8 aa, synthetic construct
  REGION 1..8: note = HCDR1 of EW 5A
GFPFGTYA

SEQ ID NO: 418 - protein, 8 aa, synthetic construct
  REGION 1..8: note = HCDR2 of EW 5A
INNDGSIT

SEQ ID NO: 419 - protein, 13 aa, synthetic construct
  REGION 1..13: note = HCDR3 of EW 5A
VKDRGSVIRDFDV

SEQ ID NO: 420 - protein, 9 aa, synthetic construct
  REGION 1..9: note = LCDR1 of EW %A
TGTVTSDYY

SEQ ID NO: 421 - skipped

SEQ ID NO: 422 - protein, 9 aa, synthetic construct
  REGION 1..9: note = LCDR3 of EW 5A
LLYCGGGQL

SEQ ID NO: 423 - protein, 8 aa, synthetic construct
  REGION 1..8: note = HCDR1 of EZ 9C
GFTLNGYA

SEQ ID NO: 424 - protein, 8 aa, synthetic construct
  REGION 1..8: note = HCDR2 of EZ 9C
LSNRGDNT

SEQ ID NO: 425 - protein, 15 aa, synthetic construct
  REGION 1..15: note = HCDR3 of EZ 9C
GKGLLSASGGLPIDD

SEQ ID NO: 426 - protein, 8 aa, synthetic construct
  REGION 1..8: note = LCDR1 of EZ 9C
SSNIGNNY

SEQ ID NO: 427 - skipped

SEQ ID NO: 428 - protein, 11 aa, synthetic construct
  REGION 1..11: note = LCDR3 of EZ 9C
ATWDSSLSAGV

SEQ ID NO: 429 - protein, 8 aa, synthetic construct
  REGION 1..8: note = HCDR1 of EZ 9A
GFTFSNYW

SEQ ID NO: 430 - protein, 8 aa, synthetic construct
  REGION 1..8: note = HCDR2 of EZ 9A
INTDGSIT

SEQ ID NO: 431 - protein, 10 aa, synthetic construct
  REGION 1..10: note = HCDR3 of EZ 9A
ARDVNRYPDY

SEQ ID NO: 432 - protein, 9 aa, synthetic construct
  REGION 1..9: note = LCDR1 of EZ 9A
YSDVGYYNY

SEQ ID NO: 433 - skipped

SEQ ID NO: 434 - protein, 10 aa, synthetic construct
  REGION 1..10: note = LCDR3 of EZ 9A
CSYVNNGAWV

SEQ ID NO: 435 - protein, 10 aa, synthetic construct
  REGION 1..10: note = HCDR1 of EY 12A
GGSISSGGYY

SEQ ID NO: 436 - protein, 7 aa, synthetic construct
  REGION 1..7: note = HCDR2 of EY 12A
TDYTGKT

SEQ ID NO: 437 - protein, 22 aa, synthetic construct
  REGION 1..22: note = HCDR3 of EY 12A
ARGMTQDDILTGFNRPHWYFDL

SEQ ID NO: 438 - protein, 9 aa, synthetic construct
  REGION 1..9: note = LCDR1 of EY 12A
SSNIGAGYD

SEQ ID NO: 439 - skipped

SEQ ID NO: 440 - protein, 12 aa, synthetic construct
  REGION 1..12: note = LCDR3 of EY 12A
QSFDSSLSDFVV

SEQ ID NO: 441 - protein, 10 aa, synthetic construct
  REGION 1..10: note = HCDR1 of FD 4C
GGSISSGGYY

SEQ ID NO: 442 - protein, 7 aa, synthetic construct
  REGION 1..7: note = HCDR2 of FD 4C
IYYSGST

SEQ ID NO: 443 - protein, 13 aa, synthetic construct
  REGION 1..13: note = HCDR3 of FD 4C
ARVRSSSSWYFDY

SEQ ID NO: 444 - protein, 9 aa, synthetic construct
  REGION 1..9: note = LCDR1 of FD 4C
SSDVGGYNY

SEQ ID NO: 445 - skipped

SEQ ID NO: 446 - protein, 8 aa, synthetic construct
  REGION 1..8: note = LCDR3 of FD 4C
SSYTSKWV

SEQ ID NO: 447 - protein, 9 aa, synthetic construct
  REGION 1..9: note = HCDR1 of EW 4C
ADSFKKGYY

SEQ ID NO: 448 - protein, 7 aa, synthetic construct
  REGION 1..7: note = HCDR2 of EW 4C
SFDSGTT

SEQ ID NO: 449 - protein, 9 aa, synthetic construct
  REGION 1..9: note = HCDR3 of EW 4C
RGTYGSGLH

SEQ ID NO: 450 - protein, 9 aa, synthetic construct
  REGION 1..9: note = LCDR1 of EW 4C
VGTVASGHY

SEQ ID NO: 451 - skipped

SEQ ID NO: 452 - protein, 9 aa, synthetic construct
  REGION 1..9: note = LCDR3 of EW 4C
FLSHNDAWV

SEQ ID NO: 453 - protein, 9 aa, synthetic construct
  REGION 1..9: note = HCDR1 of FD 6D
GYSIGSGNY

SEQ ID NO: 454 - protein, 7 aa, synthetic construct
  REGION 1..7: note = HCDR2 of FD 6D
TYHSGTT

SEQ ID NO: 455 - protein, 19 aa, synthetic construct
  REGION 1..19: note = HCDR3 of FD 6D
ARDRLLAVHYDSRGYLVDY

SEQ ID NO: 456 - protein, 12 aa, synthetic construct
  REGION 1..12: note = LCDR1 of FD 6D
QSIFYSSNNKNY

SEQ ID NO: 457 - skipped

SEQ ID NO: 458 - protein, 9 aa, synthetic construct
  REGION 1..9: note = LCDR3 of FD 6D
QQYYDIPRT

SEQ ID NO: 459 - protein, 8 aa, synthetic construct
  REGION 1..8: note = HCDR1 of EY 5A
GGSISNYY

SEQ ID NO: 460 - protein, 7 aa, synthetic construct
  REGION 1..7: note = HCDR2 of EY 5A
IYSNGNT

SEQ ID NO: 461 - protein, 14 aa, synthetic construct
  REGION 1..14: note = HCDR3 of EY 5A
ARGPGPATGGSLDY

SEQ ID NO: 462 - protein, 8 aa, synthetic construct
  REGION 1..8: note = LCDR1 of EY 5A
SSNIGINT

SEQ ID NO: 463 - skipped

SEQ ID NO: 464 - protein, 11 aa, synthetic construct
  REGION 1..11: note = LCDR3 of EY 5A
SAWDDSLNGPV

SEQ ID NO: 465 - protein, 8 aa, synthetic construct
  REGION 1..8: note = HCDR1 of EZ 7B
GGSISSYY

SEQ ID NO: 466 - protein, 7 aa, synthetic construct
  REGION 1..7: note = HCDR2 of EZ 7B
IYYSGST

SEQ ID NO: 467 - protein, 25 aa, synthetic construct
  REGION 1..25: note = HCDR3 of EZ 7B
ARRVFGPVLPSKLGGSYWGGGAFDI

SEQ ID NO: 468 - protein, 12 aa, synthetic construct
  REGION 1..12: note = LCDR1 of EZ 7B
QSVLYSSNNKNY

SEQ ID NO: 469 - skipped

SEQ ID NO: 470 - protein, 9 aa, synthetic construct
  REGION 1..9: note = LCDR3 of EZ 7B
QQYYSTPLT

SEQ ID NO: 471 - protein, 10 aa, synthetic construct
  REGION 1..10: note = HCDR1 of EZ4C kappa
GGSVSSGNNY

SEQ ID NO: 472 - protein, 7 aa, synthetic construct
  REGION 1..7: note = HCDR2 of EZ 4C kappa
IYYSGST

SEQ ID NO: 473 - protein, 22 aa, synthetic construct
  REGION 1..22: note = HCDR3 of EZ 4C kappa
ARAPSAPFGGLFDWILPKGINN

SEQ ID NO: 474 - protein, 6 aa, synthetic construct
  REGION 1..6: note = LCDR1 of EZ 4C kappa
QSIRSW

SEQ ID NO: 475 - skipped

SEQ ID NO: 476 - protein, 9 aa, synthetic construct
  REGION 1..9: note = LCDR3 of EZ 4C kappa
QQYNGYSYS

SEQ ID NO: 477 - protein, 10 aa, synthetic construct
  REGION 1..10: note = HCDR1 of EZ 4C lambda
GGSVSSGNNY

SEQ ID NO: 478 - protein, 7 aa, synthetic construct
  REGION 1..7: note = HCDR2 of EZ 4C lambda
IYYSGST

SEQ ID NO: 479 - protein, 22 aa, synthetic construct
  REGION 1..22: note = HCDR3 of EZ 4C lambda
ARAPSAPFGGLFDWILPKGIDN

SEQ ID NO: 480 - protein, 8 aa, synthetic construct
  REGION 1..8: note = LCDR1 of EZ 4C lambda
SSDMGTYA

SEQ ID NO: 481 - skipped

SEQ ID NO: 482 - protein, 10 aa, synthetic construct
  REGION 1..10: note = LCDR3 of EZ 4C lambda
IAHSSPRLGC

SEQ ID NO: 483 - protein, 10 aa, synthetic construct
  REGION 1..10: note = HCDR1 of FD 4B
GGSVSSGNNY

SEQ ID NO: 484 - protein, 7 aa, synthetic construct
  REGION 1..7: note = HCDR2 of FD 4B
LYYSGST

SEQ ID NO: 485 - protein, 22 aa, synthetic construct
  REGION 1..22: note = HCDR3 of FD 4B
ARAPSAPFGGLFDWILPKGIDS

SEQ ID NO: 486 - protein, 8 aa, synthetic construct
  REGION 1..8: note = LCDR1 of FD 4B
SSDMGTYA

SEQ ID NO: 487 - skipped

SEQ ID NO: 488 - protein, 10 aa, synthetic construct
  REGION 1..10: note = LCDR3 of FD 4B
IAHSSPRLGC

SEQ ID NO: 489 - protein, 10 aa, synthetic construct
  REGION 1..10: note = HCDR1 of EY 8A
GGSISSGSYY

SEQ ID NO: 490 - protein, 7 aa, synthetic construct
  REGION 1..7: note = HCDR2 of EY 8A
IYTSGST

SEQ ID NO: 491 - protein, 20 aa, synthetic construct
  REGION 1..20: note = HCDR3 of EY 8A
AKGHVISGYDDYYYYYGMDV

SEQ ID NO: 492 - protein, 6 aa, synthetic construct
  REGION 1..6: note = LCDR1 of EY 8A
ALPKQY

SEQ ID NO: 493 - skipped

SEQ ID NO: 494 - protein, 11 aa, synthetic construct
  REGION 1..11: note = LCDR3 of EY 8A
QSADSSGTYWV

SEQ ID NO: 495 - protein, 8 aa, synthetic construct
  REGION 1..8: note = HCDR1 of EY 2A
GYAFTQYW

SEQ ID NO: 496 - protein, 8 aa, synthetic construct
  REGION 1..8: note = HCDR2 of EY 2A
VYPDSSAV

SEQ ID NO: 497 - protein, 23 aa, synthetic construct
  REGION 1..23: note = HCDR3 of EY 2A
SDTAMYYCVRQERGSNTWYAGNS

SEQ ID NO: 498 - protein, 11 aa, synthetic construct
  REGION 1..11: note = LCDR1 of EY 2A
QSLLHTDAYNY

SEQ ID NO: 499 - skipped

SEQ ID NO: 500 - protein, 9 aa, synthetic construct
  REGION 1..9: note = LCDR3 of EY 2A
MQALQTPGT

SEQ ID NO: 501 - protein, 8 aa, synthetic construct
  REGION 1..8: note = HCDR1 of EY 3B
GYAFTQYW

SEQ ID NO: 502 - protein, 8 aa, synthetic construct
  REGION 1..8: note = HCDR2 of EY 3B
VYPDSSAV

SEQ ID NO: 503 - protein, 23 aa, synthetic construct
  REGION 1..23: note = HCDR3 of EY 3B
SDTAMYYCVRQERGSNTWYAGNS

SEQ ID NO: 504 - protein, 11 aa, synthetic construct
  REGION 1..11: note = LCDR1 of EY 3B
QSLLHTDAYNY

SEQ ID NO: 505 - skipped

SEQ ID NO: 506 - protein, 9 aa, synthetic construct
  REGION 1..9: note = LCDR3 of EY 3B
MQALQTPGT

SEQ ID NO: 507 - protein, 8 aa, synthetic construct
  REGION 1..8: note = HCDR1 of EZ 4A
GYSFTSYW

SEQ ID NO: 508 - protein, 8 aa, synthetic construct
  REGION 1..8: note = HCDR2 of EZ 4A
IYPGDSDT

SEQ ID NO: 509 - protein, 12 aa, synthetic construct
  REGION 1..12: note = HCDR3 of EZ 4A
ARSPIAADLFDY

SEQ ID NO: 510 - protein, 6 aa, synthetic construct
  REGION 1..6: note = LCDR1 of EZ 4A
QDISNY

SEQ ID NO: 511 - skipped

SEQ ID NO: 512 - protein, 9 aa, synthetic construct
  REGION 1..9: note = LCDR3 of EZ 4A
QQYDNLLFT

SEQ ID NO: 513 - DNA, 381 bp, synthetic construct
  misc_feature 1..381: note = DNA sequence encoding VH of FM 7B
  source 1..381: mol_type = other DNA
gaggtgcagctggtgcagtctggggctgaggtgaagaagcctggggcctcagtgaaggtttcctgcaaggcttctggatacaccttcactagctatgctatgcattgggtgcgccaggcccccggacaaaggcttgagtggatgggatggatcaacactggcaatggtaacacaaaatattcacagaagttccagggcagagtcaccattaccagggacacatccgcgagcacagcctacatggagctgagcagcctgagatctgaagacacggctgtgtattactgtgcgagagatcccacctattgtagtagtaccagctgctacccttttagctggttcgacccctggggccagggaaccctggtcaccgtctcctca

SEQ ID NO: 514 - DNA, 327 bp, synthetic construct
  misc_feature 1..327: note = DNA sequence encoding VL of FM 7B
  source 1..327: mol_type = other DNA
aattttatgctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtactggtgatcattcttgggtgttcggcggagggaccaagctgaccgtccta

SEQ ID NO: 515 - DNA, 393 bp, synthetic construct
  misc_feature 1..393: note = DNA sequence encoding VH of FD 8B
  source 1..393: mol_type = other DNA
gaggtgcagctgttggagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcaacagctgcagcaattaattgtagtagtaccagctgctactattactactactactacggtatggacgtctggggccaagggaccacggtcaccgtctcctca

SEQ ID NO: 516 - DNA, 336 bp, synthetic construct
  misc_feature 1..336: note = DNA sequence encoding VL of FD 8B
  source 1..336: mol_type = other DNA
gatattgtgatgacccagactccactctcctcacctgtcacccttggacagccggcctccatctcctgcaggtctagtcaaagcctcgtacacagtgatggaaacacctacttgagttggcttcagcagaggccaggccagcctccaagactcctaatttataagatttctagccggttctctggggtcccagacagattcagtggcagtggggcagggacagatttcacactgaaaatcagcagggtggaagctgaggatgtcggggtttattactgcacgcaagctacacaatttccgtacacttttggccaggggaccaaagtggatatcaaa

SEQ ID NO: 517 - DNA, 399 bp, synthetic construct
  misc_feature 1..399: note = DNA sequence encoding VH of EW 9B
  source 1..399: mol_type = other DNA
gaggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagaggatggagtagtaccagctgctaatttgatgatatcgttggaagactactactactacggtatggacgtctggggccaagggaccacggtcaccgtctcctca

SEQ ID NO: 518 - DNA, 321 bp, synthetic construct
  misc_feature 1..321: note = DNA sequence encoding VL of EW 9B
  source 1..321: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcccctcactttcggcggagggaccaaagtggatatcaaa

SEQ ID NO: 519 - DNA, 390 bp, synthetic construct
  misc_feature 1..390: note = DNA sequence encoding VH of FN 12A
  source 1..390: mol_type = other DNA
caggttcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgagatcgggctgtagtagtaccagctgcccctcaaacctttactactactactacggtatggacgtctggggccaagggaccacggtcaccgtctcctca

SEQ ID NO: 520 - DNA, 330 bp, synthetic construct
  misc_feature 1..330: note = DNA sequence encoding VL of FN 12A
  source 1..330: mol_type = other DNA
cagtctgtgctgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtgctttggtgttcggcggagggaccaagctgaccgtccta

SEQ ID NO: 521 - DNA, 369 bp, synthetic construct
  misc_feature 1..369: note = DNA sequence encoding VH of FG 12C
  source 1..369: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatatgagggtgcatgactacggtgactactactttgactactggggccagggaaccctggtcaccgtctcctca

SEQ ID NO: 522 - DNA, 324 bp, synthetic construct
  misc_feature 1..324: note = DNA sequence encoding VL of FG 12C
  source 1..324: mol_type = other DNA
tcctatgagctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggatagtagtagtgatcatccggtattcggcggagggaccaagctgaccgtccta

SEQ ID NO: 523 - DNA, 360 bp, synthetic construct
  misc_feature 1..360: note = DNA sequence encoding VH of FI 1C
  source 1..360: mol_type = other DNA
gaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggctgtgtattactgtgcgaggcgcagtaacaggtttttgattgcttttgatatctggggccaagggacaatggtcaccgtctcttca

SEQ ID NO: 524 - DNA, 333 bp, synthetic construct
  misc_feature 1..333: note = DNA Sequence encoding VL of FI 1C
  source 1..333: mol_type = other DNA
cagtctgccctgactcagcctgcctccgtgtctgggtctcctggacagacgatcaccatctcctgcactggaaccagcagtgacgttggtggttataactatgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcagctcatatacaagcagcagcactctcgtggtattcggcggagggaccaagctgaccgtccca

SEQ ID NO: 525 - DNA, 354 bp, synthetic construct
  misc_feature 1..354: note = DNA sequence encoding VH of FI 4A
  source 1..354: mol_type = other DNA
gaggtgcagctggtggagtctgggggaggcctggtcaagcctggggggtccctgagactctcctgtgcagcctctggattcaccttcagtcgctttagcatgaactgggtccgccaggctccagggaaggggctggagtgggtctcctccattagtagtagtggtagttacatatacttcgcagactcagtgaagggccgattcaccatctccagagacaacgccaagaactcactgtatctgcaaatgaacagcctgagagccgaggacacggctatatattactgtgcgacttatttatttggtgactcccatacctactggggccagggaaccctggtcaccgtctcctca

SEQ ID NO: 526 - DNA, 336 bp, synthetic construct
  misc_feature 1..336: note = DNA sequence encoding VL of FI 4A
  source 1..336: mol_type = other DNA
cagtctgtgctgacgcagccccactctgtgtcggagtctccggggaagacggtaaccatctcctgcaccggcagcagtggcagcattgccagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcagcaatctccattgggtgttcggcggagggaccaagctgaccgtccta

SEQ ID NO: 527 - DNA, 375 bp, synthetic construct
  misc_feature 1..375: note = DNA sequence encoding VH of FD 1E
  source 1..375: mol_type = other DNA
gaggtgcagctgttggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagcttagcagcagctggccccgaaacctactattactacggtatggacgtctggggccaagggacaatggtcaccgtctcttca

SEQ ID NO: 528 - DNA, 315 bp, synthetic construct
  misc_feature 1..315: note = DNA sequence encoding VL of FD 1E
  source 1..315: mol_type = other DNA
tcctatgagctgacacagccaccctcagtgtccgtgtccccaggacagacagccagcatcacctgctctggagataaattgggggataaatatgcttgctggtatcagcagaagccaggccagtcccctgtgctggtcatctatcaagatagcaagcggccctcagggatccctgagcgattctctggctccaactctgggaacacagccactctgaccatcagcgggacccaggctatggatgaggctgactattactgtcaggcgtgggacagcagcgtggtattcggcggagggaccaagctgaccgtccta

SEQ ID NO: 529 - DNA, 375 bp, synthetic construct
  misc_feature 1..375: note = DNA sequence encoding VH of FD 11E
  source 1..375: mol_type = other DNA
gaagtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagcttagcagcagctggccccgaaacctactactactacggtatggacgtctggggccaagggacaatggtcaccgtctcttca

SEQ ID NO: 530 - DNA, 315 bp, synthetic construct
  misc_feature 1..315: note = DNA sequence encoding VL of FD 11E
  source 1..315: mol_type = other DNA
tcctatgagctgactcagccaccctcagtgtccgtgtccccaggacagacagccagcatcacctgctctggagataaattgggggataaatatgcttgctggtatcagcagaagccaggccagtcccctgtgctggtcatctatcaagatagcaagcggccctcagggatccctgagcgattctctggctccaactctgggaacacagccactctgaccatcagcgggacccaggctatggatgaggctgactattactgtcaggcgtgggacagcagcgtggtattcggcggagggaccaagctgaccgtccta

SEQ ID NO: 531 - DNA, 381 bp, synthetic construct
  misc_feature 1..381: note = DNA sequence encoding VH of FN 2C
  source 1..381: mol_type = other DNA
gaggtgcagctgttggagtccgggggaggcgtggtccagcctgggaggtccctgagactctcctgtgcagcctctggattcaacttcaataactatggcatgcactgggtccgccaggctccaggcaaggggctggagtgggtggctactatttcatatgaaggaagtaaaaaattttatgtagactccgtgaagggccgattcaccatctccaaagacaattccaagaacacgctgtatctgcagatgaacagcctgagagttgacgacacggctttttattactgtgcgaaacggagggaaatattttggttgggggagccacctctctcggatgcttttgatttctggggccaagggacaatggtcaccgtctcttca

SEQ ID NO: 532 - DNA, 333 bp, synthetic construct
  misc_feature 1..333: note = DNA sequence encoding VL of FN 2C
  source 1..333: mol_type = other DNA
cagtctgtgctgactcagccgccctcagtgtctggggccccagggcagagggtcaccatctcctgcactgggagcggctccaacatcggggcaggttatgatgtacactggtaccagttccttccaggaacagccccccaactcctcatctatggtaacaacaatcgtccctcaggggtccctgaccgattctctggctccaagtctggcacctcagcctccctggccatcactgggctccaggctgaagatgaggctgattattactgccagtcctatgacagcagcctgagtggttcggtgttcggcggagggaccaagctgaccgtccta

SEQ ID NO: 533 - DNA, 363 bp, synthetic construct
  misc_feature 1..363: note = DNA sequence encoding VH of EY 6A
  source 1..363: mol_type = other DNA
gaagtgcagctggtggagtctgggggaggcgtggtccagcctgggaggtccctgagactctcctgtgcagcctctgcattcaccttcagtagctatgacatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagatgggggaaagctatgggtgtactactttgactactggggccagggaaccacggtcaccgtctcctca

SEQ ID NO: 534 - DNA, 324 bp, synthetic construct
  misc_feature 1..324: note = DNA sequence encoding VL of EY 6A
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccctcgcgctcactttcggcggagggaccaaagtggatatcaaa

SEQ ID NO: 535 - DNA, 363 bp, synthetic construct
  misc_feature 1..363: note = DNA sequence encoding VH of EY 6A-1
  source 1..363: mol_type = other DNA
gaagtgcagctggtggagtctgggggaggcgtggtccagcctgggaggtccctgagactctcctgtgcagcctctgcattcaccttcagtagctatgacatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagatgggggaaagctatgggtgtactactttgactactggggccagggaaccctggtcaccgtctcctca

SEQ ID NO: 536 - DNA, 324 bp, synthetic construct
  misc_feature 1..324: note = DNA sequence encoding VL of EY 6A-1
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccctcgcgctcactttcggcggagggaccaaggtggagatcaaa

SEQ ID NO: 537 - DNA, 384 bp, synthetic construct
  misc_feature 1..384: note = DNA sequence encoding VH of FD 11D
  source 1..384: mol_type = other DNA
gaagtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagaaggagccggcagtggctggtaccgccaccacaagccgggctactactttgactactggggccagggaaccctggtcaccgtctcctca

SEQ ID NO: 538 - DNA, 324 bp, synthetic construct
  misc_feature 1..324: note = DNA sequence encoding VL of FD 11D
  source 1..324: mol_type = other DNA
gaaattgtgttgacacagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctctaacgttcggccaagggaccaaggtggaaatcaaa

SEQ ID NO: 539 - DNA, 375 bp, synthetic construct
  misc_feature 1..375: note = DNA sequence encoding VH of EW 9C
  source 1..375: mol_type = other DNA
gaagtgcagctggtggagtcgggggggggcgtggtccagcctggggcgtccctgagactctcctgcgtagcctccggattcacctttaataattttggattccactgggtccgccaggctccaggcaaggggctggagtggctggcagtgatatcatatgagggaagtaagacttactatgcagagtcgctgaagggccgcttcaccatctccagagacacttccaagaacacggtgtatctgcagatgaacagcctgagggctgaggacacggctgtctattactgtgcgcgggcgacttcaattttttggtttggagagggccgtaactggttcgacccctggggccaagggacaatggtcaccgtctcttca

SEQ ID NO: 540 - DNA, 321 bp, synthetic construct
  misc_feature 1..321: note = DNA sequence encoding VL of EW 9C
  source 1..321: mol_type = other DNA
gaaattgtgttgacgcagtctccaggcaccgtgtctttgtctgcaggggaaagagccaccctctcctgcagggccagtcagaatgttggcaccgacttagcctggtatgttcagagacctggccaggctcccaggctcctcatctatgatacatccaataaggccactggcatcccagccaggtttagtggtcgagggtctgggacagacttcactctcaccatcgacagcctagagcctgaagactttgcagtttattactgtcaacagcgtagcaactggccgctcactttcggcggagggaccaaggtggaaatcaga

SEQ ID NO: 541 - DNA, 375 bp, synthetic construct
  misc_feature 1..375: note = DNA sequence encoding VH of EW 9C-1
  source 1..375: mol_type = other DNA
gaggtgcagctggtggagtcgggggggggcgtggtccagcctggggcgtccctgagactctcctgcgtagcctccggattcacctttaataattttggattccactgggtccgccaggctccaggcaaggggctggagtggctggcagtgatatcatatgagggaagtaagacttactatgcagagtcgctgaagggccgcttcaccatctccagagacacttccaagaacacggtgtatctgcagatgaacagcctgagggctgaggacacggctgtctattactgtgcgcgggcgacttcaattttttggtttggagagggccgtaactggttcgacccctggggccagggagccctggtcaccgtctcctca

SEQ ID NO: 542 - DNA, 321 bp, synthetic construct
  misc_feature 1..321: note = DNA sequence encoding VL of Ew 9C-1
  source 1..321: mol_type = other DNA
gaaatagtgatgacgcagtctccaggcaccgtgtctttgtctgcaggggaaagagccaccctctcctgcagggccagtcagaatgttggcaccgacttagcctggtatgttcagagacctggccaggctcccaggctcctcatctatgatacatccaatagggccactggcatcccagccaggtttagtggtcgagggtctgggacagacttcactctcaccatcgacagcctagagcctgaagactttgcagtttattactgtcaacagcgtagcaactggccgctcactttcggcggagggaccaaggtggaaatcaaa

SEQ ID NO: 543 - DNA, 363 bp, synthetic construct
  misc_feature 1..363: note = DNA sequence encoding VH of FD 1D
  source 1..363: mol_type = other DNA
gaagtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagagcgggttcggggagctacctcaactggttcgacccctggggccagggaaccctggtcaccgtctcctca

SEQ ID NO: 544 - DNA, 321 bp, synthetic construct
  misc_feature 1..321: note = DNA sequence encoding VL of FD 1D
  source 1..321: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgatcaccttcggccaagggacacgactggagattaaa

SEQ ID NO: 545 - DNA, 363 bp, synthetic construct
  misc_feature 1..363: note = DNA sequence encoding VH of FG 7A
  source 1..363: mol_type = other DNA
gaggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagatcccatagtgggagctaccgagcctcccttgactactggggccagggaaccacggtcaccgtctcctca

SEQ ID NO: 546 - DNA, 327 bp, synthetic construct
  misc_feature 1..327: note = DNA sequence encoding VL of FG 7A
  source 1..327: mol_type = other DNA
gacatccagttgacccagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctttgtacacttttggccaggggaccaaggtggaaatcaaa

SEQ ID NO: 547 - DNA, 363 bp, synthetic construct
  misc_feature 1..363: note = DNA sequence encoding VH of FM 1A
  source 1..363: mol_type = other DNA
gaggtgcagctgttggagtctgggggaggcgtggtccagcctgggaggtccctgagactctcctgtgcagcgtctggattcaccttcagtagctatggcatgcactgggtccgccaggctccaggcaaggggctggaatgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagagggggcagtgggagctactagggggtttgactactggggccagggaaccctggtcaccgtctcctca

SEQ ID NO: 548 - DNA, 324 bp, synthetic construct
  misc_feature 1..324: note = DNA sequence encoding VL of FM 1A
  source 1..324: mol_type = other DNA
tcctatgagctgacacagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcagggggtattcggcggagggaccaagctgaccgtccta

SEQ ID NO: 549 - DNA, 381 bp, synthetic construct
  misc_feature 1..381: note = DNA sequence encoding VH of FD 11A
  source 1..381: mol_type = other DNA
gaagtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgaaaggccccgatattttgactggttattataactactactactacggtatggacgtctggggccaagggacaatggtcaccgtctcttca

SEQ ID NO: 550 - DNA, 339 bp, synthetic construct
  misc_feature 1..339: note = DNA sequence encoding VL of FD 11A
  source 1..339: mol_type = other DNA
cagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggtttctatgtggtattcggcggagggaccaagctgaccgtccta

SEQ ID NO: 551 - DNA, 384 bp, synthetic construct
  misc_feature 1..384: note = DNA sequence encoding VH of FN 8C
  source 1..384: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatcatatgatggaagtaataaatactatgcagacaccgtgaagggccgattcaccatctccagagacaattccaagaacacgctgtatctgcaaatgaacagcctgagagccgaggacacggctgtgtattactgtgcgagagaacggacgtattacgatattttgactggttatagacactactacggtatggacgtctggggccaagggaccacggtcaccgtctcctca

SEQ ID NO: 552 - DNA, 324 bp, synthetic construct
  misc_feature 1..324: note = DNA sequence encoding VL of FN 8C
  source 1..324: mol_type = other DNA
tcctatgagctgacacagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcattgggtgttcggcggagggaccaagctgaccgtccta

SEQ ID NO: 553 - DNA, 366 bp, synthetic construct
  misc_feature 1..366: note = DNA sequence encoding VH of FD 5E
  source 1..366: mol_type = other DNA
gaagtgcagctggtggagtctgggggagtcgtggtacagcctggggggtccctgagactctcctgtgcagcctctggattcacctttgatgattatgccatgcactgggtccgtcaagctccggggaagggtctggagtgggtctctcttattagttgggatggtggtagcacctactatgcagactctgtgaagggtcgattcaccatctccagagacaacagcaaaaactccctgtatctgcaaatgaacagtctgagagctgaggacaccgccttgtattactgtgcaaaagattcggtacgatttcggtactactacggtatggacgtctggggccaagggaccacggtcaccgtctcctca

SEQ ID NO: 554 - DNA, 321 bp, synthetic construct
  misc_feature 1..321: note = DNA sequence encoding VL of FD 5E
  source 1..321: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctctcactttcggccctgggaccaaagtggatatcaaa

SEQ ID NO: 555 - DNA, 387 bp, synthetic construct
  misc_feature 1..387: note = DNA sequence encoding VH of FD 5D
  source 1..387: mol_type = other DNA
gaagtgcagctggtggagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatatactacgcagaccctgtgaagggccgattcaccatctccagagacaacgccaagaactcactgtatctgcaaatgaacagcctgagagccgaggacacggctgtgtattactgtgcgagtcccggagggatcacagcagctggtacatcagttctttttgggtactacggtatggacgtctggggccaagggacaatggtcaccgtctcttca

SEQ ID NO: 556 - DNA, 342 bp, synthetic construct
  misc_feature 1..342: note = DNA sequence encoding VL of FD 5D
  source 1..342: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcccatcacgtggacgttcggccaagggaccaaagtggatatcaaa

SEQ ID NO: 557 - DNA, 351 bp, synthetic construct
  misc_feature 1..351: note = DNA sequence encoding VH of FI 3A
  source 1..351: mol_type = other DNA
gaggtgcagctgttggagtctggaggaggcttgatccagcctggggggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagagatcacgtgcggcccgggatgaatatctggggccaagggacaatggtcaccgtctcttca

SEQ ID NO: 558 - DNA, 321 bp, synthetic construct
  misc_feature 1..321: note = DNA sequence encoding VL of FI 3A
  source 1..321: mol_type = other DNA
gccatccggatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatcttccggtcactttcggcggagggaccaaagtggatatcaaa

SEQ ID NO: 559 - DNA, 342 bp, synthetic construct
  misc_feature 1..342: note = DNA sequence encoding VH of FD 10A
  source 1..342: mol_type = other DNA
gaagtgcagctggtggagtccgggggaggcttagttcagcctggggggtccctgagactctcctgtgcagcctctggattcaccttcagtagctactggatgcactgggtccgccaagctccagggaaggggctggtgtgggtctcacgtattaatagtgatgggagtagcacaagctacgcggactccgtgaagggccgattcaccatctccagagacaacgccaagaacacgctgtatctgcaaatgaacagtctgagagccgaggacacggctgtgtattactgtgcaaacatggcttttgatatctggggccaagggacaatggtcaccgtctcttca

SEQ ID NO: 560 - DNA, 339 bp, synthetic construct
  misc_feature 1..339: note = DNA sequence encoding VL of FD 10A
  source 1..339: mol_type = other DNA
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactccgatcaccttcggccaagggacacgactggagattaaa

SEQ ID NO: 561 - DNA, 381 bp, synthetic construct
  misc_feature 1..381: note = DNA sequence encoding VH of FJ 4E
  source 1..381: mol_type = other DNA
caggtgcagctggtggagtcgggcccaggactggtgaagccttcagagaccctgtccctcacctgcagcgtctctggtggctccatcagtagtggtacttactactggagctggatccgccagcagccagggaagggcctggagtggattgggtacatctataacactgggagaccctactacaacccgtttctcaagagtcgaattaccatatcagtggactcgtctaagaaccagttctccctgaagctgacctctgtgactgccgcggacacggccgtgtattattgtgcgagagatgaatatgattcctctgattcggggattcaaggccactggttcgacccctggggccagggaaccctggtcaccgtctcctca

SEQ ID NO: 562 - DNA, 321 bp, synthetic construct
  misc_feature 1..321: note = DNA sequence encoding VL of FJ 4E
  source 1..321: mol_type = other DNA
gccatccggatgacccagtctccatcctccctgtctgcatctgtgggagacagagtcaccatcacttgccggacaagtcagaacattaacagttttttaaattggtatcagcagaaaccagggaaaggccctaacctcctgatctatggtgcattcactttacaaagtggggtcccatcaaggttcagtggcagtggatctgggacagatttcacactcaccatcagcagtctacaacctgaagattttgcaacttacttctgtcaacagagttacagtaccccgtggacgttcggccaagggaccaaggtggaaatcaaa

SEQ ID NO: 563 - DNA, 378 bp, synthetic construct
  misc_feature 1..378: note = DNA sequence encoding VH of FJ 1C
  source 1..378: mol_type = other DNA
caggtgcagctgcaggagtcgggcccaggactggtgaagccttcggagaccctgtccctcacctgcactgtctctggttactccatcagcagtggttactactggggctggatccggcagcccccagggaaggggctggagtggattgggagtatctatcatagtgggagcacctactacaacccgtccctcaagagtcgagtcaccatatcagtagacacgtccaagaaccagttctccctgaagctgagctctgtgaccgccgcagacacggccgtgtattactgtgcgagagataaggccttactatggttcggggagttatttaccaacctctttgactactggggccagggaaccctggtcaccgtctcctca

SEQ ID NO: 564 - DNA, 330 bp, synthetic construct
  misc_feature 1..330: note = DNA sequence encoding VL of FJ 1C
  source 1..330: mol_type = other DNA
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctaattattactgcagctcatatacaagcagcagcactctggtattcggcggagggaccaagctgaccgtccta

SEQ ID NO: 565 - DNA, 357 bp, synthetic construct
  misc_feature 1..357: note = DNA sequence encoding VH of EW 8B
  source 1..357: mol_type = other DNA
gtccagctggtacaggagtcgggcccaggactggtgaagccttcggagaccctgtccctcacctgcactgtctctggtggctccatcagcagtagtggttactactggggctggatccggcagcccccagggaaggggctggagtggattgcgaatttttattttactgggagtgtctactccaacccgtccctcaagagtcgagtcaccatatccgttgacacgtccaagaaccagctctccctgaaattgagctatctgaccgccgcagacacggctgtatattactgtgcgagacaagaggtttggggtggttttgatatctggggccaagggacaatggtcaccgtctcttca

SEQ ID NO: 566 - DNA, 321 bp, synthetic construct
  misc_feature 1..321: note = DNA sequence encoding VL of EW 8B
  source 1..321: mol_type = other DNA
gaaattgtgttgacgcagtctccaggcaccctgtctttgtctccaggggaaagagtcaccctctcctgcagggccagtcagagtcttggcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtatttctgtcagcagtatggtagctcaccgacgttcggccaagggaccaaggtggaaatcaaa

SEQ ID NO: 567 - DNA, 387 bp, synthetic construct
  misc_feature 1..387: note = DNA sequence encoding VH of FD 11C
  source 1..387: mol_type = other DNA
gaggtgcagctgttggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatcagtagacacgtccaagaaccagttctccctgaagctgagctctgtgaccgccgcggacacggccgtgtattactgtgcgagggagtattactatggttcggagacaaaaaaatactactactactacggtatggacgtctggggccaagggaccacggtcaccgtctcctca

SEQ ID NO: 568 - DNA, 315 bp, synthetic construct
  misc_feature 1..315: note = DNA sequence encoding VL of FD 11C
  source 1..315: mol_type = other DNA
tcctatgagctgactcagccaccctcagtgtccgtgtccccaggacagacagccagcatcacctgctctggagataaattgggggataaatatgcttgctggtatcagcagaagccaggccagtcccctgtgctggtcatctatcaagatagcaagcggccctcagggatccctgagcgattctctggctccaactctgggaacacagccactctgaccatcagcgggacccaggctatggatgaggctgactattactgtcaggcgtgggacagcagcaccgtattcggcggagggaccaagctgaccgtccta

SEQ ID NO: 569 - DNA, 372 bp, synthetic construct
  misc_feature 1..372: note = DNA sequence encoding VH of FD 7C
  source 1..372: mol_type = other DNA
cagctgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagattataggttcggggagttatttggaaggtttgcctggttcgacccctggggccagggaaccctggtcaccgtctcctca

SEQ ID NO: 570 - DNA, 321 bp, synthetic construct
  misc_feature 1..321: note = DNA sequence encoding VL of FD 7C
  source 1..321: mol_type = other DNA
gacatccagttgacccagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctagggctttcggccctgggaccaaggtggaaatcaga

SEQ ID NO: 571 - DNA, 378 bp, synthetic construct
  misc_feature 1..378: note = DNA sequence encoding VH of FD 7D
  source 1..378: mol_type = other DNA
gaggtgcagctggtggagtccggagcagaggtgaaaaagcccggggagtctctgaggatctcctgtaagggttctggatacagctttaccagctactggatcagctgggtgcgccagatgcccgggaaaggcctggagtggatggggaggattgatcctagtgactcttataccaactacagcccgtccttccaaggccacgtcaccatctcagctgacaagtccatcagcactgcctacctgcagtggagcagcctgaaggcctcggacaccgccatgtattactgtgcgagacattctgattgtagtagtaccagctgctatttcgtcgatgcttttgatatctggggccaagggacaatggtcaccgtctcttca

SEQ ID NO: 572 - DNA, 324 bp, synthetic construct
  misc_feature 1..324: note = DNA sequence encoding VL of FD 7D
  source 1..324: mol_type = other DNA
tcctatgagctgactcagccaccctcggtgtcagtgtccccaggacagacggccaggatcacctgctctggagatgcattgccaaagcaatatgcttattggtaccagcagaagccaggccaggcccctgtgctggtgatatataaagacagtgagaggccctcagggatccctgagcgattctctggctccagctcagggacaacagtcacgttgaccatcagtggagtccaggcagaagacgaggctgactattactgtcaatcagcagacagcagtggtacttatgtggtattcggcggagggaccaagctgaccgtccta

SEQ ID NO: 573 - DNA, 369 bp, synthetic construct
  misc_feature 1..369: note = DNA sequence encoding VH of FJ 10B
  source 1..369: mol_type = other DNA
caggttcagctggtgcagtccggagcagaggtgaaaaagcccggggagtctctgaggatctcctgtaagggttctggatacagctttaccagctactggatcagctgggtgcgccagatgcccgggaaaggcctggagtggatggggaggattgatcctagtgactcttataccaactacagcccgtccttccaaggccacgtcaccatctcagctgacaagtccatcagcactgcctacctgcagtggagcagcctgaaggcctcggacaccgccatgtattactgtgcgagactagacccccgttatgggcccgactactacggtatggacgtctggggccaagggaccacggtcaccgtctcctca

SEQ ID NO: 574 - DNA, 321 bp, synthetic construct
  misc_feature 1..321: note = DNA sequence encoding VL of FJ 10B
  source 1..321: mol_type = other DNA
gccatccggatgacccagtctccatcctccctgtctgcatctgtgggagacagagtcaccatcacttgccgggcaagtcagagcattagcagctatttaaattggtatcagcagaaaccagggaaagcccctaagctcctgatccatgctgcatccagtttgcaaagtggggtcccatcaaggttcagtggcagtggatctgggacagatttcactctcaccatgagcagtctgcaacctgaagattttgcaacttactactgtcaacagagttacagtaccccgctcactttcggcggagggaccaaggtggaaatcaga

SEQ ID NO: 575 - DNA, 387 bp, synthetic construct
  misc_feature 1..387: note = DNA sequence encoding VH of FB 9D
  source 1..387: mol_type = other DNA
caggtgcagctggtggagtctggagcagaagtgaaaaagcccggggagtctttgaagatctcctgtcagggttctggatacaggtttaacagttattggatcgcctgggtgcgccaaatgcccgggaaaggcctggagtggatggggagcatctttcctactgactctgatgtcagatataacccgtccttccaaggccaggtcaccatttcggccgacaagtccatcagtttcgcctacctgcagtggagcagcctgaaggcctcggacaccgccatatattattgtgcgagacattgggccagtatggttcggggagtaattcgtgccagtcattattatggcatggacgtctggggccaagggaccacggtcaccgtctcctca

SEQ ID NO: 576 - DNA, 336 bp, synthetic construct
  misc_feature 1..336: note = DNA sequence encoding VL of FB 9D
  source 1..336: mol_type = other DNA
cagcctgtgctgactcagcctccctccgcgtccgggtctcctggacagtcagtcaccatctcctgctctggagccagcagtgacattggtaaatataactatgtctcctggtaccaacagctcccaggcaaagcccccaaactcctgatttatgaggtcactaagcggccctcaggggtccctgatcgcttctctggctccaagtctggcaacacggcctccctgaccgtctctgggctccaggctgacgatgaggctgattattactgcagctcttatgcatttggaggcagcgacacccgggtgttcggcggagggaccaagctgaccgtccta

SEQ ID NO: 577 - DNA, 387 bp, synthetic construct
  misc_feature 1..387: note = DNA sequence encoding VH of FB 1E
  source 1..387: mol_type = other DNA
gaagtgcagctggtggagtctggagcagaagtgaaaaagcccggggagtctctgaagatctcctgtcagggttctggatacaggtttaatagttattggatcgcctgggtgcgccagatgcccgggaaaggcctggagtggatggggagcatctttcctactgactctgatatcagatataacccgtccttccaaggccaggtcaccatctcagccgacaagtccatcagtatcgcctacctgcagtggagcagcctgaaggcctcggacaccgccatatattattgtgcgagacattgggccagtatggttcggggagtaattcgtgccagtcattattatggcatggacgtctggggccaagggaccacggtcaccgtctcctca

SEQ ID NO: 578 - DNA, 336 bp, synthetic construct
  misc_feature 1..336: note = DNA sequence encoding VL of FB 1E
  source 1..336: mol_type = other DNA
cagtctgtgctgactcagcctccctccgcgtccgggtctcctggacagtcagtcaccatctcctgctctggagccagcagtgacattggtgattataactatgtctcctggtaccaacagctcccaggcaaagcccccaaactcctgatttatgaggtcactaagcggccctcaggggtccctgatcgcttctctggctccaagtctggcaacacggcctccctgaccgtctctgggctccaggctgacgatgaggctgattactactgcagctcttatgcatttggaggcagcgacatccgggtgttcggcggagggaccaagctggccgtccta

SEQ ID NO: 579 - DNA, 369 bp, synthetic construct
  misc_feature 1..369: note = DNA sequence encoding VH of EZ 7A
  source 1..369: mol_type = other DNA
gaagtgcagctggtggagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagaggatgggtttatcggggcttcccttactacggtatggacgtctggggccaagggacaatggtcaccgtctcttca

SEQ ID NO: 580 - DNA, 330 bp, synthetic construct
  misc_feature 1..330: note = DNA sequence encoding VL of EZ 7A
  source 1..330: mol_type = other DNA
cagtctgccctgactcagcctcgctcagtgtccgggtctcctggacagtcagtcaccatctcctgcactggaaccagcagtgatgttggtggttataactatgtctcctggtaccaacagcacccaggcaaagcccccaaactcatgatttatgatgtcagtaagcggccctcaggggtccctgatcgcttctctggctccaagtctggcaacacggcctccctgaccatctctgggctccaggctgaggatgaggctgattattactgctgctcatatgcaggcagctacactttggtgttcggcggagggaccaagctgaccgtccta

SEQ ID NO: 581 - DNA, 360 bp, synthetic construct
  misc_feature 1..360: note = DNA sequence encoding VH of EZ 9B
  source 1..360: mol_type = other DNA
gaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggacggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagagaggggcctacggtgacttggtggttcgacccctggggccagggaaccctggtcaccgtctcctca

SEQ ID NO: 582 - DNA, 330 bp, synthetic construct
  misc_feature 1..330: note = DNA sequence encoding VL of EZ 9B
  source 1..330: mol_type = other DNA
tcctatgagctgacacagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatccttcttgggtgttcggcggagggaccaagctgaccgtccta

SEQ ID NO: 583 - DNA, 366 bp, synthetic construct
  misc_feature 1..366: note = DNA sequence encoding VH of EZ 11C
  source 1..366: mol_type = other DNA
gaagtgcagctggtggagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcaacaactacggtgactaccccaaccgcaaactggttcgacccctggggccagggaaccctggtcaccgtctcctca

SEQ ID NO: 584 - DNA, 330 bp, synthetic construct
  misc_feature 1..330: note = DNA sequence encoding VL of EZ 11C
  source 1..330: mol_type = other DNA
cagtctgccctgactcagccgccctcagtgtctgcggccccaggacagaaggtcaccatctcctgctctggaagcagctccaacattgggaataattatgtatcctggtaccagcagctcccaggaacagcccccaaactcctcatctatgaaaataataagcgaccctcagggattcctgaccgattctctggctccaagtctggcacgtcagccaccctgggcatcaccggactccagactggggacgaggccgattattactgcggaacatgggatagcagcctgcgtcaggtagtgttcggcggagggaccaagctgaccgtccta

SEQ ID NO: 585 - DNA, 351 bp, synthetic construct
  misc_feature 1..351: note = DNA sequence encoding VH of FD 9B
  source 1..351: mol_type = other DNA
gaagtgcagctggtggagtctgggggaggcttggtccagcctggggggtccctgagactctcctgtgcaggcactggattcagctttagtagatattggatgaattgggtccgccaggctccagggaaggggctggagtgggtggccaacatggacccagatggaggtgcgaaatactatctggactctgtgaaggggcgattcaccatctccggagacaacgccaagaactcattgtatctgcaaatgaacagactgagagccgaggacacggctgtgtattactgtgtcaaattcgggcggtcggaaggcttgttttggggccgtggcaccctggtcaccgtctcctca

SEQ ID NO: 586 - DNA, 330 bp, synthetic construct
  misc_feature 1..330: note = DNA sequence encoding VL of FD 9B
  source 1..330: mol_type = other DNA
caggctgtggtgacccaggagccctcactgactgtgtccccaggagggacagtcactctcacctgtggctccagcactggagctgtcaccagtggtcattatccctactggttccagcagaagcctggccaagcccccaggacactgatttatgatacaagcaacaaacactcctggacacctgcccgattctcaggctccctccttgggggcaaagctgccctgaccctttcgggtgcgcagcctgaggatgaggctgactattactgcttcctcacctatgttggtgctcggaggttattcggcggagggaccaagctgaccgtccta

SEQ ID NO: 587 - DNA, 366 bp, synthetic construct
  misc_feature 1..366: note = DNA sequence encoding VH of EZ 11A
  source 1..366: mol_type = other DNA
gaagtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagagatgactatagtgggagctactattgggaatttgactactggggccagggaaccctggtcaccgtctcctca

SEQ ID NO: 588 - DNA, 333 bp, synthetic construct
  misc_feature 1..333: note = DNA sequence encoding VL of EZ 11A
  source 1..333: mol_type = other DNA
cagcctgtgctgactcaatcgccctctgcctctgcctccctgggagcctcggtcaagctcacctgcactctgagcagtgggcacagcagctacgccatcgcatggcatcagcagcagccagagaagggccctcggtacttgatgaagcttaacagtgatggcagccacagcaagggggacgggatccctgatcgcttctcaggctccagctctggggctgagcgctacctcaccatctccagcctccagtctgaggatgaggctgactattactgtcagacctggggcactggcatttgggtgttcggcggagggaccaagctgaccgtccta

SEQ ID NO: 589 - DNA, 345 bp, synthetic construct
  misc_feature 1..345: note = DNA sequence encoding VH of EW 10C
  source 1..345: mol_type = other DNA
gaagtgcagctggtggagtctgggggaggcttggtccagccgggggggtccctgagactctcctgtgcagcctctggattcaccttcaacaagtacaagatgacctgggtccgccaggctccagggaaggggctggagtgggtggccaacataaaggaagatggaagtgagaaaaactatgtggactctgtgaagggccgattcaccatctccagagacaacgccaggaactcagtatatatgcacctgaacaacttgagagtcgaggacacggccgtgtattactgtgcgagagggcggaccctcggcgactggggccagggaaccacggtcaccgtctcctca

SEQ ID NO: 590 - DNA, 339 bp, synthetic construct
  misc_feature 1..339: note = DNA sequence encoding VL of EW 10C
  source 1..339: mol_type = other DNA
gccatccggatgactcagtctccactctccctgcccgtcacccttggacagccggcctccatctcctgcaggtctagtcaaagcctcgtgcacagtgatggaaacacctacttgaattggtttcagcagaggccaggccaatctccaaggcgcctaatttataaggtttctaaccgggactctggggccccagacagattcagcggcagtgggtcaggcactgatttcacactgaaaatcagcagggtggaggctgaggatgttggggtttattattgcatgcaaggtacacactggcctccgattactttcggccctgggaccaaagtggatatcaaa

SEQ ID NO: 591 - DNA, 363 bp, synthetic construct
  misc_feature 1..363: note = DNA sequence encoding VH of EY 12B
  source 1..363: mol_type = other DNA
gaagtgcagctggtggagtccgggggagacttggtacagcctggcaggtccctgagactctcctgtgcagcctctggattcacctttgattattttgccatgcactgggtccggcaagttccagggaagggcctggagtgggtctcaggtattcgttggaatagtgaaaccataggctatgcggactctgtgaagggccggttcaccatctccagagacaacgccaagaaatcactgtatctggaaatgaacagtctgagaagtgaggacacggccttctattactgtgcaaaaggtcggagtggctacggccacactgcttttgatgtctggggccaagggacaatggtcaccgtctcttca

SEQ ID NO: 592 - DNA, 333 bp, synthetic construct
  misc_feature 1..333: note = DNA sequence encoding VL of EY 12B
  source 1..333: mol_type = other DNA
cagtctgccctgactcagccgccctcagtgtctggggccccagggcagagggtcaccatctcctgcactgggagcagctccaacatcggggcaaattatgatgtacactggtatcagcggcttccaggaacagcccccaaactcctcatctatgataacaacaatcggccctcgggggtccctgaccgattctctggctccaagtctggcacctcagcctccctggccatcgctgggctccaggctgaggatgaggctgattattactgccagtcctatgacagcagcctgagtgcttcagtattcggcggagggaccaagctgaccgtccta

SEQ ID NO: 593 - DNA, 366 bp, synthetic construct
  misc_feature 1..366: note = DNA sequence encoding VH of EZ 8C
  source 1..366: mol_type = other DNA
gaagtgcagctggtggagtctgggggaggcctggtcaagcctggggggtccctgagactctcctgtgcagcctctggattcaccttcagttcctataccatgaactgggtccgccaggctccagggaaggggctggagtgggtctcatcctttattgctaatagtgattacaagttctacgcagactcagtgaagggccgattcaccatctccagagacaacgccaagagctcactgtatctgcaaatgaacagcctgagagccgaggacacggctgtgtattactgtgcgagagaactaaccagttatggttcccacgatgcttttgatatctggggccaagggacaatggtcaccgtctcttca

SEQ ID NO: 594 - DNA, 330 bp, synthetic construct
  misc_feature 1..330: note = DNA sequence encoding VL of EZ 8C
  source 1..330: mol_type = other DNA
cagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcttgatttatgaggtcactaatcggccctcaggggtttctgatcgcttctctggctccaagtctgccaatgtggcgtccctgaccatctctgggctccaggctgaggacgaggctgattattactgcagctcatatacaaccaccgactccgtggttttcggcggagggaccaagctgaccgtccta

SEQ ID NO: 595 - DNA, 348 bp, synthetic construct
  misc_feature 1..348: note = DNA sequence encoding VH of EY 9C
  source 1..348: mol_type = other DNA
gaagtgcagctggtggagtctgggggaggcctggtcaagcctggggggtccctgagactctcctgtgcagcctctgaattcaccttcagtagctataccttgaactgggtccgccaggctccagggaaggggctggagtgggtctcatccattagtactagtagtgcttacatatactacgcagactcagtgaagggccgattcaccatctccagagacaacgccaagaagtcactgtctctgcaaatgaacagcctgacagccgaggacacggctgtctattactgtgcgacttggggcggtgccccctttgactactggggccaagggacaatggtcaccgtctca

SEQ ID NO: 596 - DNA, 336 bp, synthetic construct
  misc_feature 1..336: note = DNA sequence encoding VL of EY 9C
  source 1..336: mol_type = other DNA
cagtctgtgctgactcagcctgcctccgtgtctgggtctcctggacggtcgatcaccatctcctgcactgaaaccagcagtgatgttgggacttataaccttgtctcctggtaccaacagcacccaggcaaagcccccaaactcatgatttttgacgacaataagcggccctcaggggtttctaatcgcttctctggctccaagtctggcaacacggcctccctgacaatctctgggctccaggctgaggacgaggctgattattactgctgctcatatgcaggtggtaggaccttcaatgtgctattcggcggcgggaccaagctgaccgtccta

SEQ ID NO: 597 - DNA, 357 bp, synthetic construct
  misc_feature 1..357: note = DNA sequence encoding VH of EZ 8B
  source 1..357: mol_type = other DNA
gaagtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatctggggtactatggttcggggagttcctggggccagggaaccctggtcaccgtctcctca

SEQ ID NO: 598 - DNA, 318 bp, synthetic construct
  misc_feature 1..318: note = DNA sequence encoding VL of EZ 8B
  source 1..318: mol_type = other DNA
tcctatgagctgactcagccaccctcagtgtccgtgtccccaggacagacagccagcatcacctgctctggagataaattgggggataaatatgcttgctggtatcagcagaagccaggccagtcccctgtgctggtcatctatcaagatagcaagcggccctcagggatccctgagcgattctctggctccaactctgggaacacagccactctgaccatcagcgggacccaggctatggatgaggctgactattactgtcaggcgtgggacagcagcactgcggtgttcggcggagggaccaagctgaccgtccta

SEQ ID NO: 599 - DNA, 399 bp, synthetic construct
  misc_feature 1..399: note = DNA sequence encoding VH of FD 3E
  source 1..399: mol_type = other DNA
gaagtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagatccccattactatggttcggggagttattataaccagctgaggggatactactactacggtatggacgtctggggccaagggaccacggtcaccgtctcctca

SEQ ID NO: 600 - DNA, 333 bp, synthetic construct
  misc_feature 1..333: note = DNA sequence encoding VL of FD 3E
  source 1..333: mol_type = other DNA
gatattgtgatgacccagactccactctctctgtccgtcacccctggacagccggcctccatctcctgcaagtctagtcagagcctcctgcatagtgatggaaagacctatttgtattggtacctgcagaagccaggccagtctccacagctcctaatctatgaagtttccagccggttctctggagtgccagataggttcagtggcagcgggtcagggacagatttcacactgaaaatcagccgggtggaggctgaggatgttggggtttattactgcatgcaaggtatacaccttcctcactttcggcggagggaccaagtggagatcaaa

SEQ ID NO: 601 - DNA, 363 bp, synthetic construct
  misc_feature 1..363: note = DNA sequence encoding VH of EW 1A
  source 1..363: mol_type = other DNA
gaagtgcagctggtggagtctgggggaggcgtggtccagcctgggtcgtccctgagactctcctgtgaaacgtctggtttcaccttcagtggacatgccatgcactgggtccgccaggctccaggcaaggggctggagtggctggcacaaatctggttcgatggaagtgaaaaatactatgcagattccgtgaagggtcgattcaccatctccagagacaattccaagaaaatcctatatatgcaaatgaacagcctgagagtccaagacacggctgtgtattactgtgcgagagatgggcaacatctggcacctttcgctatggacgtctggggccaagggacaatggtcaccgtctcttca

SEQ ID NO: 602 - DNA, 330 bp, synthetic construct
  misc_feature 1..330: note = DNA sequence encoding VL of EW 1A
  source 1..330: mol_type = other DNA
cagtctgtgctgactcagcctgcctccgtgtctgggtctcctggacagtcaatcaccatctcctgcactggaaccagcagtgacgttggtgcctctaaccgtgtttcctggtaccaacactccccaggcgaagcccccaaactcatcatttatcaggtcactgttcggccctcaggggtgtctgatcgcttctctggctcgaagtccggcaacacggcctccctgaccatctctgggctccggactgaggacgaggctgaatattactgcaactcatttgtaagcggtgactcttgggtgttcggcggagggaccaaggtgaccgtccta

SEQ ID NO: 603 - DNA, 366 bp, synthetic construct
  misc_feature 1..366: note = DNA sequence encoding VH of FD 5B
  source 1..366: mol_type = other DNA
gaagtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggccgtctattactgtgcgagagatgaacgtagagagtcctacaatttcgtgttggactactggggccagggaaccctggtcaccgtctcctca

SEQ ID NO: 604 - DNA, 333 bp, synthetic construct
  misc_feature 1..333: note = DNA sequence encoding VL of FD 5B
  source 1..333: mol_type = other DNA
cagtctgtgctgactcagcctccctccgcgtccgggtctcctggacagtcagtcaccatctcctgcactggaaccagcagtgacgctggtggttataactatgtctcctggtaccaacagcacccaggcaaagcccccaaactcatgatttatgaggtcagtaagcggccctcaggggtccctgatcgcttctctggctccaagtctggcaacacggcctccctgaccgtctctgggctccaggctgaggatgaggctgattattactgcagctcatatgcaggcagcaacaacccctttgtcttcggaactgggaccaaggtcaccgtccta

SEQ ID NO: 605 - DNA, 396 bp, synthetic construct
  misc_feature 1..396: note = DNA sequence encoding VH of EW 9A
  source 1..396: mol_type = other DNA
gaggtgcagctgttggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgaaagatatgtgggccttatacgatattttgactggttattatacaccctactactactacggtatggacgtctggggccaagggaccacggtcaccgtctcctca

SEQ ID NO: 606 - DNA, 324 bp, synthetic construct
  misc_feature 1..324: note = DNA sequence encoding VL of EW 9A
  source 1..324: mol_type = other DNA
tcctatgagctgacacagccaccctcggtgtcagtgtccccaggacagacggccaggatcacctgctctgcagatgcattgccaaagcaatatgcttattggtaccagcagaagccaggccaggcccctgtgctggtgatatataaagacagtgagaggccctcagggatccctgagcgattctctggctccagctcagggacaacagtcacgttgaccatcagtggagtccaggcagaagacgaggctgactattactgtcaatcagcagacagcagtggtacttattgggtgttcggcggagggaccaagctgaccgtccta

SEQ ID NO: 607 - DNA, 366 bp, synthetic construct
  misc_feature 1..366: note = DNA sequence encoding VH of FD 8C
  source 1..366: mol_type = other DNA
gaggtgcagctggtggagtctgggggaggcttggtaaagccagggcggtccctgagactctcctgtacagcttctggattcacctttggtgattatgctatgagctggttccgccaggctccagggaaggggctggagtgggtaggtttcattagaagcaaagcttatggtgggacaacagaatacgccgcgtctgtgaaaggcagattcaccatctcaagagatgattccaaaagcatcgcctatctgcaaatgaacagcctgaaaaccgaggacacagccgtgtattactgtactaggaacgatttttggagtggttattatccagactactggggccagggaaccctggtcaccgtctcctca

SEQ ID NO: 608 - DNA, 339 bp, synthetic construct
  misc_feature 1..339: note = DNA sequence encoding VL of FD 8C
  source 1..339: mol_type = other DNA
gatattgtgatgactcagtctccactctccctgcccgtcacccttggacagccggcctccatctcctgcaggtctagtcaaagcctcgtacacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccccgctcactttcggcggagggaccaaagtggatatcaaac

SEQ ID NO: 609 - DNA, 360 bp, synthetic construct
  misc_feature 1..360: note = DNA sequence encoding VH of EW 5A
  source 1..360: mol_type = other DNA
gaagtgcagctggtggagtcggggggaggcttggtccagcctggggggtccctgcgactctcctgttcagtctctggattccccttcggaacatatgctatgcactgggtccgccaggctcccgggaaggggctagattatgtttcagctattaataatgatgggagtattacatactacgcagactcagtgaggggcagattcaccgtctccagagacaattccgagaacactctatatcttcgactgagcggtctgagacctgacgacacggctatctattattgtgtgaaagatcggggctccgttattcgggacttcgacgtctggggccgtggcaccctggtcaccgtctcctca

SEQ ID NO: 610 - DNA, 327 bp, synthetic construct
  misc_feature 1..327: note = DNA sequence encoding VL of EW 5A
  source 1..327: mol_type = other DNA
cagactgtggtgactcaggagccctcactgactgtctccccaggagggacagtcactctcacctgtgcttccagtactggaacagtcaccagtgattactatccaaactggttccagcagaagcctggacaggcacccaggcctctgattttcggtacagcctacagacactcctggacccctgcccgattctcaggctccctccttgggggcaaagctgccctgacagtgtcagatgtgcagcctgaggacgaggctgactattactgcctgctctactgtggtggtggtcagcttttcggcggagggaccaagctgaccgtccta

SEQ ID NO: 611 - DNA, 366 bp, synthetic construct
  misc_feature 1..366: note = DNA sequence encoding VH of EZ 9C
  source 1..366: mol_type = other DNA
gaagtgcagctggtggagtctgggggaggcttggtccagcctggggggtccctgagactctcctgttcagcctctggattcaccctcaatggctatgctatgcactgggtccgccaggctccagggaaaggactggagtatgtttcaagtctttctaatcgtggagataatacacgctacgcagagtccgtgaagggcagattcctcatctccagagacattgccaaggacacgctttatcttcagatgagcagtctgagacctgaggacacggctgtctattactgtgggaaaggccttttgtctgccagtgggggattgccgattgacgactggggccagggaaccctggtcaccgtctcctca

SEQ ID NO: 612 - DNA, 330 bp, synthetic construct
  misc_feature 1..330: note = DNA sequence encoding VL of EZ 9C
  source 1..330: mol_type = other DNA
cagtctgtgctgacgcagccgccctcagtgtctgcggccccaggacagaaggtcaccatctcctgctctggaagcagctccaacattgggaataattatgtatcctggtaccagcagttcccaggaacagcccccaaactcctcatttatgacaataataagcgaccctcagggattcctgaccgattctctggctccaagtctggcacgtcagccaccctgggcattaccggcctccagactggggacgaggccgcttattactgcgcaacatgggatagcagcctgagtgctggggtgttcggcggaggggccaagctgaccgtccta

SEQ ID NO: 613 - DNA, 351 bp, synthetic construct
  misc_feature 1..351: note = DNA sequence encoding VH of EZ 9A
  source 1..351: mol_type = other DNA
gaagtgcagctggtggagtccgggggaggcttagttcagcctggggagtcactgagagtctcctgtgcagcctctggattcaccttcagtaactactggatgcactgggtccgccaagtcccaggaaaggggccggtgtgggtctcaattattaatactgatggaagtatcacaagatatgtggactccgtgaggggccgattcaccatctccagagacaacgccaagaacacggtgcatctgcaaatgaacagcctgacagccgaggacacggctatatattattgtgcaagagatgtcaataggtaccctgactactggggccagggaaccctggtcaccgtctcctca

SEQ ID NO: 614 - DNA, 330 bp, synthetic construct
  misc_feature 1..330: note = DNA sequence of VL ofEZ 9A
  source 1..330: mol_type = other DNA
cagtctgccctgactcagcccgcctccgtgtctgggtctcctgggcagtcgatcaccatttcctgcactggaacctacagtgacgttggttattataactatgtctcctggtatcaacaacagcccggcaaggcccccaaagtcatcattcatggggacattaatcggccctttggagtttctaatcgcttttctggctccaagtctggcaacacggcctccctgacaatctctgggctccaggccgaggacgaggctgattatttctgctgctcatatgtaaataatggcgcttgggtgttcggcggagggaccaagttgaccgtccta

SEQ ID NO: 615 - DNA, 390 bp, synthetic construct
  misc_feature 1..390: note = DNA sequence encoding VH of EY 12A
  source 1..390: mol_type = other DNA
gaagtgcagctggtggagtcgggccccggactggtgaagccttcacagaccctgtccctcacctgcgctgtctccggtggctccatcagcagtggtggttactactggagttggatccgccagcccccaggaaagggcctggagttgattgggtacaccgattacactgggaagaccctctacaacccatccctcaagagtcgacttaccatatcagtggacacgtccaagaaccagttctccctgaagttgaggtctgtgactgccgcagacacggccgtctattactgtgccagagggatgacgcaagacgatattttgactggttttaataggcctcactggtatttcgatctctggggccgtggcagtctggtcaccgtctcctca

SEQ ID NO: 616 - DNA, 336 bp, synthetic construct
  misc_feature 1..336: note = DNA sequence encoding VL of EY 12A
  source 1..336: mol_type = other DNA
cagtctgtgctgactcagccgccctcagtgtctggggccccagggcagagggtcaccatctcctgcactgggagcagctccaacatcggggcaggttatgatgtacactggtaccagcagcttccaggaacagcccccaaagtcgtcatctatcgtaacatgaatcggccctcaggggtccctgaccgattctctggctccaagtctggcacctcagcctccctggccatcactgggctccaggctgaggatgaggctgattattactgccagtcctttgacagcagcctgagtgattttgtggttttcggcggagggaccaagctgaccgtccta

SEQ ID NO: 617 - DNA, 363 bp, synthetic construct
  misc_feature 1..363: note = DNA sequence encoding VH of FD 4C
  source 1..363: mol_type = other DNA
gaagtgcagctggtggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagccgagttaccatatcagtagacacgtctaagaaccagttctccctgaagctgagctctgtgactgccgcggacacggccgtgtattactgtgcgagagtccggtctagcagcagctggtactttgactactggggccagggaaccctggtcaccgtctcctca

SEQ ID NO: 618 - DNA, 324 bp, synthetic construct
  misc_feature 1..324: note = DNA sequence encoding VL of FD 4C
  source 1..324: mol_type = other DNA
cagtctgtgctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaagcggccctcaggggtttctaatcgcttctctggctccaagtctggcaacacggcctccctgaccatctctgggctccaggctgaggacgaggctgattattactgcagctcatatacaagcaaatgggtgttcggcggagggaccaagctgaccgtccta

SEQ ID NO: 619 - DNA, 348 bp, synthetic construct
  misc_feature 1..348: note = DNA sequence encoding VH of EW 4C
  source 1..348: mol_type = other DNA
gaggtgcagctggtggagtcgggcccagggctggtgaagccttcggagaccctgtccctcacctgcagtgtctctgctgactccttcaaaaaaggttactattggggctggatccggcagccccctgggaagggattggaatcgattgtcaattcttttgattccgggaccacccgctataatccgtccctctggggtcgagccaccgtatcagacatgtccaagtggcacttctccctgaagttgacctctgtgaccgccgcagacacggccgtttattattgtgtccggggaacatatggttcgggccttcactggggccagggaatcctggtcaccgtctcctca

SEQ ID NO: 620 - DNA, 327 bp, synthetic construct
  misc_feature 1..327: note = DNA sequence encoding VL of EW 4C
  source 1..327: mol_type = other DNA
cagactgtggtgacccaggagccctcactgactgtgtccccaggagggacagtcactctcacttgtggctccagcgttggaactgtcgccagtggtcattatccctactgggtccagcagaagcctggccaagcccccaggacactgatttatgatacagacaacaaacaatcctggacccctgcccggttctcaggctccctccttgggggcaaggctgccctgaccctttcgggtgcgcagcctgaggatgaggctgactattactgctttctctcccataatgatgcttgggtgttcggcggagggaccaagttgaccgtcctt

SEQ ID NO: 621 - DNA, 378 bp, synthetic construct
  misc_feature 1..378: note = DNA sequence encoding VH of FD 6D
  source 1..378: mol_type = other DNA
gaagtgcagctggtggagtcgggcccgggactggtgaaggtttcggagaccctgttcctcacctgcactgtctctgggtactccatcggcagtggtaactactggggctggatccggcagcccccagggaaggttctggagtggattgggagtacttaccacagtgggaccacctactacaatccgtccctcaagagtcgagtcaccatatcagttgactcgtccaagaatcagttctccctgaagctgacctctgtgaccgccgcagacacggccgtgtattactgtgcgagagatcggctattagctgtccattatgacagccgtggttatttagttgactactggggccaagggacaatggtcaccgtctcttca

SEQ ID NO: 622 - DNA, 339 bp, synthetic construct
  misc_feature 1..339: note = DNA sequence encoding VL of FD 6D
  source 1..339: mol_type = other DNA
gccatccggatgacccagtctccagactccctggctgtgtctctgggcgagagggccaccatcaactgcaagtccagccagagtattttttacagctccaacaataagaactatttagcttggtaccagcacaaaccgggacagcctcctaagctcctcatttactgggcatctacccgggaatccggggtccctgaccgattcagtggcagcgggtctgggacagatttcactctcaccatcagcagcctgcaggctgaagatgtggcaatttattactgtcagcaatattatgatattcctcggacgttcggccaagggaccaaggtggaaatcaga

SEQ ID NO: 623 - DNA, 360 bp, synthetic construct
  misc_feature 1..360: note = DNA sequence encoding VH of EY 5A
  source 1..360: mol_type = other DNA
gaagtgcagctggtggagtcgggcccaggactggtgaagccttcggggaccctgttcctcacctgcactgtctctggtggctccatcagtaattactactggagctggatccggcagcccccagggaaggcactagagtggattgggtttatttattccaatgggaacactgattacaacccctccctccagagtcgagtcaccatatcgggagacacgtccaagaaccagttctccctgaacctgaggtctgttaccgctgcggacacggccgtgtattactgtgcgagagggccggggccggctacggggggtagtcttgactactggggccaagggacaatggtcaccgtctcttca

SEQ ID NO: 624 - DNA, 330 bp, synthetic construct
  misc_feature 1..330: note = DNA sequence encoding VL of EY 5A
  source 1..330: mol_type = other DNA
aattttatgctgactcagccaccctcagcgtctgggacccccgggcagagggtcaccatctcttgttctggaagcagctccaacatcggaataaatactgtaaactggtaccagcacctcccaggaacggcccccaaactcctcatctatggtaataatcagcggccctcaggggtccctgaccgattctctggctccaagtctggcacctcaacctccctggccatcagtgggctccagtctgaggatgaggctgattattattgttcagcatgggatgacagcctgaatggtcctgtgttcggaggaggcacccagctgaccgtcctc

SEQ ID NO: 625 - DNA, 393 bp, synthetic construct
  misc_feature 1..393: note = DNA sequence encoding VH of EZ 7B
  source 1..393: mol_type = other DNA
gaagtgcagctggtggagtcgagcccaggactggtgaagccttcggagaccctgtccctcacctgcactgtctctggtggctccatcagtagttactactggagctggatccggcagcccccggggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagaagggttttcggccccgtcctcccttccaaactgggtgggagctactgggggggaggtgcttttgatatctggggccaagggacaatggtcaccgtctcttca

SEQ ID NO: 626 - DNA, 339 bp, synthetic construct
  misc_feature 1..339: note = DNA sequence encoding VL of EZ 7B
  source 1..339: mol_type = other DNA
gacatccagttgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcccctcactttcggccctgggaccaaggtggaaatcaga

SEQ ID NO: 627 - DNA, 390 bp, synthetic construct
  misc_feature 1..390: note = DNA sequence encoding VH of EZ 4C kappa
  source 1..390: mol_type = other DNA
gaagtgcagctggtggagtcgggcccaggactggtgaagccttcggagaccctgtccctcacctgcactgtctctggtggctccgtcagtagtggtaataactactggagctggatccggcagcccccagggaaggcactggagtggattggatatatctattacagtgggagcaccaagtacaacccctccctcaagagtcgagtcaccatgtctgtaggcacgtccaagaatcaattctctctgaaagtgaactctgtgacggctgcggacacggccatgtattactgtgccagagccccctcggctccctttgggggactttttgactggatattacctaaagggattaacaactggggccagggaaccctggtcaccgtctcctca

SEQ ID NO: 628 - DNA, 321 bp, synthetic construct
  misc_feature 1..321: note = DNA sequence encoding VL of EZ 4C kappa
  source 1..321: mol_type = other DNA
gaaattgtgttgacacagtctccttccaccctgtctgcatctgtaggagacagagtcaccatcacttgccggaccagtcagagtattcgtagctggttggcctggtatcaacaaaaaccagggaaagcccctaaactcctgatctttgaggcatctactttagaaagtggggtcccagagaggttcagcggcagtggatctggggcggaattcactctcaccatcagcagcctgcagcctgatgattttgcaacttattactgtcaacagtataatggttattcttacagttttggccaggggaccaaggtggaaatcaga

SEQ ID NO: 629 - DNA, 390 bp, synthetic construct
  misc_feature 1..390: note = DNA sequence encoding VH of EZ 4C lambda
  source 1..390: mol_type = other DNA
gttcagctggtgcaggagtcgggcccaggactggtgaagccttcggagaccctgtccctcacctgcactgtctctggtggctccgtcagtagtggtaataactactggagctggatccggcagcccccagggaaggcactggagtggattggatatatctattacagtgggagcaccaagtacaacccctccctcaagagtcgagtcaccatgtctgtagacacgtccaagaatcaattctctctgaaagtgaactctgtgacggctgcggacacggccatgtattactgtgccagagccccctcggctccctttgggggactttttgactggatattacctaaagggattgacaactggggccagggaaccctggtcaccgtctcctca

SEQ ID NO: 630 - DNA, 330 bp, synthetic construct
  misc_feature 1..330: note = DNA sequence encoding VL of EZ 4C lambda
  source 1..330: mol_type = other DNA
cagtctgccctgactcagccgccttcagtgtctgccgccccaggacagaaggtcaccatctcctactctggaaacagctccgacatggggacttatgcggtatcttggtaacagcgactcccaggaacagcccccaaactcttcatctgtgaagagaataagcgacccccagggattcctgaccgattctctggctccaagtctggcacttcagccaccctgggcatcactggcctctggcctgaggacgaggccgattattgctaaatagcatgacatagcagcccgagacttgggtgttcggcggagggaccaagctgaccgtcctag

SEQ ID NO: 631 - DNA, 390 bp, synthetic construct
  misc_feature 1..390: note = DNA sequence encoding VH of FD 4B
  source 1..390: mol_type = other DNA
cagctgcagctgcaggagtcgggcccaggactggtgaagccttcggagaccctgtccctcacctgcactgtctctggtggctccgtcagtagtggtaataactactggagctggatccggcagcccccagggaaggcactggagtggattggatatctctattacagtgggagcaccaagtacaacccctccctcaagagtcgagtcaccatgtctgtagacacgtccaagaatcaattctctctgaaagtgaactctgtgacggctgcggacacggccatgtattattgtgccagagccccctcggctccctttgggggactttttgactggatattacctaaagggattgacagctggggccagggaaccctggtcaccgtctcctca

SEQ ID NO: 632 - DNA, 330 bp, synthetic construct; identical to SEQ ID NO: 630
  misc_feature 1..330: note = DNA sequence encoding VL of FD 4B
  source 1..330: mol_type = other DNA

SEQ ID NO: 633 - DNA, 384 bp, synthetic construct
  misc_feature 1..384: note = DNA sequence encoding VH of EY 8A
  source 1..384: mol_type = other DNA
gaggtgcagctggtggagtcgggcccaggactggtgaagccttcacagaccctgtccctcacctgcactgtctctggtggctccatcagcagtggtagttactactggagctggatccggcagcccgccgggaagggactggagtggattgggcgtatctataccagtgggagcaccaactacaacccctccctcaagagtcgagtcaccatatcagtagacacgtccaagaaccagttctccctgaagctgagctctgtgaccgccgcagacacggccgtgtattactgtgcgaaaggacatgttattagtggctacgatgattactactactactacggtatggacgtctggggccaagggaccacggtcaccgtctcctca

SEQ ID NO: 634 - DNA, 324 bp, synthetic construct
  misc_feature 1..324: note = DNA sequence encoding VL of EY 8A
  source 1..324: mol_type = other DNA
tcctatgagctgactcagccaccctcggtgtcagtgtccccaggacagacggccaggatcacctgctctggagatgcattgccaaagcaatatgcttattggtaccagcagaagccaggccaggcccctgtgctggtgatatataaagacagtgagaggccctcagggatccctgagcgattctctggctccagctcagggacaacagtcacgttgaccatcagtggagtccaggcagaagacgaggctgactattactgtcaatcagcagacagcagtggtacttattgggtgttcggcggagggaccaagctgaccgtccta

SEQ ID NO: 635 - DNA, 390 bp, synthetic construct
  misc_feature 1..390: note = DNA sequence encoding VH of EY 2A
  source 1..390: mol_type = other DNA
caggtgcagctggtggagtctggagcagaggtgacaaagcccggggactctctgataatctcctgtaagggctctggatatgcatttactcaatactggatcggctgggtgcgccagaagcccgggaaaggcctggagtggatggccatggtttatcccgattcctctgccgtctttgccggtggtgcctctggcgtcagatataggccgcccttccaaggccaggtcaccatatcagccgacacgtccgtcaacaccgcctacctgcagtgggacagcctgaaggcctcggacaccgccatgtactattgtgtaagacaggaacgtgggagcaatacttggtacgcgggaaactcctggggccagggaactctggtcaccgtctcctca

SEQ ID NO: 636 - DNA, 336 bp, synthetic construct
  misc_feature 1..336: note = DNA sequence encoding VL of EY 2A
  source 1..336: mol_type = other DNA
gatattgtgatgactcagtctccactctccctgcccgtcagccctggagagccggcctccatctcctgtaggtctagtcagagcctcctccatactgatgcatacaactatttggattggtacctgcaaaagccagggcagtctccacaactcctgatctatttgggttctactcgggcctccggggtgcctgacaggttcagtggcagtggatcaggcacagactttacactgaaaatcagtagcgtgaaggctgaggatgttggggtttattactgcatgcaagctctacaaactccgggcacttttggccaggggaccaagctggagatcaaa

SEQ ID NO: 637 - DNA, 390 bp, synthetic construct
  misc_feature 1..390: note = DNA sequence encoding VH of EY 3B
  source 1..390: mol_type = other DNA
gaagtgcagctggtggagtctggagcagaggtgacaaagcccggggagtctctgataatctcctgtaaggggtctggatatgcatttactcaatactggatcggctgggtgcgccagaagcccgggaaaggcctggagtggatggccatggtgtatcccgattcctctgccgtctttgccggtggtgcctctggcgtcagatataggccgcccttccaaggccaggtcaccatctcagccgacacgtccgtcaacaccgcctacctgcagtgggacagcctgaaggcctcggacaccgccatgtactattgtgtaagacaggaacgtgggagcaacacttggtacgcgggaaactcctggggccagggaactctggtcaccgtcttctca

SEQ ID NO: 638 - DNA, 336 bp, synthetic construct
  misc_feature 1..336: note = DNA sequence encoding VL of EY 3B
  source 1..336: mol_type = other DNA
gaaattgtgttgacgcagtctccactctccctgcccgtcagccctggagagccggcctccatctcctgtaggtctagtcagagcctcctccatactgatgcatacaactatttggattggtacctgcaaaagccagggcagtctccacagctcctgatctatttgggttctactcgggcctccggggtgcctgacaggttcagtggcagtggatcaggcacagactttacactgaaaatcagtagcgtgaaggctgaggatgttggggtttattactgcatgcaagctctacaaactccgggcacttttggccaggggaccaaggtggaaatcaga

SEQ ID NO: 639 - DNA, 357 bp, synthetic construct
  misc_feature 1..357: note = DNA sequence encoding VH of EZ 4A
  source 1..357: mol_type = other DNA
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagatcgcctatagcagcagacctgtttgactactggggccagggaaccctggtcaccgtctcctca

SEQ ID NO: 640 - DNA, 321 bp, synthetic construct
  misc_feature 1..321: note = DNA sequence encoding VL of EZ 4A
  source 1..321: mol_type = other DNA
gacatccagttgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctcttattcactttcggccctgggaccaaggtggagatcaaa

(19) United States
(12) Patent Application Publication
(10) Pub. No.: US 2023/0279079 A1
(43) Pub. Date: Sep. 7, 2023

(54) NOVEL MONOCLONAL ANTIBODIES AGAINST SARS-COV-2 AND USES THEREOF

(71) Applicants: Chang Gung Memorial Hospital, Linkou, Taoyuan (TW); Medigen Vaccine Biologics Corporation, Zhubei City (TW)

(72) Inventor: Kuan-Ying HUANG, Taoyuan (TW)

(21) Appl. No.: 18/054,594

(22) Filed: Nov. 11, 2022

Related U.S. Application Data
(63) Continuation of application No. PCT/CN2021/093083, filed on May 11, 2021.
(60) Provisional application No. 63/022,844, filed on May 11, 2020.
(60) Provisional application No. 63/029,980, filed on May 26, 2020.
(60) Provisional application No. 63/070,560, filed on Aug. 26, 2020.

(51) Int. Cl. C07K 16/10; A61P 31/14; G01N 33/543; G01N 33/569

(52) U.S. Cl. CPC C07K 16/10; A61P 31/14; G01N 33/54386; G01N 33/56983; A61K 2039/505

(57) ABSTRACT

The present invention provides novel monoclonal antibodies or the antigen-binding fragments thereof, which bind to a severe acute respiratory syndrome coronavirus 2 (SARS-CoV-2) or angiotensin-converting enzyme 2 (ACE2). Also provided are a pharmaceutical composition and a kit for detecting the presence of SARS-CoV-2 in a sample comprising the novel monoclonal antibody or the antigen-binding fragments thereof. Also disclosed herein are methods for detection or prevention and/or treatment of SARS-CoV-2 or a disease mediated by a disease mediated by ACE2.

### CROSS-REFERENCE TO RELATED APPLICATIONS

This application is a continuation application of International Patent Application No. PCT/CN2021/093083, filed on May 11, 2021, which itself claims priority to U.S. Provisional Application No. 63/022,944, filed on May 11, 2020; U.S. Provisional Application No. 63/029,980, filed on May 26, 2020; and U.S. Provisional Application No. 63/070,560, filed on Aug. 26, 2020. The disclosures of the above applications are incorporated herein in their entireties by reference.

The sequence information contained in the Sequence Listing XML file, with the file name “P22-0214US.xml” created on Nov. 8, 2022 and having a file size of 791,199 bytes, is incorporated by reference herein in its entirety.

### BACKGROUND OF THE INVENTION

### 1. Field of the Invention

The present invention relates to novel monoclonal antibodies (MAbs) against severe acute respiratory syndrome coronavirus 2 (SARS-CoV-2) and/or antigen-binding fragments thereof, especially to novel MAbs binding to the spike (S) protein or the nucleocapsid (N) protein of SARS-COV-2. The present invention also provides a pharmaceutical composition comprising the novel MAbs or antigen-binding fragments thereof. In addition, the present invention provides a kit and method for detecting SARS-CoV-2 and a method for preventing or treating SARS-CoV-2 or a disease mediated by a disease mediated by ACE2, using the novel MAbs or antigen-binding fragments thereof as described herein.

### 2. Description of the Prior Art

In the end of 2019, a novel coronavirus emerged and was identified as a cause of a cluster of respiratory infection cases. It spread quickly throughout the world. It spread quickly throughout the China and the world. In March of 2020, it has been declared a pandemic by the World Health Organization, and severe acute respiratory syndrome coronavirus 2 (SARS-CoV-2) is the virus responsible for the coronavirus disease of 2019 (COVID-19). As of 6 May 2021, there have been 154,815,600 total confirmed cases of SARS-CoV-2 infection including 3,236,104 deaths in the ongoing pandemic (World Health Organization).

Although several SARS-CoV-2 vaccines are available, the average worldwide vaccination rate is still low. Besides that, some of the SARS-CoV-2 vaccines currently available require extremely low temperature for storage, while some of the other available vaccines raise concerns about safety and/or low efficacy. As a result, the emergence of the novel coronaviruses in human population remains a continuing threat. In addition, antiviral drugs for SARS-CoV-2 are unavailable in the present (Rome 2020). Conservative treatment is still considered the mainstay of treatment for the SARS-CoV-2 infection in humans. Previous reports indicated that passive immunotherapy with convalescent plasma, serum, or hyperimmune immunoglobulin containing virus-specific polyclonal antibodies may be alternative therapeutic approach toward reduction of mortality of severe respiratory viral infections (Mair-Jenkins 2015). It is also realized for the need of monoclonal antibody (MAb) preparations for the treatment or prophylaxis of viral infectious disease, since polyclonal immunoglobulins may have limited potency and disease scope (Casadevall 2004).

### SUMMARY OF THE INVENTION

The present invention provides a panel of SARS-CoV-2 spike and nucleocapsid-reactive human monoclonal antibodies, which has been produced from peripheral B cells derived from adult patients with laboratory-confirmed SARS-CoV-2 infection. The antigenic specificity of MAbs and the genetic usage in their variable domains of heavy and light chains were characterized in detail. These SARS-CoV-2-antigen-specific human MAbs offer templates for the development of diagnostic reagents and candidate prophylactic and therapeutic agents against SARS-CoV-2.

Thus, in one aspect, the present invention provides an isolated antibody against Severe Acute Respiratory Syndrome Coronavirus 2 (SARS-CoV-2) or antigen-binding fragment thereof, comprising
(i) a heavy chain variable region (V_{H}) which comprises
   (a) a first heavy chain complementarity determining region (HCDR1) having an amino acid sequence of about 90%, 91%, 92%, 93%, 94%, 95%, 96%, 97%, 98%, 99% or 100% identical to SEQ ID No: 69, SEQ ID No: 75, SEQ ID No: 81, SEQ ID No: 87, SEQ ID No: 93, SEQ ID No: 99, SEQ ID No: 105, SEQ ID No: 111, SEQ ID No: 117, SEQ ID No: 123, SEQ ID No: 129, SEQ ID No: 135, SEQ ID No: 141, SEQ ID No: 147, SEQ ID No: 153, SEQ ID No: 159, SEQ ID No: 165, SEQ ID No: 171, SEQ ID No: 177, SEQ ID No: 183, SEQ ID No: 189, SEQ ID No: 195, SEQ ID No: 201, SEQ ID No: 207, SEQ ID No: 213, SEQ ID No: 219, SEQ ID No: 225, SEQ ID No: 231, SEQ ID No: 237, SEQ ID No: 243, SEQ ID No: 249, SEQ ID No: 255, SEQ ID No: 261, SEQ ID NO: 267, SEQ ID No: 333, SEQ ID No: 339, SEQ ID No:345, SEQ ID No: 351, SEQ ID No: 357, SEQ ID No: 363, SEQ ID No: 369, SEQ ID No: 375, SEQ ID No: 381, SEQ ID No: 387, SEQ ID No: 393, SEQ ID No: 399, SEQ ID No: 405, SEQ ID No: 411, SEQ ID No: 417, SEQ ID No: 423, SEQ ID No: 429, SEQ ID No: 435, SEQ ID No: 441, SEQ ID No: 447, SEQ ID No: 453, SEQ ID No: 459, SEQ ID No: 465, SEQ ID No: 471, SEQ ID No:477, SEQ ID No: 483, SEQ ID No: 489, SEQ ID No: 495, SEQ ID No: 501, or SEQ ID No: 507;
   (b) a second heavy chain complementarity determining region (HCDR2) having an amino acid sequence of about 90%, 91%, 92%, 93%, 94%, 95%, 96%, 97%, 98%, 99% or 100% identical to SEQ ID No: 70, SEQ ID No: 76, SEQ ID No: 82, SEQ ID No: 88, SEQ ID No: 94, SEQ ID No: 100, SEQ ID No: 106, SEQ ID No: 112, SEQ ID No: 118, SEQ ID No: 124, SEQ ID No: 130, SEQ ID No: 136, SEQ ID No: 142, SEQ ID No: 148, SEQ ID No: 154, SEQ ID No: 160, SEQ ID No: 166, SEQ ID No: 172, SEQ ID No: 178, SEQ ID No: 184, SEQ ID No: 190, SEQ ID No: 196, SEQ ID No: 202, SEQ ID No: 208, SEQ ID No: 214, SEQ ID No: 220, SEQ ID No: 226, SEQ ID No: 232, SEQ ID No: 238, SEQ ID No: 244, SEQ ID No: 250, SEQ ID No: 256, SEQ ID No: 262, SEQ ID NO: 268, SEQ ID No: 334, SEQ ID No: 340, SEQ ID No:346, SEQ ID No: 352, SEQ ID No: 358, SEQ ID No: 364, SEQ ID No: 370, SEQ ID No: 376, SEQ ID No: 382, SEQ ID No: 388, SEQ ID No: 394, SEQ ID No: 400, SEQ ID No: 406, SEQ ID No: 412, SEQ ID No: 418, SEQ ID No: 424, SEQ ID No: 430, SEQ ID No: 436, SEQ ID No: 442, SEQ ID No: 448, SEQ ID No: 454, SEQ ID No: 460, SEQ ID No: 466, SEQ ID No: 472, SEQ ID No:478, SEQ ID No: 484, SEQ ID No: 490, SEQ ID No: 496, SEQ ID No: 502, or SEQ ID No: 508; and
   (c) a third heavy chain complementarity determining region (HCDR3) having an amino acid sequence of about 90%, 91%, 92%, 93%, 94%, 95%, 96%, 97%, 98%, 99% or 100% identical to SEQ ID No: 71, SEQ ID No: 77, SEQ ID No: 83, SEQ ID No: 89, SEQ ID No: 95, SEQ ID No: 101, SEQ ID No: 107, SEQ ID No: 113, SEQ ID No: 119, SEQ ID No: 125, SEQ ID No: 131, SEQ ID No: 137, SEQ ID No: 143, SEQ ID No: 149, SEQ ID No: 155, SEQ ID No: 161, SEQ ID No: 167, SEQ ID No: 173, SEQ ID No: 179, SEQ ID No: 185, SEQ ID No: 191, SEQ ID No: 197, SEQ ID No: 203, SEQ ID No: 209, SEQ ID No: 215, SEQ ID No: 221, SEQ ID No: 227, SEQ ID No: 233, SEQ ID No: 239, SEQ ID No: 245, SEQ ID No: 251, SEQ ID No: 257, SEQ ID No: 263, SEQ ID NO: 269, SEQ ID No: 335, SEQ ID No: 341, SEQ ID No:347, SEQ ID No: 353, SEQ ID No: 359, SEQ ID No: 365, SEQ ID No: 371, SEQ ID No: 377, SEQ ID No: 383, SEQ ID No: 389, SEQ ID No: 395, SEQ ID No: 401, SEQ ID No: 407, SEQ ID No: 413, SEQ ID No: 419, SEQ ID No: 425, SEQ ID No: 431, SEQ ID No: 437, SEQ ID No: 443, SEQ ID No: 449, SEQ ID No: 455, SEQ ID No: 461, SEQ ID No: 467, SEQ ID No: 473, SEQ ID No:479, SEQ ID No: 485, SEQ ID No: 491, SEQ ID No: 497, SEQ ID No: 503, or SEQ ID No: 509; and
(ii) a light chain variable region (V_{L}) which comprises
   (a) a first light chain complementarity determining region (LCDR1) having an amino acid sequence of about 90%, 91%, 92%, 93%, 94%, 95%, 96%, 97%, 98%, 99% or 100% identical to SEQ ID No: 72, SEQ ID No: 78, SEQ ID No: 84, SEQ ID No: 90, SEQ ID No: 96, SEQ ID No: 102, SEQ ID No: 108, SEQ ID No: 114, SEQ ID No: 120, SEQ ID No: 126, SEQ ID No: 132, SEQ ID No: 138, SEQ ID No: 144, SEQ ID No: 150, SEQ ID No: 156, SEQ ID No: 162, SEQ ID No: 168, SEQ ID No: 174, SEQ ID No: 180, SEQ ID No: 186, SEQ ID No: 192, SEQ ID No: 198, SEQ ID No: 204, SEQ ID No: 210, SEQ ID No: 216, SEQ ID No: 222, SEQ ID No: 228, SEQ ID No: 234, SEQ ID No: 240, SEQ ID No: 246, SEQ ID No: 252, SEQ ID No: 258, SEQ ID No: 264, SEQ ID NO: 270, SEQ ID No: 336, SEQ ID No: 342, SEQ ID No:348, SEQ ID No: 354, SEQ ID No: 360, SEQ ID No: 366, SEQ ID No: 372, SEQ ID No: 378, SEQ ID No: 384, SEQ ID No: 390, SEQ ID No: 396, SEQ ID No: 402, SEQ ID No: 408, SEQ ID No: 414, SEQ ID No: 420, SEQ ID No: 426, SEQ ID No: 432, SEQ ID No: 438, SEQ ID No: 444, SEQ ID No: 450, SEQ ID No: 456, SEQ ID No: 462, SEQ ID No: 468, SEQ ID No: 474, SEQ ID No:480, SEQ ID No: 486, SEQ ID No: 492, SEQ ID No: 498, SEQ ID No: 504, or SEQ ID No: 510;
   (b) a second light chain complementarity determining region (LCDR2) having an amino acid sequence of about 90%, 91%, 92%, 93%, 94%, 95%, 96%, 97%, 98%, 99% or 100% identical to SEQ ID No: 73, SEQ ID No: 79, SEQ ID No: 85, SEQ ID No: 91, SEQ ID No: 97, SEQ ID No: 103, SEQ ID No: 109, SEQ ID No: 115, SEQ ID No: 121, SEQ ID No: 127, SEQ ID No: 133, SEQ ID No: 139, SEQ ID No: 145, SEQ ID No: 151, SEQ ID No: 157, SEQ ID No: 163, SEQ ID No: 169, SEQ ID No: 175, SEQ ID No: 181, SEQ ID No: 187, SEQ ID No: 193, SEQ ID No: 199, SEQ ID No: 205, SEQ ID No: 211, SEQ ID No: 217, SEQ ID No: 223, SEQ ID No: 229, SEQ ID No: 235, SEQ ID No: 241, SEQ ID No: 247, SEQ ID No: 253, SEQ ID No: 259, SEQ ID No: 265, SEQ ID NO: 271, SEQ ID No: 337, SEQ ID No: 343, SEQ ID No:349, SEQ ID No: 355, SEQ ID No: 361, SEQ ID No: 367, SEQ ID No: 373, SEQ ID No: 379, SEQ ID No: 385, SEQ ID No: 391, SEQ ID No: 397, SEQ ID No: 403, SEQ ID No: 409, SEQ ID No: 415, SEQ ID No: 421, SEQ ID No: 427, SEQ ID No: 433, SEQ ID No: 439, SEQ ID No: 445, SEQ ID No: 451, SEQ ID No: 457, SEQ ID No: 463, SEQ ID No: 469, SEQ ID No: 475, SEQ ID No:481, SEQ ID No: 487, SEQ ID No: 493, SEQ ID No: 499, SEQ ID No: 505, or SEQ ID No: 511; and
   (c) a third light chain complementarity determining region (LCDR3) having an amino acid sequence of about 90%, 91%, 92%, 93%, 94%, 95%, 96%, 97%, 98%, 99% or 100% identical to SEQ ID No: 74, SEQ ID No: 80, SEQ ID No: 86, SEQ ID No: 92, SEQ ID No: 98, SEQ ID No: 104, SEQ ID No: 110, SEQ ID No: 116, SEQ ID No: 122, SEQ ID No: 128, SEQ ID No: 134, SEQ ID No: 140, SEQ ID No: 146, SEQ ID No: 152, SEQ ID No: 158, SEQ ID No: 164, SEQ ID No: 170, SEQ ID No: 176, SEQ ID No: 182, SEQ ID No: 188, SEQ ID No: 194, SEQ ID No: 200, SEQ ID No: 206, SEQ ID No: 212, SEQ ID No: 218, SEQ ID No: 224, SEQ ID No: 230, SEQ ID No: 236, SEQ ID No: 242, SEQ ID No: 248, SEQ ID No: 254, SEQ ID No: 260, SEQ ID No: 266, SEQ ID NO: 272, SEQ ID No: 338, SEQ ID No: 344, SEQ ID No:350, SEQ ID No: 356, SEQ ID No: 362, SEQ ID No: 368, SEQ ID No: 374, SEQ ID No: 380, SEQ ID No: 386, SEQ ID No: 392, SEQ ID No: 498, SEQ ID No: 404, SEQ ID No: 410, SEQ ID No: 416, SEQ ID No: 422, SEQ ID No: 428, SEQ ID No: 434, SEQ ID No: 440, SEQ ID No: 446, SEQ ID No: 452, SEQ ID No: 458, SEQ ID No: 464, SEQ ID No: 470, SEQ ID No: 476, SEQ ID No:482, SEQ ID No: 488, SEQ ID No: 494, SEQ ID No: 500, SEQ ID No: 506, or SEQ ID No: 512.

In some embodiments, the heavy chain variable region (V_{H}) comprises an amino acid sequence about 90%, 91%, 92%, 93%, 94%, 95%, 96%, 97%, 98%, 99% or 100% identical to the amino acid sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, or SEQ ID NO: 331.

In some embodiments, the light chain variable region (V_{L}) comprises an amino acid sequence about 90%, 91%, 92%, 93%, 94%, 95%, 96%, 97%, 98%, 99% or 100% identical to the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, or SEQ ID NO: 332.

In another aspect, the present invention provides a pharmaceutical composition, comprising at least one of the isolated antibodies, or antigen-binding fragments thereof, of the present invention.

In some embodiments, the pharmaceutical composition further comprises at least one pharmaceutically acceptable carrier.

In another aspect, the present invention provides a kit for detecting the presence of SARS-CoV-2 in a sample, comprising at least one of the isolated antibodies, or antigen-binding fragments thereof, of the present invention.

In some embodiments, the at least one of the isolated antibodies, or antigen-binding fragments thereof, of the present invention comprises a detectable label.

In some embodiments, the detectable label is selected from an enzymatic label, a fluorescent label, a metal label, and a radio label.

In some embodiments, the detectable label is selected from gold nanoparticles, colored latex beads, magnetic particles, carbon nanoparticles, and selenium nanoparticles.

In some embodiments, the kit is an immunoassay kit.

In some embodiments, the immunoassay kit is selected from ELISA (enzyme-linked immunosorbent assay), RIA (radioimmunoassay), FIA (fluorescence immunoassay), LIA (luminescence immunoassay), and ILMA (immunoluminometric assay).

In some embodiments, the immunoassay is a sandwich assay.

In some embodiments, the immunoassay is in a lateral flow assay format.

In yet another aspect, the present invention provides a method for detecting SARS-CoV-2 in a sample suspected of containing said SARS-CoV-2, comprising contacting the sample with at least one of the isolated antibodies, or antigen-binding fragments thereof, of the present invention, and assaying binding of the antibody with the sample.

In some embodiments, the sample is urine, stool, or taken from respiratory tract.

In some embodiments, the sample taken from the respiratory tract is a nasopharyngeal (NP) or nasal (NS) swab.

In some embodiments, the SARS-COV-2 is detected by a sandwich immunoassay or lateral flow assay.

In a further aspect, the present invention provides a method for preventing or treating a disease mediated by angiotensin-converting enzyme 2 (ACE2) in a subject, comprising a step of administering an effective amount of at least one of the isolated antibodies, or antigen-binding fragments thereof, of the present invention.

In some embodiments, the disease mediated by ACE2 is SARS-CoV-2 infection.

In still another aspect, the present invention provides a nucleic acid comprising a nucleotide sequence encoding a heavy chain variable region (V_{H}), a light chain variable region (V_{L}) or both, wherein the V_{H} and V_{L} are as described herein.

In further another aspect, the present invention provides a vector (e.g. an expression vector) comprising any of the nucleic acids described herein and a host cell comprising such a vector.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a graph illustrating the production of SARS-CoV-2 spike-reactive and nucleocapsid-reactive human monoclonal antibodies.

FIG. 2 is a line graph illustrating the Kd value and binding activity of anti-SARS-CoV-2 spike MAbs with spike protein of SARS-CoV-2, measured by ELISA. The SARS-CoV-2 therapeutic antibody (CR3022), cross reacts with SARS-CoV-2 and SARS-CoV-1, was included as a control. The OD value was presented as mean±standard error of the mean. The nonlinear regression analysis was performed to obtain the Kd value.

FIG. 3A to FIG. 3J are an assembly of line graphs showing the Kd value and binding activity of anti-SARS-CoV-2 receptor-binding domain (RBD) MAbs to the SARS-CoV-2 RBD, measured by flow cytometry (FM 7B Mab in FIG. 3A, FN 12A MAb in FIG. 3B, FI 1C MAb in FIG. 3C, FI 4A MAb in FIG. 3D, EY 6A MAb in FIG. 3E, FD 11A MAb in FIG. 3F, FD 5D MAb in FIG. 3G, FI 3AMAb in FIG. 3H, FJ 10B MAb in FIG. 3I, and EZ 7A MAb in FIG. 3J). FIG. 3K shows the Kd value and binding activity of anti-influenza H3 MAb BS-1A to SARS-CoV-2 RBD as a negative control. The binding percentage was presented as mean±standard error of the mean. The nonlinear regression analysis was performed to obtain the Kd value.

FIG. 4A is a line graph showing Ct value of virus signal in the supernatant of SARS-CoV-2 infected Vero E6 cells in an E gene-based real-time reverse-transcription PCR assay. The right shift of amplification plot reflects the increase of Ct value and the decrease of viral signal induced by EY 6A MAb, hence neutralization of the SARS-CoV-2. FIG. 4B is a line graph showing Ct value of virus signal in the supernatant of SARS-CoV-2 infected Vero E6 cells in an E gene-based real-time reverse-transcription PCR assay. The right shift of amplification plot reflects the increase of Ct value and the decrease of viral signal induced by FI 3A MAb, hence neutralization of the SARS-CoV-2.

FIG. 5A shows neutralization of wild type SARS-CoV-2 by anti-SARS-CoV-2 RBD MAbs (FD 11A, FI 3A, FI 1C, FD 5D and EY 6A) (also refer to Table 10), Neutralization assays were performed on the indicated antibodies according to the fluorescent focus-forming units microneutralization method (FMNT). Data were normalized to control (no antibody) values of foci, and the grey region comprises ±1 standard deviation the mean control values. Individual points are displayed ±1 standard deviation of technical, and curves are shown only where the data for a particular antibody fitted the standard dose-response (Hill) equation (n=3). FIG. 5B shows neutralization of wild type SARS-CoV-2 by anti-SARS-CoV-2 S1-non-RBD (FJ 1C, FD 11D, FD 11C and FD 7C) (also refer to Table 10). Neutralization assays were performed on the indicated antibodies according to the fluorescent focus-forming units microneutralization method (see methods). Data were normalized to control (no antibody) values of foci, and the grey region comprises ±1 standard deviation the mean control values. Individual points are displayed ±1 standard deviation of technical, and curves are shown only where the data for a particular antibody fitted the standard dose-response (Hill) equation (n=3).

FIG. 6A shows angiotensin-converting enzyme 2 (ACE2) blocking assays with titrations of anti-SARS-CoV-2 RBD antibodies. Assays were performed with RBD anchored and on plates (also refer to Table 8). FIG. 6B shows ACE2 blocking assays with titrations of anti-SARS-CoV-2 RBD antibodies (also refer to Table 10). Assays were performed with ACE2 anchored and on plates. Anti-SARS-CoV-2 RBD nanobody VHH72 linked to the hinge and Fc region of human IgG1 and ACE2-Fc were included as positive controls. Experiments were performed in duplicate and repeated twice. IC₅₀, 50% inhibitory concentrations.

FIG. 7A shows the prophylactic effect of a cocktail of the MAbs of the present invention against wild-type SARS-CoV-2 in Syrian hamster model. The left panel of FIG. 7A shows body weight change of the animals treated with a single dose (0.4 mg/kg, 4 mg/kg, or 40 mg/kg) of the antibody cocktail or 40 mg/kg of an isotype negative control one day prior to intranasal challenge of virus. The right panel of FIG. 7A shows infectious viral loads in the lungs measured by median tissue culture infectious dose (TCID₅₀) assay. FIG. 7B shows the therapeutic effect of the antibody cocktail against wild-type SARS-CoV-2 in Syrian hamster model. The left panel of FIG. 7B shows body weight change of the animals treated with a single dose (0.4 mg/kg, 4 mg/kg, or 40 mg/kg) of the antibody cocktail or 40 mg/kg of an isotype negative control three hours after intranasal challenge of virus. The right panel of FIG. 7B shows infectious viral loads in the lungs measured by TCID₅₀ assay. The data represents the mean±the standard error of the mean (SEM) (n=4 per group). Anti-influenza neuraminidase human IgG1 antibody Z2B3 was included as an isotype control. Statistical significance between groups was calculated by an unpaired two-sided t test. P values: *p<0.05; ns not significant.

FIG. 8A shows viral RNA of envelope (E) gene (copies per g RNA) detected in the lungs of hamsters challenged with SARS-CoV-2 (n=4 per group) at day 4 post challenge. FIG. 8B shows viral RNA of nucleocapsid (N) gene (copies per g RNA) detected in the lungs of hamsters challenged with SARS-CoV-2 (n=4 per group) at day 4 post challenge. Viral loads were determined by quantitative reverse transcription PCR for detection of SARS-CoV-2 E and N genes. The error bars represent standard deviations of the mean.

FIG. 9A shows histopathological findings of the lungs in the prophylactic treatment of antibody cocktail at 40 mg/kg, 4 mg/kg and 0.4 mg/kg in hamsters four days after SARS-CoV-2 infection. FIG. 9B shows histopathological findings of the lungs in the therapeutic treatment of antibody cocktail at 40 mg/kg, 4 mg/kg and 0.4 mg/kg in hamsters four days after SARS-CoV-2 infection. H&E stain. 40×, 100×, 400×.

### DETAILED DESCRIPTION OF THE PREFERRED EMBODIMENT

The present invention relates to novel MAbs that bind to the spike (S) protein or the nucleocapsid (N) protein of SARS-COV-2. The present invention provides such antibodies and antigen-binding fragments thereof, which are useful for detection or prevention and/or treatment of SARS-CoV-2 or a disease mediated by angiotensin-converting enzyme 2 (ACE2). The present invention also provides a pharmaceutical composition comprising the novel MAbs or antigen-binding fragments thereof. In addition, the present invention provides a kit and method for detecting SARS-CoV-2 and a method for preventing or treating SARS-CoV-2 or a disease mediated by a disease mediated by ACE2, using the novel MAbs or antigen-binding fragments thereof as described herein.

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

In order to provide a clear and ready understanding of the present invention, certain terms are first defined. Additional definitions are set forth throughout the detailed description. Unless defined otherwise, all technical and scientific terms used herein have the same meanings as is commonly understood by one of skill in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

As used herein, the term “about,” “around,” or “approximately” refers to a degree of acceptable deviation that will be understood by persons of ordinary skill in the art, which may vary to some extent depending on the context in which it is used. In general, “about,” “around,” or “approximately” may mean a numeric value having a range of 10% around the cited value. All numbers herein may be understood as modified by “about,” “around,” or “approximately.”

The term “antibody” as used herein refers to immunoglobulin molecules and immunologically active portions of immunoglobulin molecules, i.e., molecules that contain an antigen binding portion that immunospecifically binds a glycoprotein. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (V_{L}) and a constant domain (C_{L}). The heavy chain includes four domains, a variable domain (V_{H}) and three constant domains (C_{H}1, C_{H}2 and C_{H}3, collectively referred to as C_{H}). The variable regions of both light (V_{L}) and heavy (V_{H}) chains determine binding recognition and specificity to the stem cell surface glycoprotein. The light and heavy chains of an antibody each have three complementarity determining regions (CDRs), designated LCDR1, LCDR2, LCDR3 and HCDR1, HCDR2, HCDR3, respectively. An antigen-binding site, therefore, includes six CDRs, comprising the CDR set from each of a heavy and a light chain variable region. Framework Regions (FRs) refer to amino acid sequences interposed between CDRs.

Identity or homology with respect to a specified amino acid sequence of this invention is defined herein as the percentage of amino acid residues in a candidate sequence that are identical with the specified residues, after aligning the sequences and introducing gaps, if necessary, to achieve the maximum percent homology or identity, and not considering any conservative substitutions as part of the sequence homology or identity. None of N-terminal, C-terminal or internal extensions, deletions, or insertions into the specified sequence shall be construed as affecting homology or identity. Methods of alignment of sequences for comparison are well known in the art. While such alignments may be done by hand using conventional methods, various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al, Nucleic Acids Research 16:10881, 1988; and Pearson and Lipman, Proc. Natl. Acad. Sci. U.S.A. 85:2444, 1988. Altschul et al., Nature Genet. 6:119, 1994, present a detailed consideration of sequence alignment methods and homology/identity calculations. The NCBI Basic Local Alignment Search Tool (BLAST (Altschul et al, J. Mol. Biol. 215:403, 1990) is available from several sources, including the National Center for Biotechnology Information (NCBI, Bethesda, Md., USA) and on the internet, for use in connection with the sequence analysis programs blastp, blastn, blastx, tblastn and tblastx. A description of how to determine sequence identity or homology using this program is available on the NCBI website.

Antibodies of the present invention also include chimerized or humanized monoclonal antibodies generated from antibodies of the present invention. In one embodiment, humanized antibodies are antibody molecules from non-human species having one, two or all CDRs from the non-human species and one, two or all three framework regions from a human immunoglobulin molecule. A chimeric antibody is a molecule in which different portions are derived from different animal species. For example, an antibody may contain a variable region derived from a murine mAb and a human immunoglobulin constant region. Chimeric antibodies can be produced by recombinant DNA techniques. Morrison, et al., Proc Natl Acad Sci, 81:6851-6855 (1984). For example, a gene encoding a murine (or other species) antibody molecule is digested with restriction enzymes to remove the region encoding the murine Fc, and the equivalent portion of a gene encoding a human Fc constant region is then substituted into the recombinant DNA molecule. Chimeric antibodies can also be created by recombinant DNA techniques where DNA encoding murine V regions can be ligated to DNA encoding the human constant regions. Better et al., Science, 1988, 240:1041-1043. Liu et al. PNAS, 1987 84:3439-3443. Liu et al., J. Immunol., 1987, 139:3521-3526. Sun et al. PNAS, 1987, 84:214-218. Nishimura et al., Canc. Res., 1987, 47:999-1005. Wood et al. Nature, 1985, 314:446-449. Shaw et al., J. Natl. Cancer Inst., 1988, 80:1553-1559. International Patent Publication Nos. WO1987002671 and WO 86/01533. European Patent Application Nos. 184,187; 171,496; 125,023; and 173,494. U.S. Pat. No. 4,816,567.

Thus, SARS-CoV-2 antibodies of the present invention include in combination with a heavy chain or light chain variable region, a heavy chain or light chain constant region, a framework region, or any portion thereof, of non-murine origin, preferably of human origin, which can be incorporated into an antibody of the present invention.

Antibodies of the present invention are capable of modulating, decreasing, antagonizing, mitigating, alleviating, blocking, inhibiting, abrogating and/or interfering with the SARS-CoV-2 virus.

As used herein, the term “antigen-binding domain” or “antigen-binding fragment” refers to a portion or region of an intact antibody molecule that is responsible for antigen binding. An antigen-binding fragment is capable of binding to the same antigen to which the parent antibody binds. Examples of antigen-binding fragments include, but are not limited to: (i) a Fab fragment, which can be a monovalent fragment composed of a V_{H}-C_{H}1 chain and a V_{L}-C_{L} chain; (ii) a F(ab′)₂ fragment which can be a bivalent fragment composed of two Fab fragments linked by a disulfide bridge at the hinge region; (iii) a Fv fragment, composed of the V_{H} and V_{L} domains of an antibody molecule associated together by noncovalent interaction; (iv) a single chain Fv (scFv), which can be a single polypeptide chain composed of a V_{H} domain and a V_{L} domain through a peptide linker; and (v) a (scFv)₂, which can comprise two V_{H} domains linked by a peptide linker and two V_{L} domains, which are associated with the two V_{H} domains via disulfide bridges.

The antibody can be administered in a single dose treatment or in multiple dose treatments on a schedule and over a time period appropriate to the age, weight and condition of the subject, the particular composition used, and the route of administration, for prophylactic or curative purposes, etc. For example, in one embodiment, the antibody according to the invention is administered once per month, twice per month, three times per month, every other week (qow), once per week (qw), twice per week (biw), three times per week (tiw), four times per week, five times per week, six times per week, every other day (qod), daily (qd), twice a day (qid), three times a day (tid), four times a day (qid) or 6 times a day.

For ease of administration and uniformity of dosage, parenteral dosage unit form may be used. Dosage unit form as used herein refers to physically discrete units suited as unitary dosages for the subject to be treated; each unit containing a predetermined quantity of antibody calculated to produce the desired therapeutic effect.

An “effective amount,” as used herein, refers to a dose of the antibody that is sufficient to reduce the symptoms and signs of SARS-CoV-2, such as cough, fever shortness of breath, viral shedding, or pneumonia which is detectable, either clinically or radiologically through various imaging means. The term “effective amount” and “therapeutically effective amount” are used interchangeably.

The effective amount of the antibody or the conjugate depends on the subject and the condition to be treated. The specific dose level for any particular subject depends upon a variety of factors including the activity of the specific peptide, the age, body weight, general health, sex, diet, time of administration, route of administration, and rate of excretion, drug combination and the severity of the particular disease undergoing therapy and can be determined by one of ordinary skill in the art without undue experimentation.

The term “subject” may refer to a vertebrate suspected of having SARS-CoV-2 or has confirmed SARS-CoV-2 infection. Subjects include warm-blooded animals, such as mammals, such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, mouse, rabbit, rat, gerbil, guinea pig, etc.).

The term “treating” as used herein refers to the application or administration of a composition including one or more active agents to a subject afflicted with a disorder, a symptom or conditions of the disorder, or a progression of the disorder, with the purpose to cure, heal, alleviate, relieve, alter, remedy, ameliorate, improve, or affect the disorder, the symptoms or conditions of the disorder, the disabilities induced by the disorder, or the progression of the disorder or the symptom or condition thereof.

As used herein, “pharmaceutically acceptable” means that the carrier is compatible with the active ingredient in the composition, and preferably can stabilize said active ingredient and is safe to the individual receiving the treatment. Said carrier may be a diluent, vehicle, excipient, or matrix to the active ingredient. Some examples of appropriate excipients include lactose, dextrose, sucrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may additionally comprise lubricants, such as talc, magnesium stearate, and mineral oil; wetting agents; emulsifying and suspending agents; preservatives, such as methyl and propyl hydroxybenzoates; sweeteners; and flavoring agents. The composition of the present invention can provide the effect of rapid, continued, or delayed release of the active ingredient after administration to the patient.

According to the present invention, the form of said pharmaceutical composition may be tablets, pills, powder, lozenges, packets, troches, elixirs, suspensions, lotions, solutions, syrups, soft and hard gelatin capsules, suppositories, sterilized injection fluid, and packaged powder.

As used herein, the term “polypeptide” refers to a polymer composed of amino acid residues linked via peptide bonds. The term “protein” typically refers to relatively large polypeptides. The term “peptide” typically refers to relatively short polypeptides (e.g., containing up to 100, 90, 70, 50, 30, or 20 amino acid residues).

As used herein, an “isolated” substance means that it has been altered by the hand of man from the natural state. In some embodiments, the polypeptide (e.g. antibody) or nucleic acids of the present invention can be said to be “isolated” or “purified” if they are substantially free of cellular material or chemical precursors or other chemicals/components that may be involved in the process of peptides/nucleic acids preparation. It is understood that the term “isolated” or “purified” does not necessarily reflect the extent to which the peptide has been “absolutely” isolated or purified e.g. by removing all other substances (e.g., impurities or cellular components). In some cases, for example, an isolated or purified polypeptide includes a preparation containing the polypeptide having less than 50%, 40%, 30%, 20% or 10% (by weight) of other proteins (e.g. cellular proteins), having less than 50%, 40%, 30%, 20% or 10% (by volume) of culture medium, or having less than 50%, 40%, 30%, 20% or 10% (by weight) of chemical precursors or other chemicals/components involved in synthesis procedures.

As used herein, the term “specific binds” or “specifically binding” refers to a non-random binding reaction between two molecules, such as the binding of the antibody to an epitope of its target antigen. An antibody that “specifically binds” to a target antigen or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen or an epitope than it does with other targets/epitopes. An antibody “specifically binds” to a target antigen if it binds with greater affinity, avidity, more readily, and/or with greater duration than it binds to other substances. In other words, it is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means specific/preferential binding. The affinity of the binding is defined in terms of a dissociation constant (Kd). Typically, specifically binding when used with respect to an antibody can refer to an antibody that specifically binds to (recognize) its target with an Kd value less than about 10⁻⁷ M, such as about 10⁻⁸ M or less, such as about 10⁻⁹ M or less, about 10⁻¹⁰ M or less, about 10⁻¹¹ M or less, about 10⁻¹² M or less, or even less, and binds to the specific target with an affinity corresponding to a Kd that is at least ten-fold lower than its affinity for binding to a non-specific antigen (such as BSA or casein), such as at least 100 fold lower, for instance at least 1,000 fold lower, such as at least 10,000 fold lower.

As used herein, the term “Coronavirus” refers to viruses belonging to the family Coronavirinae. Coronaviruses are enveloped RNA viruses that are spherical in shape and characterized by crown-like spikes on the surface under an electron microscope, hence the name. This type of virus can be further divided into four subgroups: alpha (α), beta (β), gamma (γ), and delta (δ). There are seven human coronavirus strains, including two alpha coronaviruses (HCov-229E and HCoV-NL63), two beta coronaviruses (HCov-HKU1 and HCov-OC43), Middle East respiratory syndrome coronavirus (MERS-CoV), SARS-CoV, and the newly discovered SARS-CoV-2.

As used herein, the term “severe acute respiratory syndrome coronavirus 2 (SARS-CoV-2)” refers to the strain of coronavirus that causes Coronavirus disease 2019 (COVID-19). SARS-CoV-2 is a positive-sense single-stranded RNA virus that is a member of the genus Betacoronavirus of the family Coronavirinae. The RNA sequence of SARS-CoV-2 is approximately 30,000 bases in length. Each SARS-CoV-2 virion is 50-200 nanometres in diameter. Like other coronaviruses, SARS-CoV-2 has four structural proteins, known as the S (spike), E (envelope), M (membrane), and N (nucleocapsid) proteins; the N protein holds the RNA genome, and the S, E, and M proteins together create the viral envelope.

As used herein, the term “spike protein,” “S polypeptide,” “S protein,” “SARS-CoV-2 spike,” or “SARS-CoV-2 S protein,” which can be used interchangeably, refers to a surface structure glycoprotein on SARS CoV-2 and is responsible for allowing the virus to attach to and fuse with the membrane of a host cell. Each monomer of trimeric S protein is about 180 kDa, and contains two subunits, S1 and S2, mediating attachment and membrane fusion, respectively. Spike protein mainly enters human cells by binding to the receptor angiotensin converting enzyme 2 (ACE2).

As used herein, the term “nucleocapsid protein,” “N polypeptide,” “N protein,” “SARS-CoV-2 nucleocapsid,” or “SARS-CoV-2 N protein,” which can be used interchangeably, refers to the multi-domain RNA-binding protein of SARS CoV-2 and is critical for viral genome packaging. N protein contains three dynamic disordered regions that house putative transiently-helical binding motifs; and the two folded domains interact minimally such that full-length N protein is a flexible and multivalent RNA-binding protein. (Cubuk 2021).

The term “nucleic acid” or “polynucleotide” can refer to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form.

The term “encoding” refers to the inherent property of specific sequences of nucleotides in a polynucleotide (e.g., a gene, a cDNA, or an mRNA) to serve as templates for synthesis of other polymers and macromolecules in biological processes having either a given sequence of nucleotides (i.e., rRNA, tRNA and mRNA) or a given sequence of amino acids and the biological properties resulting therefrom. Therefore, a gene encodes a protein if transcription and translation of mRNA produced by that gene produces the protein in a cell or other biological system. It is understood by a skilled person that numerous different polynucleotides and nucleic acids can encode the same polypeptide as a result of the degeneracy of the genetic code. It is also understood that skilled persons may, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleotide sequence encoding an amino acid sequence” encompasses all nucleotide sequences that are degenerate versions of each other and that encode the same amino acid sequence.

Numerous methods conventional in this art are available for obtaining monoclonal antibodies or antigen-binding fragments thereof. In some embodiments, the monoclonal antibodies provided herein may be made by the conventional hybridoma technology. In some embodiments, the monoclonal antibodies provided herein may be prepared via recombinant technology. In some embodiments, the monoclonal antibodies provided herein may be prepared by single cell expression system based on flow cytometry and PCR cloning of antigen specific B cells (Huang 2015, Huang 2017, Huang 2019).

When a full-length antibody is desired, coding sequences of any of the V_{H} and V_{L} chains described herein can be linked to the coding sequences of the Fc region of an immunoglobulin and the resultant gene encoding a full-length antibody heavy and light chains can be expressed and assembled in a suitable host cell, e.g., a plant cell, a mammalian cell, a yeast cell, or an insect cell.

Antigen-binding fragments can be prepared via routine methods. For example, F(ab′)₂ fragments can be generated by pepsin digestion of a full-length antibody molecule, and Fab fragments that can be made by reducing the disulfide bridges of F(ab′)₂ fragments. Alternatively, such fragments can also be prepared via recombinant technology by expressing the heavy and light chain fragments in suitable host cells and have them assembled to form the desired antigen-binding fragments either in vivo or in vitro. A single-chain antibody can be prepared via recombinant technology by linking a nucleotide sequence coding for a heavy chain variable region and a nucleotide sequence coding for a light chain variable region. Preferably, a flexible linker is incorporated between the two variable regions.

In general, the method of the present invention for detecting SARS-CoV-2 in a sample suspected of containing said SARS-CoV-2 comprises contacting the sample with any of the disclosed monoclonal antibodies or any combination thereof and assaying binding of the antibody with said sample.

There are various assay formats known to those of ordinary skill in the art for using antibodies to detect an antigen or pathogen in a sample. These assays that use antibodies specific to target antigens/pathogens are generally called immunoassays. Examples of immunoassays include but are not limited to ELISA (enzyme-linked immunosorbent assay), RIA (radioimmunoassay), FIA (fluorescence immunoassay), LIA (luminescence immunoassay), or immunoluminometric assay (ILMA). Such assays can be employed to detect the presence of SARS-CoV-2 in biological samples including blood, serum, plasma, saliva, cerebrospinal fluid, urine, stool, samples taken from respiratory tract, and other tissue specimens.

In some embodiments, the samples taken from the respiratory tract are nasopharyngeal (NP) or nasal (NS) swabs.

In some embodiments, the immunoassay is a sandwich assay or in a lateral flow assay format.

The following examples of specific aspects for carrying out the present invention are offered for illustrative purposes only, and are not intended to limit the scope of the present invention in any way.

### EXAMPLES

### Example 1 Preparation and Characterization of Monoclonal Antibodies Against SARS-CoV-2

1. Study Design

In this Example, SARS-CoV-2 antigen-specific human MAbs were isolated from peripheral plasmablasts in humans with natural SARS-CoV-2 infection, and then the antigenic specificity and phenotypic activities of human MAbs were characterized. The diagnosis of acute SARS-CoV-2 infection was based on positive real-time reverse transcriptase polymerase chain reaction (PCR) results of respiratory samples. The study protocol and informed consent were approved by the ethics committee at the Chang Gung Medical Foundation (Taoyuan, Taiwan) and the Taoyuan General Hospital, Ministry of Health and Welfare (Taoyuan, Taiwan). Each patient provided signed informed consent. The study and all associated methods were carried out in accordance with the approved protocol, the Declaration of Helsinki and Good Clinical Practice guidelines.

2. Staining and Sorting of Plasmablasts

Fresh peripheral blood mononuclear cells (PBMCs) were separated from whole blood by density gradient centrifugation and cryopreserved PBMCs were thawed. PBMCs were stained with a mix of fluorescent-labeled antibodies to cellular surface markers, including anti-CD3 (BD Biosciences, USA), anti-CD19 (BD Biosciences, USA), anti-CD27 (BD Biosciences, USA), anti-CD20 (BD Biosciences, USA), anti-CD38 (BD Biosciences, USA), anti-IgG (BD Biosciences, USA) and anti-IgM (BD Biosciences, USA). Plasmablasts were selected by gating on CD3⁻ CD20⁻CD19⁺CD27^{hi}CD38^{hi}IgG⁺IgM⁻ events and were isolated in chamber as single cell as previously described (Huang 2015, Huang 2017, Huang 2019).

3. Production of Human IgG 1 Monoclonal Antibodies

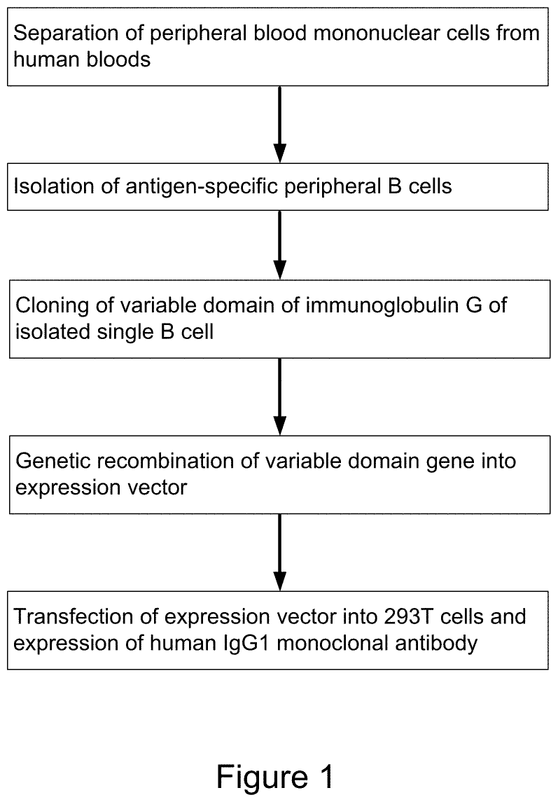

Sorted single cells were used to produce human IgG monoclonal antibodies as previously described (Huang 2015, Huang 2017, Huang 2019). Briefly, single cells were sorted directly to catch buffer and the variable region genes from each cell were amplified in a reverse transcriptase PCR (QIAGEN, Germany) using a cocktail of sense primers specific for the leader region and antisense primers to the Cγ constant region for heavy chain and Cκ and Cλ for light chain. The reverse transcriptase PCR products were amplified in separate PCR reactions for the individual heavy and light chain gene families using nested primers to incorporate restriction sites at the ends of the variable gene as previously described (Huang 2015, Huang 2017, Huang 2019). These variable genes were then cloned into expression vectors for the heavy and light chains. Plasmids were transfected into the 293T cell line for expression of recombinant full-length human IgG monoclonal antibodies in serum-free transfection medium (FIG. 1). A panel of monoclonal antibodies were further expanded and purified.

To determine the individual gene segments employed by VDJ and VJ rearrangements and the number of nucleotide mutations and amino acid replacements, the variable domain sequences were aligned with germline gene segments using the international ImMunoGeneTics (IMGT) alignment tool (http://www.imgt.org/IMGT_vquest/input).

4. Enzyme-Linked Immunosorbent Assay (ELISA)

The ELISA plates (Corning® 96-well Clear Polystyrene High Bind Stripwell™ Microplate, USA) were coated with SARS-CoV-2 antigen (Spike extracellular domain or spike S1 subunit or spike receptor binding domain (RBD) or spike S2 subunit or nucleocapsid, Sino Biological, China) or SARS antigen (Spike S1 subunit, Sino Biological, China) or Middle East respiratory syndrome coronavirus (MERS) antigen (Spike extracellular domain, Sino Biological, China) or human coronavirus OC43 antigen (Spike extracellular domain, Sino Biological, China) at optimal concentration in carbonate buffer and incubated at 4° C. overnight. The next day unbound antigens were removed by pipetting to avoid risk of forming aerosols. Nonspecific binding was blocked with the solution of phosphate-buffered saline (PBS) with 3% bovine serum albumin (BSA) at room temperature for 1 hour on a shaker. After removing blocking buffer, monoclonal antibody-containing cell culture supernatant or purified monoclonal antibody preparation were added and incubated at 37° C. for 1 hour. The non-transfected cell culture supernatant and anti-influenza human monoclonal antibody BS 1A (in house) were used as negative antibody controls for each experiment. The anti-SARS spike monoclonal antibody CR3022 and convalescent serum were used as positive antibody controls for each experiment. After incubation, the plate was washed and incubated with horseradish peroxidase-conjugated rabbit anti-human IgG (Rockland Immunochemicals, USA) as secondary antibody. After incubation, the plate was washed and developed with 3,3′,5,5′-Tetramethylbenzidine (TMB) substrate reagent (BD Biosciences, USA). Reaction was stopped by 0.5M Hydrochloric acid and the optical density was measured at OD₄₅₀ on a microplate reader. The well that yielded an OD value four times the mean absorbance of negative controls (BS 1A) was considered positive.

5. Immunofluorescence Assay

Under biosafety level 3 (BSL-3) conditions, cells were infected with 100 TCID₅₀ (median tissue culture infectious dose) SARS-CoV-2 (hCoV-19/Taiwan/CGMH-CGU-01/2020, EPI_ISL_411915). Infected cells were placed on coverslips and, and fixed with acetone at room temperature for 10 minutes. After blocking with 1% BSA at room temperature for 1 hour and washing, fixed cells were incubated with MAb-containing cell culture supernatant. The anti-influenza human monoclonal antibody BS 1A was used as negative antibody controls for each experiment. The anti-SARS spike glycoprotein MAb CR3022 and convalescent serum were used as positive antibody controls for each experiment. Following incubation and wash, cells were stained with FITC-conjugated anti-human IgG secondary antibody and Evans blue dye as counterstain. Antibody-bound infected cells demonstrated an apple-green fluorescence against a background of red fluorescing material stained by the Evans Blue counterstain. Images were acquired with original magnification 40×, scale bar 20 μm.

6. Flow Cytometry Assay

SARS-CoV-2 receptor-binding domain (RBD)-expressed Madin-Darby Canine Kidney (MDCK) cells (RBD cells) were prepared and resuspended. RBD Cells were probed with purified MAbs in 3% BSA. Bound primary antibodies were detected with FITC-conjugated anti-IgG secondary antibody. The binding activities were analyzed by BD FACSCanto™ II flow cytometer (BD Biosciences, USA). The nonlinear regression analysis was performed to obtain the Kd value of MAb against SARS-CoV-2 RBD.

7. Results

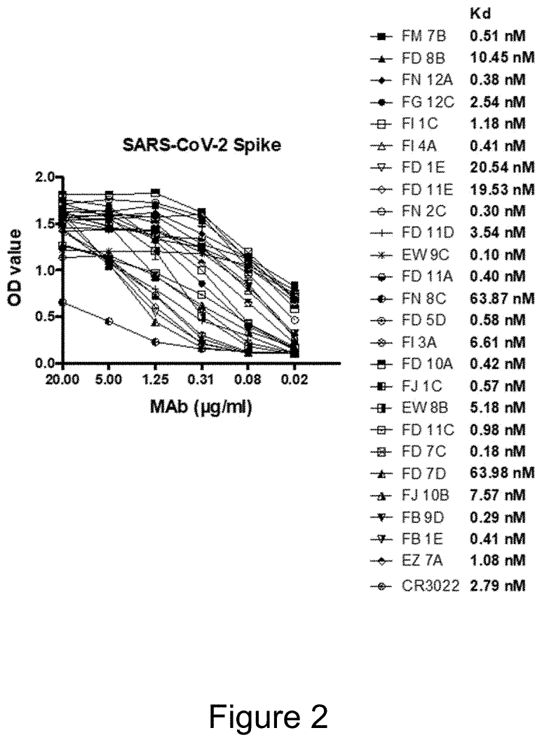

Peripheral blood samples were obtained from convalescent patients with laboratory-confirmed SARS-CoV-2 infections and circulating plasmablasts were identified by flow cytometry (Huang 2015, Huang 2017, Huang 2019). Sorted single cells were used to generate SARS-CoV-2 human monoclonal antibodies (FIG. 1). A total of 64 SARS-CoV-2 antigen-reactive human IgG1 monoclonal antibodies were produced, of them 34 were reactive to spike protein of SARS-CoV-2 (Table 1, FIG. 2) and 30 were reactive to nucleocapsid protein of SARS-CoV-2 (Table 5), as tested by binding of recombinant proteins in the enzyme-linked immunosorbent assay.

TABLE 1
Antigenic specificity of 34 SARS-CoV-2 spike
reactive human monoclonal antibodies.

| | | | | | Cross-reactive to other | | |
|---|---|---|---|---|---|---|---|
| | Antigenic specificity | | | | betacoronaviruses | | |
| | SARS- | SARS- | SARS- | SARS- | | | Human |
| | CoV-2 | CoV-2 | CoV-2 | CoV-2 | SARS spike | MERS | coronavirus |
| MAb | spike | S1 subunit | RBD | S2 | S1 subunit | spike | OC43 spike |
| FM 7B | + | + | + | − | + | − | − |
| FD 8B | + | − | − | − | − | − | − |
| EW 9B | + | − | − | − | − | − | − |
| FN 12A | + | + | + | − | ± | ± | ± |
| FG 12C | + | − | − | − | − | − | − |
| FI 1C | + | + | + | − | − | − | − |
| FI 4A | + | + | + | − | − | − | − |
| FD 1E | + | − | − | − | − | − | − |
| FD 11E | + | − | − | − | − | − | − |
| FN 2C | + | − | − | + | − | − | + |
| EY 6A | + | + | + | − | + | − | − |
| EY 6A-1* | + | + | + | − | + | − | − |
| FD 11D | + | ± | − | − | ± | ± | ± |
| EW 9C | + | − | − | + | − | ± | + |
| EW 9C-1* | + | − | − | + | − | ± | + |
| FD 1D | + | − | − | + | ± | ± | ± |
| FG 7A | + | − | − | + | − | − | − |
| FM 1A | + | − | − | + | − | − | − |
| FD 11A | + | + | + | − | − | − | − |
| FN 8C | + | − | − | − | − | − | − |
| FD 5E | + | − | − | − | − | − | − |
| FD 5D | + | + | + | − | − | − | − |
| FI 3A | + | + | + | − | − | − | − |
| FD 10A | + | − | − | + | ± | ± | ± |
| FJ 4E | + | − | − | + | ± | + | + |
| FJ 1C | + | + | − | − | ± | − | − |
| EW 8B | + | − | − | − | − | − | − |
| FD 11C | + | + | − | − | − | − | − |
| FD 7C | + | + | − | − | − | − | − |
| FD 7D | + | − | − | − | − | − | − |
| FJ 10B | + | + | + | − | + | − | − |
| FB 9D | + | − | − | + | − | + | + |
| FB 1E | + | − | − | + | − | + | + |
| EZ 7A | + | + | + | − | + | + | + |

*Both EY 6A and EW 9C have an additional pair of expression vectors.
Abbreviations: Mab, monoclonal antibody; SARS-CoV-2, severe acute respiratory syndrome coronavirus 2; RBD, receptor-binding domain; SARS, severe acute respiratory syndrome coronavirus; MERS, Middle East respiratory syndrome coronavirus.

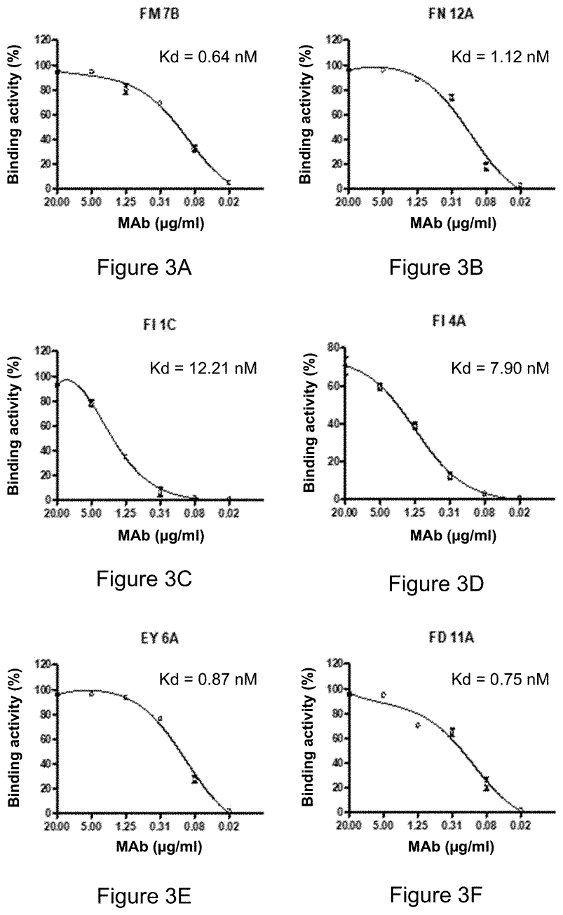

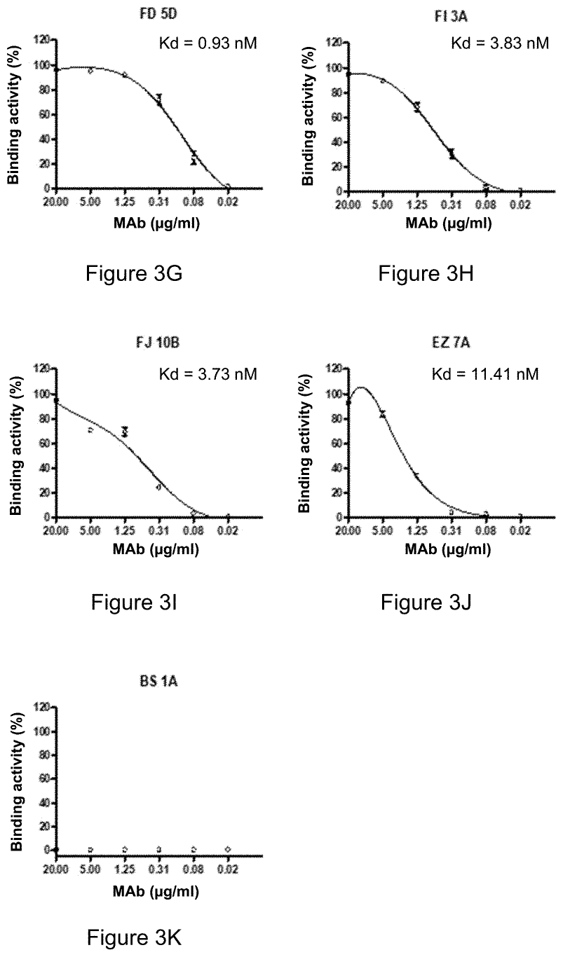

Among spike-reactive antibodies, 15 recognize the S1 subunit and 10 recognize the receptor-binding domain (RBD) of the SARS-CoV-2 spike protein (Table 1, FIGS. 3A to 3K). Thirteen (13) of these SARS-CoV-2 spike-reactive antibodies cross-react to the spike protein of other betacoronaviruses, including SARS, MERS or human coronavirus OC43 (Table 1), suggesting the presence of conserved epitopes on the spike of betacoronaviruses. Twenty-four (24) spike-reactive antibodies tested bound to viral antigens expressed on the infected cells by immunofluorescence assay, suggesting the majority of spike-reactive human antibodies may recognize complex conformational epitopes on the viral spike.

Variable domain sequences were obtained from the 34 SARS-CoV-2 spike-reactive monoclonal antibodies, each of which was unique and harbored somatic mutations (Table 2, Table 3, Table 4). Table 2 shows that SARS-CoV-2 spike-reactive monoclonal antibodies were evolved from 25 clonal groups defined by their heavy chain VDJ and light chain VJ rearrangements. Their average nucleotide somatic mutations are 6±9 in the heavy chain variable regions and 4±6 in the light chain variable regions. It was noted that 14 SARS-CoV-2 spike-reactive antibodies carry low number (less than 2) of somatic mutations in the heavy chain variable region, suggesting a de-novo B cell response to the SARS-CoV-2 virus in humans. Table 3 shows the nucleotide and amino acid sequences of the heavy chain variable regions and the light chain variable regions of the 34 SARS-CoV-2 spike-reactive monoclonal antibodies and Table 4 shows the amino acid sequences of complementarity-determining regions (CDRs) of the heavy chain variable regions and the light chain variable regions of the 34 SARS-CoV-2 spike-reactive human monoclonal antibodies.

TABLE 2
Gene usage of heavy and light chain variable domains of SARS-CoV-2
spike-reactive human monoclonal antibodies.

| | | | | | | V_{H} junction | Mut | aa | | | V_{L} Junction | Mut | aa |
|---|---|---|---|---|---|---|---|---|---|---|---|---|---|
| MAb | H-L | V_{H} | J_{H} | D_{H} | rf | sequence | # | Sub | V_{L} | J_{L} | Sequence | # | Sub |
| FM 7B | H-λ | 1-3*04 F | 5*02 F | 2-2*01 F | 2 | CARDPTYCSSTSCY | 3 | 1 | 3-21*02 F | 3*02 F | CQVWDSTGDHS | 2 | 2 |
| | | | | | | PFSWFDPW | | | | | WVF | | |
| FD 8B | H-κ | 1-24*01 F | 6*02F | 2-2*01 F | 2 | CATAAAINCSSTSC | 0 | 0 | 2-24*01 F | 2*01 F | CTQATQFPYTF | 2 | 2 |
| | | | | | | YYYYYYYGMDVW | | | | | | | |
| EW 9B | H-κ | 1-46*01 or | 6*02 F | 2-2*01 F | 3 | CAREDGVVPAANL | 2 | 2 | 3-11*01F | 4*01 F | CQQRSNWPLTF | 0 | 0 |
| | | 03 F | | | | MISLEDYYYYGMDVW | | | | | | | |
| FN 12A | H-λ | 1-69*04 or | 6*02 F | 2-2*01 F | 2 | CARSGCSSTSCPSN | 1 | 0 | 1-51*01 F | 3*02 F | CGTWDSSLSALV | 1 | 0 |
| | | 09 F | | | | LYYYYYGMDVW | | | | | F | | |
| FG 12C | H-λ | 3-9*01 F | 4*02 F | 4-17*01 F | 2 | CAKDMRVHDYGD | 0 | 0 | 3-21*01 F | 2*01 or | CQVWDSSSDHPV | 1 | 1 |
| | | | | | | YYFDYW | | | | 3*01 F | F | | |
| FI 1C | H-λ | 3-11*04 F | 3*02 F | 6-13*01 F | 1 | CARRSNRFLIAFDIW | 3 | 2 | 2-14*01 F | 2*01 or | CSSYTSSSTLVVF | 1 | 1 |
| | | | | | | | | | | 3*01 F | | | |
| FI 4A | H-λ | 3-21*01 F | 4*02 F | 2-21*02 F | 2 | CATYLFGDSHTYW | 9 | 7 | 6-57*02 F | 3*02 F | CQSYDSSNLHWV | 0 | 0 |
| | | | | | | | | | | | F | | |
| FD 1E | H-λ | 3-21*01 F | 6*02 F | 6-13*01 F | 2 | CASLAAAGPETYY | 1 | 0 | 3-1*01 F | 2*01 or | CQAWDSSVVF | 0 | 0 |
| | | | | | | YYGMDVW | | | | 3*01 F | | | |
| FD 11E | H-λ | 3-21*01 F | 6*02 F | 6-13*01 F | 2 | CASLAAAGPETYY | 0 | 0 | 3-1*01 F | 2*01 or | CQAWDSSVVF | 0 | 0 |
| | | | | | | YYGMDVW | | | | 3*01 F | | | |
| FN 2C | H-λ | 3-30*03 or | 3*01 or | 3-10*01 F | 1 | CAKRREIFWLGEPP | 22 | 14 | 1-40*01F | 3*02 F | CQSYDSSLSGSVF | 8 | 4 |
| | | 18 or 3-30- | 02 F | | | LSDAFDFW | | | | | | | |
| | | 5*01 F | | | | | | | | | | | |
| EY 6A | H-κ | 3-30*03 or | 4*02 F | 2-21*01 F | 1 | CAKDGGKLWVYYF | 6 | 5 | 1-39*01 F | 4*01 F | CQQSYSTLALTF | 0 | 0 |
| | | 18 or 3-30- | | | | DYW | | | or 1D- | | | | |
| | | 5*01 F | | | | | | | 39*01 F | | | | |
| EY 6A-1* | H-κ | 3-30*03 or | 4*02 F | 2-21*01 F | 1 | CAKDGGKLWVYYF | 6 | 5 | 1-39*01 F | 4*01 F | CQQSYSTLALTF | 0 | 0 |
| | | 18 or 3-30- | | | | DYW | | | or 1D- | | | | |
| | | 5*01 F | | | | | | | 39*01 F | | | | |
| FD 11D | H-κ | 3-30*03 or | 4*02 F | 6-19*01 F | 1 | CAKEGAGSGWYRH | 1 | 1 | 3-20*01 F | 1*01 F | CQQYGSSPLTF | 0 | 0 |
| | | 18 or 3-30- | | | | HKPGYYFDYW | | | | | | | |
| | | 5*01 F | | | | | | | | | | | |
| EW 9C | H-κ | 3-30*03 or | 5*01 or | 3-10*01 F | 1 | CARATSIFWFGEGR | 33 | 15 | 3-11*01F | 4*01 F | CQQRSNWPLTF | 25 | 13 |
| | | 18 or 3-30- | 02 F | | | NWFDPW | | | | | | | |
| | | 5*01 F | | | | | | | | | | | |
| EW 9C-1* | H-κ | 3-30*03 or | 5*01 or | 3-10*01 F | 1 | CARATSIFWFGEGR | 33 | 15 | 3-11*01 F | 4*01 F | CQQRSNWPLTF | 25 | 13 |
| | | 18 or 3-30- | 02 F | | | NWFDPW | | | | | | | |
| | | 5*01 F | | | | | | | | | | | |
| FD 1D | H-κ | 3-30*04 or | 5*02 F | 3-10*01 F | 2 | CARAGSGSYLNWF | 1 | 0 | 3-11*01 F | 5*01 F | CQQRSNWPITF | 0 | 0 |
| | | 3-30-3*03 | | | | DPW | | | | | | | |
| | | F | | | | | | | | | | | |
| FG 7A | H-κ | 3-30-3*01 | 4*02 F | 1-26*01 F | 3 | CARSHSGSYRASLD | 2 | 2 | 3-20*01 F | 2*01 F | CQQYGSSPLYTF | 0 | 0 |
| | | F | | | | YW | | | | | | | |
| FM 1A | H-λ | 3-33*01 or | 4*02 F | 1-26*01 F | 1 | CAREGAVGATRGF | 1 | 0 | 3-21*02 F | 2*01 or | CQVWDSSSDQG | 2 | 1 |
| | | 06 F | | | | DYW | | | | 3*01 F | VF | | |
| FD 11A | H-λ | 3-33*01 or | 6*02 F | 3-9*01 F | 2 | CAKGPDILTGYYNY | 2 | 2 | 1-40*01 F | 2*01 or | CQSYDSSLSGFY | 0 | 0 |
| | | 06 F | | | | YYYGMDVW | | | | 3*01 F | VVF | | |
| FN 8C | H-λ | 3-33*05 F | 6*02 F | 3-9*01 F | 2 | CARERTYYDILTGY | 1 | 1 | 3-21*02 F | 3*02 F | CQVWDSSSDHW | 1 | 1 |
| | | | | | | RHYYGMDVW | | | | | VF | | |
| FD 5E | H-κ | 3-43D*03 F | 6*02 F | 3-3*01 F | 1 | CAKDSVRFRYYYG | 0 | 0 | 3-11*01F | 3*01 F | CQQRSNWPLTF | 0 | 0 |
| | | | | | | MDVW | | | | | | | |
| FD 5D | H-κ | 3-48*04 F | 6*02 F | 6-13*01 F | 2 | CASPGGITAAGTSV | 4 | 2 | 2-28*01 or | 1*01 F | CMQALQTPITWT | 0 | 0 |
| | | | | | | LFGYYGMDVW | | | 2D-28*01 F | | F | | |
| FI 3A | H-κ | 3-53*01 F | 3*02 F | 6-6*01 F | 3 | CARDHVRPGMNIW | 2 | 2 | 1-33*01 or | 4*01 F | CQQYDNLPVTF | 1 | 0 |
| | | | | | | | | | 1D-33*01 F | | | | |
| FD 10A | H-κ | 3-74*01 F | 3*02 F | — | — | CANMAFDIW | 0 | 0 | 4-1*01 F | 5*01 F | CQQYYSTPITF | 0 | 0 |
| FJ 4E | H-κ | 4-31*06 F | 5*02 F | 3-10*01 F | 2 | CARDEYDSSDSGIQ | 20 | 15 | 1-39*01 F | 1*01F | CQQSYSTPWTF | 15 | 10 |
| | | | | | | GHWFDPW | | | or 1D- | | | | |
| | | | | | | | | | 39*01 F | | | | |
| FJ 1C | H-λ | 4-38-2*02 | 4*02 F | 3-10*01 F | 1 | CARDKALLWFGEL | 0 | 0 | 2-14*01 F | 2*01 or | CSSYTSSSTLVF | 1 | 1 |
| | | F | | | | FTNLFDYW | | | | 3*01 F | | | |
| EW 8B | H-κ | 4-39*01 F | 3*02 F | 3-16*01 F | 2 | CARQEVWGGFDIW | 20 | 12 | 3-20*01 F | 1*01 F | CQQYGSSPTF | 4 | 4 |
| FD 11C | H-λ | 4-39*07 F | 6*02 F | 3-10*01 F | 2 | CAREYYYGSETKK | 2 | 1 | 3-1*01 F | 2*01 or | CQAWDSSTVF | 0 | 0 |
| | | | | | | YYYYYGMDVW | | | | 3*01 F | | | |
| FD 7C | H-κ | 4-59*01 F | 5*02 F | 3-10*02 F | 3 | CARDYRFGELFGRF | 1 | 1 | 3-15*01 F | 1*01 F | CQQYNNWPRAF | 2 | 1 |
| | | | | | | AWFDPW | | | | | | | |
| FD 7D | H-λ | 5-10-1*03 | 3*02 F | 2-2*01 F | 2 | CARHSDCSSTSCYF | 1 | 1 | 3-25*03 F | 2*01 or | CQSADSSGTYVV | 1 | 0 |
| | | F | | | | VDAFDIW | | | | 3*01 F | F | | |
| FJ 10B | H-κ | 5-10-1*03 | 6*02 F | 3-16*01 F | 1 | CARLDPRYGPDYY | 2 | 1 | 1-39*01 F | 4*01 F | CQQSYSTPLTF | 3 | 2 |
| | | F | | | | GMDVW | | | or 1D- | | | | |
| | | | | | | | | | 39*01 F | | | | |
| FB 9D | H-λ | 5-51*01 F | 6*02 F | 3-10*01 F | 3 | CARHWASMVRGVI | 27 | 11 | 2-8*01 F | 3*02 F | CSSYAFGGSDTR | 20 | 10 |
| | | | | | | RASHYYGMDVW | | | | | VF | | |
| FB 1E | H-λ | 5-51*01 F | 6*02 F | 3-10*01 F | 3 | CARHWASMVRGVI | 22 | 11 | 2-8*01 F | 3*02 F | CSSYAFGGSDIRV | 16 | 8 |
| | | | | | | RASHYYGMDVW | | | | | F | | |
| EZ 7A | H-λ | 5-51*01 F | 6*02 F | 5-12*01 F | 3 | CARGWVYRGFPYY | 2 | 1 | 2-11*01 F | 3*02 F | CCSYAGSYTLVF | 0 | 0 |
| | | | | | | GMDVW | | | | | | | |

*Both EY 6A and EW 9C have an additional pair of expression vectors.
Abbreviations: H, heavy; K, kappa; λ, lambda; V_{H}, variable gene segment of the heavy chain variable domain; D_{H}, diversity gene segment of the heavy chain variable domain; J_{H}, joining gene segment of the heavy chain variable domain; Mut, number of nucleotide mutations; Sub, number of amino acid substitutions; V_{L}, variable gene segment of the light chain variable domain; J_{L}, joining gene segment of the light chain variable domain.

TABLE 3
Nucleotide and amino acid sequences of the heavy chain variable regions
(V_{H}) and light chain variable regions (V_{L}) of the 34 SARS-CoV-2 spike-reactive human
monoclonal antibodies.
Nucleotide sequences of V_{H} and V_{L}

| | | |
|---|---|---|
| FM 7B | V_{H} | GAGGTGCAGCTGGTGCAGTCTGGGGCTGAGGTGAAGAAGCCTGGGGCCTCAGTGAAGGTTTCCTGCAAGGCTTCT |
| | | GGATACACCTTCACTAGCTATGCTATGCATTGGGTGCGCCAGGCCCCCGGACAAAGGCTTGAGTGGATGGGATGGAT |
| | | CAACACTGGCAATGGTAACACAAAATATTCACAGAAGTTCCAGGGCAGAGTCACCATTACCAGGGACACATCCGCG |
| | | AGCACAGCCTACATGGAGCTGAGCAGCCTGAGATCTGAAGACACGGCTGTGTATTACTGTGCGAGAGATCCCACCT |
| | | ATTGTAGTAGTACCAGCTGCTACCCTTTTAGCTGGTTCGACCCCTGGGGCCAGGGAACCCTGGTCACCGTCTCCTCA |
| | | (SEQ ID NO: 513) |
| | V_{L} | AATTTTATGCTGACTCAGCCACCCTCGGTGTCAGTGGCCCCAGGACAGACGGCCAGGATTACCTGTGGGGGAAACA |
| | | ACATTGGAAGTAAAAGTGTGCACTGGTACCAGCAGAAGCCAGGCCAGGCCCCTGTGCTGGTCGTCTATGATGATAG |
| | | CGACCGGCCCTCAGGGATCCCTGAGCGATTCTCTGGCTCCAACTCTGGGAACACGGCCACCCTGACCATCAGCAGG |
| | | GTCGAAGCCGGGGATGAGGCCGACTATTACTGTCAGGTGTGGGATAGTACTGGTGATCATTCTTGGGTGTTCGGCGG |
| | | AGGGACCAAGCTGACCGTCCTA (SEQ ID NO: 514) |
| FD 8B | V_{H} | GAGGTGCAGCTGTTGGAGTCTGGGGCTGAGGTGAAGAAGCCTGGGGCCTCAGTGAAGGTCTCCTGCAAGGTTTCC |
| | | GGATACACCCTCACTGAATTATCCATGCACTGGGTGCGACAGGCTCCTGGAAAAGGGCTTGAGTGGATGGGAGGTTT |
| | | TGATCCTGAAGATGGTGAAACAATCTACGCACAGAAGTTCCAGGGCAGAGTCACCATGACCGAGGACACATCTACA |
| | | GACACAGCCTACATGGAGCTGAGCAGCCTGAGATCTGAGGACACGGCCGTGTATTACTGTGCAACAGCTGCAGCAA |
| | | TTAATTGTAGTAGTACCAGCTGCTACTATTACTACTACTACTACGGTATGGACGTCTGGGGCCAAGGGACCACGGTCA |
| | | CCGTCTCCTCA (SEQ ID NO: 515) |
| | V_{L} | GATATTGTGATGACCCAGACTCCACTCTCCTCACCTGTCACCCTTGGACAGCCGGCCTCCATCTCCTGCAGGTCTAGT |
| | | CAAAGCCTCGTACACAGTGATGGAAACACCTACTTGAGTTGGCTTCAGCAGAGGCCAGGCCAGCCTCCAAGACTCC |
| | | TAATTTATAAGATTTCTAGCCGGTTCTCTGGGGTCCCAGACAGATTCAGTGGCAGTGGGGCAGGGACAGATTTCACA |
| | | CTGAAAATCAGCAGGGTGGAAGCTGAGGATGTCGGGGTTTATTACTGCACGCAAGCTACACAATTTCCGTACACTTT |
| | | TGGCCAGGGGACCAAAGTGGATATCAAA (SEQ ID NO: 516) |
| EW 9B | V_{H} | GAGGTGCAGCTGGTGCAGTCTGGGGCTGAGGTGAAGAAGCCTGGGGCCTCAGTGAAGGTTTCCTGCAAGGCATCT |
| | | GGATACACCTTCACCAGCTACTATATGCACTGGGTGCGACAGGCCCCTGGACAAGGGCTTGAGTGGATGGGAATAAT |
| | | CAACCCTAGTGGTGGTAGCACAAGCTACGCACAGAAGTTCCAGGGCAGAGTCACCATGACCAGGGACACGTCCAC |
| | | GAGCACAGTCTACATGGAGCTGAGCAGCCTGAGATCTGAGGACACGGCCGTGTATTACTGTGCGAGAGAGGATGGA |
| | | GTAGTACCAGCTGCTAATTTGATGATATCGTTGGAAGACTACTACTACTACGGTATGGACGTCTGGGGCCAAGGGACC |
| | | ACGGTCACCGTCTCCTCA (SEQ ID NO: 517) |
| | V_{L} | GAAATTGTGTTGACACAGTCTCCAGCCACCCTGTCTTTGTCTCCAGGGGAAAGAGCCACCCTCTCCTGCAGGGCCA |
| | | GTCAGAGTGTTAGCAGCTACTTAGCCTGGTACCAACAGAAACCTGGCCAGGCTCCCAGGCTCCTCATCTATGATGCA |
| | | TCCAACAGGGCCACTGGCATCCCAGCCAGGTTCAGTGGCAGTGGGTCTGGGACAGACTTCACTCTCACCATCAGCA |
| | | GCCTAGAGCCTGAAGATTTTGCAGTTTATTACTGTCAGCAGCGTAGCAACTGGCCCCTCACTTTCGGCGGAGGGACC |
| | | AAAGTGGATATCAAA (SEQ ID NO: 518) |
| FN 12A | V_{H} | CAGGTTCAGCTGGTGCAGTCTGGGGCTGAGGTGAAGAAGCCTGGGTCCTCGGTGAAGGTCTCCTGCAAGGCTTCTG |
| | | GAGGCACCTTCAGCAGCTATGCTATCAGCTGGGTGCGACAGGCCCCTGGACAAGGGCTTGAGTGGATGGGAAGGAT |
| | | CATCCCTATCCTTGGTATAGCAAACTACGCACAGAAGTTCCAGGGCAGAGTCACGATTACCGCGGACAAATCCACGA |
| | | GCACAGCCTACATGGAGCTGAGCAGCCTGAGATCTGAGGACACGGCCGTGTATTACTGTGCGAGATCGGGCTGTAG |
| | | TAGTACCAGCTGCCCCTCAAACCTTTACTACTACTACTACGGTATGGACGTCTGGGGCCAAGGGACCACGGTCACCG |
| | | TCTCCTCA (SEQ ID NO: 519) |
| | V_{L} | CAGTCTGTGCTGACGCAGCCGCCCTCAGTGTCTGCGGCCCCAGGACAGAAGGTCACCATCTCCTGCTCTGGAAGCA |
| | | GCTCCAACATTGGGAATAATTATGTATCCTGGTACCAGCAGCTCCCAGGAACAGCCCCCAAACTCCTCATTTATGACA |
| | | ATAATAAGCGACCCTCAGGGATTCCTGACCGATTCTCTGGCTCCAAGTCTGGCACGTCAGCCACCCTGGGCATCACC |
| | | GGACTCCAGACTGGGGACGAGGCCGATTATTACTGCGGAACATGGGATAGCAGCCTGAGTGCTTTGGTGTTCGGCG |
| | | GAGGGACCAAGCTGACCGTCCTA (SEQ ID NO: 520) |
| FG 12C | V_{H} | GAAGTGCAGCTGGTGGAGTCTGGGGGAGGCTTGGTACAGCCTGGCAGGTCCCTGAGACTCTCCTGTGCAGCCTCTG |
| | | GATTCACCTTTGATGATTATGCCATGCACTGGGTCCGGCAAGCTCCAGGGAAGGGCCTGGAGTGGGTCTCAGGTATT |
| | | AGTTGGAATAGTGGTAGCATAGGCTATGCGGACTCTGTGAAGGGCCGATTCACCATCTCCAGAGACAACGCCAAGA |
| | | ACTCCCTGTATCTGCAAATGAACAGTCTGAGAGCTGAGGACACGGCCTTGTATTACTGTGCAAAAGATATGAGGGTG |
| | | CATGACTACGGTGACTACTACTTTGACTACTGGGGCCAGGGAACCCTGGTCACCGTCTCCTCA (SEQ ID NO: 521) |
| | V_{L} | TCCTATGAGCTGACTCAGCCACCCTCAGTGTCAGTGGCCCCAGGAAAGACGGCCAGGATTACCTGTGGGGGAAACA |
| | | ACATTGGAAGTAAAAGTGTGCACTGGTACCAGCAGAAGCCAGGCCAGGCCCCTGTGCTGGTCATCTATTATGATAGC |
| | | GACCGGCCCTCAGGGATCCCTGAGCGATTCTCTGGCTCCAACTCTGGGAACACGGCCACCCTGACCATCAGCAGGG |
| | | TCGAAGCCGGGGATGAGGCCGACTATTACTGTCAGGTGTGGGATAGTAGTAGTGATCATCCGGTATTCGGCGGAGGG |
| | | ACCAAGCTGACCGTCCTA (SEQ ID NO: 522) |
| FI 1C | V_{H} | GAGGTGCAGCTGGTGGAGTCTGGGGGAGGCTTGGTCAAGCCTGGAGGGTCCCTGAGACTCTCCTGTGCAGCCTCTG |
| | | GATTCACCTTCAGTGACTACTACATGAGCTGGATCCGCCAGGCTCCAGGGAAGGGGCTGGAGTGGGTTTCATACATT |
| | | AGTAGTAGTGGTAGTACCATATACTACGCAGACTCTGTGAAGGGCCGATTCACCATCTCCAGGGACAACGCCAAGAA |
| | | CTCACTGTATCTGCAAATGAACAGCCTGAGAGCCGAGGACACGGCTGTGTATTACTGTGCGAGGCGCAGTAACAGG |
| | | TTTTTGATTGCTTTTGATATCTGGGGCCAAGGGACAATGGTCACCGTCTCTTCA (SEQ ID NO: 523) |
| | V_{L} | CAGTCTGCCCTGACTCAGCCTGCCTCCGTGTCTGGGTCTCCTGGACAGACGATCACCATCTCCTGCACTGGAACCAG |
| | | CAGTGACGTTGGTGGTTATAACTATGTCTCCTGGTACCAACAGCACCCAGGCAAAGCCCCCAAACTCATGATTTATG |
| | | AGGTCAGTAATCGGCCCTCAGGGGTTTCTAATCGCTTCTCTGGCTCCAAGTCTGGCAACACGGCCTCCCTGACCATC |
| | | TCTGGGCTCCAGGCTGAGGACGAGGCTGATTATTACTGCAGCTCATATACAAGCAGCAGCACTCTCGTGGTATTCGG |
| | | CGGAGGGACCAAGCTGACCGTCCCA (SEQ ID NO: 524) |
| FI 4A | V_{H} | GAGGTGCAGCTGGTGGAGTCTGGGGGAGGCCTGGTCAAGCCTGGGGGGTCCCTGAGACTCTCCTGTGCAGCCTCTG |
| | | GATTCACCTTCAGTCGCTTTAGCATGAACTGGGTCCGCCAGGCTCCAGGGAAGGGGCTGGAGTGGGTCTCCTCCATT |
| | | AGTAGTAGTGGTAGTTACATATACTTCGCAGACTCAGTGAAGGGCCGATTCACCATCTCCAGAGACAACGCCAAGAA |
| | | CTCACTGTATCTGCAAATGAACAGCCTGAGAGCCGAGGACACGGCTATATATTACTGTGCGACTTATTTATTTGGTGA |
| | | CTCCCATACCTACTGGGGCCAGGGAACCCTGGTCACCGTCTCCTCA (SEQ ID NO: 525) |
| | V_{L} | CAGTCTGTGCTGACGCAGCCCCACTCTGTGTCGGAGTCTCCGGGGAAGACGGTAACCATCTCCTGCACCGGCAGCA |
| | | GTGGCAGCATTGCCAGCAACTATGTGCAGTGGTACCAGCAGCGCCCGGGCAGTGCCCCCACCACTGTGATCTATGAG |
| | | GATAACCAAAGACCCTCTGGGGTCCCTGATCGGTTCTCTGGCTCCATCGACAGCTCCTCCAACTCTGCCTCCCTCAC |
| | | CATCTCTGGACTGAAGACTGAGGACGAGGCTGACTACTACTGTCAGTCTTATGATAGCAGCAATCTCCATTGGGTGT |
| | | TCGGCGGAGGGACCAAGCTGACCGTCCTA (SEQ ID NO: 526) |
| FD 1E | V_{H} | GAGGTGCAGCTGTTGGAGTCTGGGGGAGGCCTGGTCAAGCCTGGGGGGTCCCTGAGACTCTCCTGTGCAGCCTCTG |
| | | GATTCACCTTCAGTAGCTATAGCATGAACTGGGTCCGCCAGGCTCCAGGGAAGGGGCTGGAGTGGGTCTCATCCATT |
| | | AGTAGTAGTAGTAGTTACATATACTACGCAGACTCAGTGAAGGGCCGATTCACCATCTCCAGAGACAACGCCAAGAA |
| | | CTCACTGTATCTGCAAATGAACAGCCTGAGAGCCGAGGACACGGCTGTGTATTACTGTGCGAGCTTAGCAGCAGCTG |
| | | GCCCCGAAACCTACTATTACTACGGTATGGACGTCTGGGGCCAAGGGACAATGGTCACCGTCTCTTCA (SEQ ID NO: |
| | | 527) |
| | V_{L} | TCCTATGAGCTGACACAGCCACCCTCAGTGTCCGTGTCCCCAGGACAGACAGCCAGCATCACCTGCTCTGGAGATA |
| | | AATTGGGGGATAAATATGCTTGCTGGTATCAGCAGAAGCCAGGCCAGTCCCCTGTGCTGGTCATCTATCAAGATAGCA |
| | | AGCGGCCCTCAGGGATCCCTGAGCGATTCTCTGGCTCCAACTCTGGGAACACAGCCACTCTGACCATCAGCGGGAC |
| | | CCAGGCTATGGATGAGGCTGACTATTACTGTCAGGCGTGGGACAGCAGCGTGGTATTCGGCGGAGGGACCAAGCTG |
| | | ACCGTCCTA (SEQ ID NO: 528) |
| FD 11E | V_{H} | GAAGTGCAGCTGGTGGAGTCTGGGGGAGGCCTGGTCAAGCCTGGGGGGTCCCTGAGACTCTCCTGTGCAGCCTCTG |
| | | GATTCACCTTCAGTAGCTATAGCATGAACTGGGTCCGCCAGGCTCCAGGGAAGGGGCTGGAGTGGGTCTCATCCATT |
| | | AGTAGTAGTAGTAGTTACATATACTACGCAGACTCAGTGAAGGGCCGATTCACCATCTCCAGAGACAACGCCAAGAA |
| | | CTCACTGTATCTGCAAATGAACAGCCTGAGAGCCGAGGACACGGCTGTGTATTACTGTGCGAGCTTAGCAGCAGCTG |
| | | GCCCCGAAACCTACTACTACTACGGTATGGACGTCTGGGGCCAAGGGACAATGGTCACCGTCTCTTCA (SEQ ID NO: |
| | | 529) |
| | V_{L} | TCCTATGAGCTGACTCAGCCACCCTCAGTGTCCGTGTCCCCAGGACAGACAGCCAGCATCACCTGCTCTGGAGATAA |
| | | ATTGGGGGATAAATATGCTTGCTGGTATCAGCAGAAGCCAGGCCAGTCCCCTGTGCTGGTCATCTATCAAGATAGCAA |
| | | GCGGCCCTCAGGGATCCCTGAGCGATTCTCTGGCTCCAACTCTGGGAACACAGCCACTCTGACCATCAGCGGGACC |
| | | CAGGCTATGGATGAGGCTGACTATTACTGTCAGGCGTGGGACAGCAGCGTGGTATTCGGCGGAGGGACCAAGCTGA |
| | | CCGTCCTA (SEQ ID NO: 530) |
| FN 2C | V_{H} | GAGGTGCAGCTGTTGGAGTCCGGGGGAGGCGTGGTCCAGCCTGGGAGGTCCCTGAGACTCTCCTGTGCAGCCTCTG |
| | | GATTCAACTTCAATAACTATGGCATGCACTGGGTCCGCCAGGCTCCAGGCAAGGGGCTGGAGTGGGTGGCTACTATT |
| | | TCATATGAAGGAAGTAAAAAATTTTATGTAGACTCCGTGAAGGGCCGATTCACCATCTCCAAAGACAATTCCAAGAA |
| | | CACGCTGTATCTGCAGATGAACAGCCTGAGAGTTGACGACACGGCTTTTTATTACTGTGCGAAACGGAGGGAAATAT |
| | | TTTGGTTGGGGGAGCCACCTCTCTCGGATGCTTTTGATTTCTGGGGCCAAGGGACAATGGTCACCGTCTCTTCA (SEQ |
| | | ID NO: 531) |
| | V_{L} | CAGTCTGTGCTGACTCAGCCGCCCTCAGTGTCTGGGGCCCCAGGGCAGAGGGTCACCATCTCCTGCACTGGGAGCG |
| | | GCTCCAACATCGGGGCAGGTTATGATGTACACTGGTACCAGTTCCTTCCAGGAACAGCCCCCCAACTCCTCATCTATG |
| | | GTAACAACAATCGTCCCTCAGGGGTCCCTGACCGATTCTCTGGCTCCAAGTCTGGCACCTCAGCCTCCCTGGCCATC |
| | | ACTGGGCTCCAGGCTGAAGATGAGGCTGATTATTACTGCCAGTCCTATGACAGCAGCCTGAGTGGTTCGGTGTTCGG |
| | | CGGAGGGACCAAGCTGACCGTCCTA (SEQ ID NO: 532) |
| EY 6A | V_{H} | GAAGTGCAGCTGGTGGAGTCTGGGGGAGGCGTGGTCCAGCCTGGGAGGTCCCTGAGACTCTCCTGTGCAGCCTCTG |
| | | CATTCACCTTCAGTAGCTATGACATGCACTGGGTCCGCCAGGCTCCAGGCAAGGGGCTGGAGTGGGTGGCAGTTATA |
| | | TCATATGATGGAAGTAATAAATACTATGCAGACTCCGTGAAGGGCCGATTCACCATCTCCAGAGACAATTCCAAGAAC |
| | | ACGCTGTATCTGCAAATGAACAGCCTGAGAGCTGAGGACACGGCTGTGTATTACTGTGCGAAAGATGGGGGAAAGC |
| | | TATGGGTGTACTACTTTGACTACTGGGGCCAGGGAACCACGGTCACCGTCTCCTCA (SEQ ID NO: 533) |
| | V_{L} | GACATCCAGATGACCCAGTCTCCATCCTCCCTGTCTGCATCTGTAGGAGACAGAGTCACCATCACTTGCCGGGCAAG |
| | | TCAGAGCATTAGCAGCTATTTAAATTGGTATCAGCAGAAACCAGGGAAAGCCCCTAAGCTCCTGATCTATGCTGCATC |
| | | CAGTTTGCAAAGTGGGGTCCCATCAAGGTTCAGTGGCAGTGGATCTGGGACAGATTTCACTCTCACCATCAGCAGTC |
| | | TGCAACCTGAAGATTTTGCAACTTACTACTGTCAACAGAGTTACAGTACCCTCGCGCTCACTTTCGGCGGAGGGACC |
| | | AAAGTGGATATCAAA (SEQ ID NO: 534) |
| EY 6A-1* | V_{H} | GAAGTGCAGCTGGTGGAGTCTGGGGGAGGCGTGGTCCAGCCTGGGAGGTCCCTGAGACTCTCCTGTGCAGCCTCTG |
| | | CATTCACCTTCAGTAGCTATGACATGCACTGGGTCCGCCAGGCTCCAGGCAAGGGGCTGGAGTGGGTGGCAGTTATA |
| | | TCATATGATGGAAGTAATAAATACTATGCAGACTCCGTGAAGGGCCGATTCACCATCTCCAGAGACAATTCCAAGAAC |
| | | ACGCTGTATCTGCAAATGAACAGCCTGAGAGCTGAGGACACGGCTGTGTATTACTGTGCGAAAGATGGGGGAAAGC |
| | | TATGGGTGTACTACTTTGACTACTGGGGCCAGGGAACCCTGGTCACCGTCTCCTCA (SEQ ID NO: 535) |
| | V_{L} | GACATCCAGATGACCCAGTCTCCATCCTCCCTGTCTGCATCTGTAGGAGACAGAGTCACCATCACTTGCCGGGCAAG |
| | | TCAGAGCATTAGCAGCTATTTAAATTGGTATCAGCAGAAACCAGGGAAAGCCCCTAAGCTCCTGATCTATGCTGCATC |
| | | CAGTTTGCAAAGTGGGGTCCCATCAAGGTTCAGTGGCAGTGGATCTGGGACAGATTTCACTCTCACCATCAGCAGTC |
| | | TGCAACCTGAAGATTTTGCAACTTACTACTGTCAACAGAGTTACAGTACCCTCGCGCTCACTTTCGGCGGAGGGACC |
| | | AAGGTGGAGATCAAA (SEQ ID NO: 536) |
| FD 11D | V_{H} | GAAGTGCAGCTGGTGGAGTCTGGGGGAGGCGTGGTCCAGCCTGGGAGGTCCCTGAGACTCTCCTGTGCAGCCTCTG |
| | | GATTCACCTTCAGTAGCTATGGCATGCACTGGGTCCGCCAGGCTCCAGGCAAGGGGCTGGAGTGGGTGGCAGTTATA |
| | | TCATATGATGGAAGTAATAAATACTATGCAGACTCCGTGAAGGGCCGATTCACCATCTCCAGAGACAATTCCAAGAAC |
| | | ACGCTGTATCTGCAAATGAACAGCCTGAGAGCTGAGGACACGGCTGTGTATTACTGTGCGAAAGAAGGAGCCGGCA |
| | | GTGGCTGGTACCGCCACCACAAGCCGGGCTACTACTTTGACTACTGGGGCCAGGGAACCCTGGTCACCGTCTCCTC |
| | | A (SEQ ID NO: 537) |
| | V_{L} | GAAATTGTGTTGACACAGTCTCCAGGCACCCTGTCTTTGTCTCCAGGGGAAAGAGCCACCCTCTCCTGCAGGGCCA |
| | | GTCAGAGTGTTAGCAGCAGCTACTTAGCCTGGTACCAGCAGAAACCTGGCCAGGCTCCCAGGCTCCTCATCTATGGT |
| | | GCATCCAGCAGGGCCACTGGCATCCCAGACAGGTTCAGTGGCAGTGGGTCTGGGACAGACTTCACTCTCACCATCA |
| | | GCAGACTGGAGCCTGAAGATTTTGCAGTGTATTACTGTCAGCAGTATGGTAGCTCACCTCTAACGTTCGGCCAAGGG |
| | | ACCAAGGTGGAAATCAAA (SEQ ID NO: 538) |
| EW 9C | V_{H} | GAAGTGCAGCTGGTGGAGTCGGGGGGGGGCGTGGTCCAGCCTGGGGCGTCCCTGAGACTCTCCTGCGTAGCCTCC |
| | | GGATTCACCTTTAATAATTTTGGATTCCACTGGGTCCGCCAGGCTCCAGGCAAGGGGCTGGAGTGGCTGGCAGTGAT |
| | | ATCATATGAGGGAAGTAAGACTTACTATGCAGAGTCGCTGAAGGGCCGCTTCACCATCTCCAGAGACACTTCCAAGA |
| | | ACACGGTGTATCTGCAGATGAACAGCCTGAGGGCTGAGGACACGGCTGTCTATTACTGTGCGCGGGCGACTTCAATT |
| | | TTTTGGTTTGGAGAGGGCCGTAACTGGTTCGACCCCTGGGGCCAAGGGACAATGGTCACCGTCTCTTCA (SEQ ID |
| | | NO: 539) |
| | V_{L} | GAAATTGTGTTGACGCAGTCTCCAGGCACCGTGTCTTTGTCTGCAGGGGAAAGAGCCACCCTCTCCTGCAGGGCCA |
| | | GTCAGAATGTTGGCACCGACTTAGCCTGGTATGTTCAGAGACCTGGCCAGGCTCCCAGGCTCCTCATCTATGATACAT |
| | | CCAATAAGGCCACTGGCATCCCAGCCAGGTTTAGTGGTCGAGGGTCTGGGACAGACTTCACTCTCACCATCGACAG |
| | | CCTAGAGCCTGAAGACTTTGCAGTTTATTACTGTCAACAGCGTAGCAACTGGCCGCTCACTTTCGGCGGAGGGACCA |
| | | AGGTGGAAATCAGA (SEQ ID NO: 540) |
| EW 9C-1* | V_{H} | GAGGTGCAGCTGGTGGAGTCGGGGGGGGGCGTGGTCCAGCCTGGGGCGTCCCTGAGACTCTCCTGCGTAGCCTCC |
| | | GGATTCACCTTTAATAATTTTGGATTCCACTGGGTCCGCCAGGCTCCAGGCAAGGGGCTGGAGTGGCTGGCAGTGAT |
| | | ATCATATGAGGGAAGTAAGACTTACTATGCAGAGTCGCTGAAGGGCCGCTTCACCATCTCCAGAGACACTTCCAAGA |
| | | ACACGGTGTATCTGCAGATGAACAGCCTGAGGGCTGAGGACACGGCTGTCTATTACTGTGCGCGGGCGACTTCAATT |
| | | TTTTGGTTTGGAGAGGGCCGTAACTGGTTCGACCCCTGGGGCCAGGGAGCCCTGGTCACCGTCTCCTCA (SEQ ID |
| | | NO: 541) |
| | V_{L} | GAAATAGTGATGACGCAGTCTCCAGGCACCGTGTCTTTGTCTGCAGGGGAAAGAGCCACCCTCTCCTGCAGGGCCA |
| | | GTCAGAATGTTGGCACCGACTTAGCCTGGTATGTTCAGAGACCTGGCCAGGCTCCCAGGCTCCTCATCTATGATACAT |
| | | CCAATAGGGCCACTGGCATCCCAGCCAGGTTTAGTGGTCGAGGGTCTGGGACAGACTTCACTCTCACCATCGACAG |
| | | CCTAGAGCCTGAAGACTTTGCAGTTTATTACTGTCAACAGCGTAGCAACTGGCCGCTCACTTTCGGCGGAGGGACCA |
| | | AGGTGGAAATCAAA (SEQ ID NO: 542) |
| FD 1D | V_{H} | GAAGTGCAGCTGGTGGAGTCTGGGGGAGGCGTGGTCCAGCCTGGGAGGTCCCTGAGACTCTCCTGTGCAGCCTCTG |
| | | GATTCACCTTCAGTAGCTATGCTATGCACTGGGTCCGCCAGGCTCCAGGCAAGGGGCTGGAGTGGGTGGCAGTTATA |
| | | TCATATGATGGAAGTAATAAATACTACGCAGACTCCGTGAAGGGCCGATTCACCATCTCCAGAGACAATTCCAAGAA |
| | | CACGCTGTATCTGCAAATGAACAGCCTGAGAGCTGAGGACACGGCTGTGTATTACTGTGCGAGAGCGGGTTCGGGG |
| | | AGCTACCTCAACTGGTTCGACCCCTGGGGCCAGGGAACCCTGGTCACCGTCTCCTCA (SEQ ID NO: 543) |
| | V_{L} | GAAATTGTGTTGACACAGTCTCCAGCCACCCTGTCTTTGTCTCCAGGGGAAAGAGCCACCCTCTCCTGCAGGGCCA |
| | | GTCAGAGTGTTAGCAGCTACTTAGCCTGGTACCAACAGAAACCTGGCCAGGCTCCCAGGCTCCTCATCTATGATGCA |
| | | TCCAACAGGGCCACTGGCATCCCAGCCAGGTTCAGTGGCAGTGGGTCTGGGACAGACTTCACTCTCACCATCAGCA |
| | | GCCTAGAGCCTGAAGATTTTGCAGTTTATTACTGTCAGCAGCGTAGCAACTGGCCGATCACCTTCGGCCAAGGGACA |
| | | CGACTGGAGATTAAA (SEQ ID NO: 544) |
| FG 7A | V_{H} | GAGGTGCAGCTGGTGGAGTCTGGGGGAGGCGTGGTCCAGCCTGGGAGGTCCCTGAGACTCTCCTGTGCAGCCTCTG |
| | | GATTCACCTTCAGTAGCTATGCTATGCACTGGGTCCGCCAGGCTCCAGGCAAGGGGCTGGAGTGGGTGGCAGTTATA |
| | | TCATATGATGGAAGCAATAAATACTACGCAGACTCCGTGAAGGGCCGATTCACCATCTCCAGAGACAATTCCAAGAA |
| | | CACGCTGTATCTGCAAATGAACAGCCTGAGAGCTGAGGACACGGCTGTGTATTACTGTGCGAGATCCCATAGTGGGA |
| | | GCTACCGAGCCTCCCTTGACTACTGGGGCCAGGGAACCACGGTCACCGTCTCCTCA (SEQ ID NO: 545) |
| | V_{L} | GACATCCAGTTGACCCAGTCTCCAGGCACCCTGTCTTTGTCTCCAGGGGAAAGAGCCACCCTCTCCTGCAGGGCCA |
| | | GTCAGAGTGTTAGCAGCAGCTACTTAGCCTGGTACCAGCAGAAACCTGGCCAGGCTCCCAGGCTCCTCATCTATGGT |
| | | GCATCCAGCAGGGCCACTGGCATCCCAGACAGGTTCAGTGGCAGTGGGTCTGGGACAGACTTCACTCTCACCATCA |
| | | GCAGACTGGAGCCTGAAGATTTTGCAGTGTATTACTGTCAGCAGTATGGTAGCTCACCTTTGTACACTTTTGGCCAGG |
| | | GGACCAAGGTGGAAATCAAA (SEQ ID NO: 546) |
| FM 1A | V_{H} | GAGGTGCAGCTGTTGGAGTCTGGGGGAGGCGTGGTCCAGCCTGGGAGGTCCCTGAGACTCTCCTGTGCAGCGTCTG |
| | | GATTCACCTTCAGTAGCTATGGCATGCACTGGGTCCGCCAGGCTCCAGGCAAGGGGCTGGAATGGGTGGCAGTTATA |
| | | TGGTATGATGGAAGTAATAAATACTATGCAGACTCCGTGAAGGGCCGATTCACCATCTCCAGAGACAATTCCAAGAA |
| | | CACGCTGTATCTGCAAATGAACAGCCTGAGAGCCGAGGACACGGCTGTGTATTACTGTGCGAGAGAGGGGGCAGTG |
| | | GGAGCTACTAGGGGGTTTGACTACTGGGGCCAGGGAACCCTGGTCACCGTCTCCTCA (SEQ ID NO: 547) |
| | V_{L} | TCCTATGAGCTGACACAGCCACCCTCGGTGTCAGTGGCCCCAGGACAGACGGCCAGGATTACCTGTGGGGGAAACA |
| | | ACATTGGAAGTAAAAGTGTGCACTGGTACCAGCAGAAGCCAGGCCAGGCCCCTGTGCTGGTCGTCTATGATGATAG |
| | | CGACCGGCCCTCAGGGATCCCTGAGCGATTCTCTGGCTCCAACTCTGGGAACACGGCCACCCTGACCATCAGCAGG |
| | | GTCGAAGCCGGGGATGAGGCCGACTATTACTGTCAGGTGTGGGATAGTAGTAGTGATCAGGGGGTATTCGGCGGAG |
| | | GGACCAAGCTGACCGTCCTA (SEQ ID NO: 548) |
| FD 11A | V_{H} | GAAGTGCAGCTGGTGGAGTCTGGGGGAGGCGTGGTCCAGCCTGGGAGGTCCCTGAGACTCTCCTGTGCAGCGTCT |
| | | GGATTCACCTTCAGTAGCTATGGCATGCACTGGGTCCGCCAGGCTCCAGGCAAGGGGCTGGAGTGGGTGGCAGTTAT |
| | | ATGGTATGATGGAAGTAATAAATACTATGCAGACTCCGTGAAGGGCCGATTCACCATCTCCAGAGACAATTCCAAGA |
| | | ACACGCTGTATCTGCAAATGAACAGCCTGAGAGCCGAGGACACGGCTGTGTATTACTGTGCGAAAGGCCCCGATATT |
| | | TTGACTGGTTATTATAACTACTACTACTACGGTATGGACGTCTGGGGCCAAGGGACAATGGTCACCGTCTCTTCA(SEQ |
| | | ID NO: 549) |
| | V_{L} | CAGTCTGTGCTGACGCAGCCGCCCTCAGTGTCTGGGGCCCCAGGGCAGAGGGTCACCATCTCCTGCACTGGGAGCA |
| | | GCTCCAACATCGGGGCAGGTTATGATGTACACTGGTACCAGCAGCTTCCAGGAACAGCCCCCAAACTCCTCATCTAT |
| | | GGTAACAGCAATCGGCCCTCAGGGGTCCCTGACCGATTCTCTGGCTCCAAGTCTGGCACCTCAGCCTCCCTGGCCAT |
| | | CACTGGGCTCCAGGCTGAGGATGAGGCTGATTATTACTGCCAGTCCTATGACAGCAGCCTGAGTGGTTTCTATGTGG |
| | | TATTCGGCGGAGGGACCAAGCTGACCGTCCTA (SEQ ID NO: 550) |
| FN 8C | V_{H} | CAGGTGCAGCTGGTGGAGTCTGGGGGAGGCGTGGTCCAGCCTGGGAGGTCCCTGAGACTCTCCTGTGCAGCGTCTG |
| | | GATTCACCTTCAGTAGCTATGGCATGCACTGGGTCCGCCAGGCTCCAGGCAAGGGGCTGGAGTGGGTGGCAGTTATA |
| | | TCATATGATGGAAGTAATAAATACTATGCAGACACCGTGAAGGGCCGATTCACCATCTCCAGAGACAATTCCAAGAA |
| | | CACGCTGTATCTGCAAATGAACAGCCTGAGAGCCGAGGACACGGCTGTGTATTACTGTGCGAGAGAACGGACGTAT |
| | | TACGATATTTTGACTGGTTATAGACACTACTACGGTATGGACGTCTGGGGCCAAGGGACCACGGTCACCGTCTCCTCA |
| | | (SEQ ID NO: 551) |
| | V_{L} | TCCTATGAGCTGACACAGCCACCCTCGGTGTCAGTGGCCCCAGGACAGACGGCCAGGATTACCTGTGGGGGAAACA |
| | | ACATTGGAAGTAAAAGTGTGCACTGGTACCAGCAGAAGCCAGGCCAGGCCCCTGTGCTGGTCGTCTATGATGATAG |
| | | CGACCGGCCCTCAGGGATCCCTGAGCGATTCTCTGGCTCCAACTCTGGGAACACGGCCACCCTGACCATCAGCAGG |
| | | GTCGAAGCCGGGGATGAGGCCGACTATTACTGTCAGGTGTGGGATAGTAGTAGTGATCATTGGGTGTTCGGCGGAGG |
| | | GACCAAGCTGACCGTCCTA (SEQ ID NO: 552) |
| FD 5E | V_{H} | GAAGTGCAGCTGGTGGAGTCTGGGGGAGTCGTGGTACAGCCTGGGGGGTCCCTGAGACTCTCCTGTGCAGCCTCTG |
| | | GATTCACCTTTGATGATTATGCCATGCACTGGGTCCGTCAAGCTCCGGGGAAGGGTCTGGAGTGGGTCTCTCTTATTA |
| | | GTTGGGATGGTGGTAGCACCTACTATGCAGACTCTGTGAAGGGTCGATTCACCATCTCCAGAGACAACAGCAAAAA |
| | | CTCCCTGTATCTGCAAATGAACAGTCTGAGAGCTGAGGACACCGCCTTGTATTACTGTGCAAAAGATTCGGTACGAT |
| | | TTCGGTACTACTACGGTATGGACGTCTGGGGCCAAGGGACCACGGTCACCGTCTCCTCA (SEQ ID NO: 553) |
| | V_{L} | GAAATTGTGTTGACACAGTCTCCAGCCACCCTGTCTTTGTCTCCAGGGGAAAGAGCCACCCTCTCCTGCAGGGCCA |
| | | GTCAGAGTGTTAGCAGCTACTTAGCCTGGTACCAACAGAAACCTGGCCAGGCTCCCAGGCTCCTCATCTATGATGCA |
| | | TCCAACAGGGCCACTGGCATCCCAGCCAGGTTCAGTGGCAGTGGGTCTGGGACAGACTTCACTCTCACCATCAGCA |
| | | GCCTAGAGCCTGAAGATTTTGCAGTTTATTACTGTCAGCAGCGTAGCAACTGGCCTCTCACTTTCGGCCCTGGGACC |
| | | AAAGTGGATATCAAA (SEQ ID NO: 554) |
| FD 5D | V_{H} | GAAGTGCAGCTGGTGGAGTCTGGGGGAGGCTTGGTACAGCCTGGGGGGTCCCTGAGACTCTCCTGTGCAGCCTCTG |
| | | GATTCACCTTCAGTAGCTATAGCATGAACTGGGTCCGCCAGGCTCCAGGGAAGGGGCTGGAGTGGGTTTCATACATT |
| | | AGTAGTAGTAGTAGTACCATATACTACGCAGACCCTGTGAAGGGCCGATTCACCATCTCCAGAGACAACGCCAAGAA |
| | | CTCACTGTATCTGCAAATGAACAGCCTGAGAGCCGAGGACACGGCTGTGTATTACTGTGCGAGTCCCGGAGGGATC |
| | | ACAGCAGCTGGTACATCAGTTCTTTTTGGGTACTACGGTATGGACGTCTGGGGCCAAGGGACAATGGTCACCGTCTC |
| | | TTCA (SEQ ID NO: 555) |
| | V_{L} | GATATTGTGATGACTCAGTCTCCACTCTCCCTGCCCGTCACCCCTGGAGAGCCGGCCTCCATCTCCTGCAGGTCTAGT |
| | | CAGAGCCTCCTGCATAGTAATGGATACAACTATTTGGATTGGTACCTGCAGAAGCCAGGGCAGTCTCCACAGCTCCT |
| | | GATCTATTTGGGTTCTAATCGGGCCTCCGGGGTCCCTGACAGGTTCAGTGGCAGTGGATCAGGCACAGATTTTACAC |
| | | TGAAAATCAGCAGAGTGGAGGCTGAGGATGTTGGGGTTTATTACTGCATGCAAGCTCTACAAACTCCCATCACGTGG |
| | | ACGTTCGGCCAAGGGACCAAAGTGGATATCAAA (SEQ ID NO: 556) |
| FI 3A | V_{H} | GAGGTGCAGCTGTTGGAGTCTGGAGGAGGCTTGATCCAGCCTGGGGGGTCCCTGAGACTCTCCTGTGCAGCCTCTG |
| | | GGTTCACCGTCAGTAGCAACTACATGAGCTGGGTCCGCCAGGCTCCAGGGAAGGGGCTGGAGTGGGTCTCAGTTAT |
| | | TTATAGCGGTGGTAGCACATACTACGCAGACTCCGTGAAGGGCCGATTCACCATCTCCAGAGACAATTCCAAGAACA |
| | | CGCTGTATCTTCAAATGAACAGCCTGAGAGCCGAGGACACGGCCGTGTATTACTGTGCGAGAGATCACGTGCGGCC |
| | | CGGGATGAATATCTGGGGCCAAGGGACAATGGTCACCGTCTCTTCA (SEQ ID NO: 557) |
| | V_{L} | GCCATCCGGATGACCCAGTCTCCATCCTCCCTGTCTGCATCTGTAGGAGACAGAGTCACCATCACTTGCCAGGCGAG |
| | | TCAGGACATTAGCAACTATTTAAATTGGTATCAGCAGAAACCAGGGAAAGCCCCTAAGCTCCTGATCTACGATGCATC |
| | | CAATTTGGAAACAGGGGTCCCATCAAGGTTCAGTGGAAGTGGATCTGGGACAGATTTTACTTTCACCATCAGCAGCC |
| | | TGCAGCCTGAAGATATTGCAACATATTACTGTCAACAGTATGATAATCTTCCGGTCACTTTCGGCGGAGGGACCAAAG |
| | | TGGATATCAAA (SEQ ID NO: 558) |
| FD 10A | V_{H} | GAAGTGCAGCTGGTGGAGTCCGGGGGAGGCTTAGTTCAGCCTGGGGGGTCCCTGAGACTCTCCTGTGCAGCCTCTG |
| | | GATTCACCTTCAGTAGCTACTGGATGCACTGGGTCCGCCAAGCTCCAGGGAAGGGGCTGGTGTGGGTCTCACGTATT |
| | | AATAGTGATGGGAGTAGCACAAGCTACGCGGACTCCGTGAAGGGCCGATTCACCATCTCCAGAGACAACGCCAAGA |
| | | ACACGCTGTATCTGCAAATGAACAGTCTGAGAGCCGAGGACACGGCTGTGTATTACTGTGCAAACATGGCTTTTGAT |
| | | ATCTGGGGCCAAGGGACAATGGTCACCGTCTCTTCA (SEQ ID NO: 559) |
| | V_{L} | GACATCGTGATGACCCAGTCTCCAGACTCCCTGGCTGTGTCTCTGGGCGAGAGGGCCACCATCAACTGCAAGTCCA |
| | | GCCAGAGTGTTTTATACAGCTCCAACAATAAGAACTACTTAGCTTGGTACCAGCAGAAACCAGGACAGCCTCCTAAG |
| | | CTGCTCATTTACTGGGCATCTACCCGGGAATCCGGGGTCCCTGACCGATTCAGTGGCAGCGGGTCTGGGACAGATTT |
| | | CACTCTCACCATCAGCAGCCTGCAGGCTGAAGATGTGGCAGTTTATTACTGTCAGCAATATTATAGTACTCCGATCAC |
| | | CTTCGGCCAAGGGACACGACTGGAGATTAAA (SEQ ID NO: 560) |
| FJ 4E | V_{H} | CAGGTGCAGCTGGTGGAGTCGGGCCCAGGACTGGTGAAGCCTTCAGAGACCCTGTCCCTCACCTGCAGCGTCTCTG |
| | | GTGGCTCCATCAGTAGTGGTACTTACTACTGGAGCTGGATCCGCCAGCAGCCAGGGAAGGGCCTGGAGTGGATTGG |
| | | GTACATCTATAACACTGGGAGACCCTACTACAACCCGTTTCTCAAGAGTCGAATTACCATATCAGTGGACTCGTCTAA |
| | | GAACCAGTTCTCCCTGAAGCTGACCTCTGTGACTGCCGCGGACACGGCCGTGTATTATTGTGCGAGAGATGAATATG |
| | | ATTCCTCTGATTCGGGGATTCAAGGCCACTGGTTCGACCCCTGGGGCCAGGGAACCCTGGTCACCGTCTCCTCA |
| | | (SEQ ID NO: 561) |
| | V_{L} | GCCATCCGGATGACCCAGTCTCCATCCTCCCTGTCTGCATCTGTGGGAGACAGAGTCACCATCACTTGCCGGACAAG |
| | | TCAGAACATTAACAGTTTTTTAAATTGGTATCAGCAGAAACCAGGGAAAGGCCCTAACCTCCTGATCTATGGTGCATT |
| | | CACTTTACAAAGTGGGGTCCCATCAAGGTTCAGTGGCAGTGGATCTGGGACAGATTTCACACTCACCATCAGCAGTC |
| | | TACAACCTGAAGATTTTGCAACTTACTTCTGTCAACAGAGTTACAGTACCCCGTGGACGTTCGGCCAAGGGACCAA |
| | | GGTGGAAATCAAA (SEQ ID NO: 562) |
| FJ 1C | V_{H} | CAGGTGCAGCTGCAGGAGTCGGGCCCAGGACTGGTGAAGCCTTCGGAGACCCTGTCCCTCACCTGCACTGTCTCTG |
| | | GTTACTCCATCAGCAGTGGTTACTACTGGGGCTGGATCCGGCAGCCCCCAGGGAAGGGGCTGGAGTGGATTGGGAG |
| | | TATCTATCATAGTGGGAGCACCTACTACAACCCGTCCCTCAAGAGTCGAGTCACCATATCAGTAGACACGTCCAAGA |
| | | ACCAGTTCTCCCTGAAGCTGAGCTCTGTGACCGCCGCAGACACGGCCGTGTATTACTGTGCGAGAGATAAGGCCTTA |
| | | CTATGGTTCGGGGAGTTATTTACCAACCTCTTTGACTACTGGGGCCAGGGAACCCTGGTCACCGTCTCCTCA (SEQ ID |
| | | NO: 563) |
| | V_{L} | CAGTCTGCCCTGACTCAGCCTGCCTCCGTGTCTGGGTCTCCTGGACAGTCGATCACCATCTCCTGCACTGGAACCAG |
| | | CAGTGACGTTGGTGGTTATAACTATGTCTCCTGGTACCAACAGCACCCAGGCAAAGCCCCCAAACTCATGATTTATG |
| | | AGGTCAGTAATCGGCCCTCAGGGGTTTCTAATCGCTTCTCTGGCTCCAAGTCTGGCAACACGGCCTCCCTGACCATC |
| | | TCTGGGCTCCAGGCTGAGGACGAGGCTAATTATTACTGCAGCTCATATACAAGCAGCAGCACTCTGGTATTCGGCGG |
| | | AGGGACCAAGCTGACCGTCCTA (SEQ ID NO: 564) |
| EW 8B | V_{H} | GTCCAGCTGGTACAGGAGTCGGGCCCAGGACTGGTGAAGCCTTCGGAGACCCTGTCCCTCACCTGCACTGTCTCTG |
| | | GTGGCTCCATCAGCAGTAGTGGTTACTACTGGGGCTGGATCCGGCAGCCCCCAGGGAAGGGGCTGGAGTGGATTGC |
| | | GAATTTTTATTTTACTGGGAGTGTCTACTCCAACCCGTCCCTCAAGAGTCGAGTCACCATATCCGTTGACACGTCCAA |
| | | GAACCAGCTCTCCCTGAAATTGAGCTATCTGACCGCCGCAGACACGGCTGTATATTACTGTGCGAGACAAGAGGTTT |
| | | GGGGTGGTTTTGATATCTGGGGCCAAGGGACAATGGTCACCGTCTCTTCA (SEQ ID NO: 565) |
| | V_{L} | GAAATTGTGTTGACGCAGTCTCCAGGCACCCTGTCTTTGTCTCCAGGGGAAAGAGTCACCCTCTCCTGCAGGGCCA |
| | | GTCAGAGTCTTGGCAGCAGCTACTTAGCCTGGTACCAGCAGAAACCTGGCCAGGCTCCCAGGCTCCTCATCTATGGT |
| | | GCATCCAGCAGGGCCACTGGCATCCCAGACAGGTTCAGTGGCAGTGGGTCTGGGACAGACTTCACTCTCACCATCA |
| | | GCAGACTGGAGCCTGAAGATTTTGCAGTGTATTTCTGTCAGCAGTATGGTAGCTCACCGACGTTCGGCCAAGGGACC |
| | | AAGGTGGAAATCAAA (SEQ ID NO: 566) |
| FD 11C | V_{H} | GAGGTGCAGCTGTTGGAGTCGGGCCCAGGACTGGTGAAGCCTTCGGAGACCCTGTCCCTCACCTGCACTGTCTCTG |
| | | GTGGCTCCATCAGCAGTAGTAGTTACTACTGGGGCTGGATCCGCCAGCCCCCAGGGAAGGGGCTGGAGTGGATTGG |
| | | GAGTATCTATTATAGTGGGAGCACCTACTACAACCCGTCCCTCAAGAGTCGAGTCACCATATCAGTAGACACGTCCAA |
| | | GAACCAGTTCTCCCTGAAGCTGAGCTCTGTGACCGCCGCGGACACGGCCGTGTATTACTGTGCGAGGGAGTATTACT |
| | | ATGGTTCGGAGACAAAAAAATACTACTACTACTACGGTATGGACGTCTGGGGCCAAGGGACCACGGTCACCGTCTCC |
| | | TCA (SEQ ID NO: 567) |
| | V_{L} | TCCTATGAGCTGACTCAGCCACCCTCAGTGTCCGTGTCCCCAGGACAGACAGCCAGCATCACCTGCTCTGGAGATAA |
| | | ATTGGGGGATAAATATGCTTGCTGGTATCAGCAGAAGCCAGGCCAGTCCCCTGTGCTGGTCATCTATCAAGATAGCAA |
| | | GCGGCCCTCAGGGATCCCTGAGCGATTCTCTGGCTCCAACTCTGGGAACACAGCCACTCTGACCATCAGCGGGACC |
| | | CAGGCTATGGATGAGGCTGACTATTACTGTCAGGCGTGGGACAGCAGCACCGTATTCGGCGGAGGGACCAAGCTGA |
| | | CCGTCCTA (SEQ ID NO: 568) |
| FD 7C | V_{H} | CAGCTGCAGCTGCAGGAGTCGGGCCCAGGACTGGTGAAGCCTTCGGAGACCCTGTCCCTCACCTGCACTGTCTCTG |
| | | GTGGCTCCATCAGTAGTTACTACTGGAGCTGGATCCGGCAGCCCCCAGGGAAGGGACTGGAGTGGATTGGGTATATC |
| | | TATTACAGTGGGAGCACCAACTACAACCCCTCCCTCAAGAGTCGAGTCACCATATCAGTAGACACGTCCAAGAACCA |
| | | GTTCTCCCTGAAGCTGAGCTCTGTGACCGCTGCGGACACGGCCGTGTATTACTGTGCGAGAGATTATAGGTTCGGGG |
| | | AGTTATTTGGAAGGTTTGCCTGGTTCGACCCCTGGGGCCAGGGAACCCTGGTCACCGTCTCCTCA(SEQ ID NO: 569) |
| | V_{L} | GACATCCAGTTGACCCAGTCTCCAGCCACCCTGTCTGTGTCTCCAGGGGAAAGAGCCACCCTCTCCTGCAGGGCCA |
| | | GTCAGAGTGTTAGCAGCAACTTAGCCTGGTACCAGCAGAAACCTGGCCAGGCTCCCAGGCTCCTCATCTATGGTGCA |
| | | TCCACCAGGGCCACTGGTATCCCAGCCAGGTTCAGTGGCAGTGGGTCTGGGACAGAGTTCACTCTCACCATCAGCA |
| | | GCCTGCAGTCTGAAGATTTTGCAGTTTATTACTGTCAGCAGTATAATAACTGGCCTAGGGCTTTCGGCCCTGGGACCA |
| | | AGGTGGAAATCAGA (SEQ ID NO: 570) |
| FD 7D | V_{H} | GAGGTGCAGCTGGTGGAGTCCGGAGCAGAGGTGAAAAAGCCCGGGGAGTCTCTGAGGATCTCCTGTAAGGGTTCT |
| | | GGATACAGCTTTACCAGCTACTGGATCAGCTGGGTGCGCCAGATGCCCGGGAAAGGCCTGGAGTGGATGGGGAGGA |
| | | TTGATCCTAGTGACTCTTATACCAACTACAGCCCGTCCTTCCAAGGCCACGTCACCATCTCAGCTGACAAGTCCATCA |
| | | GCACTGCCTACCTGCAGTGGAGCAGCCTGAAGGCCTCGGACACCGCCATGTATTACTGTGCGAGACATTCTGATTGT |
| | | AGTAGTACCAGCTGCTATTTCGTCGATGCTTTTGATATCTGGGGCCAAGGGACAATGGTCACCGTCTCTTCA (SEQ ID |
| | | NO: 571) |
| | V_{L} | TCCTATGAGCTGACTCAGCCACCCTCGGTGTCAGTGTCCCCAGGACAGACGGCCAGGATCACCTGCTCTGGAGATG |
| | | CATTGCCAAAGCAATATGCTTATTGGTACCAGCAGAAGCCAGGCCAGGCCCCTGTGCTGGTGATATATAAAGACAGT |
| | | GAGAGGCCCTCAGGGATCCCTGAGCGATTCTCTGGCTCCAGCTCAGGGACAACAGTCACGTTGACCATCAGTGGAG |
| | | TCCAGGCAGAAGACGAGGCTGACTATTACTGTCAATCAGCAGACAGCAGTGGTACTTATGTGGTATTCGGCGGAGGG |
| | | ACCAAGCTGACCGTCCTA (SEQ ID NO: 572) |
| FJ 10B | V_{H} | CAGGTTCAGCTGGTGCAGTCCGGAGCAGAGGTGAAAAAGCCCGGGGAGTCTCTGAGGATCTCCTGTAAGGGTTCTG |
| | | GATACAGCTTTACCAGCTACTGGATCAGCTGGGTGCGCCAGATGCCCGGGAAAGGCCTGGAGTGGATGGGGAGGAT |
| | | TGATCCTAGTGACTCTTATACCAACTACAGCCCGTCCTTCCAAGGCCACGTCACCATCTCAGCTGACAAGTCCATCAG |
| | | CACTGCCTACCTGCAGTGGAGCAGCCTGAAGGCCTCGGACACCGCCATGTATTACTGTGCGAGACTAGACCCCCGTT |
| | | ATGGGCCCGACTACTACGGTATGGACGTCTGGGGCCAAGGGACCACGGTCACCGTCTCCTCA (SEQ ID NO: 573) |
| | V_{L} | GCCATCCGGATGACCCAGTCTCCATCCTCCCTGTCTGCATCTGTGGGAGACAGAGTCACCATCACTTGCCGGGCAAG |
| | | TCAGAGCATTAGCAGCTATTTAAATTGGTATCAGCAGAAACCAGGGAAAGCCCCTAAGCTCCTGATCCATGCTGCATC |
| | | CAGTTTGCAAAGTGGGGTCCCATCAAGGTTCAGTGGCAGTGGATCTGGGACAGATTTCACTCTCACCATGAGCAGTC |
| | | TGCAACCTGAAGATTTTGCAACTTACTACTGTCAACAGAGTTACAGTACCCCGCTCACTTTCGGCGGAGGGACCAAG |
| | | GTGGAAATCAGA (SEQ ID NO: 574) |
| FB 9D | V_{H} | CAGGTGCAGCTGGTGGAGTCTGGAGCAGAAGTGAAAAAGCCCGGGGAGTCTTTGAAGATCTCCTGTCAGGGTTCT |
| | | GGATACAGGTTTAACAGTTATTGGATCGCCTGGGTGCGCCAAATGCCCGGGAAAGGCCTGGAGTGGATGGGGAGCA |
| | | TCTTTCCTACTGACTCTGATGTCAGATATAACCCGTCCTTCCAAGGCCAGGTCACCATTTCGGCCGACAAGTCCATCA |
| | | GTTTCGCCTACCTGCAGTGGAGCAGCCTGAAGGCCTCGGACACCGCCATATATTATTGTGCGAGACATTGGGCCAGT |
| | | ATGGTTCGGGGAGTAATTCGTGCCAGTCATTATTATGGCATGGACGTCTGGGGCCAAGGGACCACGGTCACCGTCTC |
| | | CTCA (SEQ ID NO: 575) |
| | V_{L} | CAGCCTGTGCTGACTCAGCCTCCCTCCGCGTCCGGGTCTCCTGGACAGTCAGTCACCATCTCCTGCTCTGGAGCCAG |
| | | CAGTGACATTGGTAAATATAACTATGTCTCCTGGTACCAACAGCTCCCAGGCAAAGCCCCCAAACTCCTGATTTATGA |
| | | GGTCACTAAGCGGCCCTCAGGGGTCCCTGATCGCTTCTCTGGCTCCAAGTCTGGCAACACGGCCTCCCTGACCGTCT |
| | | CTGGGCTCCAGGCTGACGATGAGGCTGATTATTACTGCAGCTCTTATGCATTTGGAGGCAGCGACACCCGGGTGTTC |
| | | GGCGGAGGGACCAAGCTGACCGTCCTA (SEQ ID NO: 576) |
| FB 1E | V_{H} | GAAGTGCAGCTGGTGGAGTCTGGAGCAGAAGTGAAAAAGCCCGGGGAGTCTCTGAAGATCTCCTGTCAGGGTTCT |
| | | GGATACAGGTTTAATAGTTATTGGATCGCCTGGGTGCGCCAGATGCCCGGGAAAGGCCTGGAGTGGATGGGGAGCAT |
| | | CTTTCCTACTGACTCTGATATCAGATATAACCCGTCCTTCCAAGGCCAGGTCACCATCTCAGCCGACAAGTCCATCAG |
| | | TATCGCCTACCTGCAGTGGAGCAGCCTGAAGGCCTCGGACACCGCCATATATTATTGTGCGAGACATTGGGCCAGTAT |
| | | GGTTCGGGGAGTAATTCGTGCCAGTCATTATTATGGCATGGACGTCTGGGGCCAAGGGACCACGGTCACCGTCTCCT |
| | | CA (SEQ ID NO: 577) |
| | V_{L} | CAGTCTGTGCTGACTCAGCCTCCCTCCGCGTCCGGGTCTCCTGGACAGTCAGTCACCATCTCCTGCTCTGGAGCCAG |
| | | CAGTGACATTGGTGATTATAACTATGTCTCCTGGTACCAACAGCTCCCAGGCAAAGCCCCCAAACTCCTGATTTATGA |
| | | GGTCACTAAGCGGCCCTCAGGGGTCCCTGATCGCTTCTCTGGCTCCAAGTCTGGCAACACGGCCTCCCTGACCGTCT |
| | | CTGGGCTCCAGGCTGACGATGAGGCTGATTACTACTGCAGCTCTTATGCATTTGGAGGCAGCGACATCCGGGTGTTC |
| | | GGCGGAGGGACCAAGCTGGCCGTCCTA (SEQ ID NO: 578) |
| EZ 7A | V_{H} | GAAGTGCAGCTGGTGGAGTCTGGAGCAGAGGTGAAAAAGCCCGGGGAGTCTCTGAAGATCTCCTGTAAGGGTTCT |
| | | GGATACAGCTTTACCAGCTACTGGATCGGCTGGGTGCGCCAGATGCCCGGGAAAGGCCTGGAGTGGATGGGGATCA |
| | | TCTATCCTGGTGACTCTGATACCAGATACAGCCCGTCCTTCCAAGGCCAGGTCACCATCTCAGCCGACAAGTCCATC |
| | | AGCACCGCCTACCTGCAGTGGAGCAGCCTGAAGGCCTCGGACACCGCCATGTATTACTGTGCGAGAGGATGGGTTT |
| | | ATCGGGGCTTCCCTTACTACGGTATGGACGTCTGGGGCCAAGGGACAATGGTCACCGTCTCTTCA (SEQ ID NO: 579) |
| | V_{L} | CAGTCTGCCCTGACTCAGCCTCGCTCAGTGTCCGGGTCTCCTGGACAGTCAGTCACCATCTCCTGCACTGGAACCAG |
| | | CAGTGATGTTGGTGGTTATAACTATGTCTCCTGGTACCAACAGCACCCAGGCAAAGCCCCCAAACTCATGATTTATGA |
| | | TGTCAGTAAGCGGCCCTCAGGGGTCCCTGATCGCTTCTCTGGCTCCAAGTCTGGCAACACGGCCTCCCTGACCATCT |
| | | CTGGGCTCCAGGCTGAGGATGAGGCTGATTATTACTGCTGCTCATATGCAGGCAGCTACACTTTGGTGTTCGGCGGA |
| | | GGGACCAAGCTGACCGTCCTA (SEQ ID NO: 580) |

Amino acid sequences of V_{H} and V_{L}

| | | |
|---|---|---|
| FM 7B | V_{H} | EVQLVQSGAEVKKPGASVKVSCKASGYTFTSYAMHWVRQAPGQRLEWMGWINTGNGNTKYSQKFQGRVTITRDTSAS |
| | | TAYMELSSLRSEDTAVYYCARDPTYCSSTSCYPFSWFDPWGQGTLVTVSS (SEQ ID NO: 1) |
| | V_{L} | NFMLTQPPSVSVAPGQTARITCGGNNIGSKSVHWYQQKPGQAPVLVVYDDSDRPSGIPERFSGSNSGNTATLTISRVEAGD |
| | | EADYYCQVWDSTGDHSWVFGGGTKLTVL (SEQ ID NO: 2) |
| FD 8B | V_{H} | EVQLLESGAEVKKPGASVKVSCKVSGYTLTELSMHWVRQAPGKGLEWMGGFDPEDGETIYAQKFQGRVTMTEDTSTD |
| | | TAYMELSSLRSEDTAVYYCATAAAINCSSTSCYYYYYYYGMDVWGQGTTVTVSS (SEQ ID NO: 3) |
| | V_{L} | DIVMTQTPLSSPVTLGQPASISCRSSQSLVHSDGNTYLSWLQQRPGQPPRLLIYKISSRFSGVPDRFSGSGAGTDFTLKISRV |
| | | EAEDVGVYYCTQATQFPYTFGQGTKVDIK (SEQ ID NO: 4) |
| EW 9B | V_{H} | EVQLVQSGAEVKKPGASVKVSCKASGYTFTSYYMHWVRQAPGQGLEWMGIINPSGGSTSYAQKFQGRVTMTRDTSTST |
| | | VYMELSSLRSEDTAVYYCAREDGVVPAANLMISLEDYYYYGMDVWGQGTTVTVSS (SEQ ID NO: 5) |
| | V_{L} | EIVLTQSPATLSLSPGERATLSCRASQSVSSYLAWYQQKPGQAPRLLIYDASNRATGIPARFSGSGSGTDFTLTISSLEPEDF |
| | | AVYYCQQRSNWPLTFGGGTKVDIK (SEQ ID NO: 6) |
| FN 12A | V_{H} | QVQLVQSGAEVKKPGSSVKVSCKASGGTFSSYAISWVRQAPGQGLEWMGRIIPILGIANYAQKFQGRVTITADKSTSTAY |
| | | MELSSLRSEDTAVYYCARSGCSSTSCPSNLYYYYYGMDVWGQGTTVTVSS (SEQ ID NO: 7) |
| | V_{L} | QSVLTQPPSVSAAPGQKVTISCSGSSSNIGNNYVSWYQQLPGTAPKLLIYDNNKRPSGIPDRFSGSKSGTSATLGITGLQTG |
| | | DEADYYCGTWDSSLSALVFGGGTKLTVL (SEQ ID NO: 8) |
| FG 12C | V_{H} | EVOLVESGGGLVQPGRSLRLSCAASGFTFDDYAMHWVRQAPGKGLEWVSGISWNSGSIGYADSVKGRFTISRDNAKNS |
| | | LYLQMNSLRAEDTALYYCAKDMRVHDYGDYYFDYWGQGTLVTVSS (SEQ ID NO: 9) |
| | V_{L} | SYELTQPPSVSVAPGKTARITCGGNNIGSKSVHWYQQKPGQAPVLVIYYDSDRPSGIPERFSGSNSGNTATLTISRVEAGDE |
| | | ADYYCQVWDSSSDHPVFGGGTKLTVL (SEQ ID NO: 10) |
| FI 1C | V_{H} | EVQLVESGGGLVKPGGSLRLSCAASGFTFSDYYMSWIRQAPGKGLEWVSYISSSGSTIYYADSVKGRFTISRDNAKNSLY |
| | | LQMNSLRAEDTAVYYCARRSNRFLIAFDIWGQGTMVTVSS (SEQ ID NO: 11) |
| | V_{L} | QSALTQPASVSGSPGQTITISCTGTSSDVGGYNYVSWYQQHPGKAPKLMIYEVSNRPSGVSNRFSGSKSGNTASLTISGLQ |
| | | AEDEADYYCSSYTSSSTLVVFGGGTKLTVP (SEQ ID NO: 12) |
| FI 4A | V_{H} | EVOLVESGGGLVKPGGSLRLSCAASGFTFSRFSMNWVRQAPGKGLEWVSSISSSGSYIYFADSVKGRFTISRDNAKNSLY |
| | | LQMNSLRAEDTAIYYCATYLFGDSHTYWGQGTLVTVSS (SEQ ID NO: 13) |
| | V_{L} | QSVLTQPHSVSESPGKTVTISCTGSSGSIASNYVQWYQQRPGSAPTTVIYEDNQRPSGVPDRFSGSIDSSSNSASLTISGLKT |
| | | EDEADYYCQSYDSSNLHWVFGGGTKLTVL (SEQ ID NO: 14) |
| FD 1E | V_{H} | EVQLLESGGGLVKPGGSLRLSCAASGFTFSSYSMNWVRQAPGKGLEWVSSISSSSSYIYYADSVKGRFTISRDNAKNSLY |
| | | LQMNSLRAEDTAVYYCASLAAAGPETYYYYGMDVWGQGTMVTVSS (SEQ ID NO: 15) |
| | V_{L} | SYELTQPPSVSVSPGQTASITCSGDKLGDKYACWYQQKPGQSPVLVIYQDSKRPSGIPERFSGSNSGNTATLTISGTQAMD |
| | | EADYYCQAWDSSVVFGGGTKLTVL (SEQ ID NO: 16) |
| FD 11E | V_{H} | EVQLVESGGGLVKPGGSLRLSCAASGFTFSSYSMNWVRQAPGKGLEWVSSISSSSSYIYYADSVKGRFTISRDNAKNSLY |
| | | LQMNSLRAEDTAVYYCASLAAAGPETYYYYGMDVWGQGTMVTVSS (SEQ ID NO: 17) |
| | V_{L} | SYELTQPPSVSVSPGQTASITCSGDKLGDKYACWYQQKPGQSPVLVIYQDSKRPSGIPERFSGSNSGNTATLTISGTQAMD |
| | | EADYYCQAWDSSVVFGGGTKLTVL (SEQ ID NO: 18) |
| FN 2C | V_{H} | EVQLLESGGGVVQPGRSLRLSCAASGFNFNNYGMHWVRQAPGKGLEWVATISYEGSKKFYVDSVKGRFTISKDNSKNT |
| | | LYLQMNSLRVDDTAFYYCAKRREIFWLGEPPLSDAFDFWGQGTMVTVSS (SEQ ID NO: 19) |
| | V_{L} | QSVLTQPPSVSGAPGQRVTISCTGSGSNIGAGYDVHWYQFLPGTAPQLLIYGNNNRPSGVPDRFSGSKSGTSASLAITGLQ |
| | | AEDEADYYCQSYDSSLSGSVFGGGTKLTVL (SEQ ID NO: 20) |
| EY 6A | V_{H} | EVQLVESGGGVVQPGRSLRLSCAASAFTFSSYDMHWVRQAPGKGLEWVAVISYDGSNKYYADSVKGRFTISRDNSKNT |
| | | LYLQMNSLRAEDTAVYYCAKDGGKLWVYYFDYWGQGTTVTVSS (SEQ ID NO: 21) |
| | V_{L} | DIQMTQSPSSLSASVGDRVTITCRASQSISSYLNWYQQKPGKAPKLLIYAASSLQSGVPSRFSGSGSGTDFTLTISSLQPEDF |
| | | ATYYCQQSYSTLALTFGGGTKVDIK (SEQ ID NO: 22) |
| EY 6A-1* | V_{H} | EVQLVESGGGVVQPGRSLRLSCAASAFTFSSYDMHWVRQAPGKGLEWVAVISYDGSNKYYADSVKGRFTISRDNSKNT |
| | | LYLQMNSLRAEDTAVYYCAKDGGKLWVYYFDYWGQGTLVTVSS (SEQ ID NO: 23) |
| | V_{L} | DIQMTQSPSSLSASVGDRVTITCRASQSISSYLNWYQQKPGKAPKLLIYAASSLQSGVPSRFSGSGSGTDFTLTISSLQPEDF |
| | | ATYYCQQSYSTLALTFGGGTKVEIK (SEQ ID NO: 24) |
| FD 11D | V_{H} | EVQLVESGGGVVQPGRSLRLSCAASGFTFSSYGMHWVRQAPGKGLEWVAVISYDGSNKYYADSVKGRFTISRDNSKNT |
| | | LYLQMNSLRAEDTAVYYCAKEGAGSGWYRHHKPGYYFDYWGQGTLVTVSS (SEQ ID NO: 25) |
| | V_{L} | EIVLTQSPGTLSLSPGERATLSCRASQSVSSSYLAWYQQKPGQAPRLLIYGASSRATGIPDRFSGSGSGTDFTLTISRLEPED |
| | | FAVYYCQQYGSSPLTFGQGTKVEIK (SEQ ID NO: 26) |
| EW 9C | V_{H} | EVOLVESGGGVVQPGASLRLSCVASGFTFNNFGFHWVRQAPGKGLEWLAVISYEGSKTYYAESLKGRFTISRDTSKNTV |
| | | YLQMNSLRAEDTAVYYCARATSIFWFGEGRNWFDPWGQGTMVTVSS (SEQ ID NO: 27) |
| | V_{L} | EIVLTQSPGTVSLSAGERATLSCRASQNVGTDLAWYVQRPGQAPRLLIYDTSNKATGIPARFSGRGSGTDFTLTIDSLEPED |
| | | FAVYYCQQRSNWPLTFGGGTKVEIR (SEQ ID NO: 28) |
| EW 9C-1* | V_{H} | EVQLVESGGGVVQPGASLRLSCVASGFTFNNFGFHWVRQAPGKGLEWLAVISYEGSKTYYAESLKGRFTISRDTSKNTV |
| | | YLQMNSLRAEDTAVYYCARATSIFWFGEGRNWFDPWGQGALVTVSS (SEQ ID NO: 29) |
| | V_{L} | EIVMTQSPGTVSLSAGERATLSCRASQNVGTDLAWYVQRPGQAPRLLIYDTSNRATGIPARFSGRGSGTDFTLTIDSLEPE |
| | | DFAVYYCQQRSNWPLTFGGGTKVEIK (SEQ ID NO: 30) |
| FD 1D | V_{H} | EVQLVESGGGVVQPGRSLRLSCAASGFTFSSYAMHWVRQAPGKGLEWVAVISYDGSNKYYADSVKGRFTISRDNSKNT |
| | | LYLQMNSLRAEDTAVYYCARAGSGSYLNWFDPWGQGTLVTVSS (SEQ ID NO: 31) |
| | V_{L} | EIVLTQSPATLSLSPGERATLSCRASQSVSSYLAWYQQKPGQAPRLLIYDASNRATGIPARFSGSGSGTDFTLTISSLEPEDF |
| | | AVYYCQQRSNWPITFGQGTRLEIK (SEQ ID NO: 32) |
| FG 7A | V_{H} | EVQLVESGGGVVQPGRSLRLSCAASGFTFSSYAMHWVRQAPGKGLEWVAVISYDGSNKYYADSVKGRFTISRDNSKNT |
| | | LYLQMNSLRAEDTAVYYCARSHSGSYRASLDYWGQGTTVTVSS (SEQ ID NO: 33) |
| | V_{L} | DIQLTQSPGTLSLSPGERATLSCRASQSVSSSYLAWYQQKPGQAPRLLIYGASSRATGIPDRFSGSGSGTDFTLTISRLEPED |
| | | FAVYYCQQYGSSPLYTFGQGTKVEIK (SEQ ID NO: 34) |
| FM 1A | V_{H} | EVQLLESGGGVVQPGRSLRLSCAASGFTFSSYGMHWVRQAPGKGLEWVAVIWYDGSNKYYADSVKGRFTISRDNSKNT |
| | | LYLQMNSLRAEDTAVYYCAREGAVGATRGFDYWGQGTLVTVSS (SEQ ID NO: 35) |
| | V_{L} | SYELTQPPSVSVAPGQTARITCGGNNIGSKSVHWYQQKPGQAPVLVVYDDSDRPSGIPERFSGSNSGNTATLTISRVEAGD |
| | | EADYYCQVWDSSSDQGVFGGGTKLTVL (SEQ ID NO: 36) |
| FD 11A | V_{H} | EVQLVESGGGVVQPGRSLRLSCAASGFTFSSYGMHWVRQAPGKGLEWVAVIWYDGSNKYYADSVKGRFTISRDNSKNT |
| | | LYLQMNSLRAEDTAVYYCAKGPDILTGYYNYYYYGMDVWGQGTMVTVSS (SEQ ID NO: 37) |
| | V_{L} | QSVLTQPPSVSGAPGQRVTISCTGSSSNIGAGYDVHWYQQLPGTAPKLLIYGNSNRPSGVPDRFSGSKSGTSASLAITGLQ |
| | | AEDEADYYCQSYDSSLSGFYVVFGGGTKLTVL (SEQ ID NO: 38) |
| FN 8C | V_{H} | QVQLVESGGGVVQPGRSLRLSCAASGFTFSSYGMHWVRQAPGKGLEWVAVISYDGSNKYYADTVKGRFTISRDNSKNT |
| | | LYLQMNSLRAEDTAVYYCARERTYYDILTGYRHYYGMDVWGQGTTVTVSS (SEQ ID NO: 39) |
| | V_{L} | SYELTQPPSVSVAPGQTARITCGGNNIGSKSVHWYQQKPGQAPVLVVYDDSDRPSGIPERFSGSNSGNTATLTISRVEAGD |
| | | EADYYCQVWDSSSDHWVFGGGTKLTVL (SEQ ID NO: 40) |
| FD 5E | V_{H} | EVQLVESGGVVVQPGGSLRLSCAASGFTFDDYAMHWVRQAPGKGLEWVSLISWDGGSTYYADSVKGRFTISRDNSKNS |
| | | LYLQMNSLRAEDTALYYCAKDSVRFRYYYGMDVWGQGTTVTVSS (SEQ ID NO: 41) |
| | V_{L} | EIVLTQSPATLSLSPGERATLSCRASQSVSSYLAWYQQKPGQAPRLLIYDASNRATGIPARFSGSGSGTDFTLTISSLEPEDF |
| | | AVYYCQQRSNWPLTFGPGTKVDIK (SEQ ID NO: 42) |
| FD 5D | V_{H} | EVOLVESGGGLVQPGGSLRLSCAASGFTFSSYSMNWVRQAPGKGLEWVSYISSSSSTIYYADPVKGRFTISRDNAKNSLY |
| | | LQMNSLRAEDTAVYYCASPGGITAAGTSVLFGYYGMDVWGQGTMVTVSS (SEQ ID NO: 43) |
| | V_{L} | DIVMTQSPLSLPVTPGEPASISCRSSQSLLHSNGYNYLDWYLQKPGQSPQLLIYLGSNRASGVPDRFSGSGSGTDFTLKISR |
| | | VEAEDVGVYYCMQALQTPITWTFGQGTKVDIK (SEQ ID NO: 44) |
| FI 3A | V_{H} | EVQLLESGGGLIQPGGSLRLSCAASGFTVSSNYMSWVRQAPGKGLEWVSVIYSGGSTYYADSVKGRFTISRDNSKNTLY |
| | | LQMNSLRAEDTAVYYCARDHVRPGMNIWGQGTMVTVSS (SEQ ID NO: 45) |
| | V_{L} | AIRMTQSPSSLSASVGDRVTITCQASQDISNYLNWYQQKPGKAPKLLIYDASNLETGVPSRFSGSGSGTDFTFTISSLQPED |
| | | IATYYCQQYDNLPVTFGGGTKVDIK (SEQ ID NO: 46) |
| FD 10A | V_{H} | EVOLVESGGGLVQPGGSLRLSCAASGFTFSSYWMHWVRQAPGKGLVWVSRINSDGSSTSYADSVKGRFTISRDNAKNT |
| | | LYLQMNSLRAEDTAVYYCANMAFDIWGQGTMVTVSS (SEQ ID NO: 47) |
| | V_{L} | DIVMTQSPDSLAVSLGERATINCKSSQSVLYSSNNKNYLAWYQQKPGQPPKLLIYWASTRESGVPDRFSGSGSGTDFTLTI |
| | | SSLQAEDVAVYYCQQYYSTPITFGQGTRLEIK (SEQ ID NO: 48) |
| FJ 4E | V_{H} | QVQLVESGPGLVKPSETLSLTCSVSGGSISSGTYYWSWIRQQPGKGLEWIGYIYNTGRPYYNPFLKSRITISVDSSKNQFSL |
| | | KLTSVTAADTAVYYCARDEYDSSDSGIQGHWFDPWGQGTLVTVSS (SEQ ID NO: 49) |
| | V_{L} | AIRMTQSPSSLSASVGDRVTITCRTSQNINSFLNWYQQKPGKGPNLLIYGAFTLQSGVPSRFSGSGSGTDFTLTISSLQPED |
| | | FATYFCQQSYSTPWTFGQGTKVEIK (SEQ ID NO: 50) |
| FJ 1C | V_{H} | QVQLQESGPGLVKPSETLSLTCTVSGYSISSGYYWGWIRQPPGKGLEWIGSIYHSGSTYYNPSLKSRVTISVDTSKNQFSL |
| | | KLSSVTAADTAVYYCARDKALLWFGELFTNLFDYWGQGTLVTVSS (SEQ ID NO: 51) |
| | V_{L} | QSALTQPASVSGSPGQSITISCTGTSSDVGGYNYVSWYQQHPGKAPKLMIYEVSNRPSGVSNRFSGSKSGNTASLTISGLQ |
| | | AEDEANYYCSSYTSSSTLVFGGGTKLTVL (SEQ ID NO: 52) |
| EW 8B | V_{H} | VQLVQESGPGLVKPSETLSLTCTVSGGSISSSGYYWGWIRQPPGKGLEWIANFYFTGSVYSNPSLKSRVTISVDTSKNQLS |
| | | LKLSYLTAADTAVYYCARQEVWGGFDIWGQGTMVTVSS (SEQ ID NO: 53) |
| | V_{L} | EIVLTQSPGTLSLSPGERVTLSCRASQSLGSSYLAWYQQKPGQAPRLLIYGASSRATGIPDRFSGSGSGTDFTLTISRLEPED |
| | | FAVYFCQQYGSSPTFGQGTKVEIK (SEQ ID NO: 54) |
| FD 11C | V_{H} | EVQLLESGPGLVKPSETLSLTCTVSGGSISSSSYYWGWIRQPPGKGLEWIGSIYYSGSTYYNPSLKSRVTISVDTSKNQFSL |
| | | KLSSVTAADTAVYYCAREYYYGSETKKYYYYYGMDVWGQGTTVTVSS (SEQ ID NO: 55) |
| | V_{L} | SYELTQPPSVSVSPGQTASITCSGDKLGDKYACWYQQKPGQSPVLVIYQDSKRPSGIPERFSGSNSGNTATLTISGTQAMD |
| | | EADYYCQAWDSSTVFGGGTKLTVL (SEQ ID NO: 56) |
| FD 7C | V_{H} | QLQLQESGPGLVKPSETLSLTCTVSGGSISSYYWSWIRQPPGKGLEWIGYIYYSGSTNYNPSLKSRVTISVDTSKNQFSLKL |
| | | SSVTAADTAVYYCARDYRFGELFGRFAWFDPWGQGTLVTVSS (SEQ ID NO: 57) |
| | V_{L} | DIQLTQSPATLSVSPGERATLSCRASQSVSSNLAWYQQKPGQAPRLLIYGASTRATGIPARFSGSGSGTEFTLTISSLQSEDF |
| | | AVYYCQQYNNWPRAFGPGTKVEIR (SEQ ID NO: 58) |
| FD 7D | V_{H} | EVQLVESGAEVKKPGESLRISCKGSGYSFTSYWISWVRQMPGKGLEWMGRIDPSDSYTNYSPSFQGHVTISADKSISTAY |
| | | LQWSSLKASDTAMYYCARHSDCSSTSCYFVDAFDIWGQGTMVTVSS (SEQ ID NO: 59) |
| | V_{L} | SYELTQPPSVSVSPGQTARITCSGDALPKQYAYWYQQKPGQAPVLVIYKDSERPSGIPERFSGSSSGTTVTLTISGVQAEDE |
| | | ADYYCQSADSSGTYVVFGGGTKLTVL (SEQ ID NO: 60) |
| FJ 10B | V_{H} | QVQLVQSGAEVKKPGESLRISCKGSGYSFTSYWISWVRQMPGKGLEWMGRIDPSDSYTNYSPSFQGHVTISADKSISTAY |
| | | LQWSSLKASDTAMYYCARLDPRYGPDYYGMDVWGQGTTVTVSS (SEQ ID NO: 61) |
| | V_{L} | AIRMTQSPSSLSASVGDRVTITCRASQSISSYLNWYQQKPGKAPKLLIHAASSLQSGVPSRFSGSGSGTDFTLTMSSLQPED |
| | | FATYYCQQSYSTPLTFGGGTKVEIR (SEQ ID NO: 62) |
| FB 9D | V_{H} | QVQLVESGAEVKKPGESLKISCQGSGYRFNSYWIAWVRQMPGKGLEWMGSIFPTDSDVRYNPSFQGQVTISADKSISFAY |
| | | LQWSSLKASDTAIYYCARHWASMVRGVIRASHYYGMDVWGQGTTVTVSS (SEQ ID NO: 63) |
| | V_{L} | QPVLTQPPSASGSPGQSVTISCSGASSDIGKYNYVSWYQQLPGKAPKLLIYEVTKRPSGVPDRFSGSKSGNTASLTVSGLQ |
| | | ADDEADYYCSSYAFGGSDTRVFGGGTKLTVL (SEQ ID NO: 64) |
| FB 1E | V_{H} | EVQLVESGAEVKKPGESLKISCQGSGYRFNSYWIAWVRQMPGKGLEWMGSIFPTDSDIRYNPSFQGQVTISADKSISIAYL |
| | | QWSSLKASDTAIYYCARHWASMVRGVIRASHYYGMDVWGQGTTVTVSS (SEQ ID NO: 65) |
| | V_{L} | QSVLTQPPSASGSPGQSVTISCSGASSDIGDYNYVSWYQQLPGKAPKLLIYEVTKRPSGVPDRFSGSKSGNTASLTVSGLQ |
| | | ADDEADYYCSSYAFGGSDIRVFGGGTKLAVL (SEQ ID NO: 66) |
| EZ 7A | V_{H} | EVQLVESGAEVKKPGESLKISCKGSGYSFTSYWIGWVRQMPGKGLEWMGIIYPGDSDTRYSPSFQGQVTISADKSISTAY |
| | | LQWSSLKASDTAMYYCARGWVYRGFPYYGMDVWGQGTMVTVSS (SEQ ID NO: 67) |
| | V_{L} | QSALTQPRSVSGSPGQSVTISCTGTSSDVGGYNYVSWYQQHPGKAPKLMIYDVSKRPSGVPDRFSGSKSGNTASLTISGL |
| | | QAEDEADYYCCSYAGSYTLVFGGGTKLTVL (SEQ ID NO: 68) |

*Both EY 6A and EW 9C have an additional pair of expression vectors.

TABLE 4
Amino acid sequences of complementarity-determining regions (CDRs) of
the heavy chain variable regions and the light chain variable regions of the SARS-COV-2
spike-reactive human monoclonal antibodies.

| | Heavy chain CDR1 | Heavy chain CDR2 | Heavy chain CDR3 | Light chain CDR1 | Light chain CDR2 | Light chain CDR3 |
|---|---|---|---|---|---|---|
| MAb | (HCDR1) | (HCDR2) | (HCDR3) | (LCDR1) | (LCDR2) | (LCDR3) |
| FM 7B | GYTFTSYA (SEQ | INTGNGNT (SEQ | ARDPTYCSSTSCY | NIGSKS (SEQ ID | DDS (SEQ ID No: | QVWDSTGDHSW |
| | ID No: 69) | ID No: 70) | PFSWFDP (SEQ ID | No: 72) | 73) | V (SEQ ID No: 74) |
| | | | No: 71) | | | |
| FD 8B | GYTLTELS (SEQ | FDPEDGET (SEQ | ATAAAINCSSTSC | QSLVHSDGNTY | KIS (SEQ ID No: | TQATQFPYT (SEQ |
| | ID No: 75) | ID No: 76) | YYYYYYYGMDV | (SEQ ID No: 78) | 79) | ID No: 80) |
| | | | (SEQ ID No: 77) | | | |
| EW 9B | GYTFTSYY (SEQ | INPSGGST (SEQ | AREDGVVPAANL | QSVSSY (SEQ ID | DAS (SEQ ID No: | QQRSNWPLT |
| | ID No: 81) | ID No: 82) | MISLEDYYYYGM | No: 84) | 85) | (SEQ ID No: 86) |
| | | | DV (SEQ ID No: | | | |
| | | | 83) | | | |
| FN 12A | GGTFSSYA (SEQ | IIPILGIA (SEQ ID | ARSGCSSTSCPSN | SSNIGNNY (SEQ | DNN (SEQ ID No: | GTWDSSLSALV |
| | ID No: 87) | No: 88) | LYYYYYGMDV | ID No: 90) | 91) | (SEQ ID No: 92) |
| | | | (SEQ ID No: 89) | | | |
| FG 12C | GFTFDDYA (SEQ | ISWNSGSI (SEQ | AKDMRVHDYGD | NIGSKS (SEQ ID | YDS (SEQ ID No: | QVWDSSSDHPV |
| | ID No: 93) | ID No: 94) | YYFDY (SEQ ID | No: 96) | 97) | (SEQ ID No: 98) |
| | | | No: 95) | | | |
| FI 1C | GFTFSDYY (SEQ | ISSSGSTI (SEQ ID | ARRSNRFLIAFDI | SSDVGGYNY | EVS (SEQ ID No: | SSYTSSSTLVV |
| | ID No: 99) | No: 100) | (SEQ ID No: 101) | (SEQ ID No: 102) | 103) | (SEQ ID No: 104) |
| FI 4A | GFTFSRFS (SEQ | ISSSGSYI (SEQ ID | ATYLFGDSHTY | SGSIASNY (SEQ | EDN (SEQ ID No: | QSYDSSNLHWV |
| | ID No: 105) | No: 106) | (SEQ ID No: 107) | ID No: 108) | 109) | (SEQ ID No: 110) |
| FD 1E | GFTFSSYS (SEQ | ISSSSSYI (SEQ ID | ASLAAAGPETYY | KLGDKY (SEQ ID | QDS (SEQ ID No: | QAWDSSVV (SEQ |
| | ID No: 111) | No: 112) | YYGMDV (SEQ ID | No: 114) | 115) | ID No: 116) |
| | | | No: 113) | | | |
| FD 11E | GFTFSSYS (SEQ | ISSSSSYI (SEQ ID | ASLAAAGPETYY | KLGDKY (SEQ ID | QDS (SEQ ID No: | QAWDSSVV (SEQ |
| | ID No: 117) | No: 118) | YYGMDV (SEQ ID | No: 120) | 121) | ID No: 122) |
| | | | No: 119) | | | |
| FN 2C | GFNFNNYG (SEQ | ISYEGSKK (SEQ | AKRREIFWLGEPP | GSNIGAGYD | GNN (SEQ ID No: | QSYDSSLSGSV |
| | ID No: 123) | ID No: 124) | LSDAFDF (SEQ ID | (SEQ ID No: 126) | 127) | (SEQ ID No: 128) |
| | | | No: 125) | | | |
| EY 6A | AFTFSSYD (SEQ | ISYDGSNK (SEQ | AKDGGKLWVYY | QSISSY (SEQ ID | AAS (SEQ ID No: | QQSYSTLALT |
| | ID No: 129) | ID No: 130) | FDY (SEQ ID No: | No: 132) | 133) | (SEQ ID No: 134) |
| | | | 131) | | | |
| EY 6A-1* | AFTFSSYD (SEQ | ISYDGSNK (SEQ | AKDGGKLWVYY | QSISSY (SEQ ID | AAS (SEQ ID No: | QQSYSTLALT |
| | ID No: 135) | ID No: 136) | FDY (SEQ ID No: | No: 138) | 139) | (SEQ ID No: 140) |
| | | | 137) | | | |
| FD 11D | GFTFSSYG (SEQ | ISYDGSNK (SEQ | AKEGAGSGWYR | QSVSSSY (SEQ ID | GAS (SEQ ID No: | QQYGSSPLT (SEQ |
| | ID No: 141) | ID No: 142) | HHKPGYYFDY | No:144) | 145) | ID No: 146) |
| | | | (SEQ ID No: 143) | | | |
| EW 9C | GFTFNNFG (SEQ | ISYEGSKT (SEQ | ARATSIFWFGEGR | QNVGTD (SEQ ID | DTS (SEQ ID No: | QQRSNWPLT |
| | ID No: 147) | ID No: 148) | NWFDP (SEQ ID | No: 150) | 151) | (SEQ ID No: 152) |
| | | | No: 149) | | | |
| EW 9C-1* | GFTFNNFG (SEQ | ISYEGSKT (SEQ | ARATSIFWFGEGR | QNVGTD (SEQ ID | DTS (SEQ ID No: | QQRSNWPLT |
| | ID No: 153) | ID No: 154) | NWFDP (SEQ ID | No: 156) | 157) | (SEQ ID No: 158) |
| | | | No: 155) | | | |
| FD 1D | GFTFSSYA (SEQ | ISYDGSNK (SEQ | ARAGSGSYLNWF | QSVSSY (SEQ ID | DAS (SEQ ID No: | QQRSNWPIT (SEQ |
| | ID No: 159) | ID No: 160) | DP (SEQ ID No: | No: 162) | 163) | ID No: 164) |
| | | | 161) | | | |
| FG 7A | GFTFSSYA (SEQ | ISYDGSNK (SEQ | ARSHSGSYRASL | QSVSSSY (SEQ ID | GAS (SEQ ID No: | QQYGSSPLYT |
| | ID No: 165) | ID No: 166) | DY (SEQ ID No: | No: 168) | 169) | (SEQ ID No: 170) |
| | | | 167) | | | |
| FM 1A | GFTFSSYG (SEQ | IWYDGSNK (SEQ | AREGAVGATRGF | NIGSKS (SEQ ID | DDS (SEQ ID No: | QVWDSSSDQGV |
| | ID No: 171) | ID No: 172) | DY (SEQ ID No: | No: 174) | 175) | (SEQ ID No: 176) |
| | | | 173) | | | |
| FD 11A | GFTFSSYG (SEQ | IWYDGSNK (SEQ | AKGPDILTGYYN | SSNIGAGYD (SEQ | GNS (SEQ ID No: | QSYDSSLSGFYV |
| | ID No: 177) | ID No: 178) | YYYYGMDV | ID No: 180) | 181) | V (SEQ ID No: 182) |
| | | | (SEQ ID No: 179) | | | |
| FN 8C | GFTFSSYG (SEQ | ISYDGSNK (SEQ | ARERTYYDILTGY | NIGSKS (SEQ ID | DDS (SEQ ID No: | QVWDSSSDHWV |
| | ID No: 183) | ID No: 184) | RHYYGMDV (SEQ | No: 186) | 187) | (SEQ ID No: 188) |
| | | | ID No: 185) | | | |
| FD 5E | GFTFDDYA | ISWDGGST (SEQ | AKDSVRFRYYYG | QSVSSY (SEQ ID | DAS (SEQ ID No: | QQRSNWPLT |
| | (SEQ ID No: 189) | ID No: 190) | MDV (SEQ ID No: | No: 192) | 193) | (SEQ ID No: 194) |
| | | | 191) | | | |
| FD 5D | GFTFSSYS | ISSSSSTI (SEQ ID | ASPGGITAAGTSV | QSLLHSNGYNY | LGS (SEQ ID No: | MQALQTPITWT |
| | (SEQ ID No: 195) | No: 196) | LFGYYGMDV | (SEQ ID No: 198) | 199) | (SEQ ID No: 200) |
| | | | (SEQ ID No: 197) | | | |
| FI 3A | GFTVSSNY (SEQ | IYSGGST (SEQ ID | ARDHVRPGMNI | QDISNY (SEQ ID | DAS (SEQ ID No: | QQYDNLPVT |
| | ID No: 201) | No: 202) | (SEQ ID No: 203) | No: 204) | 205) | (SEQ ID No: 206) |
| FD 10A | GFTFSSYW (SEQ | INSDGSST (SEQ | ANMAFDI (SEQ | QSVLYSSNNKNY | WAS (SEQ ID No: | QQYYSTPIT (SEQ |
| | ID No: 207) | ID No: 208) | ID No: 209) | (SEQ ID No: 210) | 211) | ID No: 212) |
| FJ 4E | GGSISSGTYY | TYNTGRP (SEQ ID | ARDEYDSSDSGIQ | QNINSF (SEQ ID | GAF (SEQ ID No: | QQSYSTPWT |
| | (SEQ ID No: 213) | No: 214) | GHWFDP (SEQ ID | No: 216) | 217) | (SEQ ID No: 218) |
| | | | No: 215) | | | |
| FJ 1C | GYSISSGYY (SEQ | TYHSGST (SEQ ID | ARDKALLWFGEL | SSDVGGYNY | EVS (SEQ ID No: | SSYTSSSTLV |
| | ID No: 219) | No: 220) | FTNLFDY (SEQ ID | (SEQ ID No: 222) | 223) | (SEQ ID No: 224) |
| | | | No: 221) | | | |
| EW 8B | GGSISSSGYY | FYFTGSV (SEQ ID | ARQEVWGGFDI | QSLGSSY (SEQ ID | GAS (SEQ ID No: | QQYGSSPT (SEQ |
| | (SEQ ID No: 225) | No: 226) | (SEQ ID No: 227) | No: 228) | 229) | ID No: 230) |
| FD 11C | GGSISSSSYY | TYYSGST (SEQ ID | AREYYYGSETKK | KLGDKY (SEQ ID | QDS (SEQ ID No: | QAWDSSTV (SEQ |
| | (SEQ ID No: 231) | No: 232) | YYYYYGMDV | No: 234) | 235) | ID No: 236) |
| | | | (SEQ ID No: 233) | | | |
| FD 7C | GGSISSYY (SEQ | IYYSGST (SEQ ID | ARDYRFGELFGR | QSVSSN (SEQ ID | GAS (SEQ ID No: | QQYNNWPRA |
| | ID No: 237) | No: 238) | FAWFDP (SEQ ID | No: 240) | 241) | (SEQ ID No: 242) |
| | | | No: 239) | | | |
| FD 7D | GYSFTSYW (SEQ | IDPSDSYT (SEQ | ARHSDCSSTSCYF | ALPKQY (SEQ ID | KDS (SEQ ID No: | QSADSSGTYVV |
| | ID No: 243) | ID No: 244) | VDAFDI (SEQ ID | No: 246) | 247) | (SEQ ID No: 248) |
| | | | No: 245) | | | |
| FJ 10B | GYSFTSYW (SEQ | IDPSDSYT (SEQ | ARLDPRYGPDYY | QSISSY (SEQ ID | AAS (SEQ ID No: | QQSYSTPLT (SEQ |
| | ID No: 249) | ID No: 250) | GMDV (SEQ ID | No: 252) | 253) | ID No: 254) |
| | | | No: 251) | | | |
| FB 9D | GYRFNSYW (SEQ | IFPTDSDV (SEQ | ARHWASMVRGVI | SSDIGKYNY (SEQ | EVT (SEQ ID No: | SSYAFGGSDTRV |
| | ID No: 255) | ID No: 256) | RASHYYGMDV | ID No: 258) | 259) | (SEQ ID No: 260) |
| | | | (SEQ ID No: 257) | | | |
| FB 1E | GYRFNSYW (SEQ | IFPTDSDI (SEQ ID | ARHWASMVRGVI | SSDIGDYNY (SEQ | EVT (SEQ ID No: | SSYAFGGSDIRV |
| | ID No: 261) | No: 262) | RASHYYGMDV | ID No: 264) | 265) | (SEQ ID No: 266) |
| | | | (SEQ ID No: 263) | | | |
| EZ 7A | GYSFTSYW (SEQ | IYPGDSDT (SEQ | ARGWVYRGFPY | SSDVGGYNY | DVS (SEQ ID No: | CSYAGSYTLV |
| | ID No: 267) | ID No: 268) | YGMDV (SEQ ID | (SEQ ID No: 270) | 271) | (SEQ ID No: 272) |
| | | | No: 269) | | | |

*Both EY 6A and EW 9C have an additional pair of expression vectors.

Tables 5 and 6 show that SARS-CoV-2 nucleocapsid-reactive monoclonal antibodies were evolved from 32 clonal groups defined by their heavy chain VDJ and light chain VJ rearrangements. Their average nucleotide somatic mutations are 22±30 in the heavy chain variable regions and 13±18 in the light chain variable regions.

Table 6 shows the nucleotide and amino acid sequences of the heavy chain variable regions and the light chain variable regions of the 32 SARS-CoV-2 nucleocapsid-reactive monoclonal antibodies. Table 7 shows the amino acid sequences of complementarity-determining regions (CDRs) of the heavy chain variable regions and the light chain variable regions of the SARS-CoV-2 nucleocapsid-reactive human monoclonal antibodies.

SARS-CoV-2 nucleocapsid-reactive antibodies EW 4C, EY 2A and EY 3B bound to paraformaldehyde-fixed and Triton X-100-permeabilised SARS-CoV-2 infected cells by immunofluorescence assay.

TABLE 5
Gene usage of heavy and light chain variable domains of SARS-COV-2
nucleocapsid-reactive human monoclonal antibodies.

| | | | | | | V_{H} junction | Mut | aa | | | V_{L} Junction | Mut | aa |
|---|---|---|---|---|---|---|---|---|---|---|---|---|---|
| MAb | H-L | V_{H} | J_{H} | D_{H} | rf | sequence | # | Sub | V_{L} | J_{L} | Sequence | # | Sub |
| EZ 9B | H-λ | 1-2*06 F | 5*02 F | 4-17*01 F | 3 | CAREGPTVT | 0 | 0 | 3-21*02 F | 3*02 F | CQVWDSSSDHPS | 0 | 0 |
| | | | | | | WWFDPW | | | | | WVF | | |
| EZ 11C | H-λ | 1-24*01 F | 5*02 F | 4-17*01 F | 3 | CATTTVTTPT | 0 | 0 | 1-51*02 F | 2*01 or 3*01 | CGTWDSSLRQVV | 3 | 1 |
| | | | | | | ANWFDPW | | | | F | F | | |
| FD 9B | H-λ | 3-7*01 F | 2*01F | 2-21*01 F | 1 | CVKFGRSEGL | 21 | 17 | 7-46*01 F | 2*01 or 3*01 | CFLTYVGARRLF | 6 | 4 |
| | | | | | | FW | | | | or 3*02 F | | | |
| EZ 11A | H-λ | 3-7*01 F | 4*02 F | 1-26*01 F | 3 | CARDDYSGS | 0 | 0 | 4-69*01 F | 3*02 F | CQTWGTGIWVF | 0 | 0 |
| | | | | | | YYWEFDYW | | | | | | | |
| EW 10C | H-κ | 3-7*03 F | 4*02 F | 4-23*01 | 1 | CARGRTLGD | 25 | 15 | 2-30*02 F | 3*01 F | CMQGTHWPPITF | 3 | 1 |
| | | | | ORF | | W | | | | | | | |
| EY 12B | H-λ | 3-9*01 F | 3*01 F | 5-12*01 F | 3 | CAKGRSGYG | 16 | 1 | 1-40*01 or | 2*01 or 3*01 | CQSYDSSLSASVF | 9 | 6 |
| | | | | | | HTAFDVW | | | 02 F | F | | | |
| EZ 8C | H-λ | 3-21*01 F | 3*02 F | 5-18*01 F | 3 | CARELTSYGS | 15 | 9 | 2-14*01 F | 2*01 or 3*01 | CSSYTTTDSVVF | 11 | 6 |
| | | | | | | HD AFDIW | | | | F | | | |
| EY 9C | H-λ | 3-21*01 F | 4*03 F | 2-21*01 F | 2 | CATWGGAPF | 12 | 9 | 2-23*01 or | 2*01 or 3*01 | CCSYAGGRTFNVL | 12 | 10 |
| | | | | | | DYW | | | 02 or 03 F | F | F | | |
| EZ 8B | H-λ | 3-23*04 F | 5*01 or | 3-16*01 F | 2 | CAKDLGYYG | 4 | 2 | 3-1*01 F | 3*02 F | CQAWDSSTAVF | 0 | 0 |
| | | | 02 F | | | SGSSW | | | | | | | |
| FD 3E | H-λ | 18 or 3-30- | 6*02 F | 3-10*01 F | 2 | CAKDPHYYG | 1 | 1 | 2-29*02 F | 4*01 F | CMQGIHLP#TF | 0 | 0 |
| | | 3-30*03 or | | | | SGSYYNQLRG | | | | | | | |
| | | 5*01 F | | | | YYYYGMDV | | | | | | | |
| | | | | | | W | | | | | | | |
| EW 1A | H-λ | 3-33*01 or | 3*01F | 6-13*01 F | 3 | CARDGQHLA | 32 | 17 | 2-14*01 F | 3*02 F | CNSFVSGDSWVF | 27 | 17 |
| | | 06 F | | | | PFAMDVW | | | | | | | |
| FD 5B | H-λ | 3-33*01 or | 4*02 F | 5-24*01 | 3 | CARDERRESY | 6 | 3 | 2-8*01 F | 1*01 F | CSSYAGSNNPFVF | 1 | 1 |
| | | 06 F | | ORF | | NFVLDYW | | | | | | | |
| EW 9A | H-λ | 3-33*01 or | 6*02 F | 3-9*01 F | 2 | CAKDMWALY | 1 | 1 | 3-25*03 F | 3*02 F | CQSADSSGTYWV | 1 | 1 |
| | | 06 F | | | | DILTGYYTPY | | | | | F | | |
| | | | | | | YYYGMDVW | | | | | | | |
| FD 8C | H-κ | 3-49*05 F | 4*02 F | 3-3*01 F | 2 | CTRNDFWSG | 0 | 0 | 2-30*02 F | 4*01 F | CMQGTHWP#LTF | 0 | 0 |
| | | | | | | YYPDYW | | | | | | | |
| EW 5A | H-λ | 3-64*05 or | 2*01 F | 3-3*01 F | 2 | CVKDRGSVIR | 37 | 23 | 7-43*01 F | 2*01 or 3*01 | CLLYCGGGQLF | 2 | 13 |
| | | 3-64D*06 F | | | | DFDVW | | | | or 3*02 F | | | |
| EZ 9C | H-λ | 3-64D*06 F | 4*02 F | 6-19*01 F | 1 | CGKGLLSASG | 34 | 20 | 1-51*01 F | 3*02 F | CATWDSSLSAGV | 5 | 3 |
| | | | | | | GLPIDDW | | | | | F | | |
| EZ 9A | H-λ | 3-74*01 F | 4*02 F | 4-11*01 | 2 | CARDVNRYP | 29 | 18 | 2-14*01 F | 3*02 F | CCSYVNNGAWVF | 28 | 13 |
| | | | | ORF | | DYW | | | | | | | |
| EY 12A | H-λ | 4-30-4*01 | 2*01 F | 3-9*01 F | 2 | CARGMTQDD | 22 | 12 | 1-40*01 F | 2*01 or 3*01 | CQSFDSSLSDFVV | 8 | 6 |
| | | F | | | | ILTGFNRPHW | | | | F | F | | |
| | | | | | | YFDLW | | | | | | | |
| FD 4E | H-λ | 4-31*03 F | 4*02 F | 1-26*01 F | 3 | CARGRGSYL | 0 | 0 | 6-57*01 F | 3*02 F | CQSYDSSN#VF | 2 | 2 |
| | | | | | | AGGNYYFDY | | | | | | | |
| | | | | | | W | | | | | | | |
| FD 4C | H-λ | 4-31*03 F | 4*02 F | 6-13*01 F | 1 | CARVRSSSSW | 2 | 1 | 2-14*01 F | 3*02 F | CSSYTSKWVF | 4 | 2 |
| | | | | | | YFDYW | | | | | | | |
| EW 4C | H-λ | 4-38-2*02 | 1*01F | 3-16*01 F | 2 | CVRGTYGSGL | 49 | 24 | 7-46*01 F | 3*02 F | CFLSHNDAWVF | 17 | 12 |
| | | F | | | | HW | | | | | | | |
| FD 6D | H-κ | 4-38-2*02 | 3*01 or | 3-22*01 F | 2 | CARDRLLAVH | 22 | 10 | 4-1*01 F | 1*01 F | CQQYYDIPRTF | 12 | 6 |
| | | F | 02 F | | | YDSRGYLVD | | | | | | | |
| | | | | | | YW | | | | | | | |
| EY 5A | H-λ | 4-59*01 F | 4*02 F | 4-23*01 | 3 | CARGPGPATG | 23 | 16 | 1-44*01 F | 7*01 F | CSAWDDSLNGPV | 7 | 5 |
| | | | | ORF | | GSLDYW | | | | | F | | |
| EZ 7B | H-κ | 4-59*13 F | 3*02 F | 1-26*01 F | 3 | CARRVFGPVL | 1 | 1 | 4-1*01 F | 3*01 F | CQQYYSTPLTF | 0 | 0 |
| | | | | | | PSKLGGSYW | | | | | | | |
| | | | | | | GGGAFDIW | | | | | | | |
| EZ 4C-1 | H-κ | 4-61*01 or | 4*02 F | 3-3*01 F | 1 | CARAPSAPFG | 24 | 15 | 1-5*03 F | 2*03 F | CQQYNGYSYSF | 17 | 8 |
| | | 03 F | | | | GLFDWILPKG | | | | | | | |
| | | | | | | INNW | | | | | | | |
| EZ 4C-2 | H-λ | 4-61*01 or | 4*02F | 3-3*01 F | 1 | CARAPSAPFG | 22 | 13 | 1-41*01 | 3*02 F | C*IA*HSSPR#WVF | 14 | 8 |
| | | 03 F | | | | GLFDWILPKG | | | ORF | | (2nd-CYS 104 | | |
| | | | | | | IDNW | | | | | not identified) | | |
| FD 4B | H-λ | 4-61*01 or | 5*02 F | 3-3*01 F | | CARAPSAPFG | 24 | 13 | 1-41*01 | 3*02 F | C*IA*HSSPR#WVF | 14 | 8 |
| | | 03 F | | | 1 | GLFDWILPKG | | | ORF | | (2nd-CYS 104 | | |
| | | | | | | IDSW | | | | | not identified) | | |
| EY 8A | H-λ | 4-61*02 F | 6*02 F | 5-12*01F | 3 | CAKGHVISGY | 1 | 1 | 3-25*03 F | 3*02 F | CQSADSSGTYWV | 0 | 0 |
| | | | | | | DDYYYYYGM | | | | | F | | |
| | | | | | | DVW | | | | | | | |
| EY2A | H-κ | 5-51*01 F | 4*02 F | 6-13*01 F | 1 | CVRQERGSNT | 44 | 22 | 2-28*01 or | 2*02 F | CMQALQTPGTF | 14 | 7 |
| | | | | | | WYAGNSW | | | 2D-28*01 F | | | | |
| EY 3B | H-κ | 5-51*01 F | 4*02 F | 6-13*01 F | 1 | CVRQERGSNT | 41 | 21 | 2-28*01 or | 2*02 F | CMQALQTPGTF | 13 | 7 |
| | | | | | | WYAGNSW | | | 2D-28*01F | | | | |
| EZ 4A | H-κ | 5-51*01F | 4*02 F | 6-13*01 F | 2 | CARSPIAADL | 0 | 0 | 1-33*01 or | 3*01 F | CQQYDNLLFTF | 0 | 0 |
| | | | | | | FDYW | | | 1D-33*01 F | | | | |
| EZ 8B | H-κ | 3-23*04 F | 5*01 or | 3-16*01 F | 2 | CAKDLGYYG | 4 | 2 | 3-7*04 | 1*01 F | CQQDYNS#TF | 1 | 1 |
| | | | 02F | | | SGSSW | | | ORF | | | | |

Abbreviations: H, heavy; K, kappa; A, lambda; Vh, variable gene segment of the heavy chain variable domain; Dh, diversity gene segment of the heavy chain variable domain; Jh, joining gene segment of the heavy chain variable domain; Mut, number of nucleotide mutations; Sub, number of amino acid substitutions; Vl, variable gene segment of the light chain variable domain; Jl, joining gene segment of the light chain variable domain.

TABLE 6
Nucleotide and amino acid sequences of the heavy chain variable regions
(V_{H}) and light chain variable regions (V_{L}) of the 30 SARS-CoV-2 nucleocapsid-reactive
human monoclonal antibodies.
Nucleotide sequences of V_{H} and V_{L}

| | | |
|---|---|---|
| EZ 9B | V_{H} | GAGGTGCAGCTGGTGCAGTCTGGGGCTGAGGTGAAGAAGCCTGGGGCCTCAGTGAAGGTCTCCTGCAAGGCTTCTGGA |
| | | TACACCTTCACCGGCTACTATATGCACTGGGTGCGACAGGCCCCTGGACAAGGGCTTGAGTGGATGGGACGGATCAACC |
| | | CTAACAGTGGTGGCACAAACTATGCACAGAAGTTTCAGGGCAGGGTCACCATGACCAGGGACACGTCCATCAGCACAG |
| | | CCTACATGGAGCTGAGCAGGCTGAGATCTGACGACACGGCCGTGTATTACTGTGCGAGAGAGGGGCCTACGGTGACTTG |
| | | GTGGTTCGACCCCTGGGGCCAGGGAACCCTGGTCACCGTCTCCTCA (SEQ ID No: 581) |
| | V_{L} | TCCTATGAGCTGACACAGCCACCCTCGGTGTCAGTGGCCCCAGGACAGACGGCCAGGATTACCTGTGGGGGAAACAAC |
| | | ATTGGAAGTAAAAGTGTGCACTGGTACCAGCAGAAGCCAGGCCAGGCCCCTGTGCTGGTCGTCTATGATGATAGCGACC |
| | | GGCCCTCAGGGATCCCTGAGCGATTCTCTGGCTCCAACTCTGGGAACACGGCCACCCTGACCATCAGCAGGGTCGAAGC |
| | | CGGGGATGAGGCCGACTATTACTGTCAGGTGTGGGATAGTAGTAGTGATCATCCTTCTTGGGTGTTCGGCGGAGGGACCA |
| | | AGCTGACCGTCCTA (SEQ ID No: 582) |
| EZ 11C | V_{H} | GAAGTGCAGCTGGTGGAGTCTGGGGCTGAGGTGAAGAAGCCTGGGGCCTCAGTGAAGGTCTCCTGCAAGGTTTCCGGA |
| | | TACACCCTCACTGAATTATCCATGCACTGGGTGCGACAGGCTCCTGGAAAAGGGCTTGAGTGGATGGGAGGTTTTGATCC |
| | | TGAAGATGGTGAAACAATCTACGCACAGAAGTTCCAGGGCAGAGTCACCATGACCGAGGACACATCTACAGACACAGC |
| | | CTACATGGAGCTGAGCAGCCTGAGATCTGAGGACACGGCCGTGTATTACTGTGCAACAACTACGGTGACTACCCCAACC |
| | | GCAAACTGGTTCGACCCCTGGGGCCAGGGAACCCTGGTCACCGTCTCCTCA (SEQ ID No: 583) |
| | V_{L} | CAGTCTGCCCTGACTCAGCCGCCCTCAGTGTCTGCGGCCCCAGGACAGAAGGTCACCATCTCCTGCTCTGGAAGCAGCT |
| | | CCAACATTGGGAATAATTATGTATCCTGGTACCAGCAGCTCCCAGGAACAGCCCCCAAACTCCTCATCTATGAAAATAATA |
| | | AGCGACCCTCAGGGATTCCTGACCGATTCTCTGGCTCCAAGTCTGGCACGTCAGCCACCCTGGGCATCACCGGACTCCA |
| | | GACTGGGGACGAGGCCGATTATTACTGCGGAACATGGGATAGCAGCCTGCGTCAGGTAGTGTTCGGCGGAGGGACCAAG |
| | | CTGACCGTCCTA (SEQ ID No: 584) |
| FD 9B | V_{H} | GAAGTGCAGCTGGTGGAGTCTGGGGGAGGCTTGGTCCAGCCTGGGGGGTCCCTGAGACTCTCCTGTGCAGGCACTGGA |
| | | TTCAGCTTTAGTAGATATTGGATGAATTGGGTCCGCCAGGCTCCAGGGAAGGGGCTGGAGTGGGTGGCCAACATGGACC |
| | | CAGATGGAGGTGCGAAATACTATCTGGACTCTGTGAAGGGGCGATTCACCATCTCCGGAGACAACGCCAAGAACTCATT |
| | | GTATCTGCAAATGAACAGACTGAGAGCCGAGGACACGGCTGTGTATTACTGTGTCAAATTCGGGCGGTCGGAAGGCTTG |
| | | TTTTGGGGCCGTGGCACCCTGGTCACCGTCTCCTCA (SEQ ID No: 585) |
| | V_{L} | CAGGCTGTGGTGACCCAGGAGCCCTCACTGACTGTGTCCCCAGGAGGGACAGTCACTCTCACCTGTGGCTCCAGCACT |
| | | GGAGCTGTCACCAGTGGTCATTATCCCTACTGGTTCCAGCAGAAGCCTGGCCAAGCCCCCAGGACACTGATTTATGATAC |
| | | AAGCAACAAACACTCCTGGACACCTGCCCGATTCTCAGGCTCCCTCCTTGGGGGCAAAGCTGCCCTGACCCTTTCGGGT |
| | | GCGCAGCCTGAGGATGAGGCTGACTATTACTGCTTCCTCACCTATGTTGGTGCTCGGAGGTTATTCGGCGGAGGGACCAA |
| | | GCTGACCGTCCTA (SEQ ID No: 586) |
| EZ 11A | V_{H} | GAAGTGCAGCTGGTGGAGTCTGGGGGAGGCTTGGTCCAGCCTGGGGGGTCCCTGAGACTCTCCTGTGCAGCCTCTGGA |
| | | TTCACCTTTAGTAGCTATTGGATGAGCTGGGTCCGCCAGGCTCCAGGGAAGGGGCTGGAGTGGGTGGCCAACATAAAGC |
| | | AAGATGGAAGTGAGAAATACTATGTGGACTCTGTGAAGGGCCGATTCACCATCTCCAGAGACAACGCCAAGAACTCACT |
| | | GTATCTGCAAATGAACAGCCTGAGAGCCGAGGACACGGCTGTGTATTACTGTGCGAGAGATGACTATAGTGGGAGCTAC |
| | | TATTGGGAATTTGACTACTGGGGCCAGGGAACCCTGGTCACCGTCTCCTCA (SEQ ID No: 587) |
| | V_{L} | CAGCCTGTGCTGACTCAATCGCCCTCTGCCTCTGCCTCCCTGGGAGCCTCGGTCAAGCTCACCTGCACTCTGAGCAGTG |
| | | GGCACAGCAGCTACGCCATCGCATGGCATCAGCAGCAGCCAGAGAAGGGCCCTCGGTACTTGATGAAGCTTAACAGTGA |
| | | TGGCAGCCACAGCAAGGGGGACGGGATCCCTGATCGCTTCTCAGGCTCCAGCTCTGGGGCTGAGCGCTACCTCACCATC |
| | | TCCAGCCTCCAGTCTGAGGATGAGGCTGACTATTACTGTCAGACCTGGGGCACTGGCATTTGGGTGTTCGGCGGAGGGA |
| | | CCAAGCTGACCGTCCTA (SEQ ID No: 588) |
| EW 10C | V_{H} | TTCACCTTCAACAAGTACAAGATGACCTGGGTCCGCCAGGCTCCAGGGAAGGGGCTGGAGTGGGTGGCCAACATAAAG |
| | | GAAGATGGAAGTGAGAAAAACTATGTGGACTCTGTGAAGGGCCGATTCACCATCTCCAGAGACAACGCCAGGAACTCA |
| | | GTATATATGCACCTGAACAACTTGAGAGTCGAGGACACGGCCGTGTATTACTGTGCGAGAGGGCGGACCCTCGGCGACT |
| | | GGGGCCAGGGAACCACGGTCACCGTCTCCTCA (SEQ ID No: 589) |
| | V_{L} | GCCATCCGGATGACTCAGTCTCCACTCTCCCTGCCCGTCACCCTTGGACAGCCGGCCTCCATCTCCTGCAGGTCTAGTCA |
| | | AAGCCTCGTGCACAGTGATGGAAACACCTACTTGAATTGGTTTCAGCAGAGGCCAGGCCAATCTCCAAGGCGCCTAATT |
| | | TATAAGGTTTCTAACCGGGACTCTGGGGCCCCAGACAGATTCAGCGGCAGTGGGTCAGGCACTGATTTCACACTGAAAA |
| | | TCAGCAGGGTGGAGGCTGAGGATGTTGGGGTTTATTATTGCATGCAAGGTACACACTGGCCTCCGATTACTTTCGGCCCT |
| | | GGGACCAAAGTGGATATCAAA (SEQ ID No: 590) |
| EY 12B | V_{H} | GAAGTGCAGCTGGTGGAGTCCGGGGGAGACTTGGTACAGCCTGGCAGGTCCCTGAGACTCTCCTGTGCAGCCTCTGGAT |
| | | TCACCTTTGATTATTTTGCCATGCACTGGGTCCGGCAAGTTCCAGGGAAGGGCCTGGAGTGGGTCTCAGGTATTCGTTGG |
| | | AATAGTGAAACCATAGGCTATGCGGACTCTGTGAAGGGCCGGTTCACCATCTCCAGAGACAACGCCAAGAAATCACTGT |
| | | ATCTGGAAATGAACAGTCTGAGAAGTGAGGACACGGCCTTCTATTACTGTGCAAAAGGTCGGAGTGGCTACGGCCACAC |
| | | TGCTTTTGATGTCTGGGGCCAAGGGACAATGGTCACCGTCTCTTCA (SEQ ID No: 591) |
| | V_{L} | CAGTCTGCCCTGACTCAGCCGCCCTCAGTGTCTGGGGCCCCAGGGCAGAGGGTCACCATCTCCTGCACTGGGAGCAGCT |
| | | CCAACATCGGGGCAAATTATGATGTACACTGGTATCAGCGGCTTCCAGGAACAGCCCCCAAACTCCTCATCTATGATAAC |
| | | AACAATCGGCCCTCGGGGGTCCCTGACCGATTCTCTGGCTCCAAGTCTGGCACCTCAGCCTCCCTGGCCATCGCTGGGCT |
| | | CCAGGCTGAGGATGAGGCTGATTATTACTGCCAGTCCTATGACAGCAGCCTGAGTGCTTCAGTATTCGGCGGAGGGACCA |
| | | AGCTGACCGTCCTA (SEQ ID No: 592) |
| EZ 8C | V_{H} | GAAGTGCAGCTGGTGGAGTCTGGGGGAGGCCTGGTCAAGCCTGGGGGGTCCCTGAGACTCTCCTGTGCAGCCTCTGGA |
| | | TTCACCTTCAGTTCCTATACCATGAACTGGGTCCGCCAGGCTCCAGGGAAGGGGCTGGAGTGGGTCTCATCCTTTATTGC |
| | | TAATAGTGATTACAAGTTCTACGCAGACTCAGTGAAGGGCCGATTCACCATCTCCAGAGACAACGCCAAGAGCTCACTG |
| | | TATCTGCAAATGAACAGCCTGAGAGCCGAGGACACGGCTGTGTATTACTGTGCGAGAGAACTAACCAGTTATGGTTCCC |
| | | ACGATGCTTTTGATATCTGGGGCCAAGGGACAATGGTCACCGTCTCTTCA (SEQ ID No: 593) |
| | V_{L} | CAGTCTGCCCTGACTCAGCCTGCCTCCGTGTCTGGGTCTCCTGGACAGTCGATCACCATCTCCTGCACTGGAACCAGCA |
| | | GTGACGTTGGTGGTTATAACTATGTCTCCTGGTACCAACAACACCCAGGCAAAGCCCCCAAACTCTTGATTTATGAGGTC |
| | | ACTAATCGGCCCTCAGGGGTTTCTGATCGCTTCTCTGGCTCCAAGTCTGCCAATGTGGCGTCCCTGACCATCTCTGGGCT |
| | | CCAGGCTGAGGACGAGGCTGATTATTACTGCAGCTCATATACAACCACCGACTCCGTGGTTTTCGGCGGAGGGACCAAG |
| | | CTGACCGTCCTA (SEQ ID No: 594) |
| EY 9C | V_{H} | GAAGTGCAGCTGGTGGAGTCTGGGGGAGGCCTGGTCAAGCCTGGGGGGTCCCTGAGACTCTCCTGTGCAGCCTCTGAA |
| | | TTCACCTTCAGTAGCTATACCTTGAACTGGGTCCGCCAGGCTCCAGGGAAGGGGCTGGAGTGGGTCTCATCCATTAGTAC |
| | | TAGTAGTGCTTACATATACTACGCAGACTCAGTGAAGGGCCGATTCACCATCTCCAGAGACAACGCCAAGAAGTCACTGT |
| | | CTCTGCAAATGAACAGCCTGACAGCCGAGGACACGGCTGTCTATTACTGTGCGACTTGGGGCGGTGCCCCCTTTGACTA |
| | | CTGGGGCCAAGGGACAATGGTCACCGTCTCA (SEQ ID No: 595) |
| | V_{L} | CAGTCTGTGCTGACTCAGCCTGCCTCCGTGTCTGGGTCTCCTGGACGGTCGATCACCATCTCCTGCACTGAAACCAGCA |
| | | GTGATGTTGGGACTTATAACCTTGTCTCCTGGTACCAACAGCACCCAGGCAAAGCCCCCAAACTCATGATTTTTGACGAC |
| | | AATAAGCGGCCCTCAGGGGTTTCTAATCGCTTCTCTGGCTCCAAGTCTGGCAACACGGCCTCCCTGACAATCTCTGGGCT |
| | | CCAGGCTGAGGACGAGGCTGATTATTACTGCTGCTCATATGCAGGTGGTAGGACCTTCAATGTGCTATTCGGCGGCGGGA |
| | | CCAAGCTGACCGTCCTA (SEQ ID No: 596) |
| EZ 8B | V_{H} | GAAGTGCAGCTGGTGGAGTCTGGGGGAGGCTTGGTACAGCCTGGGGGGTCCCTGAGACTCTCCTGTGCAGCCTCTGGAT |
| | | TCACCTTTAGCAGCTATGCCATGAGCTGGGTCCGCCAGGCTCCAGGGAAGGGGCTGGAGTGGGTCTCAGCTATTAGTGG |
| | | TAGTGGTGGTAGCACATACTACGCAGACTCCGTGAAGGGCCGGTTCACCATCTCCAGAGACAATTCCAAGAACACGCTG |
| | | TATCTGCAAATGAACAGCCTGAGAGCCGAGGACACGGCCGTATATTACTGTGCGAAAGATCTGGGGTACTATGGTTCGGG |
| | | GAGTTCCTGGGGCCAGGGAACCCTGGTCACCGTCTCCTCA (SEQ ID No: 597) |
| | V_{L} | TCCTATGAGCTGACTCAGCCACCCTCAGTGTCCGTGTCCCCAGGACAGACAGCCAGCATCACCTGCTCTGGAGATAAATT |
| | | GGGGGATAAATATGCTTGCTGGTATCAGCAGAAGCCAGGCCAGTCCCCTGTGCTGGTCATCTATCAAGATAGCAAGCGGC |
| | | CCTCAGGGATCCCTGAGCGATTCTCTGGCTCCAACTCTGGGAACACAGCCACTCTGACCATCAGCGGGACCCAGGCTAT |
| | | GGATGAGGCTGACTATTACTGTCAGGCGTGGGACAGCAGCACTGCGGTGTTCGGCGGAGGGACCAAGCTGACCGTCCTA |
| | | (SEQ ID No: 598) |
| FD 3E | V_{H} | GAAGTGCAGCTGGTGGAGTCTGGGGGAGGCGTGGTCCAGCCTGGGAGGTCCCTGAGACTCTCCTGTGCAGCCTCTGGA |
| | | TTCACCTTCAGTAGCTATGGCATGCACTGGGTCCGCCAGGCTCCAGGCAAGGGGCTGGAGTGGGTGGCAGTTATATCATA |
| | | TGATGGAAGTAATAAATACTATGCAGACTCCGTGAAGGGCCGATTCACCATCTCCAGAGACAATTCCAAGAACACGCTGT |
| | | ATCTGCAAATGAACAGCCTGAGAGCTGAGGACACGGCTGTGTATTACTGTGCGAAAGATCCCCATTACTATGGTTCGGGG |
| | | AGTTATTATAACCAGCTGAGGGGATACTACTACTACGGTATGGACGTCTGGGGCCAAGGGACCACGGTCACCGTCTCCTC |
| | | A (SEQ ID No: 599) |
| | V_{L} | GATATTGTGATGACCCAGACTCCACTCTCTCTGTCCGTCACCCCTGGACAGCCGGCCTCCATCTCCTGCAAGTCTAGTCA |
| | | GAGCCTCCTGCATAGTGATGGAAAGACCTATTTGTATTGGTACCTGCAGAAGCCAGGCCAGTCTCCACAGCTCCTAATCT |
| | | ATGAAGTTTCCAGCCGGTTCTCTGGAGTGCCAGATAGGTTCAGTGGCAGCGGGTCAGGGACAGATTTCACACTGAAAAT |
| | | CAGCCGGGTGGAGGCTGAGGATGTTGGGGTTTATTACTGCATGCAAGGTATACACCTTCCTCACTTTCGGCGGAGGGACC |
| | | AAGTGGAGATCAAA (SEQ ID No: 600) |
| EW 1A | V_{H} | GAAGTGCAGCTGGTGGAGTCTGGGGGAGGCGTGGTCCAGCCTGGGTCGTCCCTGAGACTCTCCTGTGAAACGTCTGGT |
| | | TTCACCTTCAGTGGACATGCCATGCACTGGGTCCGCCAGGCTCCAGGCAAGGGGCTGGAGTGGCTGGCACAAATCTGGT |
| | | TCGATGGAAGTGAAAAATACTATGCAGATTCCGTGAAGGGTCGATTCACCATCTCCAGAGACAATTCCAAGAAAATCCTA |
| | | TATATGCAAATGAACAGCCTGAGAGTCCAAGACACGGCTGTGTATTACTGTGCGAGAGATGGGCAACATCTGGCACCTTT |
| | | CGCTATGGACGTCTGGGGCCAAGGGACAATGGTCACCGTCTCTTCA (SEQ ID No: 601) |
| | V_{L} | CAGTCTGTGCTGACTCAGCCTGCCTCCGTGTCTGGGTCTCCTGGACAGTCAATCACCATCTCCTGCACTGGAACCAGCA |
| | | GTGACGTTGGTGCCTCTAACCGTGTTTCCTGGTACCAACACTCCCCAGGCGAAGCCCCCAAACTCATCATTTATCAGGTC |
| | | ACTGTTCGGCCCTCAGGGGTGTCTGATCGCTTCTCTGGCTCGAAGTCCGGCAACACGGCCTCCCTGACCATCTCTGGGCT |
| | | CCGGACTGAGGACGAGGCTGAATATTACTGCAACTCATTTGTAAGCGGTGACTCTTGGGTGTTCGGCGGAGGGACCAAG |
| | | GTGACCGTCCTA (SEQ ID No: 602) |
| FD 5B | V_{H} | GAAGTGCAGCTGGTGGAGTCTGGGGGAGGCGTGGTCCAGCCTGGGAGGTCCCTGAGACTCTCCTGTGCAGCGTCTGGA |
| | | TTCACCTTCAGTAGCTATGGCATGCACTGGGTCCGCCAGGCTCCAGGCAAGGGGCTGGAGTGGGTGGCAGTTATATGGTA |
| | | TGATGGAAGTAATAAATACTATGCAGACTCCGTGAAGGGCCGATTCACCATCTCCAGAGACAATTCCAAGAACACGCTGT |
| | | ATCTGCAAATGAACAGCCTGAGAGCCGAGGACACGGCCGTCTATTACTGTGCGAGAGATGAACGTAGAGAGTCCTACAA |
| | | TTTCGTGTTGGACTACTGGGGCCAGGGAACCCTGGTCACCGTCTCCTCA (SEQ ID No: 603) |
| | V_{L} | CAGTCTGTGCTGACTCAGCCTCCCTCCGCGTCCGGGTCTCCTGGACAGTCAGTCACCATCTCCTGCACTGGAACCAGCA |
| | | GTGACGCTGGTGGTTATAACTATGTCTCCTGGTACCAACAGCACCCAGGCAAAGCCCCCAAACTCATGATTTATGAGGTC |
| | | AGTAAGCGGCCCTCAGGGGTCCCTGATCGCTTCTCTGGCTCCAAGTCTGGCAACACGGCCTCCCTGACCGTCTCTGGGC |
| | | TCCAGGCTGAGGATGAGGCTGATTATTACTGCAGCTCATATGCAGGCAGCAACAACCCCTTTGTCTTCGGAACTGGGACC |
| | | AAGGTCACCGTCCTA (SEQ ID No: 604) |
| EW 9A | V_{H} | GAGGTGCAGCTGTTGGAGTCTGGGGGAGGCGTGGTCCAGCCTGGGAGGTCCCTGAGACTCTCCTGTGCAGCGTCTGGA |
| | | TTCACCTTCAGTAGCTATGGCATGCACTGGGTCCGCCAGGCTCCAGGCAAGGGGCTGGAGTGGGTGGCAGTTATATGGTA |
| | | TGATGGAAGTAATAAATACTATGCAGACTCCGTGAAGGGCCGATTCACCATCTCCAGAGACAATTCCAAGAACACGCTGT |
| | | ATCTGCAAATGAACAGCCTGAGAGCCGAGGACACGGCTGTGTATTACTGTGCGAAAGATATGTGGGCCTTATACGATATT |
| | | TTGACTGGTTATTATACACCCTACTACTACTACGGTATGGACGTCTGGGGCCAAGGGACCACGGTCACCGTCTCCTCA |
| | | (SEQ ID No: 605) |
| | V_{L} | TCCTATGAGCTGACACAGCCACCCTCGGTGTCAGTGTCCCCAGGACAGACGGCCAGGATCACCTGCTCTGCAGATGCAT |
| | | TGCCAAAGCAATATGCTTATTGGTACCAGCAGAAGCCAGGCCAGGCCCCTGTGCTGGTGATATATAAAGACAGTGAGAG |
| | | GCCCTCAGGGATCCCTGAGCGATTCTCTGGCTCCAGCTCAGGGACAACAGTCACGTTGACCATCAGTGGAGTCCAGGCA |
| | | GAAGACGAGGCTGACTATTACTGTCAATCAGCAGACAGCAGTGGTACTTATTGGGTGTTCGGCGGAGGGACCAAGCTGA |
| | | CCGTCCTA (SEQ ID No: 606) |
| FD 8C | V_{H} | GAGGTGCAGCTGGTGGAGTCTGGGGGAGGCTTGGTAAAGCCAGGGCGGTCCCTGAGACTCTCCTGTACAGCTTCTGGAT |
| | | TCACCTTTGGTGATTATGCTATGAGCTGGTTCCGCCAGGCTCCAGGGAAGGGGCTGGAGTGGGTAGGTTTCATTAGAAGC |
| | | AAAGCTTATGGTGGGACAACAGAATACGCCGCGTCTGTGAAAGGCAGATTCACCATCTCAAGAGATGATTCCAAAAGCA |
| | | TCGCCTATCTGCAAATGAACAGCCTGAAAACCGAGGACACAGCCGTGTATTACTGTACTAGGAACGATTTTTGGAGTGGT |
| | | TATTATCCAGACTACTGGGGCCAGGGAACCCTGGTCACCGTCTCCTCA (SEQ ID No: 607) |
| | V_{L} | GATATTGTGATGACTCAGTCTCCACTCTCCCTGCCCGTCACCCTTGGACAGCCGGCCTCCATCTCCTGCAGGTCTAGTCA |
| | | AAGCCTCGTACACAGTGATGGAAACACCTACTTGAATTGGTTTCAGCAGAGGCCAGGCCAATCTCCAAGGCGCCTAATT |
| | | TATAAGGTTTCTAACCGGGACTCTGGGGTCCCAGACAGATTCAGCGGCAGTGGGTCAGGCACTGATTTCACACTGAAAA |
| | | TCAGCAGGGTGGAGGCTGAGGATGTTGGGGTTTATTACTGCATGCAAGGTACACACTGGCCCCGCTCACTTTCGGCGGA |
| | | GGGACCAAAGTGGATATCAAAC (SEQ ID No: 608) |
| EW 5A | V_{H} | GAAGTGCAGCTGGTGGAGTCGGGGGGAGGCTTGGTCCAGCCTGGGGGGTCCCTGCGACTCTCCTGTTCAGTCTCTGGAT |
| | | TCCCCTTCGGAACATATGCTATGCACTGGGTCCGCCAGGCTCCCGGGAAGGGGCTAGATTATGTTTCAGCTATTAATAATG |
| | | ATGGGAGTATTACATACTACGCAGACTCAGTGAGGGGCAGATTCACCGTCTCCAGAGACAATTCCGAGAACACTCTATAT |
| | | CTTCGACTGAGCGGTCTGAGACCTGACGACACGGCTATCTATTATTGTGTGAAAGATCGGGGCTCCGTTATTCGGGACTT |
| | | CGACGTCTGGGGCCGTGGCACCCTGGTCACCGTCTCCTCA (SEQ ID No: 609) |
| | V_{L} | CAGACTGTGGTGACTCAGGAGCCCTCACTGACTGTCTCCCCAGGAGGGACAGTCACTCTCACCTGTGCTTCCAGTACTG |
| | | GAACAGTCACCAGTGATTACTATCCAAACTGGTTCCAGCAGAAGCCTGGACAGGCACCCAGGCCTCTGATTTTCGGTAC |
| | | AGCCTACAGACACTCCTGGACCCCTGCCCGATTCTCAGGCTCCCTCCTTGGGGGCAAAGCTGCCCTGACAGTGTCAGAT |
| | | GTGCAGCCTGAGGACGAGGCTGACTATTACTGCCTGCTCTACTGTGGTGGTGGTCAGCTTTTCGGCGGAGGGACCAAGC |
| | | TGACCGTCCTA (SEQ ID No: 610) |
| EZ 9C | V_{H} | GAAGTGCAGCTGGTGGAGTCTGGGGGAGGCTTGGTCCAGCCTGGGGGGTCCCTGAGACTCTCCTGTTCAGCCTCTGGAT |
| | | TCACCCTCAATGGCTATGCTATGCACTGGGTCCGCCAGGCTCCAGGGAAAGGACTGGAGTATGTTTCAAGTCTTTCTAAT |
| | | CGTGGAGATAATACACGCTACGCAGAGTCCGTGAAGGGCAGATTCCTCATCTCCAGAGACATTGCCAAGGACACGCTTT |
| | | ATCTTCAGATGAGCAGTCTGAGACCTGAGGACACGGCTGTCTATTACTGTGGGAAAGGCCTTTTGTCTGCCAGTGGGGG |
| | | ATTGCCGATTGACGACTGGGGCCAGGGAACCCTGGTCACCGTCTCCTCA (SEQ ID No: 611) |
| | V_{L} | CAGTCTGTGCTGACGCAGCCGCCCTCAGTGTCTGCGGCCCCAGGACAGAAGGTCACCATCTCCTGCTCTGGAAGCAGCT |
| | | CCAACATTGGGAATAATTATGTATCCTGGTACCAGCAGTTCCCAGGAACAGCCCCCAAACTCCTCATTTATGACAATAATA |
| | | AGCGACCCTCAGGGATTCCTGACCGATTCTCTGGCTCCAAGTCTGGCACGTCAGCCACCCTGGGCATTACCGGCCTCCA |
| | | GACTGGGGACGAGGCCGCTTATTACTGCGCAACATGGGATAGCAGCCTGAGTGCTGGGGTGTTCGGCGGAGGGGCCAA |
| | | GCTGACCGTCCTA (SEQ ID No: 612) |
| EZ 9A | V_{H} | GAAGTGCAGCTGGTGGAGTCCGGGGGAGGCTTAGTTCAGCCTGGGGAGTCACTGAGAGTCTCCTGTGCAGCCTCTGGA |
| | | TTCACCTTCAGTAACTACTGGATGCACTGGGTCCGCCAAGTCCCAGGAAAGGGGCCGGTGTGGGTCTCAATTATTAATAC |
| | | TGATGGAAGTATCACAAGATATGTGGACTCCGTGAGGGGCCGATTCACCATCTCCAGAGACAACGCCAAGAACACGGTG |
| | | CATCTGCAAATGAACAGCCTGACAGCCGAGGACACGGCTATATATTATTGTGCAAGAGATGTCAATAGGTACCCTGACTA |
| | | CTGGGGCCAGGGAACCCTGGTCACCGTCTCCTCA (SEQ ID No: 613) |
| | V_{L} | CAGTCTGCCCTGACTCAGCCCGCCTCCGTGTCTGGGTCTCCTGGGCAGTCGATCACCATTTCCTGCACTGGAACCTACAG |
| | | TGACGTTGGTTATTATAACTATGTCTCCTGGTATCAACAACAGCCCGGCAAGGCCCCCAAAGTCATCATTCATGGGGACAT |
| | | TAATCGGCCCTTTGGAGTTTCTAATCGCTTTTCTGGCTCCAAGTCTGGCAACACGGCCTCCCTGACAATCTCTGGGCTCC |
| | | AGGCCGAGGACGAGGCTGATTATTTCTGCTGCTCATATGTAAATAATGGCGCTTGGGTGTTCGGCGGAGGGACCAAGTTG |
| | | ACCGTCCTA (SEQ ID No: 614) |
| EY 12A | V_{H} | GAAGTGCAGCTGGTGGAGTCGGGCCCCGGACTGGTGAAGCCTTCACAGACCCTGTCCCTCACCTGCGCTGTCTCCGGTG |
| | | GCTCCATCAGCAGTGGTGGTTACTACTGGAGTTGGATCCGCCAGCCCCCAGGAAAGGGCCTGGAGTTGATTGGGTACAC |
| | | CGATTACACTGGGAAGACCCTCTACAACCCATCCCTCAAGAGTCGACTTACCATATCAGTGGACACGTCCAAGAACCAG |
| | | TTCTCCCTGAAGTTGAGGTCTGTGACTGCCGCAGACACGGCCGTCTATTACTGTGCCAGAGGGATGACGCAAGACGATA |
| | | TTTTGACTGGTTTTAATAGGCCTCACTGGTATTTCGATCTCTGGGGCCGTGGCAGTCTGGTCACCGTCTCCTCA |
| | | (SEQ ID No: 615) |
| | V_{L} | CAGTCTGTGCTGACTCAGCCGCCCTCAGTGTCTGGGGCCCCAGGGCAGAGGGTCACCATCTCCTGCACTGGGAGCAGCT |
| | | CCAACATCGGGGCAGGTTATGATGTACACTGGTACCAGCAGCTTCCAGGAACAGCCCCCAAAGTCGTCATCTATCGTAAC |
| | | ATGAATCGGCCCTCAGGGGTCCCTGACCGATTCTCTGGCTCCAAGTCTGGCACCTCAGCCTCCCTGGCCATCACTGGGCT |
| | | CCAGGCTGAGGATGAGGCTGATTATTACTGCCAGTCCTTTGACAGCAGCCTGAGTGATTTTGTGGTTTTCGGCGGAGGGA |
| | | CCAAGCTGACCGTCCTA (SEQ ID No: 616) |
| FD 4C | V_{H} | GAAGTGCAGCTGGTGGAGTCGGGCCCAGGACTGGTGAAGCCTTCACAGACCCTGTCCCTCACCTGCACTGTCTCTGGTG |
| | | GCTCCATCAGCAGTGGTGGTTACTACTGGAGCTGGATCCGCCAGCACCCAGGGAAGGGCCTGGAGTGGATTGGGTACAT |
| | | CTATTACAGTGGGAGCACCTACTACAACCCGTCCCTCAAGAGCCGAGTTACCATATCAGTAGACACGTCTAAGAACCAGT |
| | | TCTCCCTGAAGCTGAGCTCTGTGACTGCCGCGGACACGGCCGTGTATTACTGTGCGAGAGTCCGGTCTAGCAGCAGCTG |
| | | GTACTTTGACTACTGGGGCCAGGGAACCCTGGTCACCGTCTCCTCA (SEQ ID No: 617) |
| | V_{L} | CAGTCTGTGCTGACTCAGCCTGCCTCCGTGTCTGGGTCTCCTGGACAGTCGATCACCATCTCCTGCACTGGAACCAGCA |
| | | GTGACGTTGGTGGTTATAACTATGTCTCCTGGTACCAACAGCACCCAGGCAAAGCCCCCAAACTCATGATTTATGATGTC |
| | | AGTAAGCGGCCCTCAGGGGTTTCTAATCGCTTCTCTGGCTCCAAGTCTGGCAACACGGCCTCCCTGACCATCTCTGGGCT |
| | | CCAGGCTGAGGACGAGGCTGATTATTACTGCAGCTCATATACAAGCAAATGGGTGTTCGGCGGAGGGACCAAGCTGACC |
| | | GTCCTA (SEQ ID No: 618) |
| EW 4C | V_{H} | GAGGTGCAGCTGGTGGAGTCGGGCCCAGGGCTGGTGAAGCCTTCGGAGACCCTGTCCCTCACCTGCAGTGTCTCTGCT |
| | | GACTCCTTCAAAAAAGGTTACTATTGGGGCTGGATCCGGCAGCCCCCTGGGAAGGGATTGGAATCGATTGTCAATTCTTT |
| | | TGATTCCGGGACCACCCGCTATAATCCGTCCCTCTGGGGTCGAGCCACCGTATCAGACATGTCCAAGTGGCACTTCTCCC |
| | | TGAAGTTGACCTCTGTGACCGCCGCAGACACGGCCGTTTATTATTGTGTCCGGGGAACATATGGTTCGGGCCTTCACTGG |
| | | GGCCAGGGAATCCTGGTCACCGTCTCCTCA (SEQ ID No: 619) |
| | V_{L} | CAGACTGTGGTGACCCAGGAGCCCTCACTGACTGTGTCCCCAGGAGGGACAGTCACTCTCACTTGTGGCTCCAGCGTTG |
| | | GAACTGTCGCCAGTGGTCATTATCCCTACTGGGTCCAGCAGAAGCCTGGCCAAGCCCCCAGGACACTGATTTATGATACA |
| | | GACAACAAACAATCCTGGACCCCTGCCCGGTTCTCAGGCTCCCTCCTTGGGGGCAAGGCTGCCCTGACCCTTTCGGGTG |
| | | CGCAGCCTGAGGATGAGGCTGACTATTACTGCTTTCTCTCCCATAATGATGCTTGGGTGTTCGGCGGAGGGACCAAGTTG |
| | | ACCGTCCTT (SEQ ID No: 620) |
| FD 6D | V_{H} | GAAGTGCAGCTGGTGGAGTCGGGCCCGGGACTGGTGAAGGTTTCGGAGACCCTGTTCCTCACCTGCACTGTCTCTGGGT |
| | | ACTCCATCGGCAGTGGTAACTACTGGGGCTGGATCCGGCAGCCCCCAGGGAAGGTTCTGGAGTGGATTGGGAGTACTTA |
| | | CCACAGTGGGACCACCTACTACAATCCGTCCCTCAAGAGTCGAGTCACCATATCAGTTGACTCGTCCAAGAATCAGTTCT |
| | | CCCTGAAGCTGACCTCTGTGACCGCCGCAGACACGGCCGTGTATTACTGTGCGAGAGATCGGCTATTAGCTGTCCATTAT |
| | | GACAGCCGTGGTTATTTAGTTGACTACTGGGGCCAAGGGACAATGGTCACCGTCTCTTCA (SEQ ID No: 621) |
| | V_{L} | GCCATCCGGATGACCCAGTCTCCAGACTCCCTGGCTGTGTCTCTGGGCGAGAGGGCCACCATCAACTGCAAGTCCAGCC |
| | | AGAGTATTTTTTACAGCTCCAACAATAAGAACTATTTAGCTTGGTACCAGCACAAACCGGGACAGCCTCCTAAGCTCCTC |
| | | ATTTACTGGGCATCTACCCGGGAATCCGGGGTCCCTGACCGATTCAGTGGCAGCGGGTCTGGGACAGATTTCACTCTCAC |
| | | CATCAGCAGCCTGCAGGCTGAAGATGTGGCAATTTATTACTGTCAGCAATATTATGATATTCCTCGGACGTTCGGCCAAGG |
| | | GACCAAGGTGGAAATCAGA (SEQ ID No: 622) |
| EY 5A | V_{H} | GAAGTGCAGCTGGTGGAGTCGGGCCCAGGACTGGTGAAGCCTTCGGGGACCCTGTTCCTCACCTGCACTGTCTCTGGTG |
| | | GCTCCATCAGTAATTACTACTGGAGCTGGATCCGGCAGCCCCCAGGGAAGGCACTAGAGTGGATTGGGTTTATTTATTCC |
| | | AATGGGAACACTGATTACAACCCCTCCCTCCAGAGTCGAGTCACCATATCGGGAGACACGTCCAAGAACCAGTTCTCCC |
| | | TGAACCTGAGGTCTGTTACCGCTGCGGACACGGCCGTGTATTACTGTGCGAGAGGGCCGGGGCCGGCTACGGGGGGTAG |
| | | TCTTGACTACTGGGGCCAAGGGACAATGGTCACCGTCTCTTCA (SEQ ID No: 623) |
| | V_{L} | AATTTTATGCTGACTCAGCCACCCTCAGCGTCTGGGACCCCCGGGCAGAGGGTCACCATCTCTTGTTCTGGAAGCAGCTC |
| | | CAACATCGGAATAAATACTGTAAACTGGTACCAGCACCTCCCAGGAACGGCCCCCAAACTCCTCATCTATGGTAATAATC |
| | | AGCGGCCCTCAGGGGTCCCTGACCGATTCTCTGGCTCCAAGTCTGGCACCTCAACCTCCCTGGCCATCAGTGGGCTCCA |
| | | GTCTGAGGATGAGGCTGATTATTATTGTTCAGCATGGGATGACAGCCTGAATGGTCCTGTGTTCGGAGGAGGCACCCAGC |
| | | TGACCGTCCTC (SEQ ID No: 624) |
| EZ 7B | V_{H} | GAAGTGCAGCTGGTGGAGTCGAGCCCAGGACTGGTGAAGCCTTCGGAGACCCTGTCCCTCACCTGCACTGTCTCTGGT |
| | | GGCTCCATCAGTAGTTACTACTGGAGCTGGATCCGGCAGCCCCCGGGGAAGGGACTGGAGTGGATTGGGTATATCTATTA |
| | | CAGTGGGAGCACCAACTACAACCCCTCCCTCAAGAGTCGAGTCACCATATCAGTAGACACGTCCAAGAACCAGTTCTCC |
| | | CTGAAGCTGAGCTCTGTGACCGCTGCGGACACGGCCGTGTATTACTGTGCGAGAAGGGTTTTCGGCCCCGTCCTCCCTT |
| | | (SEQ ID No: 625) |
| | V_{L} | GACATCCAGTTGACCCAGTCTCCAGACTCCCTGGCTGTGTCTCTGGGCGAGAGGGCCACCATCAACTGCAAGTCCAGCC |
| | | AGAGTGTTTTATACAGCTCCAACAATAAGAACTACTTAGCTTGGTACCAGCAGAAACCAGGACAGCCTCCTAAGCTGCTC |
| | | ATTTACTGGGCATCTACCCGGGAATCCGGGGTCCCTGACCGATTCAGTGGCAGCGGGTCTGGGACAGATTTCACTCTCAC |
| | | CATCAGCAGCCTGCAGGCTGAAGATGTGGCAGTTTATTACTGTCAGCAATATTATAGTACTCCCCTCACTTTCGGCCCTGG |
| | | GACCAAGGTGGAAATCAGA (SEQ ID No: 626) |
| EZ 4C | V_{H} | GAAGTGCAGCTGGTGGAGTCGGGCCCAGGACTGGTGAAGCCTTCGGAGACCCTGTCCCTCACCTGCACTGTCTCTGGT |
| kappa | | GGCTCCGTCAGTAGTGGTAATAACTACTGGAGCTGGATCCGGCAGCCCCCAGGGAAGGCACTGGAGTGGATTGGATATAT |
| | | CTATTACAGTGGGAGCACCAAGTACAACCCCTCCCTCAAGAGTCGAGTCACCATGTCTGTAGGCACGTCCAAGAATCAA |
| | | TTCTCTCTGAAAGTGAACTCTGTGACGGCTGCGGACACGGCCATGTATTACTGTGCCAGAGCCCCCTCGGCTCCCTTTGG |
| | | GGGACTTTTTGACTGGATATTACCTAAAGGGATTAACAACTGGGGCCAGGGAACCCTGGTCACCGTCTCCTCA (SEQ ID |
| | | No: 627) |
| | V_{L} | GAAATTGTGTTGACACAGTCTCCTTCCACCCTGTCTGCATCTGTAGGAGACAGAGTCACCATCACTTGCCGGACCAGTCA |
| | | GAGTATTCGTAGCTGGTTGGCCTGGTATCAACAAAAACCAGGGAAAGCCCCTAAACTCCTGATCTTTGAGGCATCTACTT |
| | | TAGAAAGTGGGGTCCCAGAGAGGTTCAGCGGCAGTGGATCTGGGGCGGAATTCACTCTCACCATCAGCAGCCTGCAGC |
| | | CTGATGATTTTGCAACTTATTACTGTCAACAGTATAATGGTTATTCTTACAGTTTTGGCCAGGGGACCAAGGTGGAAATCA |
| | | GA (SEQ ID No: 628) |
| EZ 4C | V_{H} | GTTCAGCTGGTGCAGGAGTCGGGCCCAGGACTGGTGAAGCCTTCGGAGACCCTGTCCCTCACCTGCACTGTCTCTGGTG |
| lambda | | GCTCCGTCAGTAGTGGTAATAACTACTGGAGCTGGATCCGGCAGCCCCCAGGGAAGGCACTGGAGTGGATTGGATATATC |
| | | TATTACAGTGGGAGCACCAAGTACAACCCCTCCCTCAAGAGTCGAGTCACCATGTCTGTAGACACGTCCAAGAATCAATT |
| | | CTCTCTGAAAGTGAACTCTGTGACGGCTGCGGACACGGCCATGTATTACTGTGCCAGAGCCCCCTCGGCTCCCTTTGGG |
| | | GGACTTTTTGACTGGATATTACCTAAAGGGATTGACAACTGGGGCCAGGGAACCCTGGTCACCGTCTCCTCA |
| | | (SEQ ID No: 629) |
| | V_{L} | CAGTCTGCCCTGACTCAGCCGCCTTCAGTGTCTGCCGCCCCAGGACAGAAGGTCACCATCTCCTACTCTGGAAACAGCT |
| | | CCGACATGGGGACTTATGCGGTATCTTGGTAACAGCGACTCCCAGGAACAGCCCCCAAACTCTTCATCTGTGAAGAGAA |
| | | TAAGCGACCCCCAGGGATTCCTGACCGATTCTCTGGCTCCAAGTCTGGCACTTCAGCCACCCTGGGCATCACTGGCCTCT |
| | | GGCCTGAGGACGAGGCCGATTATTGCTAAATAGCATGACATAGCAGCCCGAGACTTGGGTGTTCGGCGGAGGGACCAAG |
| | | CTGACCGTCCTAG (SEQ ID No: 630) |
| FD 4B | V_{H} | CAGCTGCAGCTGCAGGAGTCGGGCCCAGGACTGGTGAAGCCTTCGGAGACCCTGTCCCTCACCTGCACTGTCTCTGGTG |
| | | GCTCCGTCAGTAGTGGTAATAACTACTGGAGCTGGATCCGGCAGCCCCCAGGGAAGGCACTGGAGTGGATTGGATATCT |
| | | CTATTACAGTGGGAGCACCAAGTACAACCCCTCCCTCAAGAGTCGAGTCACCATGTCTGTAGACACGTCCAAGAATCAA |
| | | TTCTCTCTGAAAGTGAACTCTGTGACGGCTGCGGACACGGCCATGTATTATTGTGCCAGAGCCCCCTCGGCTCCCTTTGG |
| | | GGGACTTTTTGACTGGATATTACCTAAAGGGATTGACAGCTGGGGCCAGGGAACCCTGGTCACCGTCTCCTCA (SEQ ID |
| | | No: 631) |
| | V_{L} | CAGTCTGCCCTGACTCAGCCGCCTTCAGTGTCTGCCGCCCCAGGACAGAAGGTCACCATCTCCTACTCTGGAAACAGCT |
| | | CCGACATGGGGACTTATGCGGTATCTTGGTAACAGCGACTCCCAGGAACAGCCCCCAAACTCTTCATCTGTGAAGAGAA |
| | | TAAGCGACCCCCAGGGATTCCTGACCGATTCTCTGGCTCCAAGTCTGGCACTTCAGCCACCCTGGGCATCACTGGCCTCT |
| | | GGCCTGAGGACGAGGCCGATTATTGCTAAATAGCATGACATAGCAGCCCGAGACTTGGGTGTTCGGCGGAGGGACCAAG |
| | | CTGACCGTCCTAG (SEQ ID No: 632) |
| EY 8A | V_{H} | GAGGTGCAGCTGGTGGAGTCGGGCCCAGGACTGGTGAAGCCTTCACAGACCCTGTCCCTCACCTGCACTGTCTCTGGTG |
| | | GCTCCATCAGCAGTGGTAGTTACTACTGGAGCTGGATCCGGCAGCCCGCCGGGAAGGGACTGGAGTGGATTGGGCGTAT |
| | | CTATACCAGTGGGAGCACCAACTACAACCCCTCCCTCAAGAGTCGAGTCACCATATCAGTAGACACGTCCAAGAACCAG |
| | | TTCTCCCTGAAGCTGAGCTCTGTGACCGCCGCAGACACGGCCGTGTATTACTGTGCGAAAGGACATGTTATTAGTGGCTA |
| | | CGATGATTACTACTACTACTACGGTATGGACGTCTGGGGCCAAGGGACCACGGTCACCGTCTCCTCA |
| | | (SEQ ID No: 633) |
| | V_{L} | TCCTATGAGCTGACTCAGCCACCCTCGGTGTCAGTGTCCCCAGGACAGACGGCCAGGATCACCTGCTCTGGAGATGCAT |
| | | TGCCAAAGCAATATGCTTATTGGTACCAGCAGAAGCCAGGCCAGGCCCCTGTGCTGGTGATATATAAAGACAGTGAGAG |
| | | GCCCTCAGGGATCCCTGAGCGATTCTCTGGCTCCAGCTCAGGGACAACAGTCACGTTGACCATCAGTGGAGTCCAGGCA |
| | | GAAGACGAGGCTGACTATTACTGTCAATCAGCAGACAGCAGTGGTACTTATTGGGTGTTCGGCGGAGGGACCAAGCTGA |
| | | CCGTCCTA (SEQ ID No: 634) |
| EY 2A | V_{H} | CAGGTGCAGCTGGTGGAGTCTGGAGCAGAGGTGACAAAGCCCGGGGACTCTCTGATAATCTCCTGTAAGGGCTCTGGAT |
| | | ATGCATTTACTCAATACTGGATCGGCTGGGTGCGCCAGAAGCCCGGGAAAGGCCTGGAGTGGATGGCCATGGTTTATCCC |
| | | GATTCCTCTGCCGTCTTTGCCGGTGGTGCCTCTGGCGTCAGATATAGGCCGCCCTTCCAAGGCCAGGTCACCATATCAGC |
| | | CGACACGTCCGTCAACACCGCCTACCTGCAGTGGGACAGCCTGAAGGCCTCGGACACCGCCATGTACTATTGTGTAAGA |
| | | CAGGAACGTGGGAGCAATACTTGGTACGCGGGAAACTCCTGGGGCCAGGGAACTCTGGTCACCGTCTCCTCA (SEQ ID |
| | | No: 635) |
| | V_{L} | GATATTGTGATGACTCAGTCTCCACTCTCCCTGCCCGTCAGCCCTGGAGAGCCGGCCTCCATCTCCTGTAGGTCTAGTCA |
| | | GAGCCTCCTCCATACTGATGCATACAACTATTTGGATTGGTACCTGCAAAAGCCAGGGCAGTCTCCACAACTCCTGATCT |
| | | ATTTGGGTTCTACTCGGGCCTCCGGGGTGCCTGACAGGTTCAGTGGCAGTGGATCAGGCACAGACTTTACACTGAAAAT |
| | | CAGTAGCGTGAAGGCTGAGGATGTTGGGGTTTATTACTGCATGCAAGCTCTACAAACTCCGGGCACTTTTGGCCAGGGG |
| | | ACCAAGCTGGAGATCAAA (SEQ ID No: 636) |
| EY 3B | V_{H} | GAAGTGCAGCTGGTGGAGTCTGGAGCAGAGGTGACAAAGCCCGGGGAGTCTCTGATAATCTCCTGTAAGGGGTCTGGA |
| | | TATGCATTTACTCAATACTGGATCGGCTGGGTGCGCCAGAAGCCCGGGAAAGGCCTGGAGTGGATGGCCATGGTGTATCC |
| | | CGATTCCTCTGCCGTCTTTGCCGGTGGTGCCTCTGGCGTCAGATATAGGCCGCCCTTCCAAGGCCAGGTCACCATCTCAG |
| | | CCGACACGTCCGTCAACACCGCCTACCTGCAGTGGGACAGCCTGAAGGCCTCGGACACCGCCATGTACTATTGTGTAAG |
| | | ACAGGAACGTGGGAGCAACACTTGGTACGCGGGAAACTCCTGGGGCCAGGGAACTCTGGTCACCGTCTTCTCA (SEQ ID |
| | | No: 637) |
| | V_{L} | GAAATTGTGTTGACGCAGTCTCCACTCTCCCTGCCCGTCAGCCCTGGAGAGCCGGCCTCCATCTCCTGTAGGTCTAGTCA |
| | | GAGCCTCCTCCATACTGATGCATACAACTATTTGGATTGGTACCTGCAAAAGCCAGGGCAGTCTCCACAGCTCCTGATCT |
| | | ATTTGGGTTCTACTCGGGCCTCCGGGGTGCCTGACAGGTTCAGTGGCAGTGGATCAGGCACAGACTTTACACTGAAAAT |
| | | CAGTAGCGTGAAGGCTGAGGATGTTGGGGTTTATTACTGCATGCAAGCTCTACAAACTCCGGGCACTTTTGGCCAGGGG |
| | | ACCAAGGTGGAAATCAGA (SEQ ID No: 638) |
| EZ 4A | V_{H} | GAGGTGCAGCTGGTGCAGTCTGGAGCAGAGGTGAAAAAGCCCGGGGAGTCTCTGAAGATCTCCTGTAAGGGTTCTGGA |
| | | TACAGCTTTACCAGCTACTGGATCGGCTGGGTGCGCCAGATGCCCGGGAAAGGCCTGGAGTGGATGGGGATCATCTATCC |
| | | TGGTGACTCTGATACCAGATACAGCCCGTCCTTCCAAGGCCAGGTCACCATCTCAGCCGACAAGTCCATCAGCACCGCCT |
| | | ACCTGCAGTGGAGCAGCCTGAAGGCCTCGGACACCGCCATGTATTACTGTGCGAGATCGCCTATAGCAGCAGACCTGTTT |
| | | GACTACTGGGGCCAGGGAACCCTGGTCACCGTCTCCTCA (SEQ ID No: 639) |
| | V_{L} | GACATCCAGTTGACCCAGTCTCCATCCTCCCTGTCTGCATCTGTAGGAGACAGAGTCACCATCACTTGCCAGGCGAGTCA |
| | | GGACATTAGCAACTATTTAAATTGGTATCAGCAGAAACCAGGGAAAGCCCCTAAGCTCCTGATCTACGATGCATCCAATTT |
| | | GGAAACAGGGGTCCCATCAAGGTTCAGTGGAAGTGGATCTGGGACAGATTTTACTTTCACCATCAGCAGCCTGCAGCCT |
| | | GAAGATATTGCAACATATTACTGTCAACAGTATGATAATCTCTTATTCACTTTCGGCCCTGGGACCAAGGTGGAGATCAAA |
| | | (SEQ ID No: 640) |

Amino acid sequences of V_{H} and V_{L}

| | | |
|---|---|---|
| EZ 9B | V_{H} | EVQLVQSGAEVKKPGASVKVSCKASGYTFTGYYMHWVRQAPGQGLEWMGRINPNSGGTNYAQKFQGRVTMTRDTSISTA |
| | | YMELSRLRSDDTAVYYCAREGPTVTWWFDPWGQGTLVTVSS (SEQ ID No: 273) |
| | V_{L} | SYELTQPPSVSVAPGQTARITCGGNNIGSKSVHWYQQKPGQAPVLVVYDDSDRPSGIPERFSGSNSGNTATLTISRVEAGDEA |
| | | DYYCQVWDSSSDHPSWVFGGGTKLTVL (SEQ ID No: 274) |
| EZ 11C | V_{H} | EVQLVESGAEVKKPGASVKVSCKVSGYTLTELSMHWVRQAPGKGLEWMGGFDPEDGETIYAQKFQGRVTMTEDTSTDTAY |
| | | MELSSLRSEDTAVYYCATTTVTTPTANWFDPWGQGTLVTVSS (SEQ ID No: 275) |
| | V_{L} | QSALTQPPSVSAAPGQKVTISCSGSSSNIGNNYVSWYQQLPGTAPKLLIYENNKRPSGIPDRFSGSKSGTSATLGITGLQTGD |
| | | EADYYCGTWDSSLRQVVFGGGTKLTVL (SEQ ID No: 276) |
| FD 9B | V_{H} | EVQLVESGGGLVQPGGSLRLSCAGTGFSFSRYWMNWVRQAPGKGLEWVANMDPDGGAKYYLDSVKGRFTISGDNAKNSL |
| | | YLQMNRLRAEDTAVYYCVKFGRSEGLFWGRGTLVTVSS (SEQ ID No: 277) |
| | V_{L} | QAVVTQEPSLTVSPGGTVTLTCGSSTGAVTSGHYPYWFQQKPGQAPRTLIYDTSNKHSWTPARFSGSLLGGKAALTLSGAQPE |
| | | DEADYYCFLTYVGARRLFGGGTKLTVL (SEQ ID No: 278) |
| EZ 11A | V_{H} | EVQLVESGGGLVQPGGSLRLSCAASGFTFSSYWMSWVRQAPGKGLEWVANIKQDGSEKYYVDSVKGRFTISRDNAKNSLYL |
| | | QMNSLRAEDTAVYYCARDDYSGSYYWEFDYWGQGTLVTVSS (SEQ ID No: 279) |
| | V_{L} | QPVLTQSPSASASLGASVKLTCTLSSGHSSYAIAWHQQQPEKGPRYLMKLNSDGSHSKGDGIPDRFSGSSSGAERYLTISSLQ |
| | | SEDEADYYCQTWGTGIWVFGGGTKLTVL (SEQ ID No: 280) |
| EW 10C | V_{H} | EVQLVESGGGLVQPGGSLRLSCAASGFTFNKYKMTWVRQAPGKGLEWVANIKEDGSEKNYVDSVKGRFTISRDNARNSVY |
| | | MHLNNLRVEDTAVYYCARGRTLGDWGQGTTVTVSS (SEQ ID No: 281) |
| | V_{L} | AIRMTQSPLSLPVTLGQPASISCRSSQSLVHSDGNTYLNWFQQRPGQSPRRLIYKVSNRDSGAPDRFSGSGSGTDFTLKISRV |
| | | EAEDVGVYYCMQGTHWPPITFGPGTKVDIK (SEQ ID No: 282) |
| EY 12B | V_{H} | EVQLVESGGDLVQPGRSLRLSCAASGFTFDYFAMHWVRQVPGKGLEWVSGIRWNSETIGYADSVKGRFTISRDNAKKSLYLE |
| | | MNSLRSEDTAFYYCAKGRSGYGHTAFDVWGQGTMVTVSS (SEQ ID No: 283) |
| | V_{L} | QSALTQPPSVSGAPGQRVTISCTGSSSNIGANYDVHWYQRLPGTAPKLLIYDNNNRPSGVPDRFSGSKSGTSASLAIAGLQAE |
| | | DEADYYCQSYDSSLSASVFGGGTKLTVL (SEQ ID No: 284) |
| EZ 8C | V_{H} | EVQLVESGGGLVKPGGSLRLSCAASGFTFSSYTMNWVRQAPGKGLEWVSSFIANSDYKFYADSVKGRFTISRDNAKSSLYLQ |
| | | MNSLRAEDTAVYYCARELTSYGSHDAFDIWGQGTMVTVSS (SEQ ID No: 285) |
| | V_{L} | QSALTQPASVSGSPGQSITISCTGTSSDVGGYNYVSWYQQHPGKAPKLLIYEVTNRPSGVSDRFSGSKSANVASLTISGLQAE |
| | | DEADYYCSSYTTTDSVVFGGGTKLTVL (SEQ ID No: 286) |
| EY 9C | V_{H} | EVQLVESGGGLVKPGGSLRLSCAASEFTFSSYTLNWVRQAPGKGLEWVSSISTSSAYIYYADSVKGRFTISRDNAKKSLSLQM |
| | | NSLTAEDTAVYYCATWGGAPFDYWGQGTMVTVS (SEQ ID No: 287) |
| | V_{L} | QSVLTQPASVSGSPGRSITISCTETSSDVGTYNLVSWYQQHPGKAPKLMIFDDNKRPSGVSNRFSGSKSGNTASLTISGLQAE |
| | | DEADYYCCSYAGGRTFNVLFGGGTKLTVL (SEQ ID No: 288) |
| EZ 8B | V_{H} | EVQLVESGGGLVQPGGSLRLSCAASGFTFSSYAMSWVRQAPGKGLEWVSAISGSGGSTYYADSVKGRFTISRDNSKNTLYLQ |
| | | MNSLRAEDTAVYYCAKDLGYYGSGSSWGQGTLVTVSS (SEQ ID No: 289) |
| | V_{L} | SYELTQPPSVSVSPGQTASITCSGDKLGDKYACWYQQKPGQSPVLVIYQDSKRPSGIPERFSGSNSGNTATLTISGTQAMDEA |
| | | DYYCQAWDSSTAVFGGGTKLTVL (SEQ ID No: 290) |
| FD 3E | V_{H} | EVOLVESGGGVVQPGRSLRLSCAASGFTFSSYGMHWVRQAPGKGLEWVAVISYDGSNKYYADSVKGRFTISRDNSKNTLYL |
| | | QMNSLRAEDTAVYYCAKDPHYYGSGSYYNQLRGYYYYGMDVWGQGTTVTVSS (SEQ ID No: 291) |
| | V_{L} | DIVMTQTPLSLSVTPGQPASISCKSSQSLLHSDGKTYLYWYLQKPGQSPQLLIYEVSSRFSGVPDRFSGSGSGTDFTLKISRV |
| | | EAEDVGVYYCMQGIHLPHFRRRDQVEIK (SEQ ID No: 292) |
| EW 1A | V_{H} | EVQLVESGGGVVQPGSSLRLSCETSGFTFSGHAMHWVRQAPGKGLEWLAQIWFDGSEKYYADSVKGRFTISRDNSKKILYM |
| | | QMNSLRVQDTAVYYCARDGQHLAPFAMDVWGQGTMVTVSS (SEQ ID No: 293) |
| | V_{L} | QSVLTQPASVSGSPGQSITISCTGTSSDVGASNRVSWYQHSPGEAPKLIIYQVTVRPSGVSDRFSGSKSGNTASLTISGLRTE |
| | | DEAEYYCNSFVSGDSWVFGGGTKVTVL (SEQ ID No: 294) |
| FD 5B | V_{H} | EVQLVESGGGVVQPGRSLRLSCAASGFTFSSYGMHWVRQAPGKGLEWVAVIWYDGSNKYYADSVKGRFTISRDNSKNTLYL |
| | | QMNSLRAEDTAVYYCARDERRESYNFVLDYWGQGTLVTVSS (SEQ ID No: 295) |
| | V_{L} | QSVLTQPPSASGSPGQSVTISCTGTSSDAGGYNYVSWYQQHPGKAPKLMIYEVSKRPSGVPDRFSGSKSGNTASLTVSGLQAE |
| | | DEADYYCSSYAGSNNPFVFGTGTKVTVL (SEQ ID No: 296) |
| EW 9A | V_{H} | EVQLLESGGGVVQPGRSLRLSCAASGFTFSSYGMHWVRQAPGKGLEWVAVIWYDGSNKYYADSVKGRFTISRDNSKNTLYL |
| | | QMNSLRAEDTAVYYCAKDMWALYDILTGYYTPYYYYGMDVWGQGTTVTVSS (SEQ ID No: 297) |
| | V_{L} | SYELTQPPSVSVSPGQTARITCSADALPKQYAYWYQQKPGQAPVLVIYKDSERPSGIPERFSGSSSGTTVTLTISGVQAEDEA |
| | | DYYCQSADSSGTYWVFGGGTKLTVL (SEQ ID No: 298) |
| FD 8C | V_{H} | EVQLVESGGGLVKPGRSLRLSCTASGFTFGDYAMSWFRQAPGKGLEWVGFIRSKAYGGTTEYAASVKGRFTISRDDSKSIAYL |
| | | QMNSLKTEDTAVYYCTRNDFWSGYYPDYWGQGTLVTVSS (SEQ ID No: 299) |
| | V_{L} | DIVMTQSPLSLPVTLGQPASISCRSSQSLVHSDGNTYLNWFQQRPGQSPRRLIYKVSNRDSGVPDRFSGSGSGTDFTLKISRV |
| | | EAEDVGVYYCMQGTHWPRSLSAEGPKWISN (SEQ ID No: 300) |
| EW 5A | V_{H} | EVQLVESGGGLVQPGGSLRLSCSVSGFPFGTYAMHWVRQAPGKGLDYVSAINNDGSITYYADSVRGRFTVSRDNSENTLYLR |
| | | LSGLRPDDTAIYYCVKDRGSVIRDFDVWGRGTLVTVSS (SEQ ID No: 301) |
| | V_{L} | QTVVTQEPSLTVSPGGTVTLTCASSTGTVTSDYYPNWFQQKPGQAPRPLIFGTAYRHSWTPARFSGSLLGGKAALTVSDVQPE |
| | | DEADYYCLLYCGGGQLFGGGTKLTVL (SEQ ID No: 302) |
| EZ 9C | V_{H} | EVQLVESGGGLVQPGGSLRLSCSASGFTLNGYAMHWVRQAPGKGLEYVSSLSNRGDNTRYAESVKGRFLISRDIAKDTLYLQ |
| | | MSSLRPEDTAVYYCGKGLLSASGGLPIDDWGQGTLVTVSS (SEQ ID No: 303) |
| | V_{L} | QSVLTQPPSVSAAPGQKVTISCSGSSSNIGNNYVSWYQQFPGTAPKLLIYDNNKRPSGIPDRFSGSKSGTSATLGITGLQTGD |
| | | EAAYYCATWDSSLSAGVFGGGAKLTVL (SEQ ID No: 304) |
| EZ 9A | V_{H} | EVQLVESGGGLVQPGESLRVSCAASGFTFSNYWMHWVRQVPGKGPVWVSIINTDGSITRYVDSVRGRFTISRDNAKNTVHL |
| | | QMNSLTAEDTAIYYCARDVNRYPDYWGQGTLVTVSS (SEQ ID No: 305) |
| | V_{L} | QSALTQPASVSGSPGQSITISCTGTYSDVGYYNYVSWYQQQPGKAPKVIIHGDINRPFGVSNRFSGSKSGNTASLTISGLQAE |
| | | DEADYFCCSYVNNGAWVFGGGTKLTVL (SEQ ID No: 306) |
| EY 12A | V_{H} | EVQLVESGPGLVKPSQTLSLTCAVSGGSISSGGYYWSWIRQPPGKGLELIGYTDYTGKTLYNPSLKSRLTISVDTSKNQFSLK |
| | | LRSVTAADTAVYYCARGMTQDDILTGFNRPHWYFDLWGRGSLVTVSS (SEQ ID No: 307) |
| | V_{L} | QSVLTQPPSVSGAPGQRVTISCTGSSSNIGAGYDVHWYQQLPGTAPKVVIYRNMNRPSGVPDRFSGSKSGTSASLAITGLQAE |
| | | DEADYYCQSFDSSLSDFVVFGGGTKLTVL (SEQ ID No: 308) |
| FD 4C | V_{H} | EVQLVESGPGLVKPSQTLSLTCTVSGGSISSGGYYWSWIRQHPGKGLEWIGYIYYSGSTYYNPSLKSRVTISVDTSKNQFSLK |
| | | LSSVTAADTAVYYCARVRSSSSWYFDYWGQGTLVTVSS (SEQ ID No: 309) |
| | V_{L} | QSVLTQPASVSGSPGQSITISCTGTSSDVGGYNYVSWYQQHPGKAPKLMIYDVSKRPSGVSNRFSGSKSGNTASLTISGLQAE |
| | | DEADYYCSSYTSKWVFGGGTKLTVL (SEQ ID No: 310) |
| EW 4C | V_{H} | EVQLVESGPGLVKPSETLSLTCSVSADSFKKGYYWGWIRQPPGKGLESIVNSFDSGTTRYNPSLWGRATVSDMSKWHFSLKL |
| | | TSVTAADTAVYYCVRGTYGSGLHWGQGILVTVSS (SEQ ID No: 311) |
| | V_{L} | QTVVTQEPSLTVSPGGTVTLTCGSSVGTVASGHYPYWVQQKPGQAPRTLIYDTDNKQSWTPARFSGSLLGGKAALTLSGAQP |
| | | EDEADYYCFLSHNDAWVFGGGTKLTVL (SEQ ID No: 312) |
| FD 6D | V_{H} | EVQLVESGPGLVKVSETLFLTCTVSGYSIGSGNYWGWIRQPPGKVLEWIGSTYHSGTTYYNPSLKSRVTISVDSSKNQFSLKL |
| | | TSVTAADTAVYYCARDRLLAVHYDSRGYLVDYWGQGTMVTVSS (SEQ ID No: 313) |
| | V_{L} | AIRMTQSPDSLAVSLGERATINCKSSQSIFYSSNNKNYLAWYQHKPGQPPKLLIYWASTRESGVPDRFSGSGSGTDFTLTISS |
| | | LQAEDVAIYYCQQYYDIPRTFGQGTKVEIR (SEQ ID No: 314) |
| EY 5A | V_{H} | EVQLVESGPGLVKPSGTLFLTCTVSGGSISNYYWSWIRQPPGKALEWIGFIYSNGNTDYNPSLQSRVTISGDTSKNQFSLNLR |
| | | SVTAADTAVYYCARGPGPATGGSLDYWGQGTMVTVSS (SEQ ID No: 315) |
| | V_{L} | NFMLTQPPSASGTPGQRVTISCSGSSSNIGINTVNWYQHLPGTAPKLLIYGNNQRPSGVPDRFSGSKSGTSTSLAISGLQSED |
| | | EADYYCSAWDDSLNGPVFGGGTQLTVL (SEQ ID No: 316) |
| EZ 7B | V_{H} | EVQLVESSPGLVKPSETLSLTCTVSGGSISSYYWSWIRQPPGKGLEWIGYIYYSGSTNYNPSLKSRVTISVDTSKNQFSLKLS |
| | | SVTAADTAVYYCARRVFGPVLPSKLGGSYWGGGAFDIWGQGTMVTVSS (SEQ ID No: 317) |
| | V_{L} | DIQLTQSPDSLAVSLGERATINCKSSQSVLYSSNNKNYLAWYQQKPGQPPKLLIYWASTRESGVPDRFSGSGSGTDFTLTISS |
| | | LQAEDVAVYYCQQYYSTPLTFGPGTKVEIR (SEQ ID No: 318) |
| EZ 4C | V_{H} | EVQLVESGPGLVKPSETLSLTCTVSGGSVSSGNNYWSWIRQPPGKALEWIGYIYYSGSTKYNPSLKSRVTMSVGTSKNQFSLK |
| kappa | | VNSVTAADTAMYYCARAPSAPFGGLFDWILPKGINNWGQGTLVTVSS (SEQ ID No: 319) |
| | V_{L} | EIVLTQSPSTLSASVGDRVTITCRTSQSIRSWLAWYQQKPGKAPKLLIFEASTLESGVPERFSGSGSGAEFTLTISSLQPDDF |
| | | ATYYCQQYNGYSYSFGQGTKVEIR (SEQ ID No: 320) |
| EZ 4C | V_{H} | VQLVQESGPGLVKPSETLSLTCTVSGGSVSSGNNYWSWIRQPPGKALEWIGYIYYSGSTKYNPSLKSRVTMSVDTSKNQFSLK |
| lambda | | VNSVTAADTAMYYCARAPSAPFGGLFDWILPKGIDNWGQGTLVTVSS (SEQ ID No: 321) |
| | V_{L} | QSALTQPPSVSAAPGQKVTISYSGNSSDMGTYAVSW- |
| | | QRLPGTAPKLFICEENKRPPGIPDRFSGSKSGTSATLGITGLWPEDEADYC-IA-HSSPRLGCSAEGPS-PS- |
| | | (SEQ ID No: 322) |
| FD 4B | V_{H} | QLQLQESGPGLVKPSETLSLTCTVSGGSVSSGNNYWSWIRQPPGKALEWIGYLYYSGSTKYNPSLKSRVTMSVDTSKNQFSL |
| | | KVNSVTAADTAMYYCARAPSAPFGGLFDWILPKGIDSWGQGTLVTVSS (SEQ ID No: 323) |
| | V_{L} | QSALTQPPSVSAAPGQKVTISYSGNSSDMGTYAVSW- |
| | | QRLPGTAPKLFICEENKRPPGIPDRFSGSKSGTSATLGITGLWPEDEADYC-IA-HSSPRLGCSAEGPS-PS- |
| | | (SEQ ID No: 324) |
| EY 8A | V_{H} | EVOLVESGPGLVKPSQTLSLTCTVSGGSISSGSYYWSWIRQPAGKGLEWIGRIYTSGSTNYNPSLKSRVTISVDTSKNQFSLK |
| | | LSSVTAADTAVYYCAKGHVISGYDDYYYYYGMDVWGQGTTVTVSS (SEQ ID No: 325) |
| | V_{L} | SYELTQPPSVSVSPGQTARITCSGDALPKQYAYWYQQKPGQAPVLVIYKDSERPSGIPERFSGSSSGTTVTLTISGVQAEDEA |
| | | DYYCQSADSSGTYWVFGGGTKLTVL (SEQ ID No: 326) |
| EY 2A | V_{H} | QVQLVESGAEVTKPGDSLIISCKGSGYAFTQYWIGWVRQKPGKGLEWMAMVYPDSSAVFAGGASGVRYRPPFQGQVTISAD |
| | | TSVNTAYLQWDSLKASDTAMYYCVRQERGSNTWYAGNSWGQGTLVTVSS (SEQ ID No: 327) |
| | V_{L} | DIVMTQSPLSLPVSPGEPASISCRSSQSLLHTDAYNYLDWYLQKPGQSPQLLIYLGSTRASGVPDRFSGSGSGTDFTLKISSV |
| | | KAEDVGVYYCMQALQTPGTFGQGTKLEIK (SEQ ID No: 328) |
| EY 3B | V_{H} | EVQLVESGAEVTKPGESLIISCKGSGYAFTQYWIGWVRQKPGKGLEWMAMVYPDSSAVFAGGASGVRYRPPFQGQVTISAD |
| | | TSVNTAYLQWDSLKASDTAMYYCVRQERGSNTWYAGNSWGQGTLVTVFS (SEQ ID No: 329) |
| | V_{L} | EIVLTQSPLSLPVSPGEPASISCRSSQSLLHTDAYNYLDWYLQKPGQSPQLLIYLGSTRASGVPDRFSGSGSGTDFTLKISSV |
| | | KAEDVGVYYCMQALQTPGTFGQGTKVEIR (SEQ ID No: 330) |
| EZ 4A | V_{H} | EVQLVQSGAEVKKPGESLKISCKGSGYSFTSYWIGWVRQMPGKGLEWMGIIYPGDSDTRYSPSFQGQVTISADKSISTAYLQ |
| | | WSSLKASDTAMYYCARSPIAADLFDYWGQGTLVTVSS (SEQ ID No: 331) |
| | V_{L} | DIQLTQSPSSLSASVGDRVTITCQASQDISNYLNWYQQKPGKAPKLLIYDASNLETGVPSRFSGSGSGTDFTFTISSLQPEDI |
| | | ATYYCQQYDNLLFTFGPGTKVEIK (SEQ ID No: 332) |

TABLE 7
Amino acid sequences of complementarity-determining regions (CDRs) of
the heavy chain variable region and the light chain variable regions of the 32 SARS-COV-2
nucleocapsid-reactive human monoclonal antibodies.

| | Heavy chain CDR1 | Heavy chain CDR2 | Heavy chain CDR3 | Light chain CDR1 | Light chain CDR2 | Light chain CDR3 |
|---|---|---|---|---|---|---|
| MAb | (HCDR1) | (HCDR2) | (HCDR3) | (LCDR1) | (LCDR2) | (LCDR3) |
| EZ 9B | GYTFTGYY(SEQ | INPNSGGT (SEQ | AREGPTVTWWF | NIGSKS (SEQ ID | DDS (SEQ ID No: | QVWDSSSDHPSW |
| | ID No: 333) | ID No: 334) | DP (SEQ ID No: | No: 336) | 337) | V (SEQ ID No: 338) |
| | | | 335) | | | |
| EZ 11C | GYTLTELS (SEQ | FDPEDGET (SEQ | ATTTVTTPTANW | SSNIGNNY (SEQ | ENN (SEQ ID No: | GTWDSSLRQVV |
| | ID No: 339) | ID No: 340) | FDP (SEQ ID No: | ID No: 342) | 343) | (SEQ ID No: 344) |
| | | | 341) | | | |
| FD 9B | GFSFSRYW (SEQ | MDPDGGAK (SEQ | VKFGRSEGLF | TGAVTSGHY | DTS (SEQ ID No: | FLTYVGARRL |
| | ID No: 345) | ID No: 346) | (SEQ ID No: 347) | (SEQ ID No: 348) | 349) | (SEQ ID No: 350) |
| EZ 11A | GFTFSSYW (SEQ | IKQDGSEK (SEQ | ARDDYSGSYYW | SGHSSYA (SEQ ID | LNSDGSH (SEQ | QTWGTGIWV |
| | ID No: 351) | ID No: 352) | EFDY (SEQ ID No: | No: 354) | ID No: 355) | (SEQ ID No: 356) |
| | | | 353) | | | |
| EW 10C | GFTFNKYK (SEQ | IKEDGSEK (SEQ | ARGRTLGD (SEQ | QSLVHSDGNTY | KVS (SEQ ID No: | MQGTHWPPIT |
| | ID No: 357) | ID No: 358) | ID No: 359) | (SEQ ID No: 360) | 361) | (SEQ ID No: 362) |
| EY 12B | GFTFDYFA (SEQ | IRWNSETI (SEQ | AKGRSGYGHTAF | SSNIGANYD (SEQ | DNN (SEQ ID No: | QSYDSSLSASV |
| | ID No: 363) | ID No: 364) | DV (SEQ ID No: | ID No: 366) | 367) | (SEQ ID No: 368) |
| | | | 365) | | | |
| EZ 8C | GFTFSSYT (SEQ | FIANSDYK (SEQ | ARELTSYGSHDA | SSDVGGYNY | EVT (SEQ ID No: | SSYTTTDSVV |
| | ID No: 369) | ID No: 370) | FDI (SEQ ID No: | (SEQ ID No: 372) | 373) | (SEQ ID No: 374) |
| | | | 371) | | | |
| EY 9C | EFTFSSYT (SEQ | ISTSSAYI (SEQ ID | ATWGGAPFDY | SSDVGTYNL | DDN (SEQ ID No: | CSYAGGRTFNVL |
| | ID No: 375) | No: 376) | (SEQ ID No: 377) | (SEQ ID No: 378) | 379) | (SEQ ID No: 380) |
| EZ 8B | GFTFSSYA (SEQ | ISGSGGST (SEQ | AKDLGYYGSGSS | KLGDKY (SEQ ID | QDS (SEQ ID No: | QAWDSSTAV |
| | ID No: 381) | ID No: 382) | (SEQ ID No: 383) | No: 384) | 385) | (SEQ ID No: 386) |
| FD 3E | GFTFSSYG (SEQ | ISYDGSNK (SEQ | AKDPHYYGSGSY | QSLLHSDGKTY | EVS (SEQ ID No: | MQGIHLPH (SEQ |
| | ID No: 387) | ID No: 388) | YNQLRGYYYYG | (SEQ ID No: 390) | 391) | ID No: 392) |
| | | | MDV (SEQ ID No: | | | |
| | | | 389) | | | |
| EW 1A | GFTFSGHA (SEQ | IWFDGSEK (SEQ | ARDGQHLAPFAM | SSDVGASNR | QVT (SEQ ID No: | NSFVSGDSWV |
| | ID No: 393) | ID No: 394) | DV (SEQ ID No: | (SEQ ID No: 396) | 397) | (SEQ ID No: 398) |
| | | | 395) | | | |
| FD 5B | GFTFSSYG (SEQ | IWYDGSNK (SEQ | ARDERRESYNFV | SSDAGGYNY | EVS (SEQ ID No: | SSYAGSNNPFV |
| | ID No: 399) | ID No: 400) | LDY (SEQ ID No: | (SEQ ID No: 402) | 403) | (SEQ ID No: 404) |
| | | | 401) | | | |
| EW 9A | GFTFSSYG (SEQ | IWYDGSNK (SEQ | AKDMWALYDILT | ALPKQY (SEQ ID | KDS (SEQ ID No: | QSADSSGTYWV |
| | ID No: 405) | ID No: 406) | GYYTPYYYYGM | No: 408) | 409) | (SEQ ID No: 410) |
| | | | DV (SEQ ID No: | | | |
| | | | 407) | | | |
| FD 8C | GFTFGDYA (SEQ | IRSKAYGGTT | TRNDFWSGYYPD | QSLVHSDGNTY | KVS (SEQ ID No: | MQGTHWPRSL |
| | ID No: 411) | (SEQ ID No: 412) | Y (SEQ ID No: 413) | (SEQ ID No: 414) | 415) | (SEQ ID No: 416) |
| EW 5A | GFPFGTYA (SEQ | INNDGSIT (SEQ | VKDRGSVIRDFD | TGTVTSDYY | GTA (SEQ ID No: | LLYCGGGQL |
| | ID No: 417) | ID No: 418) | V (SEQ ID No: 419) | (SEQ ID No: 420) | 421) | (SEQ ID No: 422) |
| EZ 9C | GFTLNGYA (SEQ | LSNRGDNT (SEQ | GKGLLSASGGLPI | SSNIGNNY (SEQ | DNN (SEQ ID No: | ATWDSSLSAGV |
| | ID No: 423) | ID No: 424) | DD (SEQ ID No: 425) | ID No: 426) | 427) | (SEQ ID No: 428) |
| EZ 9A | GFTFSNYW (SEQ | INTDGSIT (SEQ ID | ARDVNRYPDY | YSDVGYYNY | GDI (SEQ ID No: | CSYVNNGAWV |
| | ID No: 429) | No: 430) | (SEQ ID No: 431) | (SEQ ID No: 432) | 433) | (SEQ ID No: 434) |
| EY 12A | GGSISSGGYY | TDYTGKT (SEQ | ARGMTQDDILTG | SSNIGAGYD (SEQ | RNM (SEQ ID No: | QSFDSSLSDFVV |
| | (SEQ ID No: 435) | ID No: 436) | FNRPHWYFDL (SEQ | ID No: 438) | 439) | (SEQ ID No: 440) |
| | | | ID No: 437) | | | |
| FD 4C | GGSISSGGYY | IYYSGST (SEQ ID | ARVRSSSSWYFD | SSDVGGYNY | DVS (SEQ ID No: | SSYTSKWV (SEQ |
| | (SEQ ID No: 441) | No: 442) | Y (SEQ ID No: 443) | (SEQ ID No: 444) | 445) | ID No: 446) |
| EW 4C | ADSFKKGYY | SFDSGTT (SEQ ID | RGTYGSGLH | VGTVASGHY | DTD (SEQ ID No: | FLSHNDAWV |
| | (SEQ ID No: 447) | No: 448) | (SEQ ID No: 449) | (SEQ ID No: 450) | 451) | (SEQ ID No: 452) |
| FD 6D | GYSIGSGNY (SEQ | TYHSGTT (SEQ ID | ARDRLLAVHYDS | QSIFYSSNNKNY | WAS (SEQ ID No: | QQYYDIPRT (SEQ |
| | ID No: 453) | No: 454) | RGYLVDY (SEQ ID | (SEQ ID No: 456) | 457) | ID No: 458) |
| | | | No: 455) | | | |
| EY 5A | GGSISNYY (SEQ | TYSNGNT (SEQ ID | ARGPGPATGGSL | SSNIGINT (SEQ ID | GNN (SEQ ID No: | SAWDDSLNGPV |
| | ID No: 459) | No: 460) | DY (SEQ ID No: | No: 462) | 463) | (SEQ ID No: 464) |
| | | | 461) | | | |
| EZ 7B | GGSISSYY (SEQ | IYYSGST (SEQ ID | ARRVFGPVLPSKL | QSVLYSSNNKNY | WAS (SEQ ID No: | QQYYSTPLT (SEQ |
| | ID No: 465) | No: 466) | GGSYWGGGAFDI | (SEQ ID No: 468) | 469) | ID No: 470) |
| | | | (SEQ ID No: 467) | | | |
| EZ 4C | GGSVSSGNNY | IYYSGST (SEQ ID | ARAPSAPFGGLF | QSIRSW (SEQ ID | EAS (SEQ ID No: | QQYNGYSYS |
| kappa | (SEQ ID No: 471) | No: 472) | DWILPKGINN (SEQ | No: 474) | 475) | (SEQ ID No: 476) |
| | | | ID No: 473) | | | |
| EZ 4C | GGSVSSGNNY | IYYSGST (SEQ ID | ARAPSAPFGGLF | SSDMGTYA (SEQ | EEN (SEQ ID No: | IA-HSSPRLGC |
| lambda | (SEQ ID No: 477) | No: 478) | DWILPKGIDN (SEQ | ID No: 480) | 481) | (SEQ ID No: 482) |
| | | | ID No: 479) | | | |
| FD 4B | GGSVSSGNNY | LYYSGST (SEQ ID | ARAPSAPFGGLF | SSDMGTYA (SEQ | EEN (SEQ ID No: | IA-HSSPRLGC |
| | (SEQ ID No: 483) | No: 484) | DWILPKGIDS | ID No: 486) | 487) | (SEQ ID No: 488) |
| | | | (SEQ ID No: 485) | | | |
| EY 8A | GGSISSGSYY | IYTSGST (SEQ ID | AKGHVISGYDDY | ALPKQY (SEQ ID | KDS (SEQ ID No: | QSADSSGTYWV |
| | (SEQ ID No: 489) | No: 490) | YYYYGMDV (SEQ ID | No: 492) | 493) | (SEQ ID No: 494) |
| | | | No: 491) | | | |
| EY 2A | GYAFTQYW (SEQ | VYPDSSAV (SEQ | SDTAMYYCVRQE | QSLLHTDAYNY | LGS (SEQ ID No: | MQALQTPGT |
| | ID No: 495) | ID No: 496) | RGSNTWYAGNS (SEQ | (SEQ ID No: 498) | 499) | (SEQ ID No: 500) |
| | | | ID No: 497) | | | |
| EY 3B | GYAFTQYW (SEQ | VYPDSSAV (SEQ | SDTAMYYCVRQE | QSLLHTDAYNY | LGS (SEQ ID No: | MQALQTPGT |
| | ID No: 501) | ID No: 502) | RGSNTWYAGNS | (SEQ ID No: 504) | 505) | (SEQ ID No: 506) |
| | | | (SEQ ID No: 503) | | | |
| EZ 4A | GYSFTSYW (SEQ | IYPGDSDT (SEQ | ARSPIAADLFDY | QDISNY (SEQ ID | DAS (SEQ ID No: | QQYDNLLFT |
| | ID No: 507) | ID No: 508) | (SEQ ID No: 509) | No: 510) | 511) | (SEQ ID No: 512) |

### Example 2 Neutralization Assays of Monoclonal Antibodies Against SARS-CoV-2

1. Quantitative PCR-Based Neutralization Assay

Neutralization activity of MAb-containing supernatant was measured using a SARS-CoV-2 infection of Vero E6 cells. Briefly, Vero E6 cells were pre-seeded in a 96 well plate at a concentration of 10⁴ cells per well. In the following day, monoclonal antibody-containing supernatant were mixed with an equal volume of 100 TCID₅₀ virus preparation and incubated at 37° C. for 1 hour. The mixture was added into seeded Vero E6 cells and incubated at 37° C. for 5 days. The cell control, virus control, and virus back-titration were setup for each experiment. At day 5, the culture supernatant was harvested from each well and the viral RNA was extracted and determined by real-time RT-PCR targeting the E gene of SARS-CoV-2. The cycle threshold values of real-time RT-PCR were used as indicators of the copy number of SARS-CoV-2 RNA in samples with lower cycle threshold values corresponding to higher viral copy numbers.

2. Cytopathic Effect (CPE)-Based Neutralization Assay

Vero E6 cells in Dulbecco's Modified Eagle's Medium (DMEM) containing 10% FBS were added into 96-well plates and incubated at 37° C. with 5% CO₂ overnight to reach confluence. After washing with virus growth medium (VGM: DMEM containing 2% FBS), two-fold serially diluted MAbs in VGM starting at 100 μg/ml were added to each duplicated well. The plates were immediately transferred to a BSL-3 laboratory and 100 TCID₅₀ SARS-CoV-2 in VGM was added. The plates were further incubated at 37° C. with 5% CO₂ for three days and the cytopathic morphology of the cells was recorded using an ImageXpress Nano Automated Cellular Imaging System.

3. Plaque Reduction Neutralization Test (PRNT)

Confluent monolayers of Vero E6 cells in 96-well plates were incubated with SARS CoV-2 and antibodies in a 2-fold dilution series (triplicates) for 3 hours at room temperature. Inoculum was then removed, and cells were overlaid with plaque assay overlay. Cells were incubated at 37° C., 5% CO₂ for 24 hours prior to fixation with 4% paraformaldehyde at 4° C. for 30 minutes. Fixed cells were then permeabilised with 0.2% Triton-X-100 and stained with a horseradish peroxidase conjugated-antibody against virus protein for 1 hour at room temperature. TMB substrate was then added to visualize virus plaques as described previously for influenza virus. Convalescent serum from COVID-19 patients was used as a control.

4. Fluorescent Focus-Forming Units Microneutralization Assay (FMNT)

In brief, this rapid, high-throughput assay determines the concentration of antibody that produces a 50% reduction in infectious focus-forming units of authentic SARS-CoV-2 in Vero cells, as follows. Triplicate serial dilutions of antibody are pre-incubated with a fixed dose of SARS-CoV-2 in triplicate before incubation with Vero cells. A carboxymethyl cellulose-containing overlay is used to prevent satellite focus formation. Twenty hours post-infection, the monolayers are fixed with paraformaldehyde and stained for N antigen using MAb EY 2A. After development with a peroxidase-conjugated antibody and substrate, foci are enumerated by enzyme-linked immune absorbent spot reader. Data are analyzed using four-parameter logistic regression (Hill equation) in GraphPad Prism.

5. Competitive Binding Assays

Competitive binding assays were performed as described previously (Rijal 2019) with slight modifications for epitope mapping of the anti-RBD MAbs. Briefly, 0.5 μg/ml of RBD-virus like particles (VLPs) were coated on NUNC plates (50 μl per well) overnight at 4° C., washed and blocked with 300 μl of 5% (w/v) dried skimmed milk in PBS for 1 hour at room temperature prior to the assays. Antibody was biotinylated using EZ-Link Sulfo-NHS-LC-biotin (21237; Life Technologies) and then mixed with competing MAb (in at least 10-fold excess) and transferred to the blocked NUNC plates for 1 hour. A second layer Streptavidin-HRP (S911, Life Technologies) diluted 1:1,600 in PBS/0.1% BSA (37525; Thermo Fisher Scientific) was then added and incubated for another 1 hour. Plates were then washed, and signal was developed by adding POD substrate (11484281001, Roche) for 5 minutes before stopping the reaction with 1 M H₂SO₄. Absorbance (OD₄₅₀) was measured using a Clariostar plate reader (BMG, Labtech). Mean and 95% confidence interval of 4 replicate measurements were calculated. Competition was measured as: (X-minimum binding/(maximum binding-minimum binding), where X is the binding of the biotinylated MAb in the presence of competing MAb. Minimum binding is the self-blocking of the biotinylated MAb or background binding. Maximum binding is binding of biotinylated MAb in the presence of non-competing MAb (anti-influenza N1 neuraminidase MAb).

6. ACE2 Blocking Assays

Two assays were used to determine the blocking of binding of ACE2 to RBD by MAbs. RBD was anchored on the plate in the first assay whereas ACE2 was anchored for the second assay.

In the first ACE2 blocking assay, RBD-VLP (Spycatcher-mi3 VLP-particles conjugated with Spytagged-RBD recombinant protein) (Bruun 2018) was coated on ELISA plates as described for the competitive binding assay. Recombinant ACE2-Fc (18-615) protein expressed in Expi293F (Life Technologies) cells was chemically biotinylated using EZ-link Sulfo-NHS-Biotin (A39256; Life Technologies) and buffer exchanged to PBS using a Zebaspin desalting column (Thermo Fischer). MAbs were titrated in duplicate or triplicate as half-log serial dilution, 8-point series starting at 1 μM in 30 μl volume with PBS/0.1% BSA buffer. Thirty (30) μl of biotinylated ACE2-Fc at approx. 0.2 nM (40 ng/ml) was added to the antibodies. Fifty (50) μl of the mixture was transferred to the PBS-washed RBD-VLP coated plates and incubated for 1 hour at room temperature. Secondary Streptavidin-HRP antibody (S911, Life Technologies) diluted to 1:1600 was then added to the PBS-washed plates and incubated for 1 hour at room temperature. Plates were then washed four times with PBS and signal was developed by adding POD substrate (11484281001, Roche) for 5 minutes before stopping with 1 M H₂SO₄. OD₄₅₀ was measured using a Clariostar plate reader (BMG, Labtech). The control antibody (a non-blocking anti-influenza N1 MAb) or ACE2-Fc without antibody used to obtain the maximum signal and wells with PBS/BSA buffer only were used to determine the minimum signal. Graphs were plotted as % binding of biotinylated ACE2 to RBD. Binding %={(X−Min)/(Max−Min)}*100, where X=measurement of the antibody, Min=buffer only, Max=biotinylated ACE2-Fc alone. The 50% inhibitory concentrations of the antibodies against ACE2 was determined using non-linear regression curve fit using GraphPad Prism 8.

The second ACE2 blocking assay was performed as described previously (Huo 2020; Zhou 2020). Briefly, MDCK-SIAT1 cells were stably transfected to overexpress codon-optimised human ACE2 cDNA (NM_021804.1) using lentiviral vector and FACS sorted (MDCK-ACE2). Cells (3×10⁴ per well) were seeded on a flat-bottomed 96-well plate the day before the assay. RBD-6H (340-538; NITN.GPKK) was chemically biotinylated using EZ-link Sulfo-NHS-Biotin (A39256; Life Technologies). Serial half-log dilutions (starting at 1 μM) of antibodies and controls were performed in a U-bottomed 96 well plate in 30 μl volume. Thirty (30) μl of biotinylated RBD (25 nM) were mixed and 50 μl of the mixture was then transferred to the MDCK-ACE2 cells. After 1 hour a second layer Streptavidin-HRP antibody (S911, Life Technologies) diluted 1:1,600 in PBS/0.1% BSA (37525; Thermo Fisher Scientific) was added and incubated for another 1 hour. Plates were then washed four times with PBS and signal was developed by adding POD substrate (11484281001, Roche) before stopping with 1 M H₂SO₄ after 5 minutes. OD₄₅₀ was measured using a Clariostar plate reader (BMG, Labtech). The control antibody (a non-blocking anti-influenza N1 antibody) was used to obtain maximum signal and PBS only wells were used to determine background. Graphs were plotted as % binding of biotinylated RBD to ACE2. The 50% inhibitory concentration of the blocking antibody was determined as described above.

7. Statistics

The two-tailed Mann-Whitney test was performed to compare differences between two independent groups. The 50% effective concentration (EC₅₀) was determined using linear regression analysis. A p value of less than 0.05 was considered significant. Graphs were presented by Microsoft Excel and GraphPad Prism software.

8. Results

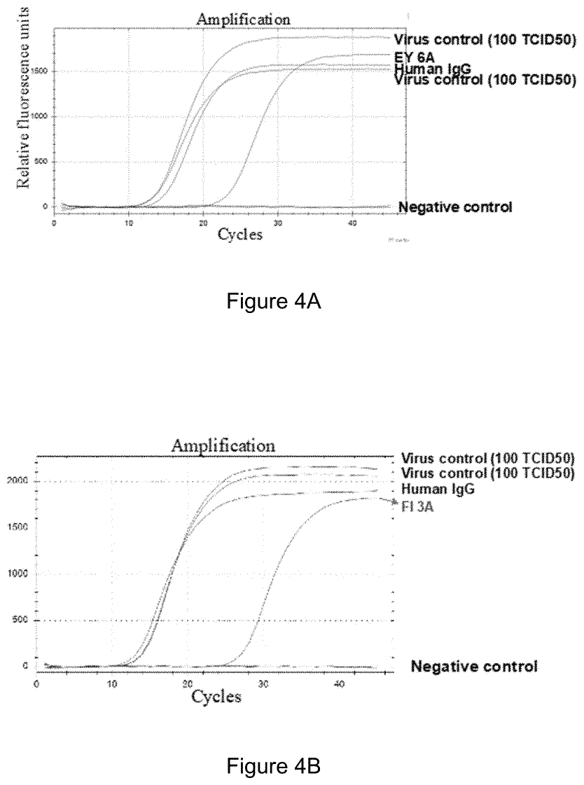

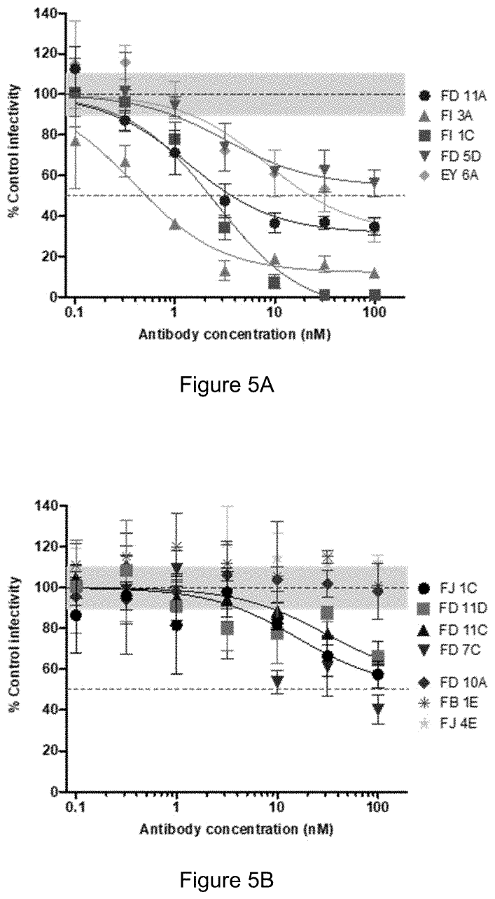

A neutralization test for EW 9C, EY 6A, FD 5D, FD 11A and FI 3A MAbs based on quantitative PCR detection of SARS-CoV-2 in the supernatant bathing infected Vero E6 cells after 5 days of culture, showed a substantial reduction in virus signal (FIG. 4A, FIG. 4B, and Table 8), suggesting these MAbs are highly effective in neutralizing SARS-CoV-2. This was further corroborated by a plaque reduction neutralization test (Table 9) using SARS-CoV-2 virus and the ND₅₀ of EW 9C and EY 6A MAbs were around 7.1 and 10.8 μg/mL, respectively (calculated according to Grist (Grist 1966)).

TABLE 8
The neutralization data of EW 9C, EY 6A, FD 5D, FD 11A and FI 3A MAbs.
Ct value of culture supernatant, after 5 days of incubation*

| | | |
|---|---|---|
| Virus control supernatant without MAb | 13.0 | |
| Anti-S2 EW 9C supernatant | 22.7 | around 832-fold reduction of virus |
| Anti-RBD EY 6A supernatant | 22.7 | around 832-fold reduction of virus |
| Anti-RBD FD 5D supernatant | 18.8 | around 56-fold reduction of virus |
| Anti-RBD FD 11A supernatant | 23.0 | around 1024-fold reduction of virus |
| Virus control supernatant without MAb | 13.9 | |
| Anti-RBD FI 03A supernatant | 27.2 | around 10085-fold reduction of virus |

*An increase of Ct value compared to the Ct value of virus control supernatant without Mab indicates a decrease in virus template. Each unit increase suggests a 2× reduction resulting from presence of MAb. An around 10× increase in Ct = around 1,024 fold reduction of virus.

TABLE 9
The half maximal neutralizing concentration
(NC₅₀) of EW 9C abd EY 6A MAbs against SARS-CoV-2
in the plaque reduction neutralization test.

| ID | Description | NC₅₀ (μg/ml) |
|---|---|---|
| Positive control | Convalescent serum | 1:784 |
| EW 9C | MAb against SARS-CoV-2 spike | 7.1 |
| EY 6A | MAb against SARS-CoV-2 RBD | 10.8 |

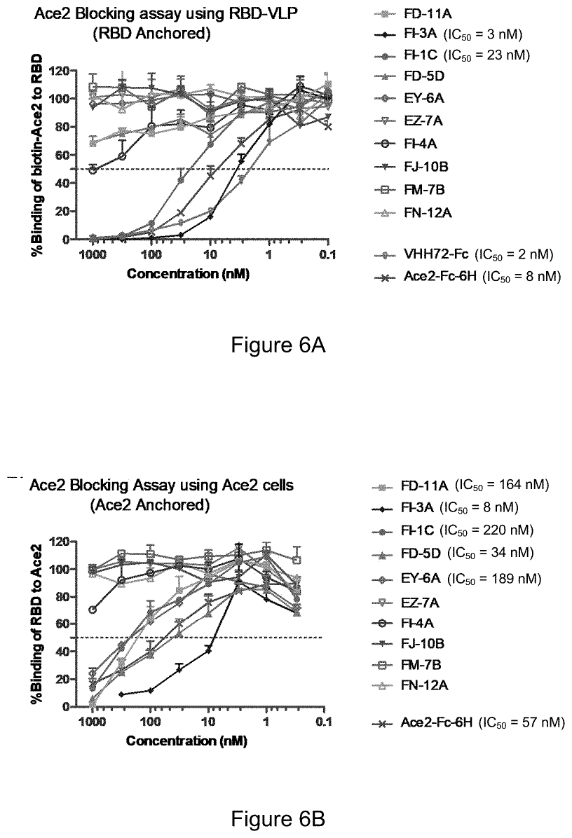

All anti-spike glycoprotein MAbs were systematically screened by plaque reduction neutralization (PRNT) assay for neutralization of wild type SARS-CoV-2 virus (Table 10). A total of 14 neutralizing antibodies distributed between different regions of the spike glycoprotein were identified: three of 13 to S1 (non-RBD), six of nine to S2, five of 10 to RBD. The EC₅₀ concentrations, as a measure of potency, ranged from 0.05 nM to around 133.33 nM (8 ng/ml-around 20 μg/ml). Neutralization was corroborated by a microneutralization test (FMNT), that measured a reduction in fluorescent focus-forming units, summarised in Table 10, FIG. 6A and FIG. 6B. The neutralization results showed that those anti-SARS-CoV-2 RBD MAbs (FD 11A, FI 3A, FI 1C, FD 5D and EY 6A) were most potent against wild-type SARS-CoV-2.

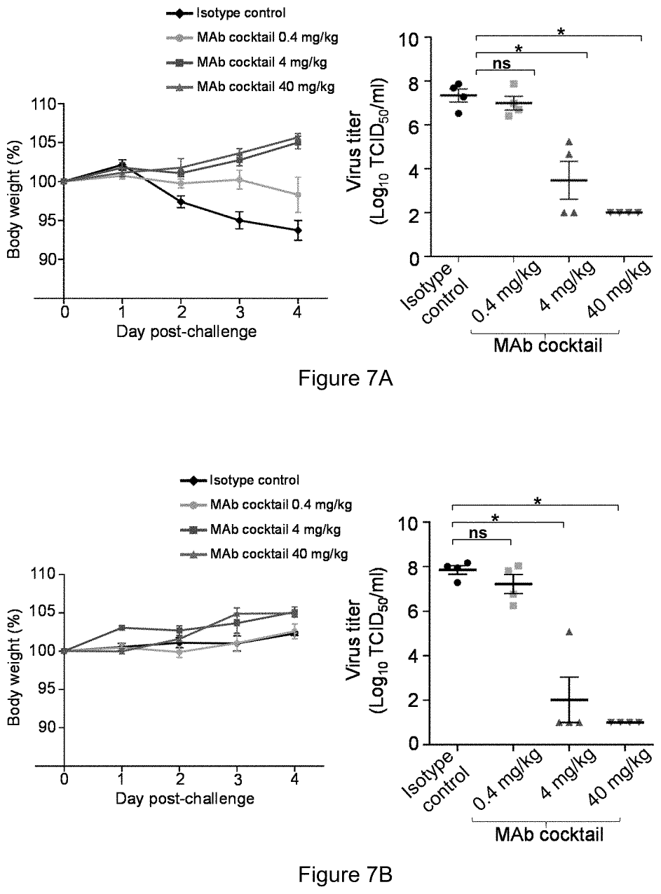

Five neutralizing MAbs (FD 11A, FI 3A, FI 1C, FD 5D and EY 6A) target the RBD and all of these partially or completely blocked the interaction between RBD and ACE2 in one or the other type of assay (Table 10, FIG. 7A, FIG. 7B). The most potent neutralizing antibodies were ACE2 blockers (FI 3A in cluster 2, and FD 11A in cluster 3), and bound independently of each other to the RBD (Table 11). MAb EY 6A has been shown to alter the binding kinetics of the interaction without full inhibition and it had a moderate effect on ACE2 binding here in the assay where ACE2 was expressed at the cell surface. These three MAbs bound independently of each other indicating the existence of at least three neutralization-sensitive epitopes within the RBD (Table 9). All five neutralizing MAbs to the RBD (FD 11A, FI 3A, FI 1C, FD 5D and EY 6A) had V gene sequences close to germline.

Six MAbs specific for SARS-CoV-2 S2 subunit showed moderate neutralization in the PRNT assay (Table 10). The antibodies FB 1E, FJ 4E and EW 9C, are moderately neutralizing (EC50 36-133.33 nM), cross-react on the spike glycoprotein from the common cold betacoronavirus OC43, and show sequence characteristics of memory cells with high numbers of somatic mutations. This indicates that memory B cells, likely primed by an endemic or epidemic betacoronavirus related to OC43, can give rise to antibodies that neutralize SARS-CoV-2, albeit modestly. The other three neutralizing antibodies specific for SARS-CoV-2 S2 subunit, FD 10A, FG 7A and FM 1A were close to germline in sequence and did not cross-react strongly with other betacoronaviruses (Table 1). FD 10A exhibits the most potent neutralizing activity in the PRNT assay and completely inhibits SARS-CoV-2-induced cytopathic effect at 8.33 nM.

Thirteen MAbs were defined that bound the non-RBD S1 region (Table 1) and three, close to germline in sequence, were neutralizing. FJ 1C showed strong neutralization (EC₅₀ 55.5 nM), whilst FD 11E (EC₅₀ 70 nM) and FD 1E (EC₅₀ 110 nM) were moderately neutralizing (Table 10).

SARS-CoV-2 nucleocapsid-reactive antibodies were also screened for binding to fixed and permeabilised infected cells for use in scoring wells in microneutralisation assays (FMNT). Antibody EY 2A performed well for this purpose.

TABLE 10
The function of 34 SARS-CoV-2 spike-reactive human monoclonal antibodies.

| | | | Neutralization | Neutralization | ACE2 Block^{c} | ACE2 Block^{c} |
|---|---|---|---|---|---|---|
| | | | by PRNT^{a} | by FMNT^{b} | RBD | ACE2 |
| Antibody | Domain | IFA | EC₅₀ (nM) | EC₅₀ (nM) | Anchored | Anchored |
| FD 11A | RBD | pos | 0.05 | 3.68 | + | +++ |
| FI 3A | RBD | pos | 8.67 | 0.51 | ++++ | ++++ |
| FI 1C | RBD | pos | 16.67 | 2.24 | ++ | +++ |
| FD 5D | RBD | pos | 133.33 | partial | + | +++ |
| EY 6A | RBD | pos | 133.33 | 22.50 | neg | ++ |
| EY 6A-1* | RBD | pos | 133.33 | 22.50 | neg | ++ |
| EZ 7A | RBD | pos | neg | neg | neg | neg |
| FI 4A | RBD | pos | neg | neg | + | neg |
| FJ 10B | RBD | neg | neg | neg | neg | neg |
| FM 7B | RBD | pos | neg | neg | neg | neg |
| FN 12A | RBD | pos | neg | neg | neg | neg |
| FJ 1C | NTD | pos | 55.50 | partial | | |
| FD 11E | non-RBD S1 | pos | 70.00 | neg | | |
| FD 1E | non-RBD S1 | pos | 110.00 | neg | | |
| EW 8B | non-RBD S1 | neg | neg | neg | | |
| FD 11D | NTD | pos | neg | partial | | |
| FD 11C | non-RBD S1 | pos | neg | partial | | |
| FD 7D | non-RBD S1 | neg | neg | neg | | |
| FD 8B | non-RBD S1 | neg | neg | neg | | |
| FD 7C | NTD | pos | neg | 35.60 | | |
| FG 12C | non-RBD S1 | pos | neg | neg | | |
| FN 8C | non-RBD S1 | neg | neg | neg | | |
| FD 5E | non-RBD S1 | pos | neg | neg | | |
| EW 9B | non-RBD S1 | neg | neg | neg | | |
| FD 10A | S2 | pos | 111.13 | neg | | |
| FB 1E# | S2 | pos | 36.00 | neg | | |
| FJ 4E# | S2 | neg | 75.33 | neg | | |
| EW 9C# | S2 | pos | 133.33 | neg | | |
| EW 9C-1#* | S2 | pos | 133.33 | neg | | |
| FG 7A | S2 | pos | 133.33 | neg | | |
| FM 1A | S2 | neg | 133.33 | neg | | |
| FB 9D# | S2 | pos | neg | neg | | |
| FD 1D | S2 | pos | neg | neg | | |
| FN 2C# | | pos | neg | neg | | |
| Controls | | | | | | |
| CR3022 | RBD | | 42.00 | | neg | neg |
| BS 1A | Flu H3 | | neg | | neg | neg |

^{a}The plaque reduction neutralization (PRNT) assay was performed with wild type SARS-CoV-2 and the half maximal effective concentration (EC₅₀) was determined using linear regression analysis.
^{b}The fluorescent focus-forming units microneutralization (FMNT) assay was performed with wild type SARS-CoV-2 and the half maximal effective concentration (EC₅₀) was determined using logistic regression model. Partial: MAb neutralizes at least ~40% viruses at 100 nM (hightest concentration tested).
^{c}ACE2 blocking activity of anti-RBD antibody compared to ACE2-Fc: +, partial; ++, IC₅₀ > ACE2-Fc; +++, IC₅₀ ~= ACE2-Fc; ++++, IC₅₀ < ACE2-Fc.
*Both EY 6A and EW 9C have an additional pair of expression vectors.
#Memory phenotype.
Abbreviations: IFA, immunofluorescence; RBD, receptor-binding domain; PRNT, plaque reduction neutralization assay; FMNT, fluorescent focus-forming units microneutralization test; ACE2, Angiotensin-Converting Enzyme 2; pos, positive; neg, negative.

TABLE 11
Competitive binding analysis of anti-SARS-CoV-2 RBD human monoclonal antibodies.^{a}

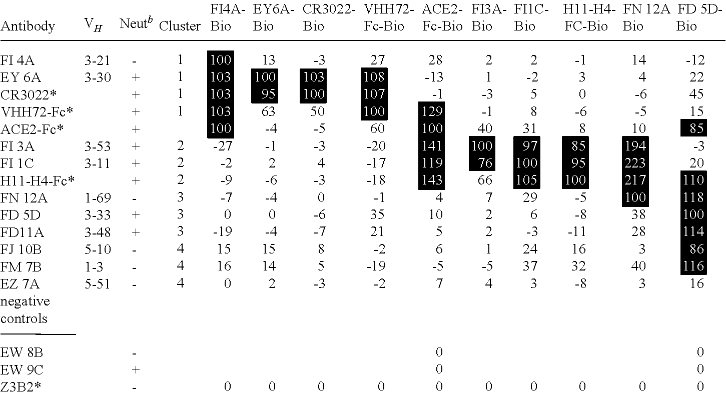

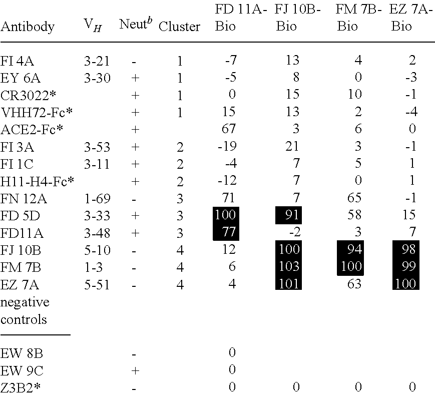

^{a}Competitive inhibition: values are shown for percentage inhibition and those with ≥75% blocking, 50-74% blocking, and <50% blocking are highlighted in black, gray and light gray, respectively.
^{b}Neutralization of antibody against wild type SARS-COV-2 was analysed in the PRNT assay (+ = positive, − = negative).
*SARS and SARS-COV-2 cross-reactive anti-RBD MAb CR3022 was included as a positive control. SARS and SARS-COV-2 cross-reactive anti-RBD nanobodies VHH72 and H11-H4 linked to the hinge and Fc region of human IgG1 were included as positive controls. ACE2-Fc was included as a positive control. Anti-influenza MAb Z3B2 was included as a negative control.

### Example 3 In Vivo Protection of Cocktail of Monoclonal Antibodies Against SARS-CoV-2

1. Test Aminals and Study Design

The prophylactic and therapeutic efficacies of a cocktail of the MAbs of the present invention (hereinafter referred to as antibody cocktail) against SARS-CoV-2 were evaluated in the Syrian hamster model. Briefly, 32 female Golden Syrian hamsters (National Laboratory Aminal Center, Taipei, Taiwan) of 8 weeks old were randomly divided into 8 groups (n=4), 4 groups for the prophylactic experiment, and the other 4 groups for the therapeutic experiment.

In the prophylactic experiment, one day prior to intranasal challenge with 1×10⁵ TCID₅₀/hamster SARS-CoV-2 (hCoV-19/Taiwan/4/2020), animals were treated with a single dose (0.4 mg/kg, 4 mg/kg, or 40 mg/kg) of the antibody cocktail or 40 mg/kg of an isotype negative control (Z3B2, anti-influenza haemagglutinin human IgG1 monoclonal antibody (Huang et al., 2019)) via intraperitoneal injection. Body weight of each animal was measured daily after challenge, and data were normalized to the initial weight of each animal. Animals were sacrificed on day 4 after viral challenge, and the right lung and trachea were collected for histopathological evaluation and viral load and titer.

In the therapeutic experiment, animals were treated with single dose (0.4 mg/kg, 4 mg/kg, or 40 mg/kg) of the antibody cocktail or 40 mg/kg of the isotype negative control via intraperitoneal injection three hours after intranasal challenge with 1×10⁵ TCID₅₀/hamster SARS-CoV-2 (hCoV-19/Taiwan/4/2020). Body weight of each animal was measured daily after challenge, and data were normalized to the initial weight of each animal. Animals were sacrificed on 4 dpi for histopathology, viral load and titer.

2. Viral load and virus titer (median tissue culture infectious dose (TCID₅₀) Assays)

The right lung tissues were weighed and homogenized in 2 ml of PBS. After centrifugation at 600×g for 5 minutes, the clarified supernatant was harvested for viral load detection and live virus titration (TCID₅₀ assay). For viral load detection, total RNAs in the tissue homogenate were extracted with RNeasy Mini kit (Qiagen). Quantitative reverse transcription PCR (qRT-PCR) for detection of SARS-CoV-2 envelope (E) and nucleocapsid (N) genes was performed to determine viral loads. For TCID₅₀ assay, serial 10-fold dilutions of each sample were inoculated in a Vero E6 cell monolayer and cultured for 4 to 7 days for observation of cytopathic effects (CPE). Viral titer was calculated with the Reed-Munch method.

3. Histopathology

Lungs and tracheas were collected and fixed in 10% PBS buffered formaldehyde for 24 hours, then processed into paraffin-embedded tissues blocks. The tissue sections in 4 μm were stained with haematoxylin and eosin (H&E) for microscopy examination.

4. Statistics

Statistical significance between groups was calculated by an unpaired two-sided t test.

5. Results

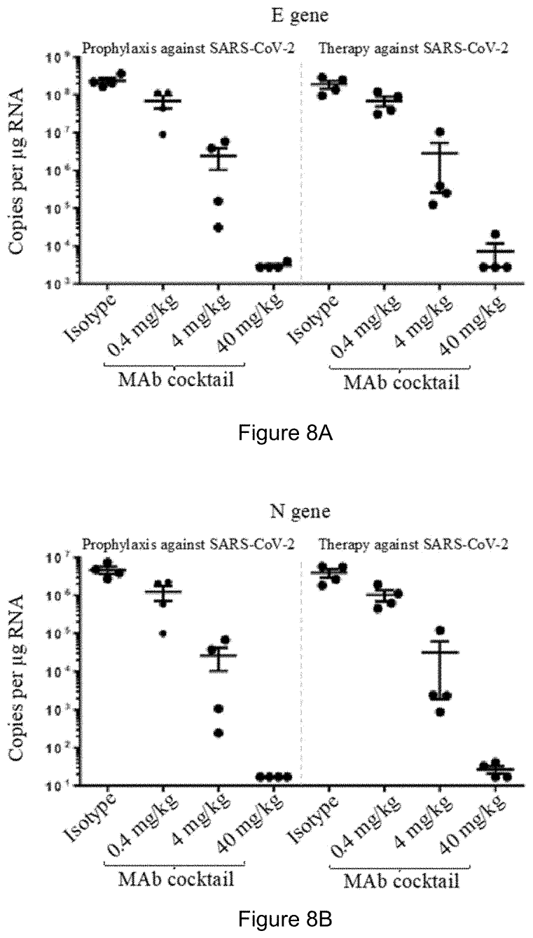

In the prophylactic experiment, administration of antibody cocktail at 40 or 4 mg/kg prior to SARS-CoV-2 challenge resulted in complete protection from weight loss (FIG. 7A, left panel). This protection was also accompanied by a great decreased of viral load in the lungs at the end of the study (4 dpi) (FIG. 7A, right panel; FIGS. 8A and 8B). It was noted that a few treated animals with 4 mg/kg of the antibody cocktail had substantial viral level in the lungs (FIG. 7A, right panel); however, these animals did not have significant weight loss compared to those with much lower viral loads (FIG. 7A, left panel). Administration of 0.4 mg/kg of the antibody cocktail prevented a sharp decrease in body weight, but treated animals failed to gain weight at the end of study (FIG. 7A, left panel). Besides that, high viral loads were observed in the lungs of 0.4 mg/kg antibody cocktail-treated animals (FIGS. 8A and 8B).

In the therapeutic experiment, animals of all doses gradually gained weight and those treated with isotype negative control had no significant weight loss (FIG. 7B, left panel). Nevertheless, it is noted that a more obvious weight gain in animals treated with 40 or 4 mg/kg of the antibody cocktail (FIG. 7B, left panel). The viral replication data demonstrated that animals treated with 40 or 4 mg/kg of the antibody cocktail had low viral loads in the lungs; by contrast, animals treated with 0.4 mg/kg antibody of the antibody cocktail or isotype negative control had similarly high viral loads in the lungs (FIG. 7B, right panel; FIGS. 8A and 8B).

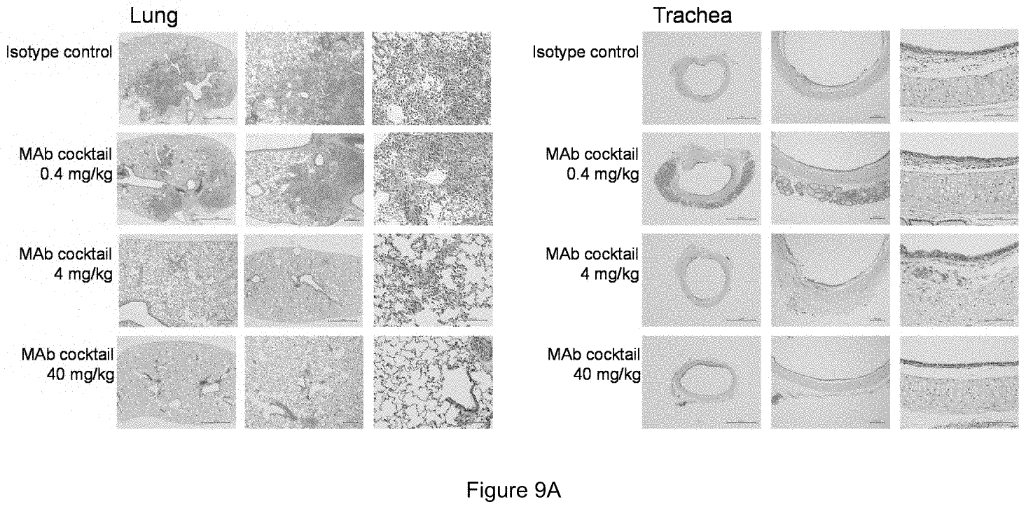

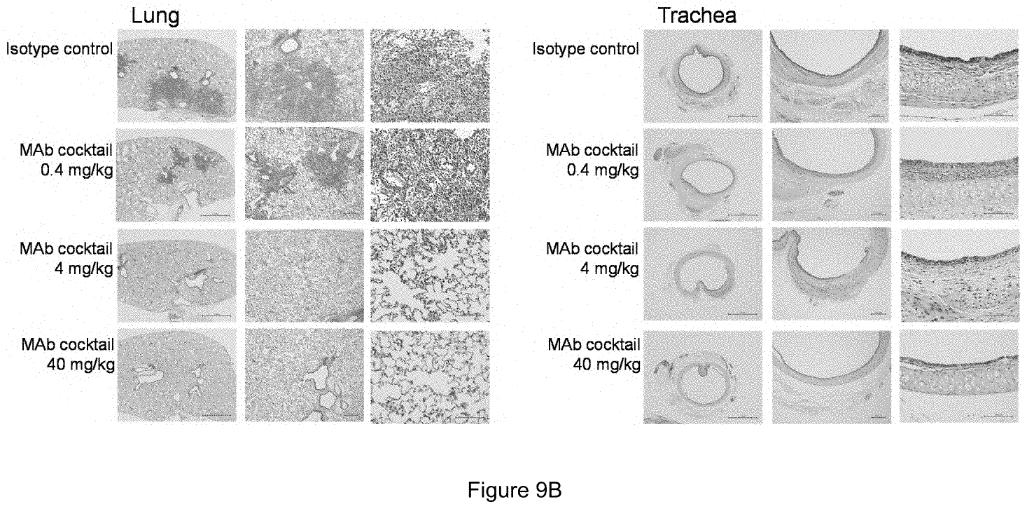

In the prophylactic experiment, there was a significantly lower amount of pulmonary inflammation or necrosis in animals treated with 40 or 4 mg/kg of the antibody cocktail when compared to those treated with 0.4 mg/kg of the antibody cocktail or isotype negative control (FIG. 9A, Tables 12 and 13). A complete recovery of pulmonary inflammation was found in animals treated with 40 mg/kg of the antibody cocktail, and the level of inflammation was significantly lower when compared to those treated with 4 mg/kg of the antibody cocktail. In addition, multifocal minimal to slight inflammation in the submucosa of the trachea was also found. There was a significantly lower level of acute tracheal inflammation in animals treated with 40 or 4 mg/kg of the antibody cocktail when compared to the 0.4 mg/kg or isotype negative control-treated group, and a complete recovery from inflammation was found in animals treated with 40 mg/kg of the antibody cocktail. Similar histopathological findings of lung and trachea were observed in the therapeutic experiment (FIG. 9B, Tables 12 and 13).

TABLE 12
Summary of pathological incidence of the lungs and trachea in the prophylactic and therapeutic
experiments of antibody cocktail treatment in hamsters 4 days after SARS-CoV-2 infection.

| | | Prophylactic¹ | | | |
|---|---|---|---|---|---|
| | | Group | | | |
| | | Isotype | Antibody | Antibody | Antibody |
| | | negative | cocktail | cocktail | cocktail |
| Organ | Histopathological findings | control | 0.4 mg/kg | 4 mg/kg | 40 mg/kg |
| Lung | Aggregation, alveolar macrophage, | 4/4⁴ | 4/4 | 0/4 | 0/4 |
| Right (3 lobes) | multifocal, minimal to slight³ | | | | |
| | Inflammation/necrosis, multifocal, minimal | 4/4 | 4/4 | 3/4 | 0/4 |
| | to moderate/severe | | | | |
| | Hemorrhage, multifocal, minimal to moderate | 4/4 | 4/4 | 0/4 | 0/4 |
| Trachea | Inflammation, submucosa, multifocal, | 2/3 | 4/4 | 1/4 | 0/4 |
| | minimal to slight | | | | |

| | | Therapeutic² | | | |
|---|---|---|---|---|---|
| | | Group | | | |
| | | Isotype | Antibody | Antibody | Antibody |
| | | negative | cocktail | cocktail | cocktail |
| Organ | Histopathological findings | control | 0.4 mg/kg | 4 mg/kg | 40 mg/kg |
| Lung | Aggregation, alveolar macrophage, | 4/4 | 4/4 | 0/4 | 0/4 |
| Right (3 lobes) | multifocal, minimal to slight³ | | | | |
| | Inflammation or necrosis, multifocal, | 4/4 | 4/4 | 4/4 | 0/4 |
| | minimal to moderate/severe | | | | |
| | Hemorrhage, multifocal, minimal to slight | 4/4 | 4/4 | 0/4 | 0/4 |
| Trachea | Inflammation, submucosa, multifocal, | 4/4 | 4/4 | 3/4 | 0/4 |
| | minimal to moderate | | | | |

¹Prophylactic experiment: isotype control or antibody cocktail via intraperitoneal injection 1 day before SARS-CoV-2 infection.
²Therapeutic experiment: isotype control or antibody cocktail via intraperitoneal injection 3 hours after SARS-CoV-2 infection.
³Degree of lesions was graded from one to five depending on severity: 1 = minimal (<1%); 2 = slight (1-25%); 3 = moderate (26-50%); 4 = moderate/severe (51-75%); 5 = severe/high (76-100%).
⁴Incidence: Affected hamsters/Total examined hamsters (n = 3-4).

TABLE 13
Summary of inflammatory scores of the lungs and trachea in the prophylactic and therapeutic
experiments of antibody cocktail treatment in hamsters 4 days after SARS-CoV-2 infection.

| | | Prophylactic¹ | | | |
|---|---|---|---|---|---|
| | | Group | | | |
| | | | Antibody | Antibody | Antibody |
| | | Isotype | cocktail | cocktail | cocktail |
| Organ | Inflammatory scores | control | 0.4 mg/kg | 4 mg/kg | 40 mg/kg |
| Lung | Aggregation, alveolar macrophage, | 1.1 ± 0.6⁴ | 1.3 ± 0.7 | 0.0 ± 0.0 | 0.0 ± 0.0 |
| Right (3 lobes) | multifocal³ | | | | |
| | Inflammation or necrosis, multifocal | 2.8 ± 1.1 | 2.4 ± 1.0 | 0.3 ± 0.5*^{, a} | 0.0 ± 0.0*^{, a, b} |
| | Hemorrhage, multifocal | 1.8 ± 0.9 | 1.7 ± 0.8 | 0.0 ± 0.0 | 0.0 ± 0.0 |
| | Subtotal mean score³ | 1.9 ± 1.1 | 1.8 ± 1.0 | 0.1 ± 0.3*^{, a} | 0.0 ± 0.0*^{, a, b} |
| Trachea | Inflammation, submucosa, multifocal | 1.0 ± 0.0 | 1.5 ± 0.5 | 0.3 ± 0.4*^{, a} | 0.0 ± 0.0*^{, a} |

Therapeutic²
Group

| | | | Antibody | Antibody | Antibody |
|---|---|---|---|---|---|
| | | Isotype | cocktail | cocktail | cocktail |
| Organ | Inflammatory scores | control | 0.4 mg/kg | 4 mg/kg | 40 mg/kg |
| Lung | Aggregation, alveolar macrophage, | 1.4 ± 0.5 | 1.3 ± 0.4 | 0.0 ± 0.0*^{, a} | 0.0 ± 0.0 |
| Right (3 lobes) | multifocal | | | | |
| | Inflammation/necrosis, multifocal | 2.3 ± 0.6 | 2.0 ± 0.4 | 0.8 ± 0.7*^{, a} | 0.0 ± 0.0*^{, a, b} |
| | Hemorrhage, multifocal | 1.8 ± 0.4 | 1.7 ± 0.4 | 0.0 ± 0.0*^{, a} | 0.0 ± 0.0 |
| | Subtotal mean score | 1.8 ± 0.6 | 1.7 ± 0.5 | 0.3 ± 0.6*^{, a} | 0.0 ± 0.0*^{, a, b} |
| Trachea | Inflammation, submucosa, multifocal | 2.0 ± 0.0 | 2.0 ± 0.7 | 0.8 ± 0.4*^{, a} | 0.0 ± 0.0*^{, a, b} |

¹Prophylactic experiment: isotype negative control or antibody cocktail via intraperitoneal injection 1 day before SARS-CoV-2 infection.
²Therapeutic experiment: isotype control or antibody cocktail via intraperitoneal injection 3 hours after SARS-CoV-2 infection.
³The final numerical score was calculated by dividing the sum of the number per grade of affected hamsters by the total number of examined hamsters (n = 4).
⁴The subtotal mean score was calculated by dividing the sum of the number per grade of each lesion of affected hamsters by the total number of examined hamsters (n = 4).
^{*}Statistically significant difference compared to the isotype control group each at p < 0.05.
^{a}Statistically significant difference between the 0.4 mg/kg antibody cocktail-treated group and the 4 or 40 mg/kg antibody cocktail-treated groups in the prophylactic and therapeutic experiments each at p < 0.05.
^{b}Statistically significant difference between the 4 mg/kg antibody cocktail-treated group and the 40 mg/kg antibody cocktail-treated groups in the prophylactic and therapeutic experiments each at p < 0.05.

Taken together, the Syrian hamster study shows that the prophylactic or therapeutic treatment with either 40 or 4 mg/kg of antibody cocktail could significantly reduce lung viral load and attenuate SARS-COV-2 virus-induced pulmonary inflammation according to histopathological examination.

In summary, a panel of SARS-CoV-2 spike and nucleocapsid-reactive human monoclonal antibodies was produced and characterized their antigenic specificities and genetic information in the variable domains of heavy and light chains. These human MAbs have held great potential for use as prophylactic or therapeutic molecules against SARS-CoV-2 and diagnostic reagents for detection of virus in the clinical samples.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

### REFERENCES

1. Bruun T U J, Andersson A C, Draper S J, Howarth M. Engineering a Rugged Nanoscaffold To Enhance Plug-and-Display Vaccination. ACS Nano. 2018; 12:8855-8866. https://doi.org/10.1021/acsnano. 8b02805 PMID: 30028591.
2. Casadevall A, Dadachova E, Pirofski L A. Passive antibody therapy for infectious diseases. Nat Rev Microbiol. 2004 September; 2(9):695-703.
3. Cubuk, J., Alston, J. J., Incicco, J. J. et al. The SARS-CoV-2 nucleocapsid protein is dynamic, disordered, and phase separates with RNA. Nat Commun 12, 1936 (2021). https://doi.org/10.1038/s41467-021-21953-3.
4. Grist, N. R. Diagnostic methods in clinical virology. Philadelphia, J. B. Lippincott & Co., 129 p. (1966); ISBN-10: 0632000112.
5. Huang K Y, Rijal P, Schimanski L, Powell T J, Lin T Y, McCauley J W, Daniels R S, Townsend A R. Focused antibody response to influenza linked to antigenic drift. J Clin Invest. 2015 Jul. 1; 125(7):2631-45. doi: 10.1172/JCI81104.
6. Huang K Y, Chen M F, Huang Y C, Shih S R, Chiu C H, Lin J J, Wang J R, Tsao K C, Lin T Y Epitope-associated and specificity-focused features of EV71-neutralizing antibody repertoires from plasmablasts of infected children. Nat Commun. 2017 Oct. 2; 8(1):762. doi: 10.1038/s41467-017-00736-9.
7. Huang K Y, Rijal P, Jiang H, Wang B, Schimanski L, Dong T, Liu Y M, Chang P, Iqbal M, Wang M C, Chen Z, Song R, Huang C C, Yang J H, Qi J, Lin T Y, Li A, Powell T J, Jan J T, Ma C, Gao G F, Shi Y, Townsend A R. Structure-function analysis of neutralizing antibodies to H7N9 influenza from naturally infected humans. Nat Microbiol. 2019 February; 4(2):306-315. doi: 10.1038/s41564-018-0303-7.
8. Huo J, Le Bas A, Ruza R R, Duyvesteyn H M E, Mikolajek H, Malinauskas T, et al. Neutralizing nanobodies bind SARS-CoV-2 spike RBD and block interaction with ACE2. Nat Struct Mol Biol. 2020; 27:846-854. https://doi.org/10.1038/s41594-020-0469-6 PMID: 32661423
9. Mair-Jenkins J, Saavedra-Campos M, Baillie J K, Cleary P, Khaw F M, Lim W S, Makki S, Rooney K D, Nguyen-Van-Tam J S, Beck C R; Convalescent Plasma Study Group. The effectiveness of convalescent plasma and hyperimmune immunoglobulin for the treatment of severe acute respiratory infections of viral etiology: a systematic review and exploratory meta-analysis. J Infect Dis. 2015 Jan. 1; 211(1):80-90. doi:10.1093/infdis/jiu396.
10. Rijal P, Elias S C, Machado S R, Xiao J, Schimanski L, O'Dowd V, et al. Therapeutic Monoclonal Anti-bodies for Ebola Virus Infection Derived from Vaccinated Humans. Cell Rep. 2019; 27:172-186.e7. https://doi.org/10.1016/j.celrep.2019.03.020 PMID: 30943399.
11. Rome B N, Avorn J. Drug Evaluation during the Covid-19 Pandemic. N Engl J Med. 2020 Apr. 14. doi: 10.1056/NEJMp2009457.
12. World Health Organization. WHO Coronoavirus (COVID-19) Dashboard. https://covid19.who.int
13. Zhou D, Duyvesteyn H M E, Chen C P, Huang C G, Chen T H, Shih S R, et al. Structural basis for the neutralization of SARS-CoV-2 by an antibody from a convalescent patient. Nat Struct Mol Biol. 2020; 27:950-958. https://doi.org/10.1038/s41594-020-0480-y PMID: 32737466.

What is claimed is:

1. An isolated antibody against Severe Acute Respiratory Syndrome Coronavirus 2 (SARS-CoV-2) or antigen-binding fragment thereof, comprising
(iii) a heavy chain variable region (V_{H}) which comprises
(a) a first heavy chain complementarity determining region (HCDR1) having an amino acid sequence of about 90%, 91%, 92%, 93%, 94%, 95%, 96%, 97%, 98%, 99% or 100% identical to SEQ ID No: 69, SEQ ID No: 75, SEQ ID No: 81, SEQ ID No: 87, SEQ ID No: 93, SEQ ID No: 99, SEQ ID No: 105, SEQ ID No: 111, SEQ ID No: 117, SEQ ID No: 123, SEQ ID No: 129, SEQ ID No: 135, SEQ ID No: 141, SEQ ID No: 147, SEQ ID No: 153, SEQ ID No: 159, SEQ ID No: 165, SEQ ID No: 171, SEQ ID No: 177, SEQ ID No: 183, SEQ ID No: 189, SEQ ID No: 195, SEQ ID No: 201, SEQ ID No: 207, SEQ ID No: 213, SEQ ID No: 219, SEQ ID No: 225, SEQ ID No: 231, SEQ ID No: 237, SEQ ID No: 243, SEQ ID No: 249, SEQ ID No: 255, SEQ ID No: 261, SEQ ID NO: 267, SEQ ID No: 333, SEQ ID No: 339, SEQ ID No:345, SEQ ID No: 351, SEQ ID No: 357, SEQ ID No: 363, SEQ ID No: 369, SEQ ID No: 375, SEQ ID No: 381, SEQ ID No: 387, SEQ ID No: 393, SEQ ID No: 399, SEQ ID No: 405, SEQ ID No: 411, SEQ ID No: 417, SEQ ID No: 423, SEQ ID No: 429, SEQ ID No: 435, SEQ ID No: 441, SEQ ID No: 447, SEQ ID No: 453, SEQ ID No: 459, SEQ ID No: 465, SEQ ID No: 471, SEQ ID No:477, SEQ ID No: 483, SEQ ID No: 489, SEQ ID No: 495, SEQ ID No: 501, or SEQ ID No: 507;
(b) a second heavy chain complementarity determining region (HCDR2) having an amino acid sequence of about 90%, 91%, 92%, 93%, 94%, 95%, 96%, 97%, 98%, 99% or 100% identical to SEQ ID No: 70, SEQ ID No: 76, SEQ ID No: 82, SEQ ID No: 88, SEQ ID No: 94, SEQ ID No: 100, SEQ ID No: 106, SEQ ID No: 112, SEQ ID No: 118, SEQ ID No: 124, SEQ ID No: 130, SEQ ID No: 136, SEQ ID No: 142, SEQ ID No: 148, SEQ ID No: 154, SEQ ID No: 160, SEQ ID No: 166, SEQ ID No: 172, SEQ ID No: 178, SEQ ID No: 184, SEQ ID No: 190, SEQ ID No: 196, SEQ ID No: 202, SEQ ID No: 208, SEQ ID No: 214, SEQ ID No: 220, SEQ ID No: 226, SEQ ID No: 232, SEQ ID No: 238, SEQ ID No: 244, SEQ ID No: 250, SEQ ID No: 256, SEQ ID No: 262, SEQ ID NO: 268, SEQ ID No: 334, SEQ ID No: 340, SEQ ID No:346, SEQ ID No: 352, SEQ ID No: 358, SEQ ID No: 364, SEQ ID No: 370, SEQ ID No: 376, SEQ ID No: 382, SEQ ID No: 388, SEQ ID No: 394, SEQ ID No: 400, SEQ ID No: 406, SEQ ID No: 412, SEQ ID No: 418, SEQ ID No: 424, SEQ ID No: 430, SEQ ID No: 436, SEQ ID No: 442, SEQ ID No: 448, SEQ ID No: 454, SEQ ID No: 460, SEQ ID No: 466, SEQ ID No: 472, SEQ ID No:478, SEQ ID No: 484, SEQ ID No: 490, SEQ ID No: 496, SEQ ID No: 502, or SEQ ID No: 508; and
(c) a third heavy chain complementarity determining region (HCDR3) having an amino acid sequence of about 90%, 91%, 92%, 93%, 94%, 95%, 96%, 97%, 98%, 99% or 100% identical to SEQ ID No: 71, SEQ ID No: 77, SEQ ID No: 83, SEQ ID No: 89, SEQ ID No: 95, SEQ ID No: 101, SEQ ID No: 107, SEQ ID No: 113, SEQ ID No: 119, SEQ ID No: 125, SEQ ID No: 131, SEQ ID No: 137, SEQ ID No: 143, SEQ ID No: 149, SEQ ID No: 155, SEQ ID No: 161, SEQ ID No: 167, SEQ ID No: 173, SEQ ID No: 179, SEQ ID No: 185, SEQ ID No: 191, SEQ ID No: 197, SEQ ID No: 203, SEQ ID No: 209, SEQ ID No: 215, SEQ ID No: 221, SEQ ID No: 227, SEQ ID No: 233, SEQ ID No: 239, SEQ ID No: 245, SEQ ID No: 251, SEQ ID No: 257, SEQ ID No: 263, SEQ ID NO: 269, SEQ ID No: 335, SEQ ID No: 341, SEQ ID No:347, SEQ ID No: 353, SEQ ID No: 359, SEQ ID No: 365, SEQ ID No: 371, SEQ ID No: 377, SEQ ID No: 383, SEQ ID No: 389, SEQ ID No: 395, SEQ ID No: 401, SEQ ID No: 407, SEQ ID No: 413, SEQ ID No: 419, SEQ ID No: 425, SEQ ID No: 431, SEQ ID No: 437, SEQ ID No: 443, SEQ ID No: 449, SEQ ID No: 455, SEQ ID No: 461, SEQ ID No: 467, SEQ ID No: 473, SEQ ID No:479, SEQ ID No: 485, SEQ ID No: 491, SEQ ID No: 497, SEQ ID No: 503, or SEQ ID No: 509; and
(iv) a light chain variable region (V_{L}) which comprises
(a) a first light chain complementarity determining region (LCDR1) having an amino acid sequence of about 90%, 91%, 92%, 93%, 94%, 95%, 96%, 97%, 98%, 99% or 100% identical to SEQ ID No: 72, SEQ ID No: 78, SEQ ID No: 84, SEQ ID No: 90, SEQ ID No: 96, SEQ ID No: 102, SEQ ID No: 108, SEQ ID No: 114, SEQ ID No: 120, SEQ ID No: 126, SEQ ID No: 132, SEQ ID No: 138, SEQ ID No: 144, SEQ ID No: 150, SEQ ID No: 156, SEQ ID No: 162, SEQ ID No: 168, SEQ ID No: 174, SEQ ID No: 180, SEQ ID No: 186, SEQ ID No: 192, SEQ ID No: 198, SEQ ID No: 204, SEQ ID No: 210, SEQ ID No: 216, SEQ ID No: 222, SEQ ID No: 228, SEQ ID No: 234, SEQ ID No: 240, SEQ ID No: 246, SEQ ID No: 252, SEQ ID No: 258, SEQ ID No: 264, SEQ ID NO: 270, SEQ ID No: 336, SEQ ID No: 342, SEQ ID No:348, SEQ ID No: 354, SEQ ID No: 360, SEQ ID No: 366, SEQ ID No: 372, SEQ ID No: 378, SEQ ID No: 384, SEQ ID No: 390, SEQ ID No: 396, SEQ ID No: 402, SEQ ID No: 408, SEQ ID No: 414, SEQ ID No: 420, SEQ ID No: 426, SEQ ID No: 432, SEQ ID No: 438, SEQ ID No: 444, SEQ ID No: 450, SEQ ID No: 456, SEQ ID No: 462, SEQ ID No: 468, SEQ ID No: 474, SEQ ID No:480, SEQ ID No: 486, SEQ ID No: 492, SEQ ID No: 498, SEQ ID No: 504, or SEQ ID No: 510;
(b) a second light chain complementarity determining region (LCDR2) having an amino acid sequence of about 90%, 91%, 92%, 93%, 94%, 95%, 96%, 97%, 98%, 99% or 100% identical to SEQ ID No: 73, SEQ ID No: 79, SEQ ID No: 85, SEQ ID No: 91, SEQ ID No: 97, SEQ ID No: 103, SEQ ID No: 109, SEQ ID No: 115, SEQ ID No: 121, SEQ ID No: 127, SEQ ID No: 133, SEQ ID No: 139, SEQ ID No: 145, SEQ ID No: 151, SEQ ID No: 157, SEQ ID No: 163, SEQ ID No: 169, SEQ ID No: 175, SEQ ID No: 181, SEQ ID No: 187, SEQ ID No: 193, SEQ ID No: 199, SEQ ID No: 205, SEQ ID No: 211, SEQ ID No: 217, SEQ ID No: 223, SEQ ID No: 229, SEQ ID No: 235, SEQ ID No: 241, SEQ ID No: 247, SEQ ID No: 253, SEQ ID No: 259, SEQ ID No: 265, SEQ ID NO: 271, SEQ ID No: 337, SEQ ID No: 343, SEQ ID No:349, SEQ ID No: 355, SEQ ID No: 361, SEQ ID No: 367, SEQ ID No: 373, SEQ ID No: 379, SEQ ID No: 385, SEQ ID No: 391, SEQ ID No: 397, SEQ ID No: 403, SEQ ID No: 409, SEQ ID No: 415, SEQ ID No: 421, SEQ ID No: 427, SEQ ID No: 433, SEQ ID No: 439, SEQ ID No: 445, SEQ ID No: 451, SEQ ID No: 457, SEQ ID No: 463, SEQ ID No: 469, SEQ ID No: 475, SEQ ID No:481, SEQ ID No: 487, SEQ ID No: 493, SEQ ID No: 499, SEQ ID No: 505, or SEQ ID No: 511; and
(c) a third light chain complementarity determining region (LCDR3) having an amino acid sequence of about 90%, 91%, 92%, 93%, 94%, 95%, 96%, 97%, 98%, 99% or 100% identical to SEQ ID No: 74, SEQ ID No: 80, SEQ ID No: 86, SEQ ID No: 92, SEQ ID No: 98, SEQ ID No: 104, SEQ ID No: 110, SEQ ID No: 116, SEQ ID No: 122, SEQ ID No: 128, SEQ ID No: 134, SEQ ID No: 140, SEQ ID No: 146, SEQ ID No: 152, SEQ ID No: 158, SEQ ID No: 164, SEQ ID No: 170, SEQ ID No: 176, SEQ ID No: 182, SEQ ID No: 188, SEQ ID No: 194, SEQ ID No: 200, SEQ ID No: 206, SEQ ID No: 212, SEQ ID No: 218, SEQ ID No: 224, SEQ ID No: 230, SEQ ID No: 236, SEQ ID No: 242, SEQ ID No: 248, SEQ ID No: 254, SEQ ID No: 260, SEQ ID No: 266, SEQ ID NO: 272, SEQ ID No: 338, SEQ ID No: 344, SEQ ID No:350, SEQ ID No: 356, SEQ ID No: 362, SEQ ID No: 368, SEQ ID No: 374, SEQ ID No: 380, SEQ ID No: 386, SEQ ID No: 392, SEQ ID No: 498, SEQ ID No: 404, SEQ ID No: 410, SEQ ID No: 416, SEQ ID No: 422, SEQ ID No: 428, SEQ ID No: 434, SEQ ID No: 440, SEQ ID No: 446, SEQ ID No: 452, SEQ ID No: 458, SEQ ID No: 464, SEQ ID No: 470, SEQ ID No: 476, SEQ ID No:482, SEQ ID No: 488, SEQ ID No: 494, SEQ ID No: 500, SEQ ID No: 506, or SEQ ID No: 512.

2. The isolated antibody or antigen-binding fragment of claim 1, wherein
(i) the heavy chain variable region (V_{H}) comprises an amino acid sequence about 90%, 91%, 92%, 93%, 94%, 95%, 96%, 97%, 98%, 99% or 100% identical to the amino acid sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, or SEQ ID NO: 331; and
(ii) the light chain variable region (V_{L}) comprises an amino acid sequence about 90%, 91%, 92%, 93%, 94%, 95%, 96%, 97%, 98%, 99% or 100% identical to the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, or SEQ ID NO: 332.

3. A pharmaceutical composition, comprising at least one of the isolated antibodies, or antigen-binding fragments thereof, of claim 1.

4. The pharmaceutical composition of claim 3, further comprising at least one pharmaceutically acceptable carrier.

5. A kit for detecting the presence of severe acute respiratory syndrome coronavirus 2 (SARS-CoV-2) in a sample, comprising at least one of the isolated antibodies, or antigen-binding fragments thereof, of claim 1.

6. The kit of claim 5, wherein the at least one of the isolated antibodies, or the antigen-binding fragments thereof, comprises a detectable label.

7. The kit of claim 6, wherein the detectable label is selected from the group consisting of an enzymatic label, a fluorescent label, a metal label, and a radio label.

8. The kit of claim 6, wherein the detectable label is selected from the group consisting of gold nanoparticles, colored latex beads, magnetic particles, carbon nanoparticles, and selenium nanoparticles.

9. The kit of claim 5, wherein the kit is an immunoassay kit selected from the group consisting of ELISA (enzyme-linked immunosorbent assay), RIA (radioimmunoassay), FIA (fluorescence immunoassay), LIA (luminescence immunoassay), and ILMA (immunoluminometric assay).

10. The kit of claim 9, wherein the immunoassay is a sandwich assay or in a lateral flow assay format.

11. A method for detecting severe acute respiratory syndrome coronavirus 2 in a sample suspected of containing said SARS-CoV-2, comprising contacting the sample with at least one of the isolated antibodies, or antigen-binding fragments thereof, of claim 1, and assaying binding of the antibody with the sample.

12. The method of claim 11, wherein the sample is urine, stool, or taken from respiratory tract.

13. The method of claim 12, wherein the sample taken from the respiratory tract is a nasopharyngeal (NP) or nasal (NS) swab.

14. The method of claim 11, wherein the SARS-COV-2 is detected by a sandwich immunoassay or lateral flow assay.

15. A method for preventing or treating a disease mediated by angiotensin-converting enzyme 2 (ACE2) in a subject, comprising a step of administering an effective amount of at least one of the isolated antibodies, or antigen-binding fragments thereof, of claim 1.

16. The method of claim 15, wherein the disease mediated by ACE2 is severe acute respiratory syndrome coronavirus 2 infection.

17. A nucleic acid comprising a nucleotide sequence encoding a heavy chain variable region (V_{H}), a light chain variable region (V_{L}) or both, wherein the V_{H} and V_{L} are as set forth in claim 1.

18. An isolated host cell comprising the nucleic acid of claim 17.
